SEQUENCE LISTING (39 sequences)

SEQ ID NO: 1 - DNA, 5454 bp, Artificial Sequence
  SARS-CoV-2 S protein cDNA construct (P91)
gtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagcacaggagatctgccaccatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatgaaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggttgaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaaggcggcgggtccggaggagactacaaagaccatgacggtgattataaagatcatgacatcgactacaaggatgacgatgacaagtag

SEQ ID NO: 2 - DNA, 5687 bp, Artificial Sequence
  SARS-CoV-2 S protein cDNA construct (P92)
gtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagcacaggagatctgccaccatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcagggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggttgaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaaggcggcgggtccggaggagactacaaagaccatgacggtgattataaagatcatgacatcgactacaaggatgacgatgacaagtag

SEQ ID NO: 3 - DNA, 5465 bp, Artificial Sequence
  SARS-CoV-2 S protein cDNA construct (P113)
gtcagatcgcctagccccctcccctcgcctcctcgccgctggcggccaccgcgtcgctccggcccgggccccaccccaggcgactctgtgaggagcggccggaggccggaggcggaggtgagcgcgacgcgagcaggtggagaggctgggcgcgggccaggcccggctgggggaggggtcgggcccgggacgcggctctttgtctcccggagcccgttcgcgggcagcggggccgctctgcctcccggcaggtgcaggcatccctcggggaggccaggggaggccgatgggggctggcggggagacccgggcgtgcgctccgggtctggagggatgcgacatcctgagcccgtggcagtcccccgctctcgaggctggcggtctgagtccctgaaggggcaaggggcaggggcgtggagatcggtcctgaattggagccgaggcgggggaggcggtgggctggggcgggcagggcctcttcgctttagggaaaagcggtggggggtgggacttggggacagcgaggagcagtggggctggcgagtgggtgtaggtgcgtgggagccgagcggatggaagccgaggccgaggtttgagtgtccatgggtggcgatgctgcgaaagggcagtgaggtagcagggtccaggtctctggaggcggcgtagctgtccagaacctgggatgcggaccggtttgtctcttcaggtgcaagatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcctggtaccccagcctccttcctcagctccgcccccatcttccctcccccttccaatacctgtccagtctcacctccactgccacctctccggggcacctgtgactcggccttctccccgcagcctttcttttccaatgttacttggttccatgctatacatgtctcaggtaacctgctccctctcccccagtctcctaagccagggttagcgtcacagagtctggaaccttttattttacacgagttggggcgcgggagcacttgcaggtcactgggcacaaattgggtgaaagccattattggtcctcagagagggcacatgcccatttcacagatgggaaaatagagacttgggaagccaaacaaagacctaggcctgagcgtggccccttctgtctccaggcaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaggtacgtgacctggagaagagtggggttcctgggcagcaaggggagccgcctcagaggtatcggtgacccttggccttctactttttctccagacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtaaggggcaccccagacccggcctggctgtgggcggggtcggaggcgaggcttcgcagctcaggggcgggacagctgggtccggggcggagcttagacaaggaggcgggaccttgaggcaggggcggggcttatcacccccacggcccacctggcgtctctccccgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataagccagaccacagccccgcctgctacaccccacccctgccttaggatccgcccctccgggtacgccgtttgttttagaccccgcctccactgccctggagccccgctgggtggattagtcttagctccctagagcctgagcctttggcctcggaggctcgggacctacccacagctttgacctaggcccgcccctcgagctccgcccctttggcctaggacacgccccgtttccccgagtcccgccccgtgtgcagtgtattgcccaccccgcacagcctgagtttgcaataaaactgggacactgggacttgca

SEQ ID NO: 4 - DNA, 5465 bp, Artificial Sequence
  SARS-CoV-2 S protein cDNA construct (P136)
gtcagatcgcctagccccctcccctcgcctcctcgccgctggcggccaccgcgtcgctccggcccgggccccaccccaggcgactctgtgaggagcggccggaggccggaggcggaggtgagcgcgacgcgagcaggtggagaggctgggcgcgggccaggcccggctgggggaggggtcgggcccgggacgcggctctttgtctcccggagcccgttcgcgggcagcggggccgctctgcctcccggcaggtgcaggcatccctcggggaggccaggggaggccgatgggggctggcggggagacccgggcgtgcgctccgggtctggagggatgcgacatcctgagcccgtggcagtcccccgctctcgaggctggcggtctgagtccctgaaggggcaaggggcaggggcgtggagatcggtcctgaattggagccgaggcgggggaggcggtgggctggggcgggcagggcctcttcgctttagggaaaagcggtggggggtgggacttggggacagcgaggagcagtggggctggcgagtgggtgtaggtgcgtgggagccgagcggatggaagccgaggccgaggtttgagtgtccatgggtggcgatgctgcgaaagggcagtgaggtagcagggtccaggtctctggaggcggcgtagctgtccagaacctgggatgcggaccggtttgtctcttcaggtgcaagatgtttgtttttcttgttttattgccactagtctcttcccaatgtgttaatctgacaaccagaactcaattaccccctgcatacactaattctttcacacgtggagtttattaccctgacaaagttttcagaagcagcgttttacattcaactcaagacttgttcctggtaccccagcctccttcctcagctccgcccccatcttccctcccccttccaatacctgtccagtctcacctccactgccacctctccggggcacctgtgactcggccttctccccgcagcctttcttttccaatgttacttggttccatgctatacatgtctcaggtaacctgctccctctcccccagtctcctaagccagggttagcgtcacagagtctggaaccttttattttacacgagttggggcgcgggagcacttgcaggtcactgggcacaaattgggtgaaagccattattggtcctcagagagggcacatgcccatttcacagatgggaaaatagagacttgggaagccaaacaaagacctaggcctgagcgtggccccttctgtctccaggcaccaatgggactaagagatttgataaccctgtcctaccatttaatgatggggtttattttgcttccactgagaagtctaacataataagaggctggatttttgggactactttggattcgaagacccagtccctacttattgttaataacgctactaatgttgttatcaaagtttgcgaatttcaattttgtaatgatccatttttgggtgtttattaccacaaaaacaacaaaagttggatggaatccgagttcagagtttattctagtgcgaataattgcacttttgaatatgtctctcaaccttttcttatggaccttgagggaaaacaggggaatttcaaaaatcttagggaatttgtgtttaagaatattgatggctattttaaaatatattctaagcacacgcctattaatttagtccgtgatctcccgcaagggttttcggctctggaaccattggtagatttgccaatcgggattaacatcactaggtttcaaactttacttgctttacatcggagttatttgactcctggggattcttcttctgggtggacagctggtgctgcggcttattacgtcggttatcttcaacctcggacttttctattaaaatataatgaaaatggaaccattaccgatgctgtagactgtgcacttgaccctctctcagaggtacgtgacctggagaagagtggggttcctgggcagcaaggggagccgcctcagaggtatcggtgacccttggccttctactttttctccagacaaagtgcacgttgaaatccttcactgtggaaaaaggaatctatcaaacttctaactttcgggtccaaccaacagaatctattgttagatttcctaatattacaaacttgtgcccttttggggaagtttttaacgccacaagatttgcatctgtttatgcttggaataggaagagaatcagcaactgtgttgctgattattctgtcctatataattccgcatcattttccacttttaaatgttatggagtgtctcctactaaattaaatgatctctgctttactaatgtctatgccgattcatttgtaattcggggggatgaagtcagacaaatcgctccagggcaaactggaaagattgctgattataattataaattaccagatgattttaccggctgcgttatcgcttggaattctaacaatcttgatagcaaagttggcgggaattataattacctgtatcggttgtttcggaagtctaatctcaaaccttttgagagagatatttcaactgaaatctatcaggccgggagcacaccttgtaatggggttgaagggtttaattgttactttcctttacaatcatatggtttccaacccactaatgggcttggttaccaaccatatagagtagtagtactttcttttgaacttctacatgcaccggcaactgtttgtggacctaaaaagtctactaatttggttaaaaacaaatgtgtcaatttcaacttcaatggtttaacgggcactggggttcttactgaatctaacaaaaaatttctgcctttccaacaatttggccgtgacattgctgacactactgatgctgtccgtgatccacaaacacttgagattcttgacattacaccatgttcttttgggggggtctccgttataacacccggaacaaatacttctaaccaagttgctgtgctgtaccaagacgttaactgcacagaagtccctgttgctattcatgccgatcaacttactcctacttggcgtgtttattctacggggtctaatgtttttcaaacacgtgcaggctgtttaatcggggctgaacatgtcaacaactcatatgagtgtgacatacccattggtgccgggatatgcgctagttatcaaactcagactaattctcctcggcgggcacgtagtgtagctagtcaatccatcattgcctacactatgtcacttggtgccgaaaattcagttgcttactctaataactctattgccatacccacaaattttactatttctgttaccaccgaaattctaccagtgtctatgaccaagacatcagtagattgtacgatgtatatctgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattacctgatccatcaaaaccaagcaagagatcatttattgaggatctactgttcaacaaagttacacttgccgatgctggcttcatcaaacaatatggggattgccttggtgatattgctgctagagacctcatttgtgcacaaaagtttaacggccttactgttttgccacctttgctcacggatgaaatgattgctcaatacacttctgcactgctggcggggacaatcacttctggttggacctttggtgcaggtaaggggcaccccagacccggcctggctgtgggcggggtcggaggcgaggcttcgcagctcaggggcgggacagctgggtccggggcggagcttagacaaggaggcgggaccttgaggcaggggcggggcttatcacccccacggcccacctggcgtctctccccgcaggtgctgcattacaaataccatttgctatgcaaatggcttatcgatttaatgggattggagttacacagaatgttctctatgagaaccaaaaattgattgccaaccaatttaatagtgctattggcaaaattcaagactcactttcttccacggcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacgggcagacttcaaagtttgcaaacatatgtgactcaacaattaattagagctgcagaaatcagagcttctgctaatcttgctgctactaaaatgtccgaatgtgtacttggacaatcaaaaagagttgatttttgtggaaagggctatcatcttatgtccttccctcaatccgcacctcatggagtagtcttcttgcatgtcacttatgtccctgcacaagaaaagaacttcacaactgctcctgccatttgtcatgatggaaaagcacactttcctcgcgagggggtctttgtttcaaatggcacacattggttcgttacacaacggaatttttatgaaccacaaatcattactacggacaacacatttgtgtctgggaactgtgatgttgtaataggaattgtcaacaacacagtttatgatcctttgcaacctgaattagactcattcaaagaggagttagataaatattttaaaaatcatacatcaccggatgttgatttaggggacatctctggcattaatgcttccgttgtaaacattcaaaaagaaattgaccgcctcaatgaggttgccaagaatttaaatgaatctctcatcgatctgcaagaacttggaaaatatgagcagtatataaagtggccttggtatatctggctggggtttatcgctggcttgattgccatagtaatggtcacaattatgctttgctgtatgactagttgttgtagttgtttgaaaggctgttgttcttgtggatcctgctgcaaatttgatgaagacgactctgagccagtgctcaaaggagtcaaattacattacacataagccagaccacagccccgcctgctacaccccacccctgccttaggatccgcccctccgggtacgccgtttgttttagaccccgcctccactgccctggagccccgctgggtggattagtcttagctccctagagcctgagcctttggcctcggaggctcgggacctacccacagctttgacctaggcccgcccctcgagctccgcccctttggcctaggacacgccccgtttccccgagtcccgccccgtgtgcagtgtattgcccaccccgcacagcctgagtttgcaataaaactgggacactgggacttgca

SEQ ID NO: 5 - DNA, 5465 bp, Artificial Sequence
  SARS-CoV-2 S protein cDNA construct (P148)
gtcagatcgcctagccccctcccctcgcctcctcgccgctggcggccaccgcgtcgctccggcccgggccccaccccaggcgactctgtgaggagcggccggaggccggaggcggaggtgagcgcgacgcgagcaggtggagaggctgggcgcgggccaggcccggctgggggaggggtcgggcccgggacgcggctctttgtctcccggagcccgttcgcgggcagcggggccgctctgcctcccggcaggtgcaggcatccctcggggaggccaggggaggccgatgggggctggcggggagacccgggcgtgcgctccgggtctggagggatgcgacatcctgagcccgtggcagtcccccgctctcgaggctggcggtctgagtccctgaaggggcaaggggcaggggcgtggagatcggtcctgaattggagccgaggcgggggaggcggtgggctggggcgggcagggcctcttcgctttagggaaaagcggtggggggtgggacttggggacagcgaggagcagtggggctggcgagtgggtgtaggtgcgtgggagccgagcggatggaagccgaggccgaggtttgagtgtccatgggtggcgatgctgcgaaagggcagtgaggtagcagggtccaggtctctggaggcggcgtagctgtccagaacctgggatgcggaccggtttgtctcttcaggtgcaagatgttcgtgttcctcgtgctgctgcctctggtgtcctcccagtgcgtcaatctgacaacaagaacacagctgccccccgcctacaccaattccttcacaagaggcgtgtactaccccgacaaggtgttcagaagctccgtgctgcacagcacccaagacctctttctggtaccccagcctccttcctcagctccgcccccatcttccctcccccttccaatacctgtccagtctcacctccactgccacctctccggggcacctgtgactcggccttctccccgcagccttttttctccaacgtcacatggttccacgctatccacgtgtcaggtaacctgctccctctcccccagtctcctaagccagggttagcgtcacagagtctggaaccttttattttacacgagttggggcgcgggagcacttgcaggtcactgggcacaaattgggtgaaagccattattggtcctcagagagggcacatgcccatttcacagatgggaaaatagagacttgggaagccaaacaaagacctaggcctgagcgtggccccttctgtctccaggcaccaacggcaccaaaaggttcgataaccccgtcctccccttcaacgatggcgtctacttcgccagcaccgagaagtccaatatcatcagaggctggatcttcggcaccacactggattccaagacccagtctctgctgatcgtgaataacgccacaaacgtggtcattaaagtgtgcgagttccagttctgcaacgaccccttcctcggcgtgtattaccacaaaaacaacaagagctggatggagtccgagtttagagtgtacagcagcgccaacaactgcacattcgagtacgtgagccagcctttcctcatggatctggagggcaagcaaggcaatttcaagaatctgagagagtttgtcttcaagaacatcgacggatacttcaagatttactccaagcacacccccattaacctcgtcagagacctcccccaaggcttttccgctctcgaacctctggtggatctgcccatcggcatcaacatcacaagattccaaaccctcctcgctctgcatagaagctatctgacccccggcgattccagctccggatggacagctggagctgccgcctactatgtgggatatctgcaacctagaacatttctgctgaagtacaacgagaacggcacaatcacagacgctgtggattgtgctctggaccccctctccgaggtacgtgacctggagaagagtggggttcctgggcagcaaggggagccgcctcagaggtatcggtgacccttggccttctactttttctccagacaaagtgtaccctcaagagctttaccgtggagaagggaatctaccagacctccaattttagggtccaacccaccgagagcatcgtgaggttccccaacatcacaaacctctgccctttcggcgaagtgttcaacgccacaaggtttgcttccgtgtacgcttggaatagaaagagaatctccaactgcgtggccgactatagcgtgctctataacagcgcctccttcagcaccttcaagtgttacggcgtgagccccaccaagctgaacgatctgtgtttcaccaacgtgtacgctgactccttcgtcattaggggcgacgaagtgagacaaatcgctcccggccagaccggcaaaatcgctgactacaactacaagctccccgacgacttcaccggctgtgtgatcgcttggaactccaacaacctcgatagcaaggtgggaggcaactacaactatctgtatagactctttagaaagtccaatctgaagcccttcgagagggacatcagcacagagatctatcaagccggatccacaccttgcaacggcgtcgagggattcaactgctacttccctctgcaatcctacggcttccagcccacaaatggcgtgggctaccagccttacagagtggtggtgctgtcctttgaactgctgcatgcccccgccacagtgtgcggacccaaaaagagcaccaacctcgtgaagaacaaatgcgtcaatttcaacttcaatggactgaccggcaccggcgtgctcaccgagtccaacaagaagtttctgcccttccagcagttcggaagagacattgccgataccacagacgccgtgagggaccctcagacactggagattctggatatcacaccttgcagcttcggcggcgtgagcgtgatcacacccggaacaaacaccagcaaccaagtggctgtgctgtaccaagacgtgaattgtacagaggtacctgtggccatccatgccgatcagctgacccccacatggagggtctacagcaccggctccaatgtctttcagacaagagctggctgtctgattggcgctgagcacgtgaacaacagctacgagtgcgacatccctatcggcgccggaatttgcgccagctaccaaacccagaccaatagccctaggagggccagatccgtcgccagccagagcatcatcgcctataccatgtctctgggcgctgagaactccgtggcctatagcaacaacagcatcgctatccccaccaacttcacaatctccgtgaccaccgagattctgcccgtgagcatgaccaagaccagcgtcgactgcaccatgtatatctgcggcgactccacagagtgctccaatctgctgctgcagtacggcagcttctgcacccaactcaatagggctctgaccggaattgctgtcgagcaagacaagaacacccaagaggtgtttgcccaagtgaaacagatttacaagaccccccccatcaaggacttcggaggcttcaatttctcccaaatcctccccgacccctccaaaccctccaagaggagctttatcgaggatctgctgttcaacaaggtgacactggctgatgccggctttatcaagcagtatggcgactgtctgggagacatcgctgctagggatctgatctgtgcccagaagtttaatggcctcaccgtgctgcctcctctgctgaccgacgagatgatcgcccagtatacaagcgctctgctggccggcacaattaccagcggatggacatttggagcaggtaaggggcaccccagacccggcctggctgtgggcggggtcggaggcgaggcttcgcagctcaggggcgggacagctgggtccggggcggagcttagacaaggaggcgggaccttgaggcaggggcggggcttatcacccccacggcccacctggcgtctctccccgcaggtgctgccctccagattcctttcgccatgcagatggcctatagattcaacggcattggcgtcacacagaacgtgctgtacgagaaccagaagctgatcgctaaccagttcaacagcgccattggcaagatccaagattccctcagctccaccgccagcgccctcggcaaactgcaagacgtcgtgaatcagaatgcccaagctctgaacacactggtgaagcagctcagcagcaattttggcgccatctcctccgtgctcaatgatattctgtctagactggacaaggtggaggccgaagtccagatcgatagactgatcaccggaagactgcagtccctccagacatacgtgacccagcagctcattagagctgccgagattagggcctccgccaatctcgctgccacaaaaatgagcgagtgcgtgctcggccagtccaaaagagtggacttctgtggcaagggctaccatctgatgtccttccctcagagcgctcctcatggcgtcgtgtttctgcatgtgacctacgtgcccgcccaagagaagaacttcacaacagcccccgctatctgtcacgacggaaaggcccacttccccagagagggcgtctttgtgtccaacggcacacactggtttgtcacccagaggaacttctatgagccccagatcatcaccaccgacaacacctttgtgagcggaaactgcgatgtggtcatcggcatcgtgaataacaccgtgtacgaccctctccagcccgagctggactccttcaaggaggagctggataagtactttaagaaccatacaagccccgacgtggacctcggcgacattagcggaatcaacgccagcgtcgtgaacatccagaaggagattgatagactcaacgaggtcgccaagaatctgaacgagtctctgattgatctgcaagagctgggcaagtacgagcagtacatcaagtggccttggtacatctggctcggattcattgccggactgatcgccatcgtcatggtgaccatcatgctctgctgcatgacaagctgttgcagctgtctgaaaggctgttgtagctgtggcagctgctgtaagttcgatgaggacgactccgagcccgtgctgaagggcgtgaagctccactacacctaagccagaccacagccccgcctgctacaccccacccctgccttaggatccgcccctccgggtacgccgtttgttttagaccccgcctccactgccctggagccccgctgggtggattagtcttagctccctagagcctgagcctttggcctcggaggctcgggacctacccacagctttgacctaggcccgcccctcgagctccgcccctttggcctaggacacgccccgtttccccgagtcccgccccgtgtgcagtgtattgcccaccccgcacagcctgagtttgcaataaaactgggacactgggacttgca

SEQ ID NO: 6 - DNA, 4997 bp, Artificial Sequence
  SARS-CoV-2 S protein cDNA construct (P143)
atgtttgtttttcttgttttattgccactagtctcttcccaatgtgttaatctgacaaccagaactcaattaccccctgcatacactaattctttcacacgtggagtttattaccctgacaaagttttcagaagcagcgttttacattcaactcaagacttgttcttacctttcttttccaatgttacttggttccatgctatacatgtctcaggtaagttgatttagaaacacttttcaagcagtcagcccatggttaccattaagttaaccctatcactgaattgctccaattttcctcttaggtaccaatgggactaagagatttgataaccctgtcctaccatttaatgatggggtttattttgcttccactgagaagtctaacataataagaggctggatttttgggactactttggattcgaagacccagtccctacttattgttaataacgctactaatgttgttatcaaagtttgcgaatttcaattttgtaatgatccatttttgggtgtttattaccacaaaaacaacaaaagttggatggaatccgagttcaggtaaggaaatttccatgagtttcactcttgaagcattggggttatttgtgccagaggctaatgacccatgctggcccttcacttttctagggtttattctagtgcgaataattgcacttttgaatatgtctctcaaccttttcttatggaccttgagggaaaacaggggaatttcaaaaatcttagggaatttgtgtttaagaatattgatggctattttaaaatatattctaagcacacgcctattaatttagtccgtgatctcccgcaagggttttcggctctggaaccattggtagatttgccaatcgggattaacatcactaggtttcaaactttacttgctttacatcggagttatttgactcctggggattcttcttcaggtaagtaatttatataccactagagattttttcatcagtttctgttataaaaataattaaaatcaacatatttttctcctttacaacaggttggacagctggtgctgcggcttattacgtcggttatcttcaacctcggacttttctattaaaatataatgaaaatggaaccattacagatgctgtagactgtgcacttgaccctctgagcgaaacaaagtgcacgttgaaatccttcactgtggaaaaaggaatctatcaaacttctaactttcgggtccaaccaacagaatctattgttagatttcctaatattacaaacttgtgcccttttggggaagtttttaacgccacaagatttgcatctgtttatgcttggaacaggtaagtagtgctgattatacacaagatattgtctagaacttgatgagactgtggatatgaatatttcactcttttctcaggaagagaatcagcaactgtgttgctgattattctgtcctatataattccgcatcattttccacttttaaatgttatggagtgtctcctactaaattaaatgatctctgctttactaatgtctatgccgattcatttgtaattcggggggatgaagtcagacaaatcgctccagggcaaactggaaagattgctgattataattataaattaccagatgattttaccggctgcgttatcgcttggaattctaacaatcttgatagcaaagttggcgggaattataattacctgtatcggttgttcaggtaaggaatgttgcactgattttcacaggattttcccaagtgatactatcttattacattgatttttggctttgttttgttttcaggaagtctaatctcaaaccttttgagagagatatttcaactgaaatctatcaggccgggagcacaccttgtaatggggttgaagggtttaattgttactttcctttacaatcatatggtttccaacccactaatggggttggttaccaaccatatagagtagtagtactttcttttgaacttctacatgcaccggcaactgtttgtggacctaaaaagtctactaatttggttaaaaacaaatgtgtcaatttcaacttcaatggtttaacgggcacaggtaagtgacttgctttcttacatcaaaaaggcatccagtgtctgtttaagaattgccttctcaatattctctgttgattcctttccaggtgttcttactgaatctaacaaaaaatttctgcctttccaacaatttggccgtgacattgctgacactactgatgctgtccgtgatccacaaacacttgagattcttgacattacaccatgttcttttgggggggtctccgttataacacccggaacaaatacttctaaccaagttgctgtgctgtaccaagacgttaactgcacagaagtccctgttgctattcatgccgatcaacttactcctacttggcgtgtttattctacaggtaagtaggagaacattttcacatacaaagccatttttactttttttttaaatttcttataatcaatatgatctttttcacaggttctaatgtttttcaaacacgtgcaggctgtttaatcggggctgaacatgtcaacaactcatatgagtgtgacatacccattggtgccgggatatgcgctagttatcaaactcagactaattctcctcggcgggcacgtagtgtagctagtcaatccatcattgcctacactatgtcacttggtgccgaaaattcagttgcttactctaataactctattgccatacccacaaattttactatttctgttaccacaggtaagttgctttctctgaatacaaaactattgtttgactgtctttaagaatattactttttcatcataacttcttctttgaaaagaaattctaccagtgtctatgaccaagacatcagtagattgtacgatgtatatctgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattacctgatccatcaaaaccaagcaagagatcatttattgaggatctactgttcaacaaagttacacttgccgatgcaggtaagtctatttcaaaaaagaatcatatatattttaaaatagcttatgtattttttacacattcatttcttatttacctactatttatccaggtttcatcaaacaatatggggattgccttggtgatattgctgctagagacctcatttgtgcacaaaagtttaacggccttactgttttgccacctttgctcacggatgaaatgattgctcaatacacttctgcactgctggcggggacaatcacttctggttggacctttggtgctggggctgcattacaaataccatttgctatgcaaatggcttacaggtaagcaaatgaaccatcatcccatcattttgagttatatccttcctttgttatatggggcttacacttatcatttctcctttgctttaggtttaatgggattggagttacacagaatgttctctatgagaaccaaaaattgattgccaaccaatttaatagtgctattggcaaaattcaagactcactttcttccacggcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggtaagtgtcttaaattcagaagacgtaaagcaaaacacggttttgaggaggcttcttattataaatcttgcattatctacttttttctaggtagacttcaaagtttgcaaacatatgtgactcaacaattaattagagctgcagaaatcagagcttctgctaatcttgctgctactaaaatgtccgaatgtgtacttggacaatcaaaaagagttgatttttgtggaaagggctatcatcttatgtccttccctcaatccgcacctcatggagtagtcttcttgcatgtcacttatgtccctgcacaagaaaagaacttcacaactgctcctgccatttgtcatgatggaaaagcacactttcccaggtaagtcattatatgaagaaaaacccaggtgcatgttttacatgaagaaaactggtatttgtttgactggttttgcttttatgttttagggagggggtctttgtttcaaatggcacacattggttcgttacacaacggaatttttatgaaccacaaatcattactacggacaacacatttgtgtctgggaactgtgatgttgtaataggaattgtcaacaacacagtttatgatcctttgcaacctgaattagactcattcaaagaggagttagataaatattttaaaaatcatacatcaccggatgttgatttaggggacatctcaggtaagttgtccaacttttcaaagatccaggttttcttttaccataaatgtgttattgtctgtactaatctataggatttctctcttttgtaggtattaatgcttccgttgtaaacattcaaaaagaaattgaccgcctcaatgaggttgccaagaatttaaatgaatctctcatcgatctgcaagaacttggaaaatatgagcagtatataaagtggccttggtatatctggctggggtttatcgctggcttgattgccatagtaatggtcacaattatgctttgctgtatgactagttgttgtagttgtttgaaaggctgttgttcttgtggatcctgctgcaaaggcggcgggtccggaggagactacaaagaccatgacggggattataaagatcatgacatcgactacaaggatgacgatgacaagtag

SEQ ID NO: 7 - DNA, 4997 bp, Artificial Sequence
  SARS-CoV-2 S protein cDNA construct (P172)
atgtttgtttttcttgttttattgccactagtctcttcccaatgtgttaatctgacaaccagaactcaattaccccctgcatacactaattctttcacacgtggagtttattaccctgacaaagttttcagaagcagcgttttacattcaactcaagacttgttcttacctttcttttccaatgttacttggttccatgctatacatgtctcaggtaagttgatttagaaacacttttcaagcagtcagcccatggttaccattaagttaaccctatcactgaattgctccaattttcctcttaggtaccaacgggaccaagcggttcgacaaccccgtcctccccttcaacgacggggtctattttgcttccactgagaagtctaacataataagaggctggatttttgggactactttggattcgaagacccagtccctacttattgttaataacgctactaatgttgttatcaaagtttgcgaatttcaattttgtaatgatccatttttgggtgtttattaccacaaaaacaacaaaagttggatggaatccgagttcaggtaaggaaatttccatgagtttcactcttgaagcattggggttatttgtgccagaggctaatgacccatgctggcccttcacttttctagggtttattctagtgcgaataattgcacttttgaatatgtctctcaaccttttcttatggaccttgagggaaaacaggggaatttcaaaaatcttagggaatttgtgtttaagaatattgatggctattttaaaatatattctaagcacacgcctattaatttagtccgtgatctcccgcaagggttttcggctctggaaccattggtagatttgccaatcgggattaacatcactaggtttcaaactttacttgctttacatcggagttatttgactcctggggattcttcttcaggtaagtaatttatataccactagagattttttcatcagtttctgttataaaaataattaaaatcaacatatttttctcctttacaacaggttggacagctggtgctgcggcttattacgtcggttatcttcaacctcggacttttctattaaaatataatgaaaatggaaccattacagatgctgtagactgtgcacttgaccctctgagcgaaacaaagtgcacgttgaaatccttcactgtggaaaaaggaatctatcaaacttctaactttcgggtccaaccaacagaatctattgttagatttcctaatattacaaacttgtgcccttttggggaagtttttaacgccacaagatttgcatctgtttatgcttggaacaggtaagtagtgctgattatacacaagatattgtctagaacttgatgagactgtggatatgaatatttcactcttttctcaggaagagaatcagcaactgtgttgctgattattctgtcctatataattccgcatcattttccacttttaaatgttatggagtgtctcctactaaattaaatgatctctgctttactaatgtctatgccgattcatttgtaattcggggggatgaagtcagacaaatcgctccagggcaaactggaaagattgctgattataattataaattaccagatgattttaccggctgcgttatcgcttggaattctaacaatcttgatagcaaagttggcgggaattataattacctgtatcggttgttcaggtaaggaatgttgcactgattttcacaggattttcccaagtgatactatcttattacattgatttttggctttgttttgttttcaggaagtctaatctcaaaccttttgagagagatatttcaactgaaatctatcaggccgggagcacaccttgtaatggggttgaagggtttaattgttactttcctttacaatcatatggtttccaacccactaatggggttggttaccaaccatatagagtagtagtactttcttttgaacttctacatgcaccggcaactgtttgtggacctaaaaagtctactaatttggttaaaaacaaatgtgtcaatttcaacttcaatggtttaacgggcacaggtaagtgacttgctttcttacatcaaaaaggcatccagtgtctgtttaagaattgccttctcaatattctctgttgattcctttccaggtgttcttactgaatctaacaaaaaatttctgcctttccaacaatttggccgtgacattgctgacactactgatgctgtccgtgatccacaaacacttgagattcttgacattacaccatgttcttttgggggggtctccgttataacacccggaacaaatacttctaaccaagttgctgtgctgtaccaagacgttaactgcacagaagtccctgttgctattcatgccgatcaacttactcctacttggcgtgtttattctacaggtaagtaggagaacattttcacatacaaagccatttttactttttttttaaatttcttataatcaatatgatctttttcacaggttctaatgtttttcaaacacgtgcaggctgtttaatcggggctgaacatgtcaacaactcatatgagtgtgacatacccattggtgccgggatatgcgctagttatcaaactcagactaattctcctcggcgggcacgtagtgtagctagtcaatccatcattgcctacactatgtcacttggtgccgaaaattcagttgcttactctaataactctattgccatacccacaaattttactatttctgttaccacaggtaagttgctttctctgaatacaaaactattgtttgactgtctttaagaatattactttttcatcataacttcttctttgaaaagaaattctaccagtgtctatgaccaagacatcagtagattgtacgatgtatatctgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattacctgatccatcaaaaccaagcaagagatcatttattgaggatctactgttcaacaaagttacacttgccgatgcaggtaagtctatttcaaaaaagaatcatatatattttaaaatagcttatgtattttttacacattcatttcttatttacctactatttatccaggtttcatcaaacaatatggggattgccttggtgatattgctgctagagacctcatttgtgcacaaaagtttaacggccttactgttttgccacctttgctcacggatgaaatgattgctcaatacacttctgcactgctggcggggacaatcacttctggttggacctttggtgctggggctgcattacaaataccatttgctatgcaaatggcttacaggtaagcaaatgaaccatcatcccatcattttgagttatatccttcctttgttatatggggcttacacttatcatttctcctttgctttaggtttaatgggattggagttacacagaatgttctctatgagaaccaaaaattgattgccaaccaatttaatagtgctattggcaaaattcaagactcactttcttccacggcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggtaagtgtcttaaattcagaagacgtaaagcaaaacacggttttgaggaggcttcttattataaatcttgcattatctacttttttctaggtagacttcaaagtttgcaaacatatgtgactcaacaattaattagagctgcagaaatcagagcttctgctaatcttgctgctactaaaatgtccgaatgtgtacttggacaatcaaaaagagttgatttttgtggaaagggctatcatcttatgtccttccctcaatccgcacctcatggagtagtcttcttgcatgtcacttatgtccctgcacaagaaaagaacttcacaactgctcctgccatttgtcatgatggaaaagcacactttcccaggtaagtcattatatgaagaaaaacccaggtgcatgttttacatgaagaaaactggtatttgtttgactggttttgcttttatgttttagggagggggtctttgtttcaaatggcacacattggttcgttacacaacggaatttttatgaaccacaaatcattactacggacaacacatttgtgtctgggaactgtgatgttgtaataggaattgtcaacaacacagtttatgatcctttgcaacctgaattagactcattcaaagaggagttagataaatattttaaaaatcatacatcaccggatgttgatttaggggacatctcaggtaagttgtccaacttttcaaagatccaggttttcttttaccataaatgtgttattgtctgtactaatctataggatttctctcttttgtaggtattaatgcttccgttgtaaacattcaaaaagaaattgaccgcctcaatgaggttgccaagaatttaaatgaatctctcatcgatctgcaagaacttggaaaatatgagcagtatataaagtggccttggtatatctggctggggtttatcgctggcttgattgccatagtaatggtcacaattatgctttgctgtatgactagttgttgtagttgtttgaaaggctgttgttcttgtggatcctgctgcaaaggcggcgggtccggaggagactacaaagaccatgacggggattataaagatcatgacatcgactacaaggatgacgatgacaagtag

SEQ ID NO: 8 - DNA, 3822 bp, Artificial Sequence
  SARS-CoV-2 S protein cDNA construct (P166)
atgttcgtgttcctcgtgctgctgcctctggtgtcctcccagtgcgtcaatctgacaacaagaacacagctgccccccgcctacaccaattccttcacaagaggcgtgtactaccccgacaaggtgttcagaagctccgtgctgcacagcacccaagacctctttctgccctttttctccaacgtcacatggttccacgctatccacgtgagcggaaccaacggcaccaaaaggttcgataaccccgtcctccccttcaacgatggcgtctacttcgccagcaccgagaagtccaatatcatcagaggctggatcttcggcaccacactggattccaagacccagtctctgctgatcgtgaataacgccacaaacgtggtcattaaagtgtgcgagttccagttctgcaacgaccccttcctcggcgtgtattaccacaaaaacaacaagagctggatggagtccgagtttagagtgtacagcagcgccaacaactgcacattcgagtacgtgagccagcctttcctcatggatctggagggcaagcaaggcaatttcaagaatctgagagagtttgtcttcaagaacatcgacggatacttcaagatttactccaagcacacccccattaacctcgtcagagacctcccccaaggcttttccgctctcgaacctctggtggatctgcccatcggcatcaacatcacaagattccaaaccctcctcgctctgcatagaagctatctgacccccggcgattccagctccggatggacagctggagctgccgcctactatgtgggatatctgcaacctagaacatttctgctgaagtacaacgagaacggcacaatcacagacgctgtggattgtgctctggaccccctctccgagaccaagtgtaccctcaagagctttaccgtggagaagggaatctaccagacctccaattttagggtccaacccaccgagagcatcgtgaggttccccaacatcacaaacctctgccctttcggcgaagtgttcaacgccacaaggtttgcttccgtgtacgcttggaatagaaagagaatctccaactgcgtggccgactatagcgtgctctataacagcgcctccttcagcaccttcaagtgttacggcgtgagccccaccaagctgaacgatctgtgtttcaccaacgtgtacgctgactccttcgtcattaggggcgacgaagtgagacaaatcgctcccggccagaccggcaaaatcgctgactacaactacaagctccccgacgacttcaccggctgtgtgatcgcttggaactccaacaacctcgatagcaaggtgggaggcaactacaactatctgtatagactctttagaaagtccaatctgaagcccttcgagagggacatcagcacagagatctatcaagccggatccacaccttgcaacggcgtcgagggattcaactgctacttccctctgcaatcctacggcttccagcccacaaatggcgtgggctaccagccttacagagtggtggtgctgtcctttgaactgctgcatgcccccgccacagtgtgcggacccaaaaagagcaccaacctcgtgaagaacaaatgcgtcaatttcaacttcaatggactgaccggcaccggcgtgctcaccgagtccaacaagaagtttctgcccttccagcagttcggaagagacattgccgataccacagacgccgtgagggaccctcagacactggagattctggatatcacaccttgcagcttcggcggcgtgagcgtgatcacacccggaacaaacaccagcaaccaagtggctgtgctgtaccaagacgtgaattgtacagaggtacctgtggccatccatgccgatcagctgacccccacatggagggtctacagcaccggctccaatgtctttcagacaagagctggctgtctgattggcgctgagcacgtgaacaacagctacgagtgcgacatccctatcggcgccggaatttgcgccagctaccaaacccagaccaatagccctaggagggccagatccgtcgccagccagagcatcatcgcctataccatgtctctgggcgctgagaactccgtggcctatagcaacaacagcatcgctatccccaccaacttcacaatctccgtgaccaccgagattctgcccgtgagcatgaccaagaccagcgtcgactgcaccatgtatatctgcggcgactccacagagtgctccaatctgctgctgcagtacggcagcttctgcacccaactcaatagggctctgaccggaattgctgtcgagcaagacaagaacacccaagaggtgtttgcccaagtgaaacagatttacaagaccccccccatcaaggacttcggaggcttcaatttctcccaaatcctccccgacccctccaaaccctccaagaggagctttatcgaggatctgctgttcaacaaggtgacactggctgatgccggctttatcaagcagtatggcgactgtctgggagacatcgctgctagggatctgatctgtgcccagaagtttaatggcctcaccgtgctgcctcctctgctgaccgacgagatgatcgcccagtatacaagcgctctgctggccggcacaattaccagcggatggacatttggagccggcgctgccctccagattcctttcgccatgcagatggcctatagattcaacggcattggcgtcacacagaacgtgctgtacgagaaccagaagctgatcgctaaccagttcaacagcgccattggcaagatccaagattccctcagctccaccgccagcgccctcggcaaactgcaagacgtcgtgaatcagaatgcccaagctctgaacacactggtgaagcagctcagcagcaattttggcgccatctcctccgtgctcaatgatattctgtctagactggacaaggtggaggccgaagtccagatcgatagactgatcaccggaagactgcagtccctccagacatacgtgacccagcagctcattagagctgccgagattagggcctccgccaatctcgctgccacaaaaatgagcgagtgcgtgctcggccagtccaaaagagtggacttctgtggcaagggctaccatctgatgtccttccctcagagcgctcctcatggcgtcgtgtttctgcatgtgacctacgtgcccgcccaagagaagaacttcacaacagcccccgctatctgtcacgacggaaaggcccacttccccagagagggcgtctttgtgtccaacggcacacactggtttgtcacccagaggaacttctatgagccccagatcatcaccaccgacaacacctttgtgagcggaaactgcgatgtggtcatcggcatcgtgaataacaccgtgtacgaccctctccagcccgagctggactccttcaaggaggagctggataagtactttaagaaccatacaagccccgacgtggacctcggcgacattagcggaatcaacgccagcgtcgtgaacatccagaaggagattgatagactcaacgaggtcgccaagaatctgaacgagtctctgattgatctgcaagagctgggcaagtacgagcagtacatcaagtggccttggtacatctggctcggattcattgccggactgatcgccatcgtcatggtgaccatcatgctctgctgcatgacaagctgttgcagctgtctgaaaggctgttgtagctgtggcagctgctgtaagttcgatgaggacgactccgagcccgtgctgaagggcgtgaagctccactacacctaa

SEQ ID NO: 9 - DNA, 4084 bp, Artificial Sequence
  SARS-CoV-2 S protein cDNA construct (P205)
atgttcgtgttcctcgtgctgctgcctctggtgtcctcccagtgcgtcaatctgacaacaagaacacagctgccccccgcctacaccaattccttcacaagaggcgtgtactaccccgacaaggtgttcagaagctccgtgctgcacagcacccaagacctctttctgccctttttctccaacgtcacatggttccacgctatccacgtgagcggaaccaacggcaccaaaaggttcgataaccccgtcctccccttcaacgatggcgtctacttcgccagcaccgagaagtccaatatcatcagaggctggatcttcggcaccacactggattccaagacccagtctctgctgatcgtgaataacgccacaaacgtggtcattaaagtgtgcgagttccagttctgcaacgaccccttcctcggcgtgtattaccacaaaaacaacaagagctggatggagtccgagtttagagtgtacagcagcgccaacaactgcacattcgagtacgtgagccagcctttcctcatggatctggagggcaagcaaggcaatttcaagaatctgagagagtttgtcttcaagaacatcgacggatacttcaagatttactccaagcacacccccattaacctcgtcagagacctcccccaaggcttttccgctctcgaacctctggtggatctgcccatcggcatcaacatcacaagattccaaaccctcctcgctctgcatagaagctatctgacccccggcgattccagctcaggtaagtaatttatataccactagagattttttcatcagtttctgttataaaaataattaaaatcaacatatttttctcctttacaacaggttggacagctggagctgccgcctactatgtgggatatctgcaacctagaacatttctgctgaagtacaacgagaacggcacaatcacagacgctgtggattgtgctctggaccccctctccgagaccaagtgtaccctcaagagctttaccgtggagaagggaatctaccagacctccaattttagggtccaacccaccgagagcatcgtgaggttccccaacatcacaaacctctgccctttcggcgaagtgttcaacgccacaaggtttgcttccgtgtacgcttggaatagaaagagaatctccaactgcgtggccgactatagcgtgctctataacagcgcctccttcagcaccttcaagtgttacggcgtgagccccaccaagctgaacgatctgtgtttcaccaacgtgtacgctgactccttcgtcattaggggcgacgaagtgagacaaatcgctcccggccagaccggcaaaatcgctgactacaactacaagctccccgacgacttcaccggctgtgtgatcgcttggaactccaacaacctcgatagcaaggtgggaggcaactacaactatctgtatagactctttagaaagtccaatctgaagcccttcgagagggacatcagcacagagatctatcaagccggatccacaccttgcaacggcgtcgagggattcaactgctacttccctctgcaatcctacggcttccagcccacaaatggcgtgggctaccagccttacagagtggtggtgctgtcctttgaactgctgcatgcccccgccacagtgtgcggacccaaaaagagcaccaacctcgtgaagaacaaatgcgtcaatttcaacttcaatggactgaccggcaccggcgtgctcaccgagtccaacaagaagtttctgcccttccagcagttcggaagagacattgccgataccacagacgccgtgagggaccctcagacactggagattctggatatcacaccttgcagcttcggcggcgtgagcgtgatcacacccggaacaaacaccagcaaccaagtggctgtgctgtaccaagacgtgaattgtacagaggtacctgtggccatccatgccgatcagctgacccccacatggagggtctacagcacaggtaagtaggagaacattttcacatacaaagccatttttactttttttttaaatttcttataatcaatatgatctttttcacaggttccaatgtctttcagacaagagctggctgtctgattggcgctgagcacgtgaacaacagctacgagtgcgacatccctatcggcgccggaatttgcgccagctaccaaacccagaccaatagccctaggagggccagatccgtcgccagccagagcatcatcgcctataccatgtctctgggcgctgagaactccgtggcctatagcaacaacagcatcgctatccccaccaacttcacaatctccgtgaccaccgagattctgcccgtgagcatgaccaagaccagcgtcgactgcaccatgtatatctgcggcgactccacagagtgctccaatctgctgctgcagtacggcagcttctgcacccaactcaatagggctctgaccggaattgctgtcgagcaagacaagaacacccaagaggtgtttgcccaagtgaaacagatttacaagaccccccccatcaaggacttcggaggcttcaatttctcccaaatcctccccgacccctccaaaccctccaagaggagctttatcgaggatctgctgttcaacaaggtgacactggctgatgccggctttatcaagcagtatggcgactgtctgggagacatcgctgctagggatctgatctgtgcccagaagtttaatggcctcaccgtgctgcctcctctgctgaccgacgagatgatcgcccagtatacaagcgctctgctggccggcacaattaccagcggatggacatttggagccggcgctgccctccagattcctttcgccatgcagatggcctatagattcaacggcattggcgtcacacagaacgtgctgtacgagaaccagaagctgatcgctaaccagttcaacagcgccattggcaagatccaagattccctcagctccaccgccagcgccctcggcaaactgcaagacgtcgtgaatcagaatgcccaagctctgaacacactggtgaagcagctcagcagcaattttggcgccatctcctccgtgctcaatgatattctgtctagactggacaaggtggaggccgaagtccagatcgatagactgatcacaggtaagtgtcttaaattcagaagacgtaaagcaaaacacggttttgaggaggcttcttattataaatcttgcattatctacttttttctaggtagactgcagtccctccagacatacgtgacccagcagctcattagagctgccgagattagggcctccgccaatctcgctgccacaaaaatgagcgagtgcgtgctcggccagtccaaaagagtggacttctgtggcaagggctaccatctgatgtccttccctcagagcgctcctcatggcgtcgtgtttctgcatgtgacctacgtgcccgcccaagagaagaacttcacaacagcccccgctatctgtcacgacggaaaggcccacttccccagagagggcgtctttgtgtccaacggcacacactggtttgtcacccagaggaacttctatgagccccagatcatcaccaccgacaacacctttgtgagcggaaactgcgatgtggtcatcggcatcgtgaataacaccgtgtacgaccctctccagcccgagctggactccttcaaggaggagctggataagtactttaagaaccatacaagccccgacgtggacctcggcgacattagcggaatcaacgccagcgtcgtgaacatccagaaggagattgatagactcaacgaggtcgccaagaatctgaacgagtctctgattgatctgcaagagctgggcaagtacgagcagtacatcaagtggccttggtacatctggctcggattcattgccggactgatcgccatcgtcatggtgaccatcatgctctgctgcatgacaagctgttgcagctgtctgaaaggctgttgtagctgtggcagctgctgtaagttcgatgaggacgactccgagcccgtgctgaagggcgtgaagctccactacacctaa

SEQ ID NO: 10 - DNA, 4445 bp, Artificial Sequence
  SARS-CoV-2 S protein cDNA construct (P204)
atgttcgtgttcctcgtgctgctgcctctggtgtcctcccagtgcgtcaatctgacaacaagaacacagctgccccccgcctacaccaattccttcacaagaggcgtgtactaccccgacaaggtgttcagaagctccgtgctgcacagcacccaagacctctttctgccctttttctccaacgtcacatggttccacgctatccacgtgtcaggtaagttgatttagaaacacttttcaagcagtcagcccatggttaccattaagttaaccctatcactgaattgctccaattttcctcttaggtaccaacggcaccaaaaggttcgataaccccgtcctccccttcaacgatggcgtctacttcgccagcaccgagaagtccaatatcatcagaggctggatcttcggcaccacactggattccaagacccagtctctgctgatcgtgaataacgccacaaacgtggtcattaaagtgtgcgagttccagttctgcaacgaccccttcctcggcgtgtattaccacaaaaacaacaagagctggatggagtccgagtttagagtgtacagcagcgccaacaactgcacattcgagtacgtgagccagcctttcctcatggatctggagggcaagcaaggcaatttcaagaatctgagagagtttgtcttcaagaacatcgacggatacttcaagatttactccaagcacacccccattaacctcgtcagagacctcccccaaggcttttccgctctcgaacctctggtggatctgcccatcggcatcaacatcacaagattccaaaccctcctcgctctgcatagaagctatctgacccccggcgattccagctcaggtaagtaatttatataccactagagattttttcatcagtttctgttataaaaataattaaaatcaacatatttttctcctttacaacaggttggacagctggagctgccgcctactatgtgggatatctgcaacctagaacatttctgctgaagtacaacgagaacggcacaatcacagacgctgtggattgtgctctggaccccctctccgagaccaagtgtaccctcaagagctttaccgtggagaagggaatctaccagacctccaattttagggtccaacccaccgagagcatcgtgaggttccccaacatcacaaacctctgccctttcggcgaagtgttcaacgccacaaggtttgcttccgtgtacgcttggaatagaaagagaatctccaactgcgtggccgactatagcgtgctctataacagcgcctccttcagcaccttcaagtgttacggcgtgagccccaccaagctgaacgatctgtgtttcaccaacgtgtacgctgactccttcgtcattaggggcgacgaagtgagacaaatcgctcccggccagaccggcaaaatcgctgactacaactacaagctccccgacgacttcaccggctgtgtgatcgcttggaactccaacaacctcgatagcaaggtgggaggcaactacaactatctgtatagactcttcaggtaaggaatgttgcactgattttcacaggattttcccaagtgatactatcttattacattgatttttggctttgttttgttttcaggaagtccaatctgaagcccttcgagagggacatcagcacagagatctatcaagccggatccacaccttgcaacggcgtcgagggattcaactgctacttccctctgcaatcctacggcttccagcccacaaatggcgtgggctaccagccttacagagtggtggtgctgtcctttgaactgctgcatgcccccgccacagtgtgcggacccaaaaagagcaccaacctcgtgaagaacaaatgcgtcaatttcaacttcaatggactgaccggcaccggcgtgctcaccgagtccaacaagaagtttctgcccttccagcagttcggaagagacattgccgataccacagacgccgtgagggaccctcagacactggagattctggatatcacaccttgcagcttcggcggcgtgagcgtgatcacacccggaacaaacaccagcaaccaagtggctgtgctgtaccaagacgtgaattgtacagaggtacctgtggccatccatgccgatcagctgacccccacatggagggtctacagcacaggtaagtaggagaacattttcacatacaaagccatttttactttttttttaaatttcttataatcaatatgatctttttcacaggttccaatgtctttcagacaagagctggctgtctgattggcgctgagcacgtgaacaacagctacgagtgcgacatccctatcggcgccggaatttgcgccagctaccaaacccagaccaatagccctaggagggccagatccgtcgccagccagagcatcatcgcctataccatgtctctgggcgctgagaactccgtggcctatagcaacaacagcatcgctatccccaccaacttcacaatctccgtgaccaccgagattctgcccgtgagcatgaccaagaccagcgtcgactgcaccatgtatatctgcggcgactccacagagtgctccaatctgctgctgcagtacggcagcttctgcacccaactcaatagggctctgaccggaattgctgtcgagcaagacaagaacacccaagaggtgtttgcccaagtgaaacagatttacaagaccccccccatcaaggacttcggaggcttcaatttctcccaaatcctccccgacccctccaaaccctccaagaggagctttatcgaggatctgctgttcaacaaggtgacactggctgatgcaggtaagtctatttcaaaaaagaatcatatatattttaaaatagcttatgtattttttacacattcatttcttatttacctactatttatccaggttttatcaagcagtatggcgactgtctgggagacatcgctgctagggatctgatctgtgcccagaagtttaatggcctcaccgtgctgcctcctctgctgaccgacgagatgatcgcccagtatacaagcgctctgctggccggcacaattaccagcggatggacatttggagccggcgctgccctccagattcctttcgccatgcagatggcctatagattcaacggcattggcgtcacacagaacgtgctgtacgagaaccagaagctgatcgctaaccagttcaacagcgccattggcaagatccaagattccctcagctccaccgccagcgccctcggcaaactgcaagacgtcgtgaatcagaatgcccaagctctgaacacactggtgaagcagctcagcagcaattttggcgccatctcctccgtgctcaatgatattctgtctagactggacaaggtggaggccgaagtccagatcgatagactgatcacaggtaagtgtcttaaattcagaagacgtaaagcaaaacacggttttgaggaggcttcttattataaatcttgcattatctacttttttctaggtagactgcagtccctccagacatacgtgacccagcagctcattagagctgccgagattagggcctccgccaatctcgctgccacaaaaatgagcgagtgcgtgctcggccagtccaaaagagtggacttctgtggcaagggctaccatctgatgtccttccctcagagcgctcctcatggcgtcgtgtttctgcatgtgacctacgtgcccgcccaagagaagaacttcacaacagcccccgctatctgtcacgacggaaaggcccacttccccagagagggcgtctttgtgtccaacggcacacactggtttgtcacccagaggaacttctatgagccccagatcatcaccaccgacaacacctttgtgagcggaaactgcgatgtggtcatcggcatcgtgaataacaccgtgtacgaccctctccagcccgagctggactccttcaaggaggagctggataagtactttaagaaccatacaagccccgacgtggacctcggcgacatttcaggtaagttgtccaacttttcaaagatccaggttttcttttaccataaatgtgttattgtctgtactaatctataggatttctctcttttgtaggtatcaacgccagcgtcgtgaacatccagaaggagattgatagactcaacgaggtcgccaagaatctgaacgagtctctgattgatctgcaagagctgggcaagtacgagcagtacatcaagtggccttggtacatctggctcggattcattgccggactgatcgccatcgtcatggtgaccatcatgctctgctgcatgacaagctgttgcagctgtctgaaaggctgttgtagctgtggcagctgctgtaagttcgatgaggacgactccgagcccgtgctgaagggcgtgaagctccactacacctaa

SEQ ID NO: 11 - DNA, 4967 bp, Artificial Sequence
  SARS-CoV-2 S protein cDNA construct(P171)
atgttcgtgttcctcgtgctgctgcctctggtgtcctcccagtgcgtcaatctgacaacaagaacacagctgccccccgcctacaccaattccttcacaagaggcgtgtactaccccgacaaggtgttcagaagctccgtgctgcacagcacccaagacctctttctgccctttttctccaacgtcacatggttccacgctatccacgtgtcaggtaagttgatttagaaacacttttcaagcagtcagcccatggttaccattaagttaaccctatcactgaattgctccaattttcctcttaggtaccaacggcaccaaaaggttcgataaccccgtcctccccttcaacgatggcgtctacttcgccagcaccgagaagtccaatatcatcagaggctggatcttcggcaccacactggattccaagacccagtctctgctgatcgtgaataacgccacaaacgtggtcattaaagtgtgcgagttccagttctgcaacgaccccttcctcggcgtgtattaccacaaaaacaacaagagctggatggagtccgagttcaggtaaggaaatttccatgagtttcactcttgaagcattggggttatttgtgccagaggctaatgacccatgctggcccttcacttttctagggtgtacagcagcgccaacaactgcacattcgagtacgtgagccagcctttcctcatggatctggagggcaagcaaggcaatttcaagaatctgagagagtttgtcttcaagaacatcgacggatacttcaagatttactccaagcacacccccattaacctcgtcagagacctcccccaaggcttttccgctctcgaacctctggtggatctgcccatcggcatcaacatcacaagattccaaaccctcctcgctctgcatagaagctatctgacccccggcgattccagctcaggtaagtaatttatataccactagagattttttcatcagtttctgttataaaaataattaaaatcaacatatttttctcctttacaacaggttggacagctggagctgccgcctactatgtgggatatctgcaacctagaacatttctgctgaagtacaacgagaacggcacaatcacagacgctgtggattgtgctctggaccccctctccgagaccaagtgtaccctcaagagctttaccgtggagaagggaatctaccagacctccaattttagggtccaacccaccgagagcatcgtgaggttccccaacatcacaaacctctgccctttcggcgaagtgttcaacgccacaaggtttgcttccgtgtacgcttggaacaggtaagtagtgctgattatacacaagatattgtctagaacttgatgagactgtggatatgaatatttcactcttttctcaggaagagaatctccaactgcgtggccgactatagcgtgctctataacagcgcctccttcagcaccttcaagtgttacggcgtgagccccaccaagctgaacgatctgtgtttcaccaacgtgtacgctgactccttcgtcattaggggcgacgaagtgagacaaatcgctcccggccagaccggcaaaatcgctgactacaactacaagctccccgacgacttcaccggctgtgtgatcgcttggaactccaacaacctcgatagcaaggtgggaggcaactacaactatctgtatagactcttcaggtaaggaatgttgcactgattttcacaggattttcccaagtgatactatcttattacattgatttttggctttgttttgttttcaggaagtccaatctgaagcccttcgagagggacatcagcacagagatctatcaagccggatccacaccttgcaacggcgtcgagggattcaactgctacttccctctgcaatcctacggcttccagcccacaaatggcgtgggctaccagccttacagagtggtggtgctgtcctttgaactgctgcatgcccccgccacagtgtgcggacccaaaaagagcaccaacctcgtgaagaacaaatgcgtcaatttcaacttcaatggactgaccggcacaggtaagtgacttgctttcttacatcaaaaaggcatccagtgtctgtttaagaattgccttctcaatattctctgttgattcctttccaggtgtgctcaccgagtccaacaagaagtttctgcccttccagcagttcggaagagacattgccgataccacagacgccgtgagggaccctcagacactggagattctggatatcacaccttgcagcttcggcggcgtgagcgtgatcacacccggaacaaacaccagcaaccaagtggctgtgctgtaccaagacgtgaattgtacagaggtacctgtggccatccatgccgatcagctgacccccacatggagggtctacagcacaggtaagtaggagaacattttcacatacaaagccatttttactttttttttaaatttcttataatcaatatgatctttttcacaggttccaatgtctttcagacaagagctggctgtctgattggcgctgagcacgtgaacaacagctacgagtgcgacatccctatcggcgccggaatttgcgccagctaccaaacccagaccaatagccctaggagggccagatccgtcgccagccagagcatcatcgcctataccatgtctctgggcgctgagaactccgtggcctatagcaacaacagcatcgctatccccaccaacttcacaatctccgtgaccacaggtaagttgctttctctgaatacaaaactattgtttgactgtctttaagaatattactttttcatcataacttcttctttgaaaagaaattctgcccgtgagcatgaccaagaccagcgtcgactgcaccatgtatatctgcggcgactccacagagtgctccaatctgctgctgcagtacggcagcttctgcacccaactcaatagggctctgaccggaattgctgtcgagcaagacaagaacacccaagaggtgtttgcccaagtgaaacagatttacaagaccccccccatcaaggacttcggaggcttcaatttctcccaaatcctccccgacccctccaaaccctccaagaggagctttatcgaggatctgctgttcaacaaggtgacactggctgatgcaggtaagtctatttcaaaaaagaatcatatatattttaaaatagcttatgtattttttacacattcatttcttatttacctactatttatccaggttttatcaagcagtatggcgactgtctgggagacatcgctgctagggatctgatctgtgcccagaagtttaatggcctcaccgtgctgcctcctctgctgaccgacgagatgatcgcccagtatacaagcgctctgctggccggcacaattaccagcggatggacatttggagccggcgctgccctccagattcctttcgccatgcagatggcctacaggtaagcaaatgaaccatcatcccatcattttgagttatatccttcctttgttatatggggcttacacttatcatttctcctttgctttaggttcaacggcattggcgtcacacagaacgtgctgtacgagaaccagaagctgatcgctaaccagttcaacagcgccattggcaagatccaagattccctcagctccaccgccagcgccctcggcaaactgcaagacgtcgtgaatcagaatgcccaagctctgaacacactggtgaagcagctcagcagcaattttggcgccatctcctccgtgctcaatgatattctgtctagactggacaaggtggaggccgaagtccagatcgatagactgatcacaggtaagtgtcttaaattcagaagacgtaaagcaaaacacggttttgaggaggcttcttattataaatcttgcattatctacttttttctaggtagactgcagtccctccagacatacgtgacccagcagctcattagagctgccgagattagggcctccgccaatctcgctgccacaaaaatgagcgagtgcgtgctcggccagtccaaaagagtggacttctgtggcaagggctaccatctgatgtccttccctcagagcgctcctcatggcgtcgtgtttctgcatgtgacctacgtgcccgcccaagagaagaacttcacaacagcccccgctatctgtcacgacggaaaggcccacttccccaggtaagtcattatatgaagaaaaacccaggtgcatgttttacatgaagaaaactggtatttgtttgactggttttgcttttatgttttagggagggcgtctttgtgtccaacggcacacactggtttgtcacccagaggaacttctatgagccccagatcatcaccaccgacaacacctttgtgagcggaaactgcgatgtggtcatcggcatcgtgaataacaccgtgtacgaccctctccagcccgagctggactccttcaaggaggagctggataagtactttaagaaccatacaagccccgacgtggacctcggcgacatttcaggtaagttgtccaacttttcaaagatccaggttttcttttaccataaatgtgttattgtctgtactaatctataggatttctctcttttgtaggtatcaacgccagcgtcgtgaacatccagaaggagattgatagactcaacgaggtcgccaagaatctgaacgagtctctgattgatctgcaagagctgggcaagtacgagcagtacatcaagtggccttggtacatctggctcggattcattgccggactgatcgccatcgtcatggtgaccatcatgctctgctgcatgacaagctgttgcagctgtctgaaaggctgttgtagctgtggcagctgctgtaagttcgatgaggacgactccgagcccgtgctgaagggcgtgaagctccactacacctaa

SEQ ID NO: 12 - DNA, 5049 bp, Artificial Sequence
  SARS-CoV-2 S protein cDNA construct (P231)
atgttcgtgttcctcgtgctgctgcctctggtgtcctcccagtgcgtcaatctgacaacaagaacacagctgccccccgcctacaccaattccttcacaagaggcgtgtactaccccgacaaggtgttcagaagctccgtgctgcacagcacccaagacctctttctgccctttttctccaacgtcacatggttccacgctatccacgtgtcaggtaagttgatttagaaacacttttcaagcagtcagcccatggttaccattaagttaaccctatcactgaattgctccaattttcctcttaggtaccaacggcaccaaaaggttcgataaccccgtcctccccttcaacgatggcgtctacttcgccagcaccgagaagtccaatatcatcagaggctggatcttcggcaccacactggattccaagacccagtctctgctgatcgtgaataacgccacaaacgtggtcattaaagtgtgcgagttccagttctgcaacgaccccttcctcggcgtgtattaccacaaaaacaacaagagctggatggagtccgagttcaggtaaggaaatttccatgagtttcactcttgaagcattggggttatttgtgccagaggctaatgacccatgctggcccttcacttttctagggtgtacagcagcgccaacaactgcacattcgagtacgtgagccagcctttcctcatggatctggagggcaagcaaggcaatttcaagaatctgagagagtttgtcttcaagaacatcgacggatacttcaagatttactccaagcacacccccattaacctcgtcagagacctcccccaaggtaagtgtgatgtggctaataatttatgtgtttatcaatttgtcgtttatgttaaataaaataatcatatactttttttcaggtttttccgctctcgaacctctggtggatctgcccatcggcatcaacatcacaagattccaaaccctcctcgctctgcatagaagctatctgacccccggcgattccagctcaggtaagtaatttatataccactagagattttttcatcagtttctgttataaaaataattaaaatcaacatatttttctcctttacaacaggttggacagctggagctgccgcctactatgtgggatatctgcaacctagaacatttctgctgaagtacaacgagaacggcacaatcacagacgctgtggattgtgctctggaccccctctccgagaccaagtgtaccctcaagagctttaccgtggagaagggaatctaccagacctccaattttagggtccaacccaccgagagcatcgtgaggttccccaacatcacaaacctctgccctttcggcgaagtgttcaacgccacaaggtttgcttccgtgtacgcttggaacaggtaagtagtgctgattatacacaagatattgtctagaacttgatgagactgtggatatgaatatttcactcttttctcaggaagagaatctccaactgcgtggccgactatagcgtgctctataacagcgcctccttcagcaccttcaagtgttacggcgtgagccccaccaagctgaacgatctgtgtttcaccaacgtgtacgctgactccttcgtcattaggggcgacgaagtgagacaaatcgctcccggccagaccggcaaaatcgctgactacaactacaagctccccgacgacttcaccggctgtgtgatcgcttggaactccaacaacctcgatagcaaggtgggaggcaactacaactatctgtatagactcttcaggtaaggaatgttgcactgattttcacaggattttcccaagtgatactatcttattacattgatttttggctttgttttgttttcaggaagtccaatctgaagcccttcgagagggacatcagcacagagatctatcaagccggatccacaccttgcaacggcgtcgagggattcaactgctacttccctctgcaatcctacggcttccagcccacaaatggcgtgggctaccagccttacagagtggtggtgctgtcctttgaactgctgcatgcccccgccacagtgtgcggacccaaaaagagcaccaacctcgtgaagaacaaatgcgtcaatttcaacttcaatggactgaccggcacaggtaagtgacttgctttcttacatcaaaaaggcatccagtgtctgtttaagaattgccttctcaatattctctgttgattcctttccaggtgtgctcaccgagtccaacaagaagtttctgcccttccagcagttcggaagagacattgccgataccacagacgccgtgagggaccctcagacactggagattctggatatcacaccttgcagcttcggcggcgtgagcgtgatcacacccggaacaaacaccagcaaccaagtggctgtgctgtaccaagacgtgaattgtacagaggtacctgtggccatccatgccgatcagctgacccccacatggagggtctacagcacaggtaagtaggagaacattttcacatacaaagccatttttactttttttttaaatttcttataatcaatatgatctttttcacaggttccaatgtctttcagacaagagctggctgtctgattggcgctgagcacgtgaacaacagctacgagtgcgacatccctatcggcgccggaatttgcgccagctaccaaacccagaccaatagccctaggagggccagatccgtcgccagccagagcatcatcgcctataccatgtctctgggcgctgagaactccgtggcctatagcaacaacagcatcgctatccccaccaacttcacaatctccgtgaccacaggtaagttgctttctctgaatacaaaactattgtttgactgtctttaagaatattactttttcatcataacttcttctttgaaaagaaattctgcccgtgagcatgaccaagaccagcgtcgactgcaccatgtatatctgcggcgactccacagagtgctccaatctgctgctgcagtacggcagcttctgcacccaactcaatagggctctgaccggaattgctgtcgagcaagacaagaacacccaagaggtgtttgcccaagtgaaacagatttacaagaccccccccatcaaggacttcggaggcttcaatttctcccaaatcctccccgacccctccaaaccctccaagaggagctttatcgaggatctgctgttcaacaaggtgacactggctgatgcaggtaagtctatttcaaaaaagaatcatatatattttaaaatagcttatgtattttttacacattcatttcttatttacctactatttatccaggttttatcaagcagtatggcgactgtctgggagacatcgctgctagggatctgatctgtgcccagaagtttaatggcctcaccgtgctgcctcctctgctgaccgacgagatgatcgcccagtatacaagcgctctgctggccggcacaattaccagcggatggacatttggagccggcgctgccctccagattcctttcgccatgcagatggcctacaggtaagcaaatgaaccatcatcccatcattttgagttatatccttcctttgttatatggggcttacacttatcatttctcctttgctttaggttcaacggcattggcgtcacacagaacgtgctgtacgagaaccagaagctgatcgctaaccagttcaacagcgccattggcaagatccaagattccctcagctccaccgccagcgccctcggcaaactgcaagacgtcgtgaatcagaatgcccaagctctgaacacactggtgaagcagctcagcagcaattttggcgccatctcctccgtgctcaatgatattctgtctagactggacaaggtggaggccgaagtccagatcgatagactgatcacaggtaagtgtcttaaattcagaagacgtaaagcaaaacacggttttgaggaggcttcttattataaatcttgcattatctacttttttctaggtagactgcagtccctccagacatacgtgacccagcagctcattagagctgccgagattagggcctccgccaatctcgctgccacaaaaatgagcgagtgcgtgctcggccagtccaaaagagtggacttctgtggcaagggctaccatctgatgtccttccctcagagcgctcctcatggcgtcgtgtttctgcatgtgacctacgtgcccgcccaagagaagaacttcacaacagcccccgctatctgtcacgacggaaaggcccacttccccaggtaagtcattatatgaagaaaaacccaggtgcatgttttacatgaagaaaactggtatttgtttgactggttttgcttttatgttttagggagggcgtctttgtgtccaacggcacacactggtttgtcacccagaggaacttctatgagccccagatcatcaccaccgacaacacctttgtgagcggaaactgcgatgtggtcatcggcatcgtgaataacaccgtgtacgaccctctccagcccgagctggactccttcaaggaggagctggataagtactttaagaaccatacaagccccgacgtggacctcggcgacatttcaggtaagttgtccaacttttcaaagatccaggttttcttttaccataaatgtgttattgtctgtactaatctataggatttctctcttttgtaggtatcaacgccagcgtcgtgaacatccagaaggagattgatagactcaacgaggtcgccaagaatctgaacgagtctctgattgatctgcaagagctgggcaagtacgagcagtacatcaagtggccttggtacatctggctcggattcattgccggactgatcgccatcgtcatggtgaccatcatgctctgctgcatgacaagctgttgcagctgtctgaaaggctgttgtagctgtggcagctgctgtaagttcgatgaggacgactccgagcccgtgctgaagggcgtgaagctccactacacctaa

SEQ ID NO: 13 - DNA, 5222 bp, Artificial Sequence
  SARS-CoV-2 S protein cDNA construct (P232)
atgttcgtgttcctcgtgctgctgcctctggtgtcctcccagtgcgtcaatctgacaacaagaacacagctgccccccgcctacaccaattccttcacaagaggcgtgtactaccccgacaaggtgttcagaagctccgtgctgcacagcacccaagacctctttctgccctttttctccaacgtcacatggttccacgctatccacgtgtcaggtaagttgatttagaaacacttttcaagcagtcagcccatggttaccattaagttaaccctatcactgaattgctccaattttcctcttaggtaccaacggcaccaaaaggttcgataaccccgtcctccccttcaacgatggcgtctacttcgccagcaccgagaagtccaatatcatcagaggctggatcttcggcaccacactggattccaagacccagtctctgctgatcgtgaataacgccacaaacgtggtcattaaagtgtgcgagttccagttctgcaacgaccccttcctcggcgtgtattaccacaaaaacaacaagagctggatggagtccgagttcaggtaaggaaatttccatgagtttcactcttgaagcattggggttatttgtgccagaggctaatgacccatgctggcccttcacttttctagggtgtacagcagcgccaacaactgcacattcgagtacgtgagccagcctttcctcatggatctggagggcaagcaaggtaagttaataccctttttaattaaaatgaattagtatttgccatttacttttactatttaagagatgtaaaattgcttttcaggtaatttcaagaatctgagagagtttgtcttcaagaacatcgacggatacttcaagatttactccaagcacacccccattaacctcgtcagagacctcccccaaggtaagtgtgatgtggctaataatttatgtgtttatcaatttgtcgtttatgttaaataaaataatcatatactttttttcaggtttttccgctctcgaacctctggtggatctgcccatcggcatcaacatcaccaggtaagtaataggaagtactgcatttcttcttcaaggacaaaattaatatctagcctaaaaaattaattttcatcttttaaatatttcaggttccaaaccctcctcgctctgcatagaagctatctgacccccggcgattccagctcaggtaagtaatttatataccactagagattttttcatcagtttctgttataaaaataattaaaatcaacatatttttctcctttacaacaggttggacagctggagctgccgcctactatgtgggatatctgcaacctagaacatttctgctgaagtacaacgagaacggcacaatcacagacgctgtggattgtgctctggaccccctctccgagaccaagtgtaccctcaagagctttaccgtggagaagggaatctaccagacctccaattttagggtccaacccaccgagagcatcgtgaggttccccaacatcacaaacctctgccctttcggcgaagtgttcaacgccacaaggtttgcttccgtgtacgcttggaacaggtaagtagtgctgattatacacaagatattgtctagaacttgatgagactgtggatatgaatatttcactcttttctcaggaagagaatctccaactgcgtggccgactatagcgtgctctataacagcgcctccttcagcaccttcaagtgttacggcgtgagccccaccaagctgaacgatctgtgtttcaccaacgtgtacgctgactccttcgtcattaggggcgacgaagtgagacaaatcgctcccggccagaccggcaaaatcgctgactacaactacaagctccccgacgacttcaccggctgtgtgatcgcttggaactccaacaacctcgatagcaaggtgggaggcaactacaactatctgtatagactcttcaggtaaggaatgttgcactgattttcacaggattttcccaagtgatactatcttattacattgatttttggctttgttttgttttcaggaagtccaatctgaagcccttcgagagggacatcagcacagagatctatcaagccggatccacaccttgcaacggcgtcgagggattcaactgctacttccctctgcaatcctacggcttccagcccacaaatggcgtgggctaccagccttacagagtggtggtgctgtcctttgaactgctgcatgcccccgccacagtgtgcggacccaaaaagagcaccaacctcgtgaagaacaaatgcgtcaatttcaacttcaatggactgaccggcacaggtaagtgacttgctttcttacatcaaaaaggcatccagtgtctgtttaagaattgccttctcaatattctctgttgattcctttccaggtgtgctcaccgagtccaacaagaagtttctgcccttccagcagttcggaagagacattgccgataccacagacgccgtgagggaccctcagacactggagattctggatatcacaccttgcagcttcggcggcgtgagcgtgatcacacccggaacaaacaccagcaaccaagtggctgtgctgtaccaagacgtgaattgtacagaggtacctgtggccatccatgccgatcagctgacccccacatggagggtctacagcacaggtaagtaggagaacattttcacatacaaagccatttttactttttttttaaatttcttataatcaatatgatctttttcacaggttccaatgtctttcagacaagagctggctgtctgattggcgctgagcacgtgaacaacagctacgagtgcgacatccctatcggcgccggaatttgcgccagctaccaaacccagaccaatagccctaggagggccagatccgtcgccagccagagcatcatcgcctataccatgtctctgggcgctgagaactccgtggcctatagcaacaacagcatcgctatccccaccaacttcacaatctccgtgaccacaggtaagttgctttctctgaatacaaaactattgtttgactgtctttaagaatattactttttcatcataacttcttctttgaaaagaaattctgcccgtgagcatgaccaagaccagcgtcgactgcaccatgtatatctgcggcgactccacagagtgctccaatctgctgctgcagtacggcagcttctgcacccaactcaatagggctctgaccggaattgctgtcgagcaagacaagaacacccaagaggtgtttgcccaagtgaaacagatttacaagaccccccccatcaaggacttcggaggcttcaatttctcccaaatcctccccgacccctccaaaccctccaagaggagctttatcgaggatctgctgttcaacaaggtgacactggctgatgcaggtaagtctatttcaaaaaagaatcatatatattttaaaatagcttatgtattttttacacattcatttcttatttacctactatttatccaggttttatcaagcagtatggcgactgtctgggagacatcgctgctagggatctgatctgtgcccagaagtttaatggcctcaccgtgctgcctcctctgctgaccgacgagatgatcgcccagtatacaagcgctctgctggccggcacaattaccagcggatggacatttggagccggcgctgccctccagattcctttcgccatgcagatggcctacaggtaagcaaatgaaccatcatcccatcattttgagttatatccttcctttgttatatggggcttacacttatcatttctcctttgctttaggttcaacggcattggcgtcacacagaacgtgctgtacgagaaccagaagctgatcgctaaccagttcaacagcgccattggcaagatccaagattccctcagctccaccgccagcgccctcggcaaactgcaagacgtcgtgaatcagaatgcccaagctctgaacacactggtgaagcagctcagcagcaattttggcgccatctcctccgtgctcaatgatattctgtctagactggacaaggtggaggccgaagtccagatcgatagactgatcacaggtaagtgtcttaaattcagaagacgtaaagcaaaacacggttttgaggaggcttcttattataaatcttgcattatctacttttttctaggtagactgcagtccctccagacatacgtgacccagcagctcattagagctgccgagattagggcctccgccaatctcgctgccacaaaaatgagcgagtgcgtgctcggccagtccaaaagagtggacttctgtggcaagggctaccatctgatgtccttccctcagagcgctcctcatggcgtcgtgtttctgcatgtgacctacgtgcccgcccaagagaagaacttcacaacagcccccgctatctgtcacgacggaaaggcccacttccccaggtaagtcattatatgaagaaaaacccaggtgcatgttttacatgaagaaaactggtatttgtttgactggttttgcttttatgttttagggagggcgtctttgtgtccaacggcacacactggtttgtcacccagaggaacttctatgagccccagatcatcaccaccgacaacacctttgtgagcggaaactgcgatgtggtcatcggcatcgtgaataacaccgtgtacgaccctctccagcccgagctggactccttcaaggaggagctggataagtactttaagaaccatacaagccccgacgtggacctcggcgacatttcaggtaagttgtccaacttttcaaagatccaggttttcttttaccataaatgtgttattgtctgtactaatctataggatttctctcttttgtaggtatcaacgccagcgtcgtgaacatccagaaggagattgatagactcaacgaggtcgccaagaatctgaacgagtctctgattgatctgcaagagctgggcaagtacgagcagtacatcaagtggccttggtacatctggctcggattcattgccggactgatcgccatcgtcatggtgaccatcatgctctgctgcatgacaagctgttgcagctgtctgaaaggctgttgtagctgtggcagctgctgtaagttcgatgaggacgactccgagcccgtgctgaagggcgtgaagctccactacacctaa

SEQ ID NO: 14 - DNA, 4960 bp, Artificial Sequence
  SARS-CoV-2 S protein cDNA construct (P186)
atgttcgtgttcctcgtgctgctgcctctggtgtcctcccagtgcgtcaatctgacaacaagaacacagctgccccccgcctacaccaattccttcacaagaggcgtgtactaccccgacaaggtgttcagaagctccgtgctgcacagcacccaagacctctttctgccctttttctccaacgtcacatggttccacgctatccacgtgtcaggtaagtatttaaagaagattctatttatactgtatatgtatcatttatttatttctccaggttcatattgcatgatttttctgttttcaggtaccaacggcaccaaaaggttcgataaccccgtcctccccttcaacgatggcgtctacttcgccagcaccgagaagtccaatatcatcagaggctggatcttcggcaccacactggattccaagacccagtctctgctgatcgtgaataacgccacaaacgtggtcattaaagtgtgcgagttccagttctgcaacgaccccttcctcggcgtgtattaccacaaaaacaacaagagctggatggagtccgagttcaggtaagtacagaagccatcaaacttttatatctgttttattcattttcaaataattataaaaataatattcttactaatatttatttcagggtgtacagcagcgccaacaactgcacattcgagtacgtgagccagcctttcctcatggatctggagggcaagcaaggcaatttcaagaatctgagagagtttgtcttcaagaacatcgacggatacttcaagatttactccaagcacacccccattaacctcgtcagagacctcccccaaggcttttccgctctcgaacctctggtggatctgcccatcggcatcaacatcacaagattccaaaccctcctcgctctgcatagaagctatctgacccccggcgattccagctcaggtaagtgtgatgtggctaataatttatgtgtttatcaatttgtcgtttatgttaaataaaataatcatatactttttttcaggttggacagctggagctgccgcctactatgtgggatatctgcaacctagaacatttctgctgaagtacaacgagaacggcacaatcacagacgctgtggattgtgctctggaccccctctccgagaccaagtgtaccctcaagagctttaccgtggagaagggaatctaccagacctccaattttagggtccaacccaccgagagcatcgtgaggttccccaacatcacaaacctctgccctttcggcgaagtgttcaacgccacaaggtttgcttccgtgtacgcttggaacaggtaagtactttcttaaatcaattctttagagcctttttaatttaaaaaatgtgcatacttcttttaaaatactatgtatattttcaggaagagaatctccaactgcgtggccgactatagcgtgctctataacagcgcctccttcagcaccttcaagtgttacggcgtgagccccaccaagctgaacgatctgtgtttcaccaacgtgtacgctgactccttcgtcattaggggcgacgaagtgagacaaatcgctcccggccagaccggcaaaatcgctgactacaactacaagctccccgacgacttcaccggctgtgtgatcgcttggaactccaacaacctcgatagcaaggtgggaggcaactacaactatctgtatagactcttcaggtaagtattgatttaaatgtaattacatttccactcatctacttaatttaagaattaggaattcgtatcttctttttgaacctcttaatctctttacaggaagtccaatctgaagcccttcgagagggacatcagcacagagatctatcaagccggatccacaccttgcaacggcgtcgagggattcaactgctacttccctctgcaatcctacggcttccagcccacaaatggcgtgggctaccagccttacagagtggtggtgctgtcctttgaactgctgcatgcccccgccacagtgtgcggacccaaaaagagcaccaacctcgtgaagaacaaatgcgtcaatttcaacttcaatggactgaccggcacaggtaagtcgattccttgcttatgtatatatctcacagtttgtattttgaatttttaaaaaatatttttcttttttttcttttttcttacaggtgtgctcaccgagtccaacaagaagtttctgcccttccagcagttcggaagagacattgccgataccacagacgccgtgagggaccctcagacactggagattctggatatcacaccttgcagcttcggcggcgtgagcgtgatcacacccggaacaaacaccagcaaccaagtggctgtgctgtaccaagacgtgaattgtacagaggtacctgtggccatccatgccgatcagctgacccccacatggagggtctacagcacaggtaagtagaagcttagattattttataaaactgtatgcacttctttaaaaatacttttactaacataaaattgtgattttacaggttccaatgtctttcagacaagagctggctgtctgattggcgctgagcacgtgaacaacagctacgagtgcgacatccctatcggcgccggaatttgcgccagctaccaaacccagaccaatagccctaggagggccagatccgtcgccagccagagcatcatcgcctataccatgtctctgggcgctgagaactccgtggcctatagcaacaacagcatcgctatccccaccaacttcacaatctccgtgaccacaggtaagtgacatgtgtcttaaattaatttattaaaaaacatataaataatttactatatctaaaatctaactgaaattcttaacattttctttcagaaattctgcccgtgagcatgaccaagaccagcgtcgactgcaccatgtatatctgcggcgactccacagagtgctccaatctgctgctgcagtacggcagcttctgcacccaactcaatagggctctgaccggaattgctgtcgagcaagacaagaacacccaagaggtgtttgcccaagtgaaacagatttacaagaccccccccatcaaggacttcggaggcttcaatttctcccaaatcctccccgacccctccaaaccctccaagaggagctttatcgaggatctgctgttcaacaaggtgacactggctgatgcaggtaagtagcttatttttctttattaaatatttactgagttaatattattcaacttaagtaatgaaaagttttggttcacttacaggttttatcaagcagtatggcgactgtctgggagacatcgctgctagggatctgatctgtgcccagaagtttaatggcctcaccgtgctgcctcctctgctgaccgacgagatgatcgcccagtatacaagcgctctgctggccggcacaattaccagcggatggacatttggagccggcgctgccctccagattcctttcgccatgcagatggcctacaggtaagttaataccctttttaattaaaatgaattagtatttgccatttacttttactatttaagagatgtaaaattgcttttcaggttcaacggcattggcgtcacacagaacgtgctgtacgagaaccagaagctgatcgctaaccagttcaacagcgccattggcaagatccaagattccctcagctccaccgccagcgccctcggcaaactgcaagacgtcgtgaatcagaatgcccaagctctgaacacactggtgaagcagctcagcagcaattttggcgccatctcctccgtgctcaatgatattctgtctagactggacaaggtggaggccgaagtccagatcgatagactgatcacaggtaagtaataggaagtactgcatttcttcttcaaggacaaaattaatatctagcctaaaaaattaattttcatcttttaaatatttcaggtagactgcagtccctccagacatacgtgacccagcagctcattagagctgccgagattagggcctccgccaatctcgctgccacaaaaatgagcgagtgcgtgctcggccagtccaaaagagtggacttctgtggcaagggctaccatctgatgtccttccctcagagcgctcctcatggcgtcgtgtttctgcatgtgacctacgtgcccgcccaagagaagaacttcacaacagcccccgctatctgtcacgacggaaaggcccacttccccaggtaagtttaaaaaaaaaaaaggcatctttttgcaaaggttacaacatgtgtggacttatgtttatgatatttatatttcagggagggcgtctttgtgtccaacggcacacactggtttgtcacccagaggaacttctatgagccccagatcatcaccaccgacaacacctttgtgagcggaaactgcgatgtggtcatcggcatcgtgaataacaccgtgtacgaccctctccagcccgagctggactccttcaaggaggagctggataagtactttaagaaccatacaagccccgacgtggacctcggcgacatttcaggtaagttcattcttgaatgatttcaaaacagaagtatttgcttttataagaagatcattttacatatattttatcacttacaggtatcaacgccagcgtcgtgaacatccagaaggagattgatagactcaacgaggtcgccaagaatctgaacgagtctctgattgatctgcaagagctgggcaagtacgagcagtacatcaagtggccttggtacatctggctcggattcattgccggactgatcgccatcgtcatggtgaccatcatgctctgctgcatgacaagctgttgcagctgtctgaaaggctgttgtagctgtggcagctgctgtaagttcgatgaggacgactccgagcccgtgctgaagggcgtgaagctccactacacctaa

SEQ ID NO: 15 - DNA, 4963 bp, Artificial Sequence
  SARS-CoV-2 S protein cDNA construct (P226)
atgttcgtgttcctcgtgctgctgcctctggtgtcctcccagtgcgtcaatctgacaacaagaacacagctgccccccgcctacaccaattccttcacaagaggcgtgtactaccccgacaaggtgttcagaagctccgtgctgcacagcacccaagacctctttctgccctttttctccaacgtcacatggttccacgctatccacgtgtcaggtaagttgatttagaaacacttttcaagcagtcagcccatggttaccattaagttaaccctatcactgaattgctccaattttcctcttaggtaccaacggcaccaaaaggttcgataaccccgtcctccccttcaacgatggcgtctacttcgccagcaccgagaagtccaatatcatcagaggctggatcttcggcaccacactggattccaagacccagtctctgctgatcgtgaataacgccacaaacgtggtcattaaagtgtgcgagttccagttctgcaacgaccccttcctcggcgtgtattaccacaaaaacaacaagagctggatggagtccgagttcaggtaaggaaatttccatgagtttcactcttgaagcattggggttatttgtgccagaggctaatgacccatgctggcccttcacttttctagggtgtacagcagcgccaacaactgcacattcgagtacgtgagccagcctttcctcatggatctggagggcaagcaaggcaatttcaagaatctgagagagtttgtcttcaagaacatcgacggatacttcaagatttactccaagcacacccccattaacctcgtcagagacctcccccaaggcttttccgctctcgaacctctggtggatctgcccatcggcatcaacatcacaagattccaaaccctcctcgctctgcatagaagctatctgacccccggcgattccagctcaggtaagtatccagattttacttcatatatttgcctttttctgtgctccgacttactaacattgtattctccccttcttcattttaggttggacagctggagctgccgcctactatgtgggatatctgcaacctagaacatttctgctgaagtacaacgagaacggcacaatcacagacgctgtggattgtgctctggaccccctctccgagaccaagtgtaccctcaagagctttaccgtggagaagggaatctaccagacctccaattttagggtccaacccaccgagagcatcgtgaggttccccaacatcacaaacctctgccctttcggcgaagtgttcaacgccacaaggtttgcttccgtgtacgcttggaacaggtaagtagtgctgattatacacaagatattgtctagaacttgatgagactgtggatatgaatatttcactcttttctcaggaagagaatctccaactgcgtggccgactatagcgtgctctataacagcgcctccttcagcaccttcaagtgttacggcgtgagccccaccaagctgaacgatctgtgtttcaccaacgtgtacgctgactccttcgtcattaggggcgacgaagtgagacaaatcgctcccggccagaccggcaaaatcgctgactacaactacaagctccccgacgacttcaccggctgtgtgatcgcttggaactccaacaacctcgatagcaaggtgggaggcaactacaactatctgtatagactcttcaggtaaggaatgttgcactgattttcacaggattttcccaagtgatactatcttattacattgatttttggctttgttttgttttcaggaagtccaatctgaagcccttcgagagggacatcagcacagagatctatcaagccggatccacaccttgcaacggcgtcgagggattcaactgctacttccctctgcaatcctacggcttccagcccacaaatggcgtgggctaccagccttacagagtggtggtgctgtcctttgaactgctgcatgcccccgccacagtgtgcggacccaaaaagagcaccaacctcgtgaagaacaaatgcgtcaatttcaacttcaatggactgaccggcacaggtaagtgacttgctttcttacatcaaaaaggcatccagtgtctgtttaagaattgccttctcaatattctctgttgattcctttccaggtgtgctcaccgagtccaacaagaagtttctgcccttccagcagttcggaagagacattgccgataccacagacgccgtgagggaccctcagacactggagattctggatatcacaccttgcagcttcggcggcgtgagcgtgatcacacccggaacaaacaccagcaaccaagtggctgtgctgtaccaagacgtgaattgtacagaggtacctgtggccatccatgccgatcagctgacccccacatggagggtctacagcacaggtaagtaggagaacattttcacatacaaagccatttttactttttttttaaatttcttataatcaatatgatctttttcacaggttccaatgtctttcagacaagagctggctgtctgattggcgctgagcacgtgaacaacagctacgagtgcgacatccctatcggcgccggaatttgcgccagctaccaaacccagaccaatagccctaggagggccagatccgtcgccagccagagcatcatcgcctataccatgtctctgggcgctgagaactccgtggcctatagcaacaacagcatcgctatccccaccaacttcacaatctccgtgaccacaggtaagttgctttctctgaatacaaaactattgtttgactgtctttaagaatattactttttcatcataacttcttctttgaaaagaaattctgcccgtgagcatgaccaagaccagcgtcgactgcaccatgtatatctgcggcgactccacagagtgctccaatctgctgctgcagtacggcagcttctgcacccaactcaatagggctctgaccggaattgctgtcgagcaagacaagaacacccaagaggtgtttgcccaagtgaaacagatttacaagaccccccccatcaaggacttcggaggcttcaatttctcccaaatcctccccgacccctccaaaccctccaagaggagctttatcgaggatctgctgttcaacaaggtgacactggctgatgcaggtaagtctatttcaaaaaagaatcatatatattttaaaatagcttatgtattttttacacattcatttcttatttacctactatttatccaggttttatcaagcagtatggcgactgtctgggagacatcgctgctagggatctgatctgtgcccagaagtttaatggcctcaccgtgctgcctcctctgctgaccgacgagatgatcgcccagtatacaagcgctctgctggccggcacaattaccagcggatggacatttggagccggcgctgccctccagattcctttcgccatgcagatggcctacaggtaagcaaatgaaccatcatcccatcattttgagttatatccttcctttgttatatggggcttacacttatcatttctcctttgctttaggttcaacggcattggcgtcacacagaacgtgctgtacgagaaccagaagctgatcgctaaccagttcaacagcgccattggcaagatccaagattccctcagctccaccgccagcgccctcggcaaactgcaagacgtcgtgaatcagaatgcccaagctctgaacacactggtgaagcagctcagcagcaattttggcgccatctcctccgtgctcaatgatattctgtctagactggacaaggtggaggccgaagtccagatcgatagactgatcacaggtaagtgtcttaaattcagaagacgtaaagcaaaacacggttttgaggaggcttcttattataaatcttgcattatctacttttttctaggtagactgcagtccctccagacatacgtgacccagcagctcattagagctgccgagattagggcctccgccaatctcgctgccacaaaaatgagcgagtgcgtgctcggccagtccaaaagagtggacttctgtggcaagggctaccatctgatgtccttccctcagagcgctcctcatggcgtcgtgtttctgcatgtgacctacgtgcccgcccaagagaagaacttcacaacagcccccgctatctgtcacgacggaaaggcccacttccccaggtaagtcattatatgaagaaaaacccaggtgcatgttttacatgaagaaaactggtatttgtttgactggttttgcttttatgttttagggagggcgtctttgtgtccaacggcacacactggtttgtcacccagaggaacttctatgagccccagatcatcaccaccgacaacacctttgtgagcggaaactgcgatgtggtcatcggcatcgtgaataacaccgtgtacgaccctctccagcccgagctggactccttcaaggaggagctggataagtactttaagaaccatacaagccccgacgtggacctcggcgacatttcaggtaagttgtccaacttttcaaagatccaggttttcttttaccataaatgtgttattgtctgtactaatctataggatttctctcttttgtaggtatcaacgccagcgtcgtgaacatccagaaggagattgatagactcaacgaggtcgccaagaatctgaacgagtctctgattgatctgcaagagctgggcaagtacgagcagtacatcaagtggccttggtacatctggctcggattcattgccggactgatcgccatcgtcatggtgaccatcatgctctgctgcatgacaagctgttgcagctgtctgaaaggctgttgtagctgtggcagctgctgtaagttcgatgaggacgactccgagcccgtgctgaagggcgtgaagctccactacacctaa

SEQ ID NO: 16 - DNA, 9920 bp, Artificial Sequence
  SARS-CoV-2 S protein cDNA construct (P227)
atgttcgtgttcctcgtgctgctgcctctggtgtcctcccagtgcgtcaatctgacaacaagaacacagctgccccccgcctacaccaattccttcacaagaggcgtgtactaccccgacaaggtgttcagaagctccgtgctgcacagcacccaagacctctttctgccctttttctccaacgtcacatggttccacgctatccacgtgtcaggtaagttgatttagaaacacttttcaagcagtcagcccatggttaccattaagttaaccctatcactgaattgctccaattttcctcttaggtaccaacggcaccaaaaggttcgataaccccgtcctccccttcaacgatggcgtctacttcgccagcaccgagaagtccaatatcatcagaggctggatcttcggcaccacactggattccaagacccagtctctgctgatcgtgaataacgccacaaacgtggtcattaaagtgtgcgagttccagttctgcaacgaccccttcctcggcgtgtattaccacaaaaacaacaagagctggatggagtccgagttcaggtaaggaaatttccatgagtttcactcttgaagcattggggttatttgtgccagaggctaatgacccatgctggcccttcacttttctagggtgtacagcagcgccaacaactgcacattcgagtacgtgagccagcctttcctcatggatctggagggcaagcaaggcaatttcaagaatctgagagagtttgtcttcaagaacatcgacggatacttcaagatttactccaagcacacccccattaacctcgtcagagacctcccccaaggcttttccgctctcgaacctctggtggatctgcccatcggcatcaacatcacaagattccaaaccctcctcgctctgcatagaagctatctgacccccggcgattccagctcaggtaagtgcatatgtcaaaaaaagggaatttttgaaaatttaatttaatcataaaaagaaaataaatttcattattttttgcaggttggacagctggagctgccgcctactatgtgggatatctgcaacctagaacatttctgctgaagtacaacgagaacggcacaatcacagacgctgtggattgtgctctggaccccctctccgagaccaagtgtaccctcaagagctttaccgtggagaagggaatctaccagacctccaattttagggtccaacccaccgagagcatcgtgaggttccccaacatcacaaacctctgccctttcggcgaagtgttcaacgccacaaggtttgcttccgtgtacgcttggaacaggtaagtagtgctgattatacacaagatattgtctagaacttgatgagactgtggatatgaatatttcactcttttctcaggaagagaatctccaactgcgtggccgactatagcgtgctctataacagcgcctccttcagcaccttcaagtgttacggcgtgagccccaccaagctgaacgatctgtgtttcaccaacgtgtacgctgactccttcgtcattaggggcgacgaagtgagacaaatcgctcccggccagaccggcaaaatcgctgactacaactacaagctccccgacgacttcaccggctgtgtgatcgcttggaactccaacaacctcgatagcaaggtgggaggcaactacaactatctgtatagactcttcaggtaaggaatgttgcactgattttcacaggattttcccaagtgatactatcttattacattgatttttggctttgttttgttttcaggaagtccaatctgaagcccttcgagagggacatcagcacagagatctatcaagccggatccacaccttgcaacggcgtcgagggattcaactgctacttccctctgcaatcctacggcttccagcccacaaatggcgtgggctaccagccttacagagtggtggtgctgtcctttgaactgctgcatgcccccgccacagtgtgcggacccaaaaagagcaccaacctcgtgaagaacaaatgcgtcaatttcaacttcaatggactgaccggcacaggtaagtgacttgctttcttacatcaaaaaggcatccagtgtctgtttaagaattgccttctcaatattctctgttgattcctttccaggtgtgctcaccgagtccaacaagaagtttctgcccttccagcagttcggaagagacattgccgataccacagacgccgtgagggaccctcagacactggagattctggatatcacaccttgcagcttcggcggcgtgagcgtgatcacacccggaacaaacaccagcaaccaagtggctgtgctgtaccaagacgtgaattgtacagaggtacctgtggccatccatgccgatcagctgacccccacatggagggtctacagcacaggtaagtaggagaacattttcacatacaaagccatttttactttttttttaaatttcttataatcaatatgatctttttcacaggttccaatgtctttcagacaagagctggctgtctgattggcgctgagcacgtgaacaacagctacgagtgcgacatccctatcggcgccggaatttgcgccagctaccaaacccagaccaatagccctaggagggccagatccgtcgccagccagagcatcatcgcctataccatgtctctgggcgctgagaactccgtggcctatagcaacaacagcatcgctatccccaccaacttcacaatctccgtgaccacaggtaagttgctttctctgaatacaaaactattgtttgactgtctttaagaatattactttttcatcataacttcttctttgaaaagaaattctgcccgtgagcatgaccaagaccagcgtcgactgcaccatgtatatctgcggcgactccacagagtgctccaatctgctgctgcagtacggcagcttctgcacccaactcaatagggctctgaccggaattgctgtcgagcaagacaagaacacccaagaggtgtttgcccaagtgaaacagatttacaagaccccccccatcaaggacttcggaggcttcaatttctcccaaatcctccccgacccctccaaaccctccaagaggagctttatcgaggatctgctgttcaacaaggtgacactggctgatgcaggtaagtctatttcaaaaaagaatcatatatattttaaaatagcttatgtattttttacacattcatttcttatttacctactatttatccaggttttatcaagcagtatggcgactgtctgggagacatcgctgctagggatctgatctgtgcccagaagtttaatggcctcaccgtgctgcctcctctgctgaccgacgagatgatcgcccagtatacaagcgctctgctggccggcacaattaccagcggatggacatttggagccggcgctgccctccagattcctttcgccatgcagatggcctacaggtaagcaaatgaaccatcatcccatcattttgagttatatccttcctttgttatatggggcttacacttatcatttctcctttgctttaggttcaacggcattggcgtcacacagaacgtgctgtacgagaaccagaagctgatcgctaaccagttcaacagcgccattggcaagatccaagattccctcagctccaccgccagcgccctcggcaaactgcaagacgtcgtgaatcagaatgcccaagctctgaacacactggtgaagcagctcagcagcaattttggcgccatctcctccgtgctcaatgatattctgtctagactggacaaggtggaggccgaagtccagatcgatagactgatcacaggtaagtgtcttaaattcagaagacgtaaagcaaaacacggttttgaggaggcttcttattataaatcttgcattatctacttttttctaggtagactgcagtccctccagacatacgtgacccagcagctcattagagctgccgagattagggcctccgccaatctcgctgccacaaaaatgagcgagtgcgtgctcggccagtccaaaagagtggacttctgtggcaagggctaccatctgatgtccttccctcagagcgctcctcatggcgtcgtgtttctgcatgtgacctacgtgcccgcccaagagaagaacttcacaacagcccccgctatctgtcacgacggaaaggcccacttccccaggtaagtcattatatgaagaaaaacccaggtgcatgttttacatgaagaaaactggtatttgtttgactggttttgcttttatgttttagggagggcgtctttgtgtccaacggcacacactggtttgtcacccagaggaacttctatgagccccagatcatcaccaccgacaacacctttgtgagcggaaactgcgatgtggtcatcggcatcgtgaataacaccgtgtacgaccctctccagcccgagctggactccttcaaggaggagctggataagtactttaagaaccatacaagccccgacgtggacctcggcgacatttcaggtaagttgtccaacttttcaaagatccaggttttcttttaccataaatgtgttattgtctgtactaatctataggatttctctcttttgtaggtatcaacgccagcgtcgtgaacatccagaaggagattgatagactcaacgaggtcgccaagaatctgaacgagtctctgattgatctgcaagagctgggcaagtacgagcagtacatcaagtggccttggtacatctggctcggattcattgccggactgatcgccatcgtcatggtgaccatcatgctctgctgcatgacaagctgttgcagctgtctgaaaggctgttgtagctgtggcagctgctgtaagttcgatgaggacgactccgagcccgtgctgaagggcgtgaagctccactacacctaaatgttcgtgttcctcgtgctgctgcctctggtgtcctcccagtgcgtcaatctgacaacaagaacacagctgccccccgcctacaccaattccttcacaagaggcgtgtactaccccgacaaggtgttcagaagctccgtgctgcacagcacccaagacctctttctgccctttttctccaacgtcacatggttccacgctatccacgtgtcaggtaagttgatttagaaacacttttcaagcagtcagcccatggttaccattaagttaaccctatcactgaattgctccaattttcctcttaggtaccaacggcaccaaaaggttcgataaccccgtcctccccttcaacgatggcgtctacttcgccagcaccgagaagtccaatatcatcagaggctggatcttcggcaccacactggattccaagacccagtctctgctgatcgtgaataacgccacaaacgtggtcattaaagtgtgcgagttccagttctgcaacgaccccttcctcggcgtgtattaccacaaaaacaacaagagctggatggagtccgagttcaggtaaggaaatttccatgagtttcactcttgaagcattggggttatttgtgccagaggctaatgacccatgctggcccttcacttttctagggtgtacagcagcgccaacaactgcacattcgagtacgtgagccagcctttcctcatggatctggagggcaagcaaggcaatttcaagaatctgagagagtttgtcttcaagaacatcgacggatacttcaagatttactccaagcacacccccattaacctcgtcagagacctcccccaaggcttttccgctctcgaacctctggtggatctgcccatcggcatcaacatcacaagattccaaaccctcctcgctctgcatagaagctatctgacccccggcgattccagctcaggtaagtagtttgggaaaacctttaaatttacgtcaaattttacataagaccgatatgttttaattattaaatattttgcaggttggacagctggagctgccgcctactatgtgggatatctgcaacctagaacatttctgctgaagtacaacgagaacggcacaatcacagacgctgtggattgtgctctggaccccctctccgagaccaagtgtaccctcaagagctttaccgtggagaagggaatctaccagacctccaattttagggtccaacccaccgagagcatcgtgaggttccccaacatcacaaacctctgccctttcggcgaagtgttcaacgccacaaggtttgcttccgtgtacgcttggaacaggtaagtagtgctgattatacacaagatattgtctagaacttgatgagactgtggatatgaatatttcactcttttctcaggaagagaatctccaactgcgtggccgactatagcgtgctctataacagcgcctccttcagcaccttcaagtgttacggcgtgagccccaccaagctgaacgatctgtgtttcaccaacgtgtacgctgactccttcgtcattaggggcgacgaagtgagacaaatcgctcccggccagaccggcaaaatcgctgactacaactacaagctccccgacgacttcaccggctgtgtgatcgcttggaactccaacaacctcgatagcaaggtgggaggcaactacaactatctgtatagactcttcaggtaaggaatgttgcactgattttcacaggattttcccaagtgatactatcttattacattgatttttggctttgttttgttttcaggaagtccaatctgaagcccttcgagagggacatcagcacagagatctatcaagccggatccacaccttgcaacggcgtcgagggattcaactgctacttccctctgcaatcctacggcttccagcccacaaatggcgtgggctaccagccttacagagtggtggtgctgtcctttgaactgctgcatgcccccgccacagtgtgcggacccaaaaagagcaccaacctcgtgaagaacaaatgcgtcaatttcaacttcaatggactgaccggcacaggtaagtgacttgctttcttacatcaaaaaggcatccagtgtctgtttaagaattgccttctcaatattctctgttgattcctttccaggtgtgctcaccgagtccaacaagaagtttctgcccttccagcagttcggaagagacattgccgataccacagacgccgtgagggaccctcagacactggagattctggatatcacaccttgcagcttcggcggcgtgagcgtgatcacacccggaacaaacaccagcaaccaagtggctgtgctgtaccaagacgtgaattgtacagaggtacctgtggccatccatgccgatcagctgacccccacatggagggtctacagcacaggtaagtaggagaacattttcacatacaaagccatttttactttttttttaaatttcttataatcaatatgatctttttcacaggttccaatgtctttcagacaagagctggctgtctgattggcgctgagcacgtgaacaacagctacgagtgcgacatccctatcggcgccggaatttgcgccagctaccaaacccagaccaatagccctaggagggccagatccgtcgccagccagagcatcatcgcctataccatgtctctgggcgctgagaactccgtggcctatagcaacaacagcatcgctatccccaccaacttcacaatctccgtgaccacaggtaagttgctttctctgaatacaaaactattgtttgactgtctttaagaatattactttttcatcataacttcttctttgaaaagaaattctgcccgtgagcatgaccaagaccagcgtcgactgcaccatgtatatctgcggcgactccacagagtgctccaatctgctgctgcagtacggcagcttctgcacccaactcaatagggctctgaccggaattgctgtcgagcaagacaagaacacccaagaggtgtttgcccaagtgaaacagatttacaagaccccccccatcaaggacttcggaggcttcaatttctcccaaatcctccccgacccctccaaaccctccaagaggagctttatcgaggatctgctgttcaacaaggtgacactggctgatgcaggtaagtctatttcaaaaaagaatcatatatattttaaaatagcttatgtattttttacacattcatttcttatttacctactatttatccaggttttatcaagcagtatggcgactgtctgggagacatcgctgctagggatctgatctgtgcccagaagtttaatggcctcaccgtgctgcctcctctgctgaccgacgagatgatcgcccagtatacaagcgctctgctggccggcacaattaccagcggatggacatttggagccggcgctgccctccagattcctttcgccatgcagatggcctacaggtaagcaaatgaaccatcatcccatcattttgagttatatccttcctttgttatatggggcttacacttatcatttctcctttgctttaggttcaacggcattggcgtcacacagaacgtgctgtacgagaaccagaagctgatcgctaaccagttcaacagcgccattggcaagatccaagattccctcagctccaccgccagcgccctcggcaaactgcaagacgtcgtgaatcagaatgcccaagctctgaacacactggtgaagcagctcagcagcaattttggcgccatctcctccgtgctcaatgatattctgtctagactggacaaggtggaggccgaagtccagatcgatagactgatcacaggtaagtgtcttaaattcagaagacgtaaagcaaaacacggttttgaggaggcttcttattataaatcttgcattatctacttttttctaggtagactgcagtccctccagacatacgtgacccagcagctcattagagctgccgagattagggcctccgccaatctcgctgccacaaaaatgagcgagtgcgtgctcggccagtccaaaagagtggacttctgtggcaagggctaccatctgatgtccttccctcagagcgctcctcatggcgtcgtgtttctgcatgtgacctacgtgcccgcccaagagaagaacttcacaacagcccccgctatctgtcacgacggaaaggcccacttccccaggtaagtcattatatgaagaaaaacccaggtgcatgttttacatgaagaaaactggtatttgtttgactggttttgcttttatgttttagggagggcgtctttgtgtccaacggcacacactggtttgtcacccagaggaacttctatgagccccagatcatcaccaccgacaacacctttgtgagcggaaactgcgatgtggtcatcggcatcgtgaataacaccgtgtacgaccctctccagcccgagctggactccttcaaggaggagctggataagtactttaagaaccatacaagccccgacgtggacctcggcgacatttcaggtaagttgtccaacttttcaaagatccaggttttcttttaccataaatgtgttattgtctgtactaatctataggatttctctcttttgtaggtatcaacgccagcgtcgtgaacatccagaaggagattgatagactcaacgaggtcgccaagaatctgaacgagtctctgattgatctgcaagagctgggcaagtacgagcagtacatcaagtggccttggtacatctggctcggattcattgccggactgatcgccatcgtcatggtgaccatcatgctctgctgcatgacaagctgttgcagctgtctgaaaggctgttgtagctgtggcagctgctgtaagttcgatgaggacgactccgagcccgtgctgaagggcgtgaagctccactacacctaa

SEQ ID NO: 17 - DNA, 4959 bp, Artificial Sequence
  SARS-CoV-2 S protein cDNA construct (P228)
atgttcgtgttcctcgtgctgctgcctctggtgtcctcccagtgcgtcaatctgacaacaagaacacagctgccccccgcctacaccaattccttcacaagaggcgtgtactaccccgacaaggtgttcagaagctccgtgctgcacagcacccaagacctctttctgccctttttctccaacgtcacatggttccacgctatccacgtgtcaggtaagttgatttagaaacacttttcaagcagtcagcccatggttaccattaagttaaccctatcactgaattgctccaattttcctcttaggtaccaacggcaccaaaaggttcgataaccccgtcctccccttcaacgatggcgtctacttcgccagcaccgagaagtccaatatcatcagaggctggatcttcggcaccacactggattccaagacccagtctctgctgatcgtgaataacgccacaaacgtggtcattaaagtgtgcgagttccagttctgcaacgaccccttcctcggcgtgtattaccacaaaaacaacaagagctggatggagtccgagttcaggtaaggaaatttccatgagtttcactcttgaagcattggggttatttgtgccagaggctaatgacccatgctggcccttcacttttctagggtgtacagcagcgccaacaactgcacattcgagtacgtgagccagcctttcctcatggatctggagggcaagcaaggcaatttcaagaatctgagagagtttgtcttcaagaacatcgacggatacttcaagatttactccaagcacacccccattaacctcgtcagagacctcccccaaggcttttccgctctcgaacctctggtggatctgcccatcggcatcaacatcacaagattccaaaccctcctcgctctgcatagaagctatctgacccccggcgattccagctcaggtaagtagtttgggaaaacctttaaatttacgtcaaattttacataagaccgatatgttttaattattaaatattttgcaggttggacagctggagctgccgcctactatgtgggatatctgcaacctagaacatttctgctgaagtacaacgagaacggcacaatcacagacgctgtggattgtgctctggaccccctctccgagaccaagtgtaccctcaagagctttaccgtggagaagggaatctaccagacctccaattttagggtccaacccaccgagagcatcgtgaggttccccaacatcacaaacctctgccctttcggcgaagtgttcaacgccacaaggtttgcttccgtgtacgcttggaacaggtaagtagtgctgattatacacaagatattgtctagaacttgatgagactgtggatatgaatatttcactcttttctcaggaagagaatctccaactgcgtggccgactatagcgtgctctataacagcgcctccttcagcaccttcaagtgttacggcgtgagccccaccaagctgaacgatctgtgtttcaccaacgtgtacgctgactccttcgtcattaggggcgacgaagtgagacaaatcgctcccggccagaccggcaaaatcgctgactacaactacaagctccccgacgacttcaccggctgtgtgatcgcttggaactccaacaacctcgatagcaaggtgggaggcaactacaactatctgtatagactcttcaggtaaggaatgttgcactgattttcacaggattttcccaagtgatactatcttattacattgatttttggctttgttttgttttcaggaagtccaatctgaagcccttcgagagggacatcagcacagagatctatcaagccggatccacaccttgcaacggcgtcgagggattcaactgctacttccctctgcaatcctacggcttccagcccacaaatggcgtgggctaccagccttacagagtggtggtgctgtcctttgaactgctgcatgcccccgccacagtgtgcggacccaaaaagagcaccaacctcgtgaagaacaaatgcgtcaatttcaacttcaatggactgaccggcacaggtaagtgacttgctttcttacatcaaaaaggcatccagtgtctgtttaagaattgccttctcaatattctctgttgattcctttccaggtgtgctcaccgagtccaacaagaagtttctgcccttccagcagttcggaagagacattgccgataccacagacgccgtgagggaccctcagacactggagattctggatatcacaccttgcagcttcggcggcgtgagcgtgatcacacccggaacaaacaccagcaaccaagtggctgtgctgtaccaagacgtgaattgtacagaggtacctgtggccatccatgccgatcagctgacccccacatggagggtctacagcacaggtaagtaggagaacattttcacatacaaagccatttttactttttttttaaatttcttataatcaatatgatctttttcacaggttccaatgtctttcagacaagagctggctgtctgattggcgctgagcacgtgaacaacagctacgagtgcgacatccctatcggcgccggaatttgcgccagctaccaaacccagaccaatagccctaggagggccagatccgtcgccagccagagcatcatcgcctataccatgtctctgggcgctgagaactccgtggcctatagcaacaacagcatcgctatccccaccaacttcacaatctccgtgaccacaggtaagttgctttctctgaatacaaaactattgtttgactgtctttaagaatattactttttcatcataacttcttctttgaaaagaaattctgcccgtgagcatgaccaagaccagcgtcgactgcaccatgtatatctgcggcgactccacagagtgctccaatctgctgctgcagtacggcagcttctgcacccaactcaatagggctctgaccggaattgctgtcgagcaagacaagaacacccaagaggtgtttgcccaagtgaaacagatttacaagaccccccccatcaaggacttcggaggcttcaatttctcccaaatcctccccgacccctccaaaccctccaagaggagctttatcgaggatctgctgttcaacaaggtgacactggctgatgcaggtaagtctatttcaaaaaagaatcatatatattttaaaatagcttatgtattttttacacattcatttcttatttacctactatttatccaggttttatcaagcagtatggcgactgtctgggagacatcgctgctagggatctgatctgtgcccagaagtttaatggcctcaccgtgctgcctcctctgctgaccgacgagatgatcgcccagtatacaagcgctctgctggccggcacaattaccagcggatggacatttggagccggcgctgccctccagattcctttcgccatgcagatggcctacaggtaagcaaatgaaccatcatcccatcattttgagttatatccttcctttgttatatggggcttacacttatcatttctcctttgctttaggttcaacggcattggcgtcacacagaacgtgctgtacgagaaccagaagctgatcgctaaccagttcaacagcgccattggcaagatccaagattccctcagctccaccgccagcgccctcggcaaactgcaagacgtcgtgaatcagaatgcccaagctctgaacacactggtgaagcagctcagcagcaattttggcgccatctcctccgtgctcaatgatattctgtctagactggacaaggtggaggccgaagtccagatcgatagactgatcacaggtaagtgtcttaaattcagaagacgtaaagcaaaacacggttttgaggaggcttcttattataaatcttgcattatctacttttttctaggtagactgcagtccctccagacatacgtgacccagcagctcattagagctgccgagattagggcctccgccaatctcgctgccacaaaaatgagcgagtgcgtgctcggccagtccaaaagagtggacttctgtggcaagggctaccatctgatgtccttccctcagagcgctcctcatggcgtcgtgtttctgcatgtgacctacgtgcccgcccaagagaagaacttcacaacagcccccgctatctgtcacgacggaaaggcccacttccccaggtaagtcattatatgaagaaaaacccaggtgcatgttttacatgaagaaaactggtatttgtttgactggttttgcttttatgttttagggagggcgtctttgtgtccaacggcacacactggtttgtcacccagaggaacttctatgagccccagatcatcaccaccgacaacacctttgtgagcggaaactgcgatgtggtcatcggcatcgtgaataacaccgtgtacgaccctctccagcccgagctggactccttcaaggaggagctggataagtactttaagaaccatacaagccccgacgtggacctcggcgacatttcaggtaagttgtccaacttttcaaagatccaggttttcttttaccataaatgtgttattgtctgtactaatctataggatttctctcttttgtaggtatcaacgccagcgtcgtgaacatccagaaggagattgatagactcaacgaggtcgccaagaatctgaacgagtctctgattgatctgcaagagctgggcaagtacgagcagtacatcaagtggccttggtacatctggctcggattcattgccggactgatcgccatcgtcatggtgaccatcatgctctgctgcatgacaagctgttgcagctgtctgaaaggctgttgtagctgtggcagctgctgtaagttcgatgaggacgactccgagcccgtgctgaagggcgtgaagctccactacacctaa

SEQ ID NO: 18 - DNA, 4969 bp, Artificial Sequence
  SARS-CoV-2 S protein cDNA construct (P229)
atgttcgtgttcctcgtgctgctgcctctggtgtcctcccagtgcgtcaatctgacaacaagaacacagctgccccccgcctacaccaattccttcacaagaggcgtgtactaccccgacaaggtgttcagaagctccgtgctgcacagcacccaagacctctttctgccctttttctccaacgtcacatggttccacgctatccacgtgtcaggtaagttgatttagaaacacttttcaagcagtcagcccatggttaccattaagttaaccctatcactgaattgctccaattttcctcttaggtaccaacggcaccaaaaggttcgataaccccgtcctccccttcaacgatggcgtctacttcgccagcaccgagaagtccaatatcatcagaggctggatcttcggcaccacactggattccaagacccagtctctgctgatcgtgaataacgccacaaacgtggtcattaaagtgtgcgagttccagttctgcaacgaccccttcctcggcgtgtattaccacaaaaacaacaagagctggatggagtccgagttcaggtaaggaaatttccatgagtttcactcttgaagcattggggttatttgtgccagaggctaatgacccatgctggcccttcacttttctagggtgtacagcagcgccaacaactgcacattcgagtacgtgagccagcctttcctcatggatctggagggcaagcaaggcaatttcaagaatctgagagagtttgtcttcaagaacatcgacggatacttcaagatttactccaagcacacccccattaacctcgtcagagacctcccccaaggcttttccgctctcgaacctctggtggatctgcccatcggcatcaacatcacaagattccaaaccctcctcgctctgcatagaagctatctgacccccggcgattccagctcaggtaagttctatataccaagaaaaatagacccctttgtccttttagacgtagaagtgatgagaacattttatgctattttttatcccttcaggttggacagctggagctgccgcctactatgtgggatatctgcaacctagaacatttctgctgaagtacaacgagaacggcacaatcacagacgctgtggattgtgctctggaccccctctccgagaccaagtgtaccctcaagagctttaccgtggagaagggaatctaccagacctccaattttagggtccaacccaccgagagcatcgtgaggttccccaacatcacaaacctctgccctttcggcgaagtgttcaacgccacaaggtttgcttccgtgtacgcttggaacaggtaagtagtgctgattatacacaagatattgtctagaacttgatgagactgtggatatgaatatttcactcttttctcaggaagagaatctccaactgcgtggccgactatagcgtgctctataacagcgcctccttcagcaccttcaagtgttacggcgtgagccccaccaagctgaacgatctgtgtttcaccaacgtgtacgctgactccttcgtcattaggggcgacgaagtgagacaaatcgctcccggccagaccggcaaaatcgctgactacaactacaagctccccgacgacttcaccggctgtgtgatcgcttggaactccaacaacctcgatagcaaggtgggaggcaactacaactatctgtatagactcttcaggtaaggaatgttgcactgattttcacaggattttcccaagtgatactatcttattacattgatttttggctttgttttgttttcaggaagtccaatctgaagcccttcgagagggacatcagcacagagatctatcaagccggatccacaccttgcaacggcgtcgagggattcaactgctacttccctctgcaatcctacggcttccagcccacaaatggcgtgggctaccagccttacagagtggtggtgctgtcctttgaactgctgcatgcccccgccacagtgtgcggacccaaaaagagcaccaacctcgtgaagaacaaatgcgtcaatttcaacttcaatggactgaccggcacaggtaagtgacttgctttcttacatcaaaaaggcatccagtgtctgtttaagaattgccttctcaatattctctgttgattcctttccaggtgtgctcaccgagtccaacaagaagtttctgcccttccagcagttcggaagagacattgccgataccacagacgccgtgagggaccctcagacactggagattctggatatcacaccttgcagcttcggcggcgtgagcgtgatcacacccggaacaaacaccagcaaccaagtggctgtgctgtaccaagacgtgaattgtacagaggtacctgtggccatccatgccgatcagctgacccccacatggagggtctacagcacaggtaagtaggagaacattttcacatacaaagccatttttactttttttttaaatttcttataatcaatatgatctttttcacaggttccaatgtctttcagacaagagctggctgtctgattggcgctgagcacgtgaacaacagctacgagtgcgacatccctatcggcgccggaatttgcgccagctaccaaacccagaccaatagccctaggagggccagatccgtcgccagccagagcatcatcgcctataccatgtctctgggcgctgagaactccgtggcctatagcaacaacagcatcgctatccccaccaacttcacaatctccgtgaccacaggtaagttgctttctctgaatacaaaactattgtttgactgtctttaagaatattactttttcatcataacttcttctttgaaaagaaattctgcccgtgagcatgaccaagaccagcgtcgactgcaccatgtatatctgcggcgactccacagagtgctccaatctgctgctgcagtacggcagcttctgcacccaactcaatagggctctgaccggaattgctgtcgagcaagacaagaacacccaagaggtgtttgcccaagtgaaacagatttacaagaccccccccatcaaggacttcggaggcttcaatttctcccaaatcctccccgacccctccaaaccctccaagaggagctttatcgaggatctgctgttcaacaaggtgacactggctgatgcaggtaagtctatttcaaaaaagaatcatatatattttaaaatagcttatgtattttttacacattcatttcttatttacctactatttatccaggttttatcaagcagtatggcgactgtctgggagacatcgctgctagggatctgatctgtgcccagaagtttaatggcctcaccgtgctgcctcctctgctgaccgacgagatgatcgcccagtatacaagcgctctgctggccggcacaattaccagcggatggacatttggagccggcgctgccctccagattcctttcgccatgcagatggcctacaggtaagcaaatgaaccatcatcccatcattttgagttatatccttcctttgttatatggggcttacacttatcatttctcctttgctttaggttcaacggcattggcgtcacacagaacgtgctgtacgagaaccagaagctgatcgctaaccagttcaacagcgccattggcaagatccaagattccctcagctccaccgccagcgccctcggcaaactgcaagacgtcgtgaatcagaatgcccaagctctgaacacactggtgaagcagctcagcagcaattttggcgccatctcctccgtgctcaatgatattctgtctagactggacaaggtggaggccgaagtccagatcgatagactgatcacaggtaagtgtcttaaattcagaagacgtaaagcaaaacacggttttgaggaggcttcttattataaatcttgcattatctacttttttctaggtagactgcagtccctccagacatacgtgacccagcagctcattagagctgccgagattagggcctccgccaatctcgctgccacaaaaatgagcgagtgcgtgctcggccagtccaaaagagtggacttctgtggcaagggctaccatctgatgtccttccctcagagcgctcctcatggcgtcgtgtttctgcatgtgacctacgtgcccgcccaagagaagaacttcacaacagcccccgctatctgtcacgacggaaaggcccacttccccaggtaagtcattatatgaagaaaaacccaggtgcatgttttacatgaagaaaactggtatttgtttgactggttttgcttttatgttttagggagggcgtctttgtgtccaacggcacacactggtttgtcacccagaggaacttctatgagccccagatcatcaccaccgacaacacctttgtgagcggaaactgcgatgtggtcatcggcatcgtgaataacaccgtgtacgaccctctccagcccgagctggactccttcaaggaggagctggataagtactttaagaaccatacaagccccgacgtggacctcggcgacatttcaggtaagttgtccaacttttcaaagatccaggttttcttttaccataaatgtgttattgtctgtactaatctataggatttctctcttttgtaggtatcaacgccagcgtcgtgaacatccagaaggagattgatagactcaacgaggtcgccaagaatctgaacgagtctctgattgatctgcaagagctgggcaagtacgagcagtacatcaagtggccttggtacatctggctcggattcattgccggactgatcgccatcgtcatggtgaccatcatgctctgctgcatgacaagctgttgcagctgtctgaaaggctgttgtagctgtggcagctgctgtaagttcgatgaggacgactccgagcccgtgctgaagggcgtgaagctccactacacctaa

SEQ ID NO: 19 - DNA, 4959 bp, Artificial Sequence
  SARS-CoV-2 S protein cDNA construct (P230)
atgttcgtgttcctcgtgctgctgcctctggtgtcctcccagtgcgtcaatctgacaacaagaacacagctgccccccgcctacaccaattccttcacaagaggcgtgtactaccccgacaaggtgttcagaagctccgtgctgcacagcacccaagacctctttctgccctttttctccaacgtcacatggttccacgctatccacgtgtcaggtaagttgatttagaaacacttttcaagcagtcagcccatggttaccattaagttaaccctatcactgaattgctccaattttcctcttaggtaccaacggcaccaaaaggttcgataaccccgtcctccccttcaacgatggcgtctacttcgccagcaccgagaagtccaatatcatcagaggctggatcttcggcaccacactggattccaagacccagtctctgctgatcgtgaataacgccacaaacgtggtcattaaagtgtgcgagttccagttctgcaacgaccccttcctcggcgtgtattaccacaaaaacaacaagagctggatggagtccgagttcaggtaaggaaatttccatgagtttcactcttgaagcattggggttatttgtgccagaggctaatgacccatgctggcccttcacttttctagggtgtacagcagcgccaacaactgcacattcgagtacgtgagccagcctttcctcatggatctggagggcaagcaaggcaatttcaagaatctgagagagtttgtcttcaagaacatcgacggatacttcaagatttactccaagcacacccccattaacctcgtcagagacctcccccaaggcttttccgctctcgaacctctggtggatctgcccatcggcatcaacatcacaagattccaaaccctcctcgctctgcatagaagctatctgacccccggcgattccagctcaggtaagtaatcgttcataagtaggtaaagctaaagtactaactaatttaaacaacgtaccttttttctttcctttttctcaggttggacagctggagctgccgcctactatgtgggatatctgcaacctagaacatttctgctgaagtacaacgagaacggcacaatcacagacgctgtggattgtgctctggaccccctctccgagaccaagtgtaccctcaagagctttaccgtggagaagggaatctaccagacctccaattttagggtccaacccaccgagagcatcgtgaggttccccaacatcacaaacctctgccctttcggcgaagtgttcaacgccacaaggtttgcttccgtgtacgcttggaacaggtaagtagtgctgattatacacaagatattgtctagaacttgatgagactgtggatatgaatatttcactcttttctcaggaagagaatctccaactgcgtggccgactatagcgtgctctataacagcgcctccttcagcaccttcaagtgttacggcgtgagccccaccaagctgaacgatctgtgtttcaccaacgtgtacgctgactccttcgtcattaggggcgacgaagtgagacaaatcgctcccggccagaccggcaaaatcgctgactacaactacaagctccccgacgacttcaccggctgtgtgatcgcttggaactccaacaacctcgatagcaaggtgggaggcaactacaactatctgtatagactcttcaggtaaggaatgttgcactgattttcacaggattttcccaagtgatactatcttattacattgatttttggctttgttttgttttcaggaagtccaatctgaagcccttcgagagggacatcagcacagagatctatcaagccggatccacaccttgcaacggcgtcgagggattcaactgctacttccctctgcaatcctacggcttccagcccacaaatggcgtgggctaccagccttacagagtggtggtgctgtcctttgaactgctgcatgcccccgccacagtgtgcggacccaaaaagagcaccaacctcgtgaagaacaaatgcgtcaatttcaacttcaatggactgaccggcacaggtaagtgacttgctttcttacatcaaaaaggcatccagtgtctgtttaagaattgccttctcaatattctctgttgattcctttccaggtgtgctcaccgagtccaacaagaagtttctgcccttccagcagttcggaagagacattgccgataccacagacgccgtgagggaccctcagacactggagattctggatatcacaccttgcagcttcggcggcgtgagcgtgatcacacccggaacaaacaccagcaaccaagtggctgtgctgtaccaagacgtgaattgtacagaggtacctgtggccatccatgccgatcagctgacccccacatggagggtctacagcacaggtaagtaggagaacattttcacatacaaagccatttttactttttttttaaatttcttataatcaatatgatctttttcacaggttccaatgtctttcagacaagagctggctgtctgattggcgctgagcacgtgaacaacagctacgagtgcgacatccctatcggcgccggaatttgcgccagctaccaaacccagaccaatagccctaggagggccagatccgtcgccagccagagcatcatcgcctataccatgtctctgggcgctgagaactccgtggcctatagcaacaacagcatcgctatccccaccaacttcacaatctccgtgaccacaggtaagttgctttctctgaatacaaaactattgtttgactgtctttaagaatattactttttcatcataacttcttctttgaaaagaaattctgcccgtgagcatgaccaagaccagcgtcgactgcaccatgtatatctgcggcgactccacagagtgctccaatctgctgctgcagtacggcagcttctgcacccaactcaatagggctctgaccggaattgctgtcgagcaagacaagaacacccaagaggtgtttgcccaagtgaaacagatttacaagaccccccccatcaaggacttcggaggcttcaatttctcccaaatcctccccgacccctccaaaccctccaagaggagctttatcgaggatctgctgttcaacaaggtgacactggctgatgcaggtaagtctatttcaaaaaagaatcatatatattttaaaatagcttatgtattttttacacattcatttcttatttacctactatttatccaggttttatcaagcagtatggcgactgtctgggagacatcgctgctagggatctgatctgtgcccagaagtttaatggcctcaccgtgctgcctcctctgctgaccgacgagatgatcgcccagtatacaagcgctctgctggccggcacaattaccagcggatggacatttggagccggcgctgccctccagattcctttcgccatgcagatggcctacaggtaagcaaatgaaccatcatcccatcattttgagttatatccttcctttgttatatggggcttacacttatcatttctcctttgctttaggttcaacggcattggcgtcacacagaacgtgctgtacgagaaccagaagctgatcgctaaccagttcaacagcgccattggcaagatccaagattccctcagctccaccgccagcgccctcggcaaactgcaagacgtcgtgaatcagaatgcccaagctctgaacacactggtgaagcagctcagcagcaattttggcgccatctcctccgtgctcaatgatattctgtctagactggacaaggtggaggccgaagtccagatcgatagactgatcacaggtaagtgtcttaaattcagaagacgtaaagcaaaacacggttttgaggaggcttcttattataaatcttgcattatctacttttttctaggtagactgcagtccctccagacatacgtgacccagcagctcattagagctgccgagattagggcctccgccaatctcgctgccacaaaaatgagcgagtgcgtgctcggccagtccaaaagagtggacttctgtggcaagggctaccatctgatgtccttccctcagagcgctcctcatggcgtcgtgtttctgcatgtgacctacgtgcccgcccaagagaagaacttcacaacagcccccgctatctgtcacgacggaaaggcccacttccccaggtaagtcattatatgaagaaaaacccaggtgcatgttttacatgaagaaaactggtatttgtttgactggttttgcttttatgttttagggagggcgtctttgtgtccaacggcacacactggtttgtcacccagaggaacttctatgagccccagatcatcaccaccgacaacacctttgtgagcggaaactgcgatgtggtcatcggcatcgtgaataacaccgtgtacgaccctctccagcccgagctggactccttcaaggaggagctggataagtactttaagaaccatacaagccccgacgtggacctcggcgacatttcaggtaagttgtccaacttttcaaagatccaggttttcttttaccataaatgtgttattgtctgtactaatctataggatttctctcttttgtaggtatcaacgccagcgtcgtgaacatccagaaggagattgatagactcaacgaggtcgccaagaatctgaacgagtctctgattgatctgcaagagctgggcaagtacgagcagtacatcaagtggccttggtacatctggctcggattcattgccggactgatcgccatcgtcatggtgaccatcatgctctgctgcatgacaagctgttgcagctgtctgaaaggctgttgtagctgtggcagctgctgtaagttcgatgaggacgactccgagcccgtgctgaagggcgtgaagctccactacacctaa

SEQ ID NO: 20 - DNA, 4961 bp, Artificial Sequence
  SARS-CoV-2 S protein cDNA construct (P241)
atgttcgtgttcctcgtgctgctgcctctggtgtcctcccagtgcgtcaatctgacaacaagaacacagctgccccccgcctacaccaattccttcacaagaggcgtgtactaccccgacaaggtgttcagaagctccgtgctgcacagcacccaagacctctttctgccctttttctccaacgtcacatggttccacgctatccacgtgtcaggtaagttgatttagaaacacttttcaagcagtcagcccatggttaccattaagttaaccctatcactgaattgctccaattttcctcttaggtaccaacggcaccaaaaggttcgataaccccgtcctccccttcaacgatggcgtctacttcgccagcaccgagaagtccaatatcatcagaggctggatcttcggcaccacactggattccaagacccagtctctgctgatcgtgaataacgccacaaacgtggtcattaaagtgtgcgagttccagttctgcaacgaccccttcctcggcgtgtattaccacaaaaacaacaagagctggatggagtccgagttcaggtaaggaaatttccatgagtttcactcttgaagcattggggttatttgtgccagaggctaatgacccatgctggcccttcacttttctagggtgtacagcagcgccaacaactgcacattcgagtacgtgagccagcctttcctcatggatctggagggcaagcaaggcaatttcaagaatctgagagagtttgtcttcaagaacatcgacggatacttcaagatttactccaagcacacccccattaacctcgtcagagacctcccccaaggcttttccgctctcgaacctctggtggatctgcccatcggcatcaacatcacaagattccaaaccctcctcgctctgcatagaagctatctgacccccggcgattccagctcaggtaagttagaagtattactaatgaagataatatcctgactaatagttaatagataatctttttctttcttttttttcctacaggttggacagctggagctgccgcctactatgtgggatatctgcaacctagaacatttctgctgaagtacaacgagaacggcacaatcacagacgctgtggattgtgctctggaccccctctccgagaccaagtgtaccctcaagagctttaccgtggagaagggaatctaccagacctccaattttagggtccaacccaccgagagcatcgtgaggttccccaacatcacaaacctctgccctttcggcgaagtgttcaacgccacaaggtttgcttccgtgtacgcttggaacaggtaagtagtgctgattatacacaagatattgtctagaacttgatgagactgtggatatgaatatttcactcttttctcaggaagagaatctccaactgcgtggccgactatagcgtgctctataacagcgcctccttcagcaccttcaagtgttacggcgtgagccccaccaagctgaacgatctgtgtttcaccaacgtgtacgctgactccttcgtcattaggggcgacgaagtgagacaaatcgctcccggccagaccggcaaaatcgctgactacaactacaagctccccgacgacttcaccggctgtgtgatcgcttggaactccaacaacctcgatagcaaggtgggaggcaactacaactatctgtatagactcttcaggtaaggaatgttgcactgattttcacaggattttcccaagtgatactatcttattacattgatttttggctttgttttgttttcaggaagtccaatctgaagcccttcgagagggacatcagcacagagatctatcaagccggatccacaccttgcaacggcgtcgagggattcaactgctacttccctctgcaatcctacggcttccagcccacaaatggcgtgggctaccagccttacagagtggtggtgctgtcctttgaactgctgcatgcccccgccacagtgtgcggacccaaaaagagcaccaacctcgtgaagaacaaatgcgtcaatttcaacttcaatggactgaccggcacaggtaagtgacttgctttcttacatcaaaaaggcatccagtgtctgtttaagaattgccttctcaatattctctgttgattcctttccaggtgtgctcaccgagtccaacaagaagtttctgcccttccagcagttcggaagagacattgccgataccacagacgccgtgagggaccctcagacactggagattctggatatcacaccttgcagcttcggcggcgtgagcgtgatcacacccggaacaaacaccagcaaccaagtggctgtgctgtaccaagacgtgaattgtacagaggtacctgtggccatccatgccgatcagctgacccccacatggagggtctacagcacaggtaagtaggagaacattttcacatacaaagccatttttactttttttttaaatttcttataatcaatatgatctttttcacaggttccaatgtctttcagacaagagctggctgtctgattggcgctgagcacgtgaacaacagctacgagtgcgacatccctatcggcgccggaatttgcgccagctaccaaacccagaccaatagccctaggagggccagatccgtcgccagccagagcatcatcgcctataccatgtctctgggcgctgagaactccgtggcctatagcaacaacagcatcgctatccccaccaacttcacaatctccgtgaccacaggtaagttgctttctctgaatacaaaactattgtttgactgtctttaagaatattactttttcatcataacttcttctttgaaaagaaattctgcccgtgagcatgaccaagaccagcgtcgactgcaccatgtatatctgcggcgactccacagagtgctccaatctgctgctgcagtacggcagcttctgcacccaactcaatagggctctgaccggaattgctgtcgagcaagacaagaacacccaagaggtgtttgcccaagtgaaacagatttacaagaccccccccatcaaggacttcggaggcttcaatttctcccaaatcctccccgacccctccaaaccctccaagaggagctttatcgaggatctgctgttcaacaaggtgacactggctgatgcaggtaagtctatttcaaaaaagaatcatatatattttaaaatagcttatgtattttttacacattcatttcttatttacctactatttatccaggttttatcaagcagtatggcgactgtctgggagacatcgctgctagggatctgatctgtgcccagaagtttaatggcctcaccgtgctgcctcctctgctgaccgacgagatgatcgcccagtatacaagcgctctgctggccggcacaattaccagcggatggacatttggagccggcgctgccctccagattcctttcgccatgcagatggcctacaggtaagcaaatgaaccatcatcccatcattttgagttatatccttcctttgttatatggggcttacacttatcatttctcctttgctttaggttcaacggcattggcgtcacacagaacgtgctgtacgagaaccagaagctgatcgctaaccagttcaacagcgccattggcaagatccaagattccctcagctccaccgccagcgccctcggcaaactgcaagacgtcgtgaatcagaatgcccaagctctgaacacactggtgaagcagctcagcagcaattttggcgccatctcctccgtgctcaatgatattctgtctagactggacaaggtggaggccgaagtccagatcgatagactgatcacaggtaagtgtcttaaattcagaagacgtaaagcaaaacacggttttgaggaggcttcttattataaatcttgcattatctacttttttctaggtagactgcagtccctccagacatacgtgacccagcagctcattagagctgccgagattagggcctccgccaatctcgctgccacaaaaatgagcgagtgcgtgctcggccagtccaaaagagtggacttctgtggcaagggctaccatctgatgtccttccctcagagcgctcctcatggcgtcgtgtttctgcatgtgacctacgtgcccgcccaagagaagaacttcacaacagcccccgctatctgtcacgacggaaaggcccacttccccaggtaagtcattatatgaagaaaaacccaggtgcatgttttacatgaagaaaactggtatttgtttgactggttttgcttttatgttttagggagggcgtctttgtgtccaacggcacacactggtttgtcacccagaggaacttctatgagccccagatcatcaccaccgacaacacctttgtgagcggaaactgcgatgtggtcatcggcatcgtgaataacaccgtgtacgaccctctccagcccgagctggactccttcaaggaggagctggataagtactttaagaaccatacaagccccgacgtggacctcggcgacatttcaggtaagttgtccaacttttcaaagatccaggttttcttttaccataaatgtgttattgtctgtactaatctataggatttctctcttttgtaggtatcaacgccagcgtcgtgaacatccagaaggagattgatagactcaacgaggtcgccaagaatctgaacgagtctctgattgatctgcaagagctgggcaagtacgagcagtacatcaagtggccttggtacatctggctcggattcattgccggactgatcgccatcgtcatggtgaccatcatgctctgctgcatgacaagctgttgcagctgtctgaaaggctgttgtagctgtggcagctgctgtaagttcgatgaggacgactccgagcccgtgctgaagggcgtgaagctccactacacctaa

SEQ ID NO: 21 - DNA, 711 bp, Artificial Sequence
  SARS-CoV-2 S protein cDNA construct (P233)
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtga

SEQ ID NO: 22 - DNA, 797 bp, Artificial Sequence
  SARS-CoV-2 S protein cDNA construct (P234)
atggtgagcaagggcgaggaggataacatggccatcatcaaggagttcatgcgcttcaaggtgcacatggagggctccgtgaacggccacgagttcgagatcgagggcgaaggtaaggaatgttgcactgattttcacaggattttcccaagtgatactatcttattacattgatttttggctttgttttgttttcaggtgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtga

SEQ ID NO: 23 - DNA, 975 bp, Artificial Sequence
  SARS-CoV-2 S protein cDNA construct (P235)
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtaagtaggagaacattttcacatacaaagccatttttactttttttttaaatttcttataatcaatatgatctttttcacaggttaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtaagtctatttcaaaaaagaatcatatatattttaaaatagcttatgtattttttacacattcatttcttatttacctactatttatccaggttaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgccaggtaagtcattatatgaagaaaaacccaggtgcatgttttacatgaagaaaactggtatttgtttgactggttttgcttttatgttttaggtgcctacaacgtcaacatcaagttggacatcacctcccacaacgaggactacaccatcgtggaacagtacgaacgcgccgagggccgccactccaccggcggcatggacgagctgtacaagtga

SEQ ID NO: 24 - DNA, 1067 bp, Artificial Sequence
  SARS-CoV-2 S protein cDNA construct (P236)
atggtgagcaagggcgaggaggataacatggccatcatcaaggagttcatgcgcttcaaggtaagtaatttatataccactagagattttttcatcagtttctgttataaaaataattaaaatcaacatatttttctcctttacaacaggttcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgaaggtaagttgctttctctgaatacaaaactattgtttgactgtctttaagaatattactttttcatcataacttcttctttgaaaaggtttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaaaggtaagtgtcttaaattcagaagacgtaaagcaaaacacggttttgaggaggcttcttattataaatcttgcattatctacttttttctaggtgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgaaggtaagttgtccaacttttcaaagatccaggttttcttttaccataaatgtgttattgtctgtactaatctataggatttctctcttttgtaggtcgccactccaccggcggcatggacgagctgtacaagtga

SEQ ID NO: 25 - DNA, 1331 bp, Artificial Sequence
  SARS-CoV-2 S protein cDNA construct (P237)
atggtgagcaagggcgaggaggataacatggccatcatcaaggagttcatgcgcttcaaggtaagtaatttatataccactagagattttttcatcagtttctgttataaaaataattaaaatcaacatatttttctcctttacaacaggttcacatggagggctccgtgaacggccacgagttcgagatcgagggcgagggcgagggccgcccctacgagggcacccagaccgccaagctgaaggtaagtaggagaacattttcacatacaaagccatttttactttttttttaaatttcttataatcaatatgatctttttcacaggttaccaagggtggccccctgcccttcgcctgggacatcctgtcccctcagttcatgtacggctccaaggcctacgtgaagcaccccgccgacatccccgactacttgaagctgtccttccccgaaggtaagttgctttctctgaatacaaaactattgtttgactgtctttaagaatattactttttcatcataacttcttctttgaaaaggtttcaagtgggagcgcgtgatgaacttcgaggacggcggcgtggtgaccgtgacccaggactcctccctgcaggacggcgagttcatctacaaggtaagtctatttcaaaaaagaatcatatatattttaaaatagcttatgtattttttacacattcatttcttatttacctactatttatccaggttaagctgcgcggcaccaacttcccctccgacggccccgtaatgcagaagaagaccatgggctgggaggcctcctccgagcggatgtaccccgaggacggcgccctgaaaggtaagtgtcttaaattcagaagacgtaaagcaaaacacggttttgaggaggcttcttattataaatcttgcattatctacttttttctaggtgagatcaagcagaggctgaagctgaaggacggcggccactacgacgctgaggtcaagaccacctacaaggccaagaagcccgtgcagctgccaggtaagtcattatatgaagaaaaacccaggtgcatgttttacatgaagaaaactggtatttgtttgactggttttgcttttatgttttaggtgcctacaacgtcaacatcaagttggacatcacctcccacaacgaggactacaccatcgtggaacagtacgaacgcgccgaaggtaagttgtccaacttttcaaagatccaggttttcttttaccataaatgtgttattgtctgtactaatctataggatttctctcttttgtaggtcgccactccaccggcggcatggacgagctgtacaagtga

SEQ ID NO: 26 - DNA, 1417 bp, Artificial Sequence
  SARS-CoV-2 S protein cDNA construct (P238)
atggtgagcaagggcgaggaggataacatggccatcatcaaggagttcatgcgcttcaaggtaagtaatttatataccactagagattttttcatcagtttctgttataaaaataattaaaatcaacatatttttctcctttacaacaggttcacatggagggctccgtgaacggccacgagttcgagatcgagggcgaaggtaaggaatgttgcactgattttcacaggattttcccaagtgatactatcttattacattgatttttggctttgttttgttttcaggtgagggccgcccctacgagggcacccagaccgccaagctgaaggtaagtaggagaacattttcacatacaaagccatttttactttttttttaaatttcttataatcaatatgatctttttcacaggttaccaagggtggccccctgcccttcgcctgggacatcctgtcccctcagttcatgtacggctccaaggcctacgtgaagcaccccgccgacatccccgactacttgaagctgtccttccccgaaggtaagttgctttctctgaatacaaaactattgtttgactgtctttaagaatattactttttcatcataacttcttctttgaaaaggtttcaagtgggagcgcgtgatgaacttcgaggacggcggcgtggtgaccgtgacccaggactcctccctgcaggacggcgagttcatctacaaggtaagtctatttcaaaaaagaatcatatatattttaaaatagcttatgtattttttacacattcatttcttatttacctactatttatccaggttaagctgcgcggcaccaacttcccctccgacggccccgtaatgcagaagaagaccatgggctgggaggcctcctccgagcggatgtaccccgaggacggcgccctgaaaggtaagtgtcttaaattcagaagacgtaaagcaaaacacggttttgaggaggcttcttattataaatcttgcattatctacttttttctaggtgagatcaagcagaggctgaagctgaaggacggcggccactacgacgctgaggtcaagaccacctacaaggccaagaagcccgtgcagctgccaggtaagtcattatatgaagaaaaacccaggtgcatgttttacatgaagaaaactggtatttgtttgactggttttgcttttatgttttaggtgcctacaacgtcaacatcaagttggacatcacctcccacaacgaggactacaccatcgtggaacagtacgaacgcgccgaaggtaagttgtccaacttttcaaagatccaggttttcttttaccataaatgtgttattgtctgtactaatctataggatttctctcttttgtaggtcgccactccaccggcggcatggacgagctgtacaagtga

SEQ ID NO: 27 - DNA, 2418 bp, Artificial Sequence
  SARS-CoV-2 S protein cDNA construct (P95)
atgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccatatggctgattgtttttggagttgtgatgggagtgatagtggttggcattgtcatcctgatcttcactgggatcagagatcggaagaagaaaaataaagcaagaagtggagaaaatccttatgcctccatcgatattagcaaaggagaaaataatccaggattccaaaacactgatgatgttcagacctccttttag

SEQ ID NO: 28 - DNA, 2687 bp, Artificial Sequence
  SARS-CoV-2 S protein cDNA construct (P223)
atgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtaagttgtccaacttttcaaagatccaggttttcttttaccataaatgtgttattgtctgtactaatctataggatttctctcttttgtaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggtaagttgctttctctgaatacaaaactattgtttgactgtctttaagaatattactttttcatcataacttcttctttgaaaaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatgtaagtctatttcaaaaaagaatcatatatattttaaaatagcttatgtattttttacacattcatttcttatttacctactatttatccagatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccatatggctgattgtttttggagttgtgatgggagtgatagtggttggcattgtcatcctgatcttcactgggatcagagatcggaagaagaaaaataaagcaagaagtggagaaaatccttatgcctccatcgatattagcaaaggagaaaataatccaggattccaaaacactgatgatgttcagacctccttttag

SEQ ID NO: 29 - DNA, 2949 bp, Artificial Sequence
  SARS-CoV-2 S protein cDNA construct (P242)
atgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtaagttgtccaacttttcaaagatccaggttttcttttaccataaatgtgttattgtctgtactaatctataggatttctctcttttgtaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagaggtaagtaggagaacattttcacatacaaagccatttttactttttttttaaatttcttataatcaatatgatctttttcacagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggtaagttgctttctctgaatacaaaactattgtttgactgtctttaagaatattactttttcatcataacttcttctttgaaaaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagtaagtacagaagccatcaaacttttatatctgttttattcattttcaaataattataaaaataatattcttactaatatttatttcaggcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatgtaagtctatttcaaaaaagaatcatatatattttaaaatagcttatgtattttttacacattcatttcttatttacctactatttatccagatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggtaagtgtcttaaattcagaagacgtaaagcaaaacacggttttgaggaggcttcttattataaatcttgcattatctacttttttctaggatgtcccggagccgtatcaatgatgctttccgtctgaatgacaacagcctagagtttctggggatacagccaacacttggacctcctaaccagccccctgtttccatatggctgattgtttttggagttgtgatgggagtgatagtggttggcattgtcatcctgatcttcactgggatcagagatcggaagaagaaaaataaagcaagaagtggagaaaatccttatgcctccatcgatattagcaaaggagaaaataatccaggattccaaaacactgatgatgttcagacctccttttag

SEQ ID NO: 30 - DNA, 3214 bp, Artificial Sequence
  SARS-CoV-2 S protein cDNA construct (P243)
atgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatggtaagtaatttatataccactagagattttttcatcagtttctgttataaaaataattaaaatcaacatatttttctcctttacaacagaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtaagttgtccaacttttcaaagatccaggttttcttttaccataaatgtgttattgtctgtactaatctataggatttctctcttttgtaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagaggtaagtaggagaacattttcacatacaaagccatttttactttttttttaaatttcttataatcaatatgatctttttcacagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggtaaggaatgttgcactgattttcacaggattttcccaagtgatactatcttattacattgatttttggctttgttttgttttcaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggtaagttgctttctctgaatacaaaactattgtttgactgtctttaagaatattactttttcatcataacttcttctttgaaaaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagtaagtacagaagccatcaaacttttatatctgttttattcattttcaaataattataaaaataatattcttactaatatttatttcaggcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttgtaagtcgattccttgcttatgtatatatctcacagtttgtattttgaatttttaaaaaatatttttcttttttttcttttttcttacagcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatgtaagtctatttcaaaaaagaatcatatatattttaaaatagcttatgtattttttacacattcatttcttatttacctactatttatccagatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggtaagtgtcttaaattcagaagacgtaaagcaaaacacggttttgaggaggcttcttattataaatcttgcattatctacttttttctaggatgtcccggagccgtatcaatgatgctttccgtctgaatgacaacagcctagagtttctggggatacagccaacacttggacctcctaaccagccccctgtttccatatggctgattgtttttggagttgtgatgggagtgatagtggttggcattgtcatcctgatcttcactgggatcagagatcggaagaagaaaaataaagcaagaagtggagaaaatccttatgcctccatcgatattagcaaaggagaaaataatccaggattccaaaacactgatgatgttcagacctccttttag

SEQ ID NO: 31 - DNA, 20 bp, Artificial Sequence
  5′ UTR_Beta-globin_intron_F Primer
atccacgctgttttgacctc

SEQ ID NO: 32 - DNA, 22 bp, Artificial Sequence
  FLAG_R Primer
tcatcgtcatccttgtagtcga

SEQ ID NO: 33 - DNA, 44 bp, Artificial Sequence
  5′ UTR_PRR36_F Primer
gtttagtgaaccgtcagatcgcctagccccctcccctcgcctcc

SEQ ID NO: 34 - DNA, 19 bp, Artificial Sequence
  3′ UTR_PRR36_R Primer
caatacactgcacacgggg

SEQ ID NO: 35 - DNA, 32 bp, Artificial Sequence
  5′ UTR_EFS_F Primer
ggtaccgcagagacaggagatctgccaccatg

SEQ ID NO: 36 - DNA, 22 bp, Artificial Sequence
  3′ UTR_Beta-globin polyA_R Primer
caaggcccttcataatatcccc

SEQ ID NO: 37 - RNA, 19 nt, Artificial Sequence
  Polypyrimidine tract (PPT)
uuuuuuucccuuuuuuucc

SEQ ID NO: 38 - DNA, 17 bp, Artificial Sequence
  M13F Primer
gtaaaacgacggccagt

SEQ ID NO: 39 - DNA, 17 bp, Artificial Sequence
  M13R Primer
caggaaacagctatgac

(19) United States
(12) Patent Application Publication
(10) Pub. No.: US 2024/0271145 A1
(43) Pub. Date: Aug. 15, 2024

(54) METHODS OF EUKARYOTIC GENE EXPRESSION

(71) Applicant: CAMBRIDGE ENTERPRISE LIMITED, Cambridge (GB)

(72) Inventors: Kärt TOMBERG, Cambridge (GB); Allan BRADLEY, Cambridge (GB)

(21) Appl. No.: 18/568,457

(22) PCT Filed: Jun. 20, 2022
(86) PCT No.: PCT/EP2022/066763

§ 371 Date: Dec. 8, 2023

(30) Foreign Application Priority Data
Jun. 21, 2021 (GB) 2108855.4

(51) Int. Cl. C12N 15/67; C12N 15/10; C12N 15/79

(52) U.S. Cl. CPC C12N 15/67; C12N 15/10; C12N 15/79

(57) ABSTRACT

This invention related to methods of adapting or modifying a complementary DNA (cDNA) sequence for expression in a eukaryotic cell. A nucleic acid molecule is provided that comprises a cDNA sequence that includes two or more splicing consensus motifs that divide the cDNA sequence into exon regions of 50 to 1200 nucleotides. Heterologous introns are then inserted into the splicing consensus motifs of the cDNA sequence, wherein each heterologous intron comprises a 3′ region having a GC content that is equal to or lower than the GC content of a 5′ region of the immediately downstream exon region. This produces a nucleic acid molecule comprising a modified cDNA sequence for expression in a eukaryotic cell. Methods, recombinant nucleic acid comprising a cDNA sequence, expression vectors comprising the recombinant nucleic acid and eukaryotic cells comprising a recombinant nucleic acid or expression vector are provided.

### FIELD

The present invention relates to the engineering of transgene cDNA sequences to increase expression in eukaryotic cells.

### BACKGROUND

The knowledge of the cis-acting elements required for gene expression has been built up over many decades starting from an initial understanding of bacteriophage and bacteria systems and extending these to eukaryotic viruses and ultimately eukaryotic genomes. Knowledge has been progressively enhanced and refined by transferring ectopic transcription units from one genome to another. Initially cultured mammalian cell lines have been used for this purpose but beginning in the 1980s transgenic animals have provided a convenient assay system for exploring the regulatory aspects of transgene expression. Transgenic mice have been used to define cis-acting regulatory elements in terms of their ability to direct appropriate levels of expression in the correct tissues and time. Most conveniently this investigation has used transgenes obtained from other species (such as LacZ, GFP) which label the cells in which expression occurs (Chalfie et al. 1994, Schmidt et al. 1998). Through such methods the promoters and enhancers which respond to the endogenous regulatory circuits have been determined for many genes. Moreover, other important elements have been recognized such as locus control regions, often found at substantial distances from genes, which enable copy number dependent gene expression for transgenes integrated in ectopic locations. At the nucleotide level, enhancements have been achieved by optimizing translation—for instance a “Kozak” consensus start site (Kozak 1984) is almost universally used.

Mammalian genes are typically large, their coding sequences are distributed over tens to hundreds kilobases of genomic DNA and regulatory elements required to maximize transgene expression can often lie at substantial distances from the transcription unit. Consequently, transgenes designed to express such sequences are typically reduced to their bare minimum size by removal of sequences with indeterminate or poorly understood contributions to gene expression, such as introns and 5′ and 3′ untranslated sequences, even though these are features of virtually every mammalian gene. Such transgene “trimming” has the advantage that the transgene can be squeezed into viral vector systems like adeno associated viruses with packaging size limits.

When used as naked DNA smaller size can result in more efficient transfection either as a result of more cells up-taking DNA and/or more copies inserting into the host cell genome. A larger transgene copy number is often considered advantageous as this in principle can result in greater levels of gene expression. Indeed, methods to select for cells with increased copy numbers of the transfected DNA are often used where gene expression levels have a commercial benefit. Examples of this include the use of genes like DHFR and GS which can be used to select for clones with amplified copies of a transgene sited directly upstream of the selection cassette (Urlaub et al. 1980, Cockett et al. 1990).

Other methods of improving gene expression include the use of regulatory sequences that are better matched to the target cell—in other words using promoters from the Chinese hamster genome to drive expression in a CHO cell. Removal of prokaryotic sequences is also considered advantageous in preventing loss of transgene expression (Haruyama et al. 2009). Similarly, the coding sequences may be “optimized” to introduce a balance of codons that are more like those of the species of the destination cell lines/organism, rather than those used by the source species (Gustafsson et al. 2004). By removing rare codons translation speed is in principle enhanced, though this may have other less desirable features—as folding complex molecules may be more rate limiting than translation per se.

Despite these and other innovations, the process of isolating a high-yielding cell line with stable expression over many generations is tedious, slow and expensive, and typically many thousands of clones must be screened to find one with the appropriate features. Such cell lines are invariably empirically derived, although in some cases features of the integration site are also examined—for instance transgene integration in a so called “methylation canyon” is less likely to be susceptible to silencing than integration in more methylated regions.

The importance of intronic sequences in the context of eukaryotic gene expression was recognised over 40 years ago (Hamer et al. 1979) and since then various related processes have been shown to be affected by introns including initial transcription of the gene, rate of transcription, polyadenylation, nuclear export, RNA editing, translational efficiency, and mRNA decay (Le Hir et al. 2003, Shaul 2017). This understanding has also led to the current common practice of including a 5′UTR intron into standard transgene expression systems.

There are numerous examples of intron-mediated expression enhancement, but still the understanding in the field is incomplete with various conflicting results reported. For example, in some cases different introns positioned identically within a single gene would result in opposite effects on protein expression (Bourdon et al. 2001) and sometimes the same intron placed within different positions of the cDNA sequence also yielded opposing results (Buchman et al. 1988, Bourdon et al. 2001). There are examples of introns that directly or indirectly have a negative effect on gene expression (Gromak 2012, Jin et al. 2017) and the magnitude of intron-dependent positive effects have also varied tremendously, from almost nothing to more than a 400-fold increase in mRNA levels (Buchman et al. 1988, Bourdon et al. 2001). In an effort to understand the underlying conflicts, a recent publication concluded that introns only improve expression of AT-rich cDNA sequences, but do not benefit GC-rich sequences (Mordstein et al. 2020).

While most endogenous genes in higher eukaryotes contain many introns (Piovesan et al. 2019), the expression benefits from adding multiple introns into transgenes is controversial and has not been implemented into common practice. A few reports have described expression enhancement using constructs with multiple endogenous introns a.k.a. minigenes (Virts et al. 2001). The use of two heterologous introns in mammalian cells (Lacy-Hulbert et al. 2001) and multiple introns in plants (Marillonnet et al. 2010, Grutzner et al. 2021) has been reported to improve mRNA and protein expression but the basis of effect described by Lacy-Hulbert et al. was not understood, appears to be specific to the reported case and cannot be applied to similar situations. Various reports have detailed that the addition of more introns did not bring added benefit to expression levels (Crane et al. 2019). U.S. Pat. No. 9,708,636B2 (Enenkel 2017) reports insertion of one or more artificial introns to enhance gene expression, and advises to preferably use only one intron, in order to reduce the risk of alternative splicing. Furthermore, their intronization examples are limited to endogenous intronic locations within a cDNA.

It is remarkable that 20 years after the human genome was deciphered and most gene structures were defined at the nucleotide level, the underlying rules that enable transcripts to be correctly spliced are not understood. Even though the intron-exon boundaries are highly or absolutely conserved in species as distant as humans and mice (around 100 million years of evolution), it remains impossible to predict with any certainty where introns lie in a genomic sequence without accessing the mRNA sequence and aligning this to the genome. It has therefore not been possible to design a gene structure de novo that reliably and reproducibly produces a designed spliced product in an experimental setting. The fact that intron/exon junctions are so highly conserved across species teaches that there is very strong evolutionary selection for maintaining the status quo. Moreover, this conservation places a very severe impediment to deciphering the rules that enable a cell to determine what to splice and what to retain in a transcript.

Recent reports show that endogenous intron and exon definitions in humans and other vertebrates are not uniform and different splicing factors are used within different genomic context (Amit et al. 2012, Lemaire et al. 2019) highlighting the fact that introns are not uniform in the genome and may not perform well within a different genomic context, such as a transgene. Amit et al. observed that genes in low GC % genomic regions tend to have large AT-rich introns with a clear GC % gradient at intron-exon interface (Amit et al. 2012). Wang et al. (2014) arXiv: 1404.2487 [q-bio.GN] reported that grouping exons by the GC content of their flanking introns indicates that the average exon size is positively correlated with GC content.

### SUMMARY

The present inventors have developed methods for modifying transgenes to increase their expression in eukaryotic cells through the incorporation of multiple heterologous introns to generate exon regions of defined length with defined gradients of GC content across intron/exon boundaries. These methods may be useful in the in vitro and in vivo expression of proteins, for example, in the production of recombinant proteins, gene therapy and nucleic acid or virus-based vaccination. These methods may also be useful in in vitro and in vivo transfection systems, for example to generate transgenic animals or re-program or engineer cells, such as T cells and other immune cells, for example through recombinant expression of a chimeric antigen receptor or other antigen receptor.

A first aspect of the invention provides a method of adapting or modifying a complementary DNA (cDNA) sequence for expression in a eukaryotic cell comprising;
providing a nucleic acid molecule comprising a cDNA sequence
wherein the cDNA sequence comprises two or more splicing consensus motifs that divide the cDNA sequence into exon regions of 50 to 1200 nucleotides,
inserting heterologous introns into the splicing consensus motifs of the cDNA sequence,
wherein each heterologous intron comprises a 3′ region having a GC content that is equal to or lower than the GC content of a 5′ region of the immediately downstream exon region,
thereby producing a nucleic acid molecule comprising a modified cDNA sequence for expression in a eukaryotic cell.

A second aspect of the invention provides a recombinant nucleic acid comprising a cDNA sequence for expression in a eukaryotic cell,
wherein the cDNA sequence comprises two or more heterologous introns and three or more exon regions of 50 to 1200 nucleotides,
wherein each heterologous intron comprises a 3′ region having a GC content that is equal to or lower than the GC content of a 5′ region of the immediately downstream exon region.

A third aspect of the invention provides an expression vector comprising a recombinant nucleic acid of the second aspect.

A fourth aspect of the invention provides a eukaryotic cell comprising a recombinant nucleic acid of the second aspect or an expression vector of the third aspect.

Other aspects and embodiments of the invention are described in more detail below.

### BRIEF DESCRIPTION OF THE FIGURES

FIG. 1 shows how intronization of SARS-COV-2 Spike protein with incorrect GC % landscape leads to alternatively spliced mRNA products. Insertion of one [A] or two [B] commonly used 5′UTR introns into the full length SARS-COV-2 S protein CDS sequence (wt) in addition to a 5′UTR β-globin intron resulted in a few strongly preferred alternatively spliced mRNA products guided by the intronic sequences. The same was observed for a S construct carrying all the introns from the human gene PRR36 [C]. The problem of cryptic splicing persisted when predicted splice sites were removed from the S protein CDS (wt+ss) as well as after codon-optimisation (c-o) of the S protein CDS [C]. Sliding a window of GC % across each construct illustrates the GC content of every exon and intron (shaded).

FIG. 2 shows that introduction of GC % landscape enables clear definition of exons and introns. Insertion of 13 short introns from human TTN gene into the wt S protein CDS with removed predicted splice sites (wt+ss) lead to various alternatively spliced products, most of which excluded exon 2. Maximising the GC content in the first 60 bp of exon 2 by codon-optimization was sufficient to ensure inclusion of that region into all identified splicing outcomes [A]. Extending this strategy throughout the S protein CDS (c-o) resulted not only in correct splicing of the transgene but also in improved protein expression over the equivalent intronless transgene [B].

FIG. 3 shows an overview of GC % landscape in 29 neighbouring intron-exon pairs from 3 different functional constructs. For each intron-exon pair GC % was calculated for different length segments (10 to 80 bp, plus full length of the elements) measured from the interface outwards [A]. The overall range of GC % in exons (20-80%) and introns (10-52%) was very wide and overlapping [B] but when neighbouring intron-exon pairs were considered, the exon had at least equal and in most cases higher GC % compared to the preceding intron [C].

FIG. 4 shows that adding more introns gradually improves expression outcomes until reaching the optimal exon length. Five constructs with increasing number of introns (3-15) introduced into S-protein CDS were generated. Addition of more introns gradually improved protein expression and performance in a pseudotyped virus infection assay until the smallest internal exon size was reduced to 55 bp (15 introns construct) [A]. The same outcome was observed with 5 constructs containing increasing number of introns (1-8) introduced into mCherry CDS [B]. Gradual improvement in expression was also observed with three intronized constructs of ACE2 CDS [C].

FIG. 5 shows that the correct intron-exon landscape can be achieved with endogenous, exogenous, or artificial introns. In addition to a S protein construct containing 13 intron sequences from human TTN gene, a construct with 13 mixed endogenous introns (each from a different human gene) was generated [A]. Additionally, exogenous introns from various species [B] as well as two different artificial introns [C] were introduced into the TTN construct replacing TTN intron 196. All the above S protein constructs expressed functional full-length S protein, with similar high performance in the pseudotyped virus infection assay [D].

FIG. 6 shows that intronization is a successful strategy for various constructs and across species. Successful addition of multiple introns was achieved in context of various transgenes, examples given here for SARS-COV-2 Spike protein CDS, fluorescent protein mCherry CDS, and human ACE2 CDS [A]. All the intronized constructs had higher expression outcomes in comparison to their intronless version, assessed in human embryonic kidney cell line Hek293 [B]. This was also observed in mouse embryonic cell line JM8 [C] and mouse colon adenocarcinoma cell line MC38 [D]. The transfection assay data is shown both as in % cells transfected as well as the median expression increase in the population, normalized to intronless construct.

### DETAILED DESCRIPTION

The methods described herein relate to the modification of a transgene for expression in a eukaryotic cell. The transgene may comprise a cDNA sequence. Heterologous introns are inserted into the splicing consensus motifs of the cDNA sequence such that the cDNA sequence is divided into exon regions of a defined length. All or part of each heterologous intron nucleic acid has a sequence that has a GC content that is equal or lower than the GC content of all or part of the immediately downstream exon region. In some embodiments, a gradient of GC content may be generated across the intron/exon boundaries of the modified cDNA sequence.

A modified cDNA sequence that is produced as described herein may display increased expression in a eukaryotic cell relative to the unmodified cDNA sequence. In some embodiments, the amount of cryptic splicing that occurs when the modified cDNA sequence is expressed in a eukaryotic cell may be less than the amount that occurs when the unmodified cDNA sequence is expressed. This reduction in cryptic splicing may lead to increased production of correctly spliced transcripts and increased expression in eukaryotic cells. For example, a modified cDNA sequence may display an increase in expression of at least 10%, at least 20%, at least 30%, at least 40%, at least 50%, at least 100%, at least 200%, or at least 500% relative to the unmodified cDNA sequence.

Expression of a cDNA sequence may be determined by any suitable technique at either the mRNA or protein expression level.

In some embodiments, the expression of a cDNA sequence may be determined by measuring the level or amount of mRNA transcribed from the cDNA. For example, a steady state transcript count of full-length cytoplasmic mRNA transcribed from the cDNA may be compared to a standard or set of standards.

Cytoplasmic full-length mRNAs may be captured by standard techniques, such as RNA sequencing, either without amplification, with low amplification or with controls for amplification bias. In some embodiments, Shashimi plots may be used to visualize read density across exons as well as splicing artefacts.

In other embodiments, the expression of a cDNA sequence may be determined by measuring the level or amount of protein produced from the cDNA sequence. For example, the level or amount of a secreted protein may be determined as a molecules per cell per day compared to a standard or set of standards. The level or amount of protein may be determined using routine techniques, such as ELISA or surface plasmon resonance (SPR), western blots, mass spectrometry, size exclusion chromatography (SEC) and comparisons to a standard curve. In some embodiments, biological activity may be assessed compared to a standard. For example, factor VIII may be quantified in a thrombin generation assay [TGA] and viral proteins, such viral spike proteins, may be quantified in a pseudotyped viral assay. The level or amount of a protein that is retained on the surface of cells may be determined by any suitable technique, such as antibody staining and a shift in mean intensity of a population of transfected cells. Improved expression may also be indicated by a higher transfection efficiency as more cells achieve the threshold by which the transgene product is detectable in an assay.

A cDNA sequence as described herein is the nucleotide sequence of the exons of a gene. The cDNA may correspond sequence of an mRNA that is expressed as DNA bases. A cDNA may be produced by any suitable technique and is not limited to sequences generated by reverse transcription of mRNA.

A cDNA sequence may be expressed to produce a gene product, such as a protein or non-coding RNA molecule, for example a shRNA or long non-coding RNA (lncRNA). A cDNA sequence for a non-coding RNA may consist of a non-coding nucleotide sequence that is transcribed in the eukaryotic cell but are not translated.

In some preferred embodiments, the cDNA sequence may comprise a coding sequence that encodes the amino acid sequence of a protein. The cDNA sequence may be transcribed and translated in a eukaryotic cell following expression of the cDNA to generate the encoded protein. The cDNA sequence may further comprise one or more non-coding sequences that are transcribed in the eukaryotic cell but are not translated. Non-coding sequences may include 5′ and 3′ untranslated regions (UTRs) and a polyA tail. In some embodiments, the cDNA sequence may be devoid of endogenous introns from the gene. For example, the unmodified cDNA sequence may consist of the contiguous nucleotide sequence of the exons of the gene. In other embodiments, the cDNA sequence may further comprise one or more endogenous introns from the gene. Suitable endogenous introns display the GC content and spacing of the heterologous introns described herein. For example, in addition to two or more heterologous introns, a modified cDNA sequence as described herein may further comprise one or more endogenous introns.

The coding sequence of the cDNA sequence may encode a gene product, such as a protein. The cDNA sequence may encode any protein for which increased expression or overexpression is desired. Suitable gene products include therapeutic proteins, such as clotting factors, enzymes, toxins, hormones, antibody molecules, cytokines, receptors, such as PD-1, T cell receptors and chimeric antigen receptors. In other instances, suitable gene products include industrially relevant proteins, for example proteins that have a non-therapeutic application, such as proteins involved in the production of chemicals, fragrances, and food. Modification of the cDNA sequence as described herein may be useful in maximizing yields in manufacturing of the therapeutic or non-therapeutic protein; or increasing the expression of the therapeutic or non-therapeutic protein in vivo. Other suitable gene products include antigenic proteins, such as viral, bacterial and parasite protein antigens, and tumour antigens. Viral protein antigens may include coronavirus proteins, such as coronavirus Spike (S) protein (e.g. SARS-COV-2 S protein). Tumour antigens may include tumour-specific and tumour-associated antigens. Other suitable gene products include research proteins, for example gene editing proteins, such as Cas9 and fluorescent proteins, such as GFP.

The cDNA sequence may be any suitable length to encode a gene product of interest. For example, suitable cDNA sequences may be 200 nucleotides or more, 240 nucleotides or more, 300 nucleotides or more, 400 nucleotides or more, 500 nucleotides or more, 1000 nucleotides or more, 1500 nucleotides or more or 2000 or more nucleotides in length. In some embodiments, longer cDNA sequences, such as 1000 nucleotides or more, may be preferred for intronization as described herein.

A cDNA sequence suitable for modification as described herein may be from any source. For example, the cDNA sequence may be an artificial sequence; an archaebacterial sequence; a viral sequence; a bacterial sequence; or a eukaryotic sequence, such as a protozoan or mesozoan sequence, such as a mammalian sequence. In some embodiments, cDNA sequence suitable for modification as described herein may be from a source in which it is not exposed to a cell nucleus, such as a bacterial cDNA sequence or a cytoplasmic viral cDNA sequence.

A suitable cDNA sequence may be codon optimised for expression in a host eukaryotic cell. For example, the codons within the cDNA sequence of the cDNA may be modified to reflect the codon usage bias of the host eukaryotic cell. Techniques for codon optimisation are readily available in the art.

A cDNA sequence as described herein may be operably linked to a suitable regulatory element to form a transgene.

The cDNA sequence is modified as described herein by the incorporation of heterologous introns. The incorporation of heterologous introns as described herein may be referred to as “intronization”. An intronized cDNA sequence may be transcribed in eukaryotic cells to produce a pre-mRNA molecule that comprises heterologous introns. The introns are subsequently removed from the pre-mRNA during splicing in the eukaryotic cells to generate an mRNA molecule that comprises a cDNA sequence for translation, along with a 5′CAP, 5′ and 3′ untranslated regions (UTRs) and a polyA tail.

A heterologous nucleic acid is a nucleic acid that is foreign to a particular gene, or other biological system, and is not naturally present in that system. A heterologous nucleic acid, such as a heterologous intron, may be introduced to the gene or other biological system by artificial means, for example using recombinant techniques. For example, a heterologous intron is inserted into the cDNA sequence of a gene at a position in which it is not naturally present.

A heterologous intron may be artificial or may be naturally occurring. For example, a heterologous intron may occur naturally in a different gene from the cDNA sequence. The different gene may be in the same or different species as the cDNA sequence, for example, the different gene may be the corresponding gene in a different species from the cDNA sequence. In some embodiments, a heterologous intron may occur naturally in the same gene in the same species as the cDNA sequence but inserted in a different location within the cDNA sequence. For example, the order of the introns in a modified cDNA sequence may be changed relative to the gene in which the introns and cDNA sequence naturally occur.

A cDNA sequence modified as described herein may be expressed in a eukaryotic cell. Suitable eukaryotic cells include higher eukaryotic cells, for example higher plant cells or metazoan cells, such as insect cells and mammalian cells.

Suitable eukaryotic cells include isolated cell lines used for the production of recombinant proteins, for example mammalian cells such as Chinese Hamster ovary (CHO) cells, Baby hamster kidney cells (BHK), mouse myeloma cells (NS/O), and Human embryonic kidney (HEK) cells.

Other suitable eukaryotic cells include host cells in vivo, for example cells in a human or non-human individual. Expression of a cDNA sequence modified as described herein in host cells in vivo may be useful for example in gene therapy, immunotherapy, such as vaccination, and the production of transgenic non-human animals.

Other suitable eukaryotic cells include host cells ex vivo, for example cells obtained from a human or non-human individual. Expression of a cDNA sequence modified as described herein in host cells ex vivo may be useful for example in producing cells for cell therapy, such as hematopoietic stem cells and immune cells, such as T-cells and NK-cells.

Suitable eukaryotic cells include isolated cell lines used for the industrial production of recombinant proteins, for example yeast cells, such as S. cerevisiae cells or Pichia pastoris cells and insect cells, such as Trichoplusia ni cells.

The cDNA sequence of a transgene is modified as described herein to correspond more closely to the architecture of endogenous genes in eukaryotic cells. Without being bound by theory, the mimicry of endogenous gene architecture may reduce the amount of cryptic splicing that occurs during expression of the cDNA sequence in a eukaryotic system and increase the amount of gene product produced. A modified cDNA sequence may be of any suitable length for cloning and delivery into a eukaryotic cell.

The heterologous introns divide the cDNA sequence into exon regions, each heterologous intron having an upstream (5′) and a (3′) downstream exon region. Splicing of the heterologous introns during expression in a eukaryotic cell removes the introns and re-connects the exon regions to generate an mRNA molecule comprising the exon regions in a contiguous sequence.

The number of heterologous introns inserted into the cDNA sequence depends on the size of the cDNA sequence and the number of introns required to divide it into exon regions of 50 to 1200 nucleotides. For example, the cDNA sequence may be modified to comprise 2, 3, 4, 5, 6, 7, 8, 9, 10 or more heterologous introns.

A cDNA sequence suitable for modification as described herein may comprises 2, 3, 4, 5, 6, 7, 8, 9, 10 or more splicing consensus motifs. The splicing consensus motifs are the sites into which the heterologous introns are inserted into the cDNA sequence. The heterologous introns may be inserted in splicing consensus motifs within the cDNA sequence or UTRs of the cDNA sequence.

A splicing consensus motif is a nucleotide sequence within the cDNA sequence that comprises the exon element of a donor splice site that occurs at the 5′ end of an intron (5′ exon element) and the exon element of an acceptor splice site that occurs at the 3′ end of an intron (3′ exon element). A heterologous intron may be inserted into a splicing consensus motif between the 5′ and 3′ exon elements to generate an intronized cDNA sequence comprising the heterologous intron with a donor splice site at its 5′ end and an acceptor splice site at its 3′ end. Splicing consensus motifs may be frame independent and may occur in any reading frame of the cDNA sequence. Suitable splicing consensus motifs are known in the art and may comprise the nucleotide sequence (C/A/G)AG_{↑}G(T/N)(T/N), preferably CAG_{↑}GTT (site of insertion of heterologous intron between the 5′ and 3′ exon elements is indicated). Other suitable splicing consensus motifs include ATG_{↑}AAT, CAG_{↑}GTT, GAG_{↑}ATT, CAG_{↑}GCC, CAG_{↑}GAT, GAA_{↑}GCG, GTT_{↑}CAA, CAT_{↑}ATG, and CAG_{↑}GAT. Splicing consensus motifs may be readily identified in a cDNA sequence using standard techniques.

The splicing consensus motifs may divide the cDNA sequence into exon regions of 50 to 1200 nucleotides, more preferably 80 to 380 nucleotides in length. In some preferred embodiments, the exon regions in the modified cDNA sequence may be 50 to 250 or 100 to 150 nucleotides in length.

Exon regions may be artificial exons generated in the cDNA sequence by the insertion of heterologous introns into consensus splice motifs. The cDNA sequence is divided by the heterologous introns into exon regions that together encode the gene product. In some embodiments, the cDNA sequence may comprise one or more endogenous introns that define one or more of the exon regions of the modified cDNA sequence.

In some embodiments, suitable splicing consensus motifs to divide the cDNA sequence into exon regions may be present or pre-existing in the cDNA sequence. A method described herein may comprise identifying splicing consensus motifs in the cDNA sequence. Sequence analysis tools for the identification of splicing consensus motifs is readily available in the art.

In other embodiments, the cDNA sequence may lack one or more of the splicing consensus motifs required to divide the cDNA sequence into exon regions. Splicing consensus motifs may be generated in the cDNA sequence by the introduction of one or more mutations to alter the existing cDNA sequence. Preferably, the one or more mutations generate one or more splicing consensus motifs without altering the sequence of the encoded protein. In some embodiments, the one or more mutations may also optimise the codons in the cDNA sequence for expression in a eukaryotic cell. In other embodiments, the one or more mutations may alter the sequence of the encoded protein, for example to increase or modify its activity.

A heterologous intron may be inserted between the 5′ and 3′ exon elements of a splicing consensus motif of the cDNA sequence.

Suitable heterologous introns may be 30 to 400 nucleotides in length, preferably 60 to 120 nucleotides or 80 to 100 nucleotides. The optimal intron length may be dependent on the eukaryotic host cell and may be optimised for expression in any specific eukaryotic host cell.

A heterologous intron may comprise
a 5′ splice-donor sequence;
a 3′ splice-acceptor sequence;
a polypyrimidine tract (PPT);
a branch point sequence; and
a 3′ region having a GC content that is equal to or lower than a 5′ region of the exon region immediately downstream of the splicing consensus motif into which the intron is inserted.

The heterologous intron may comprise a splice-donor sequence and a splice-acceptor sequence at the 5′ end and the 3′ end of the intron, respectively. The splice-donor sequence defines the 5′ end of the intron and the splice-acceptor sequence defines the 3′ end of an intron. Suitable splice-donor sequences may for example comprise a GT dinucleotide. Suitable splice-donor sequences may for example comprise an AG dinucleotide. The splice-donor and splice acceptor sequences of the heterologous intron may be optimised for the eukaryotic cell in which the cDNA sequence is expressed

The heterologous intron may further comprise a polypyrimidine tract (PPT). The polypyrimidine tract may be located upstream of the 3′ end of the heterologous intron, for example 5 to 40 nucleotides upstream of the 3′ end. The polypyrimidine tract may comprise a sequence of 15-20 nucleotides that is rich in pyrimidines (C and U). Suitable PPTs include 5′-UUUUUUUCCCUUUUUUUCC-3′ and variants thereof. Other suitable PPTs are known in the art (see for example Wagner et al 2001 Mol Cell Biol 21(10):3281-3288; WO2017171654A1).

The heterologous intron may further comprise a branch point sequence. The branch point sequence may be located upstream of the 3′ end of the intron nucleic acid and may for example be 20 to 50 nucleotides upstream of the 3′ end. The branch point sequence may comprise the sequence YURAC or YNURAC, where R=purine, Y=pyrimidine and N=any nucleotide. Suitable branch point sequences include 5′-UACUAACA-3′ and are known in the art (see for example Gao et al Nucl Acid Res 2008 36(7) 2257-2267; US20060094675).

GC content is the proportion of guanine or cytosine nucleotides in a nucleic acid sequence (i.e. (G+C)/total nucleotides) and is commonly expressed as a percentage (GC %). In some embodiments, insertion of a heterologous intron as described herein may generate a GC content gradient between the heterologous intron and the immediately downstream exon region (i.e. the exon region immediately adjacent the 3′ end of the heterologous intron). For example, a heterologous intron inserted into a splicing consensus motif may create a GC content gradient between the 3′ region of the heterologous intron and the 5′ region of the following exon region. The heterologous intron may comprise a 3′ region with a GC content that is lower than the 5′ region of the immediately downstream exon region. In other embodiments, the heterologous intron may comprise a 3′ region with a GC content that is the same as the 5′ region of the immediately downstream exon region. A gradient of GC content may not be generated between the heterologous intron and the immediately downstream exon region by insertion of the heterologous intron as described herein.

GC content may be measured starting from the interface in 3′ to 5′ direction for the intron and in 5′ to 3′ direction for the exon. Suitable tools for measuring GC content are readily available in the art.

The 3′ region of a heterologous intron inserted into the cDNA sequence may have a GC content that is equal to or at least 1%, at least 2%, at least 4%, at least 6%, at least 8%, at least 10%, at least 15% or at least 20% lower than the 5′ region of the immediately downstream exon region. In some embodiments, the 3′ region of the heterologous intron inserted into the cDNA sequence may have a GC content that is 0% to 46%, 2% to 40% or 5% to 35% lower than 5′ region of the immediately downstream exon region.

The size of the 3′ region of intron and the 5′ region of the downstream exon region (i.e. the window over which GC content is determined) may be 30 nucleotides or more, 40 nucleotides or more, 50 nucleotides or more, 60 nucleotides or more, 70 nucleotides or more, 80 nucleotides or more, 90 nucleotides or more or 100 nucleotides or more. In some embodiments, GC content may be determined across the whole of the intron and downstream exon region (i.e. the 3′ region of intron and the 5′ region of the downstream exon region may consist of the whole of the intron and exon region respectively). The GC content of the 3′ region of the heterologous intron may be equal to or lower than 5′ region of the immediately downstream exon region as described herein for 3′ and 5′ regions of any size.

In some preferred embodiments, the 3′ region of the heterologous intron and the 5′ region of the downstream exon region consist of 30 nucleotides. For example, the 30 nucleotides at the 3′ end of the heterologous intron may have a GC content that is equal to or lower, preferably up to 30%, 40%, 45%, 50% or 60% lower, than the 30 nucleotides at the 5′ end of the downstream exon region.

The sequence of a heterologous intron depends on the position within the cDNA sequence into which it is inserted. The GC content of the 5′ region of an exon region downstream of a splicing consensus motif may be determined. An intron sequence for insertion into the splicing consensus motif may then be designed that comprises a 3′ region with a GC content that is equal to or lower than the 5′ region of the exon region downstream of the splicing consensus motif, as described herein.

In some embodiments, the nucleotide sequence of a heterologous intron may be found in a naturally occurring intron, for example an intron from a different gene or a different position in the same gene.

In other embodiments, the nucleotide sequence of a heterologous intron may be artificial i.e. is not found in a naturally occurring intron. An artificial intron sequence may be designed using any convenient technique. For example, splice donor and splice acceptor sites may be positioned at the 5′ and 3′ ends of a nascent intron sequence. A branch point may be introduced to the middle of the nascent sequence. A random combination of T and C may be added to the nascent sequence to generate a pyrimidine tract of about 20 nucleotides. A random sequence of 50 or more nucleotides may be added between the pyrimidine tract and the branch point. Additional nucleotides may be added between the splice donor site and the branch point of the nascent sequence. The additional nucleotides may be random sequence with the A/T content adjusted to generate a GC % content equal to or lower than the 5′ region of the exon region downstream of the splicing consensus motif into which the intron is to be inserted. Suitable artificial introns may be 80-85 nucleotides in length. Suitable intron sequences for use as described herein are highlighted (lower case) in SEQ ID Nos: 1 to 30.

A suitable heterologous intron for insertion into a splicing consensus motif may be produced using standard synthetic or recombinant techniques. A method described herein may comprise providing heterologous introns for insertion into the two or more splicing consensus motifs in the cDNA sequence.

In addition to the insertion of heterologous introns, one or more further mutations may be introduced into the cDNA sequence, for example to remove cryptic splice sites. Cryptic splice sites may be identified by computational prediction tools that are readily available in the art (see for example Alternative Splice Site Predictor (Wang M. and Marín A. (2006) Gene 366: 219-227). Cryptic splice sites are preferably removed without altering the sequence of the gene product.

In some embodiments, a method described herein may comprise providing a nucleic acid comprising a cDNA sequence and inserting heterologous introns into the cDNA sequence of the nucleic acid as described herein to generate a nucleic acid comprising a modified cDNA sequence. Heterologous introns may be synthesised and inserted using standard techniques.

In other embodiments, a cDNA sequence that is modified to include heterologous introns may be designed and a nucleic acid comprising the modified cDNA sequence synthesised or assembled. For example, a method of adapting a cDNA sequence for expression in a eukaryotic cell comprising;
i) providing a cDNA sequence, wherein the cDNA sequence comprises two or more splicing consensus motifs that divide the cDNA sequence into exon regions of 50 to 1200 base pairs,
(ii) generating heterologous introns for insertion into each splicing consensus motif of the cDNA sequence, wherein each said intron comprises a 3′ region having a GC content that is equal to or lower than the GC content of the 5′ region of the immediately downstream exon region,
(iii) generating a modified cDNA sequence comprising the generated introns inserted into the splicing consensus motifs; and
(iv) synthesising a nucleic acid molecule comprising the modified cDNA sequence.

Steps 1 to 3 may be computer implemented, for example using standard sequence analysis software tools.

Examples of cDNA sequences modified as described herein are shown in SEQ ID NO: 6, SEQ ID NO: 7, SEQ ID NOs: 9-15, SEQ ID NOs: 17-21, SEQ ID NO: 25, SEQ ID NO: 27, and SEQ ID NOs: 28-30.

Also provided are cDNA sequences, nucleic acids, and transgenes modified as described herein. A recombinant nucleic acid as described herein may comprise a cDNA sequence for expression in a eukaryotic cell,
wherein the cDNA sequence comprises two or more heterologous introns and three or more exon regions of 50 to 1200 base pairs,
wherein each said heterologous intron comprises a 3′ region having a GC content equal to or lower than a 5′ region of the immediately downstream exon region.

The cDNA sequence of the recombinant nucleic acid may be produced by a method described herein. In some embodiments, a recombinant nucleic acid or transgene comprising a modified cDNA sequence as described herein may be directly inserted into the genome of a eukaryotic cell. For example, a modified cDNA sequence may be knocked into an endogenous gene locus. Suitable techniques for the random or targeted insertion into a genome are well-known in the art and include for example CRISPR-, Lox/Cre-, or transposon-based techniques.

In other embodiments, a recombinant nucleic acid or transgene comprising a modified cDNA sequence as described herein may be cloned and/or incorporated into a nucleic acid construct or vector, such as an expression vector. For example, the cDNA sequence may be operably linked to one or more control elements or regulatory sequences capable of directing the expression of the cDNA sequence. Suitable control elements or regulatory sequences to drive the expression of heterologous nucleic acid cDNA sequences in eukaryotic cells, preferably mammalian cells are well-known in the art and include constitutive promoters, for example viral promoters such as CMV or SV40; and tissue specific promoters, for example promoters such as the human thyroxine binding globulin (TBG) promoter or system specific promoters such as hypoxia responsive promoters.

Further provided are constructs in the form of plasmids, vectors (e.g. expression vectors), such as viral vectors e.g. phage, or phagemid vectors, transcription or expression cassettes or other delivery systems which comprise an adapted or intronized cDNA sequence as described herein. For example, the modified or intronized cDNA sequence may be contained in an expression vector. Suitable expression vectors can be chosen or constructed, containing appropriate regulatory sequences, including promoter sequences, terminator fragments, polyadenylation sequences, enhancer sequences, marker genes and other sequences as appropriate. A vector may also comprise sequences, such as origins of replication, promoter regions and selectable markers, which allow for its selection, expression and replication in bacterial hosts, such as E. coli.

Preferred vectors may be tropic for the cell type in which expression is required and may comprise suitable control and regulatory elements to enhance specific expression within that cell type.

Vectors may be plasmids, viral e.g. phage, or phagemid, as appropriate. For example, cosmids, BACs, or YACs may be used to accommodate long modified cDNA sequences. For further details see, for example, Molecular Cloning: a Laboratory Manual: 3rd edition, Russell et al., 2001, Cold Spring Harbor Laboratory Press. Many known techniques and protocols for manipulation of nucleic acid, for example in preparation of nucleic acid constructs, mutagenesis, sequencing, introduction of DNA into cells and gene expression, are described in detail in Current Protocols in Molecular Biology, Ausubel et al. eds. John Wiley & Sons, 1992. In some preferred embodiments, the expression vector may be a viral vector, such as a lentivirus or adeno-associated virus (AAV) vector.

The recombinant nucleic acid, transgene or expression vector may be introduced into a eukaryotic cell. The introduction may employ any available technique. Suitable techniques may depend on the vector and cell type and may include calcium phosphate transfection, DEAE-Dextran, electroporation, liposome-mediated transfection and transduction using retrovirus or other virus, e.g. vaccinia.

Nucleic acid may be introduced into the host eukaryotic cell using a viral or a plasmid-based system. The plasmid system may be maintained episomally or may be incorporated into the host cell or into an artificial chromosome. Incorporation may be either by random or targeted integration of one or more copies at single or multiple loci.

The introduction may be followed by causing or allowing expression of the modified cDNA sequence, e.g. by culturing host cells under conditions for expression of the gene.

Also provided are recombinant eukaryotic cells, for example recombinant mammalian cells, that comprise a recombinant nucleic acid or vector with a modified cDNA sequence as described herein. The cDNA sequence may be expressed in the cells to produce the gene product.

Systems for cloning and expression of nucleic acid in a variety of different host eukaryotic cells are well known. Suitable host cells include mammalian, insect and yeast systems. Mammalian cell lines available in the art for expression of a heterologous protein include Chinese Hamster ovary (CHO) cells, Baby hamster kidney cells (BHK), mouse myeloma cells (NS/O). and Human embryonic kidney (HEK) cells and many others.

Also provided are methods of expressing a cDNA sequence in a eukaryotic cell comprising;
modifying a cDNA sequence by a method described herein to produce a modified cDNA sequence,
incorporating the modified cDNA sequence into an expression vector.
introducing the expression vector into a eukaryotic cell and
causing or allowing expression from the modified cDNA sequence to produce a gene product.

The cDNA sequence may encode a gene product. Following production by expression of a nucleic acid comprising a modified cDNA sequence, the gene product may be isolated and/or purified using any suitable technique, then used as appropriate. For example, a method of production may further comprise formulating the product into a composition including at least one additional component, such as a pharmaceutically acceptable excipient.

Other aspects and embodiments of the invention provide the aspects and embodiments described above with the term “comprising” replaced by the term “consisting of” and the aspects and embodiments described above with the term “comprising” replaced by the term “consisting essentially of”.

The term “downstream” as used herein refers to the 5′ to 3′ direction in a nucleic acid described herein and the term “upstream” as used herein refers to the 3′ to 5′ direction in a nucleic acid described herein

Reference to a nucleotide sequence as set out herein encompasses a DNA molecule with the specified sequence, and encompasses a RNA molecule with the specified sequence in which U is substituted for T, unless context requires otherwise.

It is to be understood that the application discloses all combinations of any of the above aspects and embodiments described above with each other, unless the context demands otherwise. Similarly, the application discloses all combinations of the preferred and/or optional features either singly or together with any of the other aspects, unless the context demands otherwise.

Modifications of the above embodiments, further embodiments and modifications thereof will be apparent to the skilled person on reading this disclosure, and as such, these are within the scope of the present invention.

All documents and sequence database entries mentioned in this specification are incorporated herein by reference in their entirety for all purposes.

“and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

### Experimental

### Materials and Methods

### Transgene Constructs

Table 1 gives an overview of all the transgene constructs with the relevant 5′ and 3′ elements and the used plasmid backbone. Full DNA sequences of these constructs are given below. Wildtype (wt) SARS-COV-2 S protein CDS sequence refers to the S protein cDNA sequence from the Wuhan-Hu-1 isolate (Genbank: MN908947.3) while “18F” refers to the removal of the last 18 amino acids of the S protein C-terminus (ER retention sequence) and the addition of a FLAG tag. The DNA sequence for codon-optimized (c-o) SARS-CoV-2 S protein was obtained from the National Institute for Biological Standards and Control website (nibsc.org, CFAR #100976). mCherry CDS refers to the “synthetic construct monomeric red fluorescent protein gene” (Genbank: AY678264.1), with the stop codon changed from ‘TAA’ to ‘TGA’ while human ACE2 CDS refers to the “Homo sapiens angiotensin converting enzyme 2, mRNA transcript variant 2” (Genbank: NM_021804.3).

All constructs were assembled by Gibson cloning of relevant PCR products and/or custom designed gene blocks (gBlocks Gene Fragments, Integrated DNA Technologies) according to manufacturer's protocol (Gibson Assembly Master Mix, NEB).

### GC % Calculations Overall and at Intron-Exon Interface

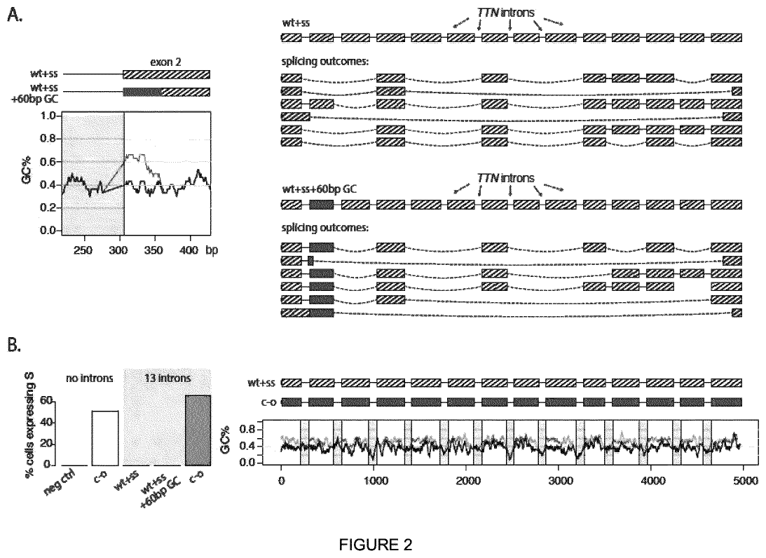

Construct GC % was calculated using a sliding window of 30 base pairs (bp) across the sequence, resetting at each element (intron or exon) to highlight their GC % difference. For a given 30 bp window, the frequency of G and C nucleotides was measured (equals total count of G and C nucleotides in sequence divided by sequence length). Then the window would slide by 1 bp, moving in 5′ to 3′ direction. Last measurement for an element would be calculated when the sliding window hits the start of the next element. Then the window would jump 30 bp to start measuring GC % at the beginning of the next element. This gap in GC % measurement is visualized as a dashed line in FIG. 2A.

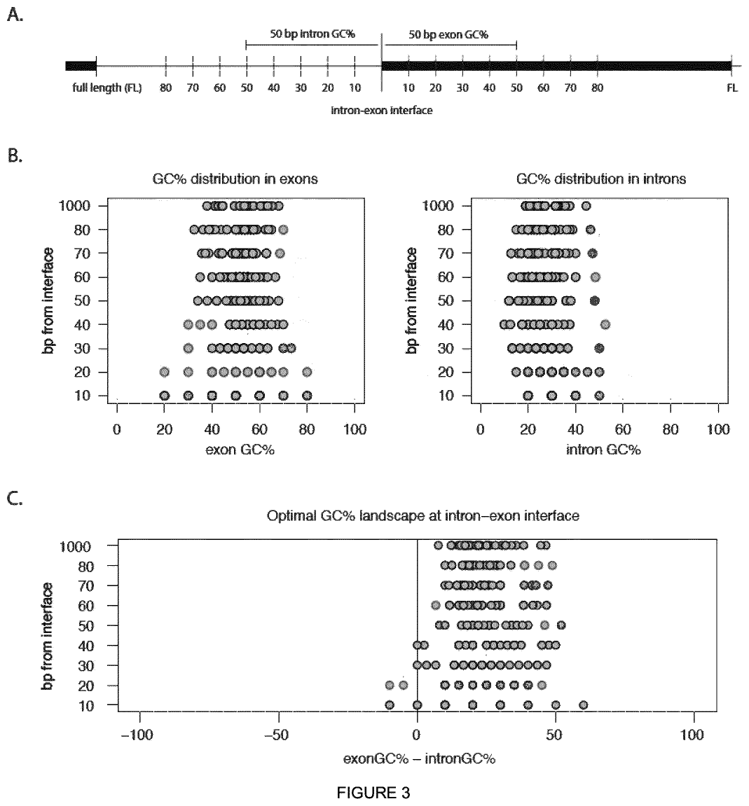

The GC % at intron-exon interface was calculated for every intron-exon pair (intron and it's following exon). The GC % was measured as above for various sequence lengths starting from the interface in 3′ to 5′ direction for the intron and in 5′ to 3′ direction for the exon, illustrated for 50 bp segments in FIG. 3A. All calculations were carried out using in-house python scripts and plotted in R.

### Cell Lines

293FT cells were obtained from Dr. Kosuke Yusa's Lab. 293FT.Cas9 cell lines were generated through lentiviral integration of EF1a-Cas9-T2A-BlastR construct at low MOI to achieve single-copy integration. To generate cell lines permissive to Spike-Pseudotyped lentiviral infection, 293FT.Cas9 cells were engineered to stably express SARS-COV-2 receptors ACE2 and TMPRSS2. PiggyBac transposition was used to integrate EF1a-ACE2-T2A-TMPRSS2 constructs followed by single-cell cloning. This resulted in 293FT.Cas9.ACE2/TMPRSS2 clonal cell lines. Clones C10 and D10 were used in this work. 293T cells were obtained from Dr. Ravindra Gupta Lab and used mostly for Spike-Pseudotyped lentivirus production. JM8 mouse embryonic stem cell line was derived from B57BL/6N blastocyst (Pettitt et al. 2009). MC38 cells were purchased from Kerafast (cat. 2388609). All cell lines have tested negative for mycoplasma contamination.

### Cell Culture Conditions

Unless stated otherwise, all lines were maintained at 5% CO2 and 37° C. 293FT, 293T and MC38 cell lines were routinely cultured in M10 media (DMEM, 10% FBS and 2 mM L-glutamine). Cas9 expressing cell lines were maintained in M10 supplemented with 10 μg/mL Blasticidin. JM8 cells were maintained in M15 medium (DMEM, 15% FBS, 100 μM b-mercaptoethanol, and 2 mM L-glutamine), on a layer of irradiated feeder fibroblasts (SNL76/7).

### Cell Transfections

Cell transfection was carried out using Lipofectamine LTX Reagent (Invitrogen) according to the manufacturer's instructions. For 6-well format transfections, Lipofectamine:DNA complexes were formulated using 750 ng DNA, 5 μL Plus reagent and 10 μL Lipofectamine LTX. These were then used to transfect 1.5 million cells per reaction. For analysis of transgene expression, cells were harvested with trypsin generally 48 h post-transfection. Samples were kept as frozen cell pellet for cDNA analysis or used directly for flow cytometry assays. For MC38 cells, transfections were performed using Amaxa Nucleofector (Lonza) using programme H-022 and Nucleofector kit V, according to the manufacturer's instructions. For the transfections, 0.41 pmols of DNA were transfected into one million cells per reaction.

cDNA Analysis

RNA was extracted from the frozen cell pellets using RNeasy Mini Kit (Qiagen) and treated with ezDNase (ThermoFisher) before applying oligo(dT) guided 1st strand cDNA synthesis using SuperScript VI reverse transcriptase (ThermoFisher), all according to manufactures' recommendations. RT-PCR was carried out using GoTaq Green Master Mix (Promega), following recommended protocol. PCR primers used to capture the entire length of investigated transgenes are enlisted in Table 2.

PCR products were both visualized on an agarose gel as well as TA-cloned using TA Cloning Kit with pCR2.1 vector and OneShot TOP10 Chemically Competent E. coli (ThermoFisher) according to kit instructions. After overnight growth on LB plates containing 100 μg/ml ampicillin at 37° C., single colonies were picked into 20 μl of PBS and the respective vector insert was PCR amplified with M13F (GTAAAACGACGGCCAGT) and M13R (CAGGAAACAGCTATGAC) primers, using GoTaq Green Master Mix. These PCR products were purified using AmPure XP magnetic beads (Beckman Coulter) following manufacture's recommendations and submitted to Sanger Sequencing (supplied by Source BioScience Inc) using the above M13F/M13R primers. On average, 24 clones per construct were assessed by PCR and 8 clones further selected for Sanger sequencing. All reads were mapped back to the original construct DNA sequence using SnapGene software to assess individual mRNA splicing events.

### Flow Cytometry Assays

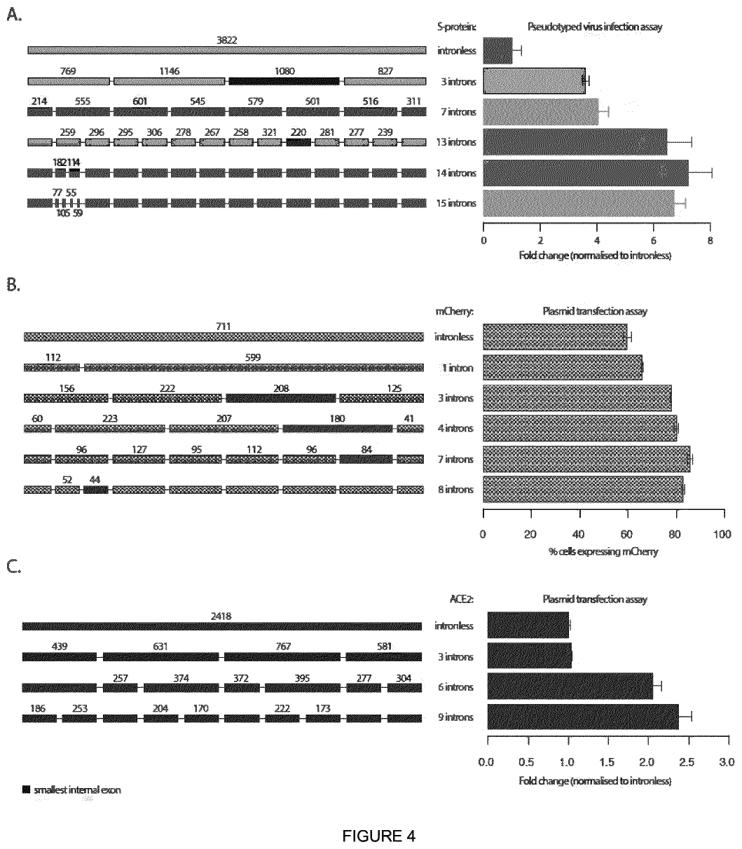

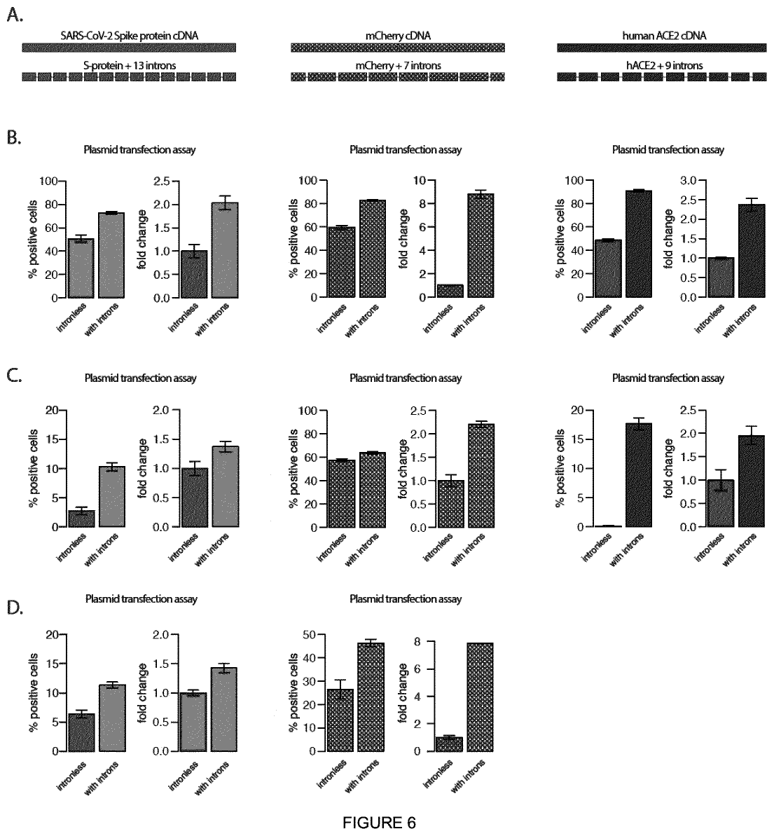

Cells were harvested 48 h post-transfection, using trypsin dissociation. For analysis of mCherry expression, cells were directly assessed by flow cytometry. When surface staining was needed, upon harvesting, cells were washed twice with staining buffer (see Table 3). They were then incubated with the appropriate dilution of primary antibody (in staining buffer) for 30 min at the indicated temperature. Cells were washed twice and incubated with secondary antibody (1:500) for 30 min on ice (for non-conjugated primary antibodies). Following another set of two washes, cells were analysed by flow cytometry using Cytoflex (BD Biosciences). Data analysis was performed using FlowJo software (BD Biosciences). S protein expression data is plotted as % of positively stained cells (FIG. 2). mCherry is shown as % cells expressing mCherry (FIG. 4) and as the population mCherry intensity median value normalized to intronless construct to highlight the shift in population intensity (FIG. 6). Same visualization is used for S protein and ACE2 constructs in FIG. 6.

### Pseudotyped Lentivirus Production

Pseudotyped Lentivirus was produced by transfection of 293T cells using lipofectamine LTX according to the manufacturer's instructions. All S protein constructs were tested using three independent virus productions. Briefly, 1 million 293T cells were seeded into gelatinized 6-well plates one day ahead of transfection. For transfection, 1 μg of lentiviral transfer vector (pCSGW-GFP), were mixed with 0.72 μg of gag-pol expressing plasmid p8.9 and 68.33 fmol of S protein expressing construct in 500 μL of optiMEM media followed by the addition of 2 μL of PLUS reagent and incubation for 5 minutes at room temperature. 6 μL of Lipofectamine LTX reagent were then added to the mix and incubated for 10 minutes. Medium was aspirated from the plates and the Lipofectamine:DNA complexes were added dropwise and topped-up with 1.5 mL of M10. Production was carried out at 5% CO2 and 32° C. Medium was changed the following morning to 2.5 mL of fresh M10 and the supernatant was harvested 56 hours later. Virus-containing supernatant was spun down at 500 g for 5 minutes to remove cell debris and used directly to infect permissive cell lines or aliquoted and frozen at −80° ° C.

### Permissive Cell Line Transduction

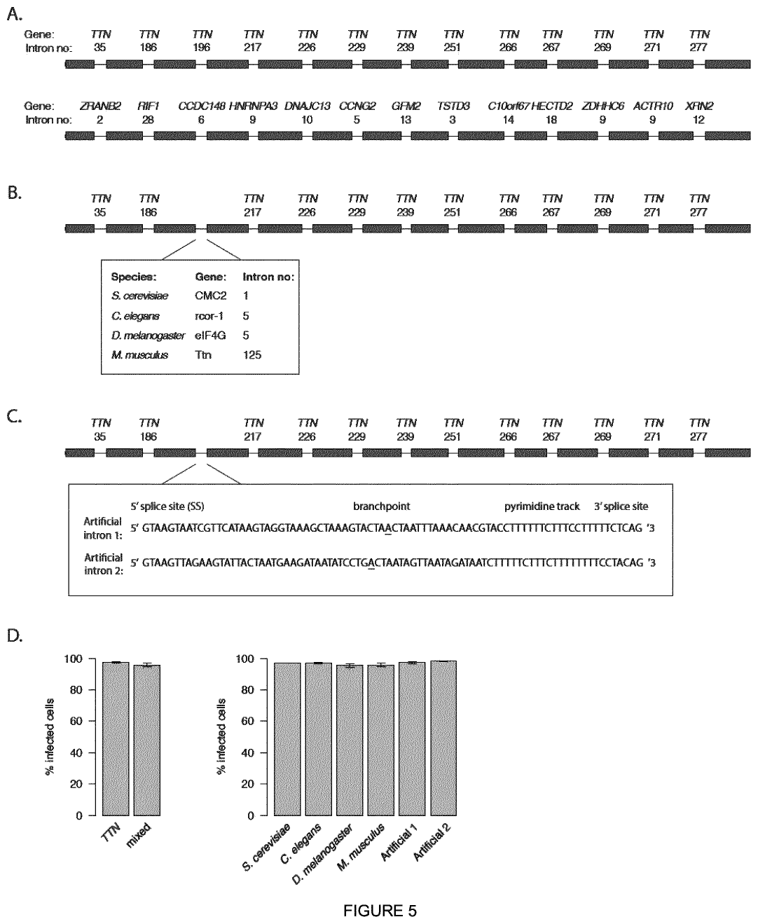

Transductions were carried-out in 96-well plates, in duplicates for each independent virus sample. For pseudotyped lentivirus titrations, a dilution series was prepared ranging from 100% virus-containing supernatant to 1:500 dilution in a total volume of 200 μL M10 medium. 293FT.Cas9.ACE2/TMPRSS2 clonal cell lines were harvested by trypsinization and resuspend at a density of 70.000 cells per 30 μL. They were then seeded, 30 μL per well, mixed and incubated at 37° C. Viral infection efficiency was measured 48-72 h later, assessed by the percentage of GFP positive cells on flow cytometry. Data was analysed using FlowJo software (BD Biosciences). Pseudotyped lentivirus infection assay data is displayed either as % cells infected with the full dose of pseudotyped virus (FIG. 5) or at 1:500 dilution, normalized to the intronless construct infection rates (FIG. 4).

### Results

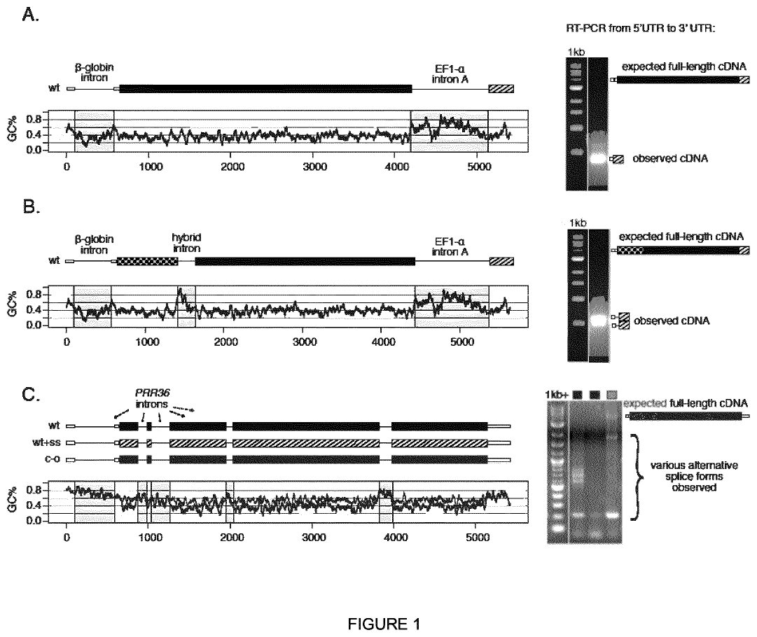

Addition of multiple introns to SARS-COV-2 Spike protein leads to alternatively spliced mRNA products. Wildtype (wt) SARS-COV-2 Spike (S) protein coding sequence (CDS) has proved difficult to be express as a transgene (Chen Ling 2020), similar to its related species SARS-COV Spike protein (Callendret et al. 2007). To improve its expression, two constructs with additional introns added to the wt S CDS were generated. While various intron insertion sites exist in endogenous genes (as well as in functional transgenes here), there is a slight preference in human canonical splicing consensus motifs for a sequence ‘(C/A)AG’ before intron and ‘G(T/N)(T/N)’ after intron (Sibley et al. 2016). For optimal placement of these introns, we looked for the presence of ‘CAGGTT’ nucleotide sequences in wt S protein, or for an opportunity to achieve that sequence using codon-optimisation. In wt S-protein, amino acid sequence ‘SGW’ in position 256-258 is encoded by ‘TCA-G|-intron-|GT-TGG’, containing the desired sequence (underlined) and hence providing an intron insertion site at the indicated location in G257. An opportunity for codon-optimised insertion site is available at amino acid sequence ‘DRL’ in position 1184-1186, where original nucleotide sequence: ‘GAC-CGC-CTC’ could be codon-optimised into an optimal intron insertion site: ‘GAC-AG|-intron-|G-TTG’ at amino acid R1185. The first generated construct (SEQ ID NO: 1 (P91), FIG. 1A) had an EF1-α intron A (sequence from EF1-α promoter) inserted in-between R1185 and a 5′UTR β-globin intron. The second construct (SEQ ID NO: 2 (P92), FIG. 1B) had a hybrid chicken β-actin/minute virus of mice intron (sequence from CBh promoter, (Gray et al. 2011)) inserted to G257.

S protein expression was measured 48 h after transfection into Hek293 cells but was not detected on the surface of the cells (data not shown). To investigate the reason, RT-PCR was conducted which detected a few strongly preferred alternatively spliced cDNA products. Sanger sequencing identified these products to consist of correct external exons of the S CDS construct, while the internal coding sequence was not incorporated in the mature transcript. It was removed via alternative splicing using either the canonical or cryptic splice sites within the introduced introns (FIG. 1A-B). While these introns are frequently and very successfully spliced within their 5′UTR context, they are not automatically recognized as independent intronic units outside that context and end up interacting with neighbouring introns.

As the above-used introns do not exist together within the same gene in a natural setting, consecutive introns from endogenous human genes were tested next. The gene PRR36 (Genbank: NM_001190467) was identified as a potentially good intron donor due to its short introns but similar length CDS in relation to S protein. A vector was generated in which all PRR36 introns were inserted into S CDS, maintaining their endogenous 5′ to 3′ order and their nucleotide sequence setting (3 bp before and after intron, where possible). To some extent the exon length was consistent with the PRR36 structure (SEQ ID NO: 3 (P113), FIG. 1C). Finally, the 5′UTR β-globin intron was replaced by the PRR36 5′UTR sequence to avoid any exogenous intron interactions. Despite using consecutive endogenous introns in SEQ ID NO: 3 (P113), the cryptic splicing outcomes persisted, displaying use of both canonical and cryptic splice sites within the inserted introns as well as in S CDS in various combinations (FIG. 1C). A second attempt involved using all introns from human gene EMILIN1 (NM_007046) and a third attempt was made using a sub-set of introns from human gene TTN (NC_000002.12) in wt S CDS (data not shown). In neither case was expression achieved at measurable levels and extensive cryptic splicing persisted.

To assess if the wt S CDS was driving the observed cryptic splicing in SEQ ID NO: 3 (P113), two more constructs were generated. First, 170 point mutations were introduced to wt S CDS to eliminate all cryptic splice sites identified in wt sequence using Alternative Splice Site Predictor (http://wangcomputing.com/assp/index.html) while retaining identical amino acid sequence and maintaining a similar GC % landscape (wt+ss, SEQ ID NO: 4 (P136), FIG. 1C). Second, the entire S CDS was codon-optimized (c-o, SEQ ID NO: 5 (P1486), FIG. 1C), a common practice to enhance transgene expression that does result in improved expression of S in intronless setting (FIG. 2B). Despite making these changes to the S CDS, the cryptic splicing continued with no or very little full-length cDNA observed (FIG. 1C).

Taking these data together, we confirm the addition of multiple introns, either well-defined ones from the literature and data bases or well used ones that are present in widely used expression systems, does not result in improved transgene expression due an underlying problem of alternative splicing. In other words, the assumption that a mammalian gene architecture that is sufficient for robust gene expression can be assembled merely by inserting numerous introns and removing cryptic sites was shown to be manifestly incorrect.

GC % Landscape that Enables Clear Definition of Exons and Intron

Amit et al. observed that genes in low GC % genomic regions tend to have large AT-rich introns with a clear GC % gradient at intron-exon interface (Amit et al. 2012). Whether this merely reflects the underlying bias between coding exons and non-coding introns in low GC % regions or is functionally significant requires experimental assessment. To test the effect of intron-exon GC % gradient in the context of transgenes with relatively short introns, a local and systematic change was introduced to a non-functional construct SEQ ID NO: 6 (P143). This construct consisted of 13 short introns from human TTN gene inserted into the wt-ss S sequence. Transfection of SEQ ID NO: 6 (P143) resulted in various alternative splicing outcomes (FIG. 2A) and no measurable full-size S protein (FIG. 2B). In this construct the intron 1/exon 2 interface had a very similar GC % profile. The GC % of the first 60 bp of exon 2 was increased from 38% to 60% (SEQ ID NO: 7 (P172)) by choosing codons with maximum number of G/C nucleotides where possible. The splicing outcomes were markedly affected by this change. From the analysis of cryptically spliced mRNA produced by SEQ ID NO: 6 (P143), a failure to recognize and therefore include exon 2 into the final mRNA transcript was apparent. However, the GC % increase of exon 2 in SEQ ID NO: 7 (P172) was sufficient to now include that region into all identified splicing outcomes (FIG. 2A). Extending the same strategy across the entire length of all exons (SEQ ID NO: 11 (P171)), resulted in correct splicing of all 13 introns and furthermore improved the expression of S protein compared to all previous attempts of intronization as well as the intronless transgene with identical CDS sequence (FIG. 2B). Taken together, an intron-exon GC % gradient can successfully define intron-exon borders in a transgene setting. Such a gradient can in principle be achieved by either increasing GC % of exons using codon-optimization (applied here in SEQ ID NO: 11 (P171)), or by inserting introns with lower GC % into an unchanged CDS sequence (applied here in SEQ ID NO: 6 (P143)), or a combination of both.

In order to further characterize what defines a functional intron-exon interface, GC % was calculated for different length segments of DNA (10-80 bp+full length of the element) measured from the interface outwards for 29 neighbouring intron-exon pairs from 3 different correctly splicing constructs (SEQ ID NO: 11 (P171), SEQ ID NO: 14 (P186), SEQ ID NO: 25 (P237), SEQ ID NO: 30 (P243), FIG. 3A). The proportion of G/C nucleotides in exons varied both within and in-between different transgenes (20-80%) similar to inserted introns where the overall GC % range was both wide (10-52%) and overlapping with exons (FIG. 3B). When neighbouring introns and exons were assessed in pairs, the exons were with at least equal, and usually higher GC % compared to the preceding intron (FIG. 3C). This feature was consistently present at every segment length starting from 30 bp, indicating that length of 10-20 bp might be too short of a measurement window for accurate assessment of GC % landscape.

### Definition of Optimal Exon Length

After solving the critical landscape requirements for correct intron and exon recognition in transgenes, the number of optimal introns could be addressed. An increasing number of introns were inserted into the SEQ ID NO: 8 (P166) sequence: 3, 7, 13, 14, 15 introns (SEQ ID NO: 9 (P205), SEQ ID NO: 10 (P204), SEQ ID NO: 11 (P171), SEQ ID NO: 12 (P231), SEQ ID NO: 13 (P232), FIG. 4A) and the effect on S protein expression was assessed in a functional assay of pseudotyped S protein virus infections where S protein is expressed to produce an infective but replication defective virion. The infectiveness of these viral particles will depend on the density and function of the S protein on their surface and is conveniently assessed if the packaged viral genome carries a reporter. The results in FIG. 4A are displayed in relation to the construct without introns. Improvement in expression was seen with addition of a few introns and gradually improved until one of the internal exons of S was reduced to 55 bp (15 intron construct).

A similar outcome was observed when intronizing mCherry CDS with 1, 3, 4, 7 or 8 introns (SEQ ID NO: 21 (P233) to SEQ ID NO: 25 (P237), FIG. 4B). mCherry CDS is a relatively short sequence compared to S (711 bp versus 3822 bp) and therefore the number of introns required to achieve similar internal exon sizes is lower. Nevertheless, the expression gradually improved with more introns until smallest internal exons were reduced to ˜50 bp, shown as % cells expressing mCherry (FIG. 4B).

Similar improvement in expression was seen when intronizing human ACE2 CDS, most prominently with the addition of 6 and 9 introns (SEQ ID NO: 27 (P95), SEQ ID NO: 28 (P223), SEQ ID NO: 29 (P242)-SEQ ID NO: 30 (P243), FIG. 4C). In this case, the exons only reached the optimal size range and hence no downward trend was observed.

Taken together, transgene expression could be improved with internal exons as large as 501 bp-1146 bp, but the optimal expression outcome required internal exon sizes to be between 84 bp-372 bp. These data are consistent with previous findings in human endogenous genes demonstrating the optimal exon length for efficient splicing to be between 50 bp and 250 bp (Movassat et al. 2019).

### Minimal Intron Requirements

To explore the type of intronic sequences that enable the formation of the correct intron-exon landscape in a multiple intron setting, a series of constructs were generated with different introns embedded into c-o S CDS (FIG. 5A-C). The functional performance of the S expressed from these constructs was assessed by infection rates of the respective pseudotyped viruses (FIG. 5D). First, 13 endogenous introns, each originating from a different human gene, were inserted into S protein CDS (SEQ ID NO: 14 (P186), FIG. 5A). These introns were selected based on their short length, low GC % and presence of canonical splice site sequences. The construct containing these mixed introns resulted in equivalent S protein levels (FIG. 5D), confirming that introns do not need to originate from the same gene and operate as independent units.

Next, a number of exogenous introns with similar criteria were introduced into SEQ ID NO: 11 (P171) sequence, substituting the third intron (TTN intron 196). This included an intron from unicellular yeast (S. cerevisiae, CMC2, intron 1, SEQ ID NO: 15 (P226)), a nematode (C. elegans, rcor-1, intron 5, SEQ ID NO: 16 (P227)), a fruit fly (D. melanogaster, eIF4G, intron 5, SEQ ID NO: 17 (P228)) and a mouse (M. musculus, Ttn, intron 125, SEQ ID NO: 18 (P229)) (FIG. 5B). All the above constructs also resulted in similarly expressed S protein levels, highlighting the fact that the origin of the intronic sequences is an unimportant feature.

Given the above, we next introduced two artificial intronic sequences into the SEQ ID NO: 11 (P171) third intron position (SEQ ID NO: 19 (P230), SEQ ID NO: 20 (P241), FIG. 5C). Besides the commonly known intronic elements (splice sites, branchpoint and a pyrimidine track), the intronic sequences were created at random and solely guided by the overall GC % of the intron, following the above-established guidelines (FIG. 3). Both artificial introns performed equally well in comparison to the other constructs (FIG. 5D) showing that optimal GC % in addition to known intronic elements was sufficient for an intron to be spliced correctly and thus establishing the minimal requirements for a functional intron within an intronized transgene setting.

Intronization Leads to Improved Expression Levels within Various Contexts

Above-developed rules for optimal transgene expression using multiple introns was tested in the context of different transgenes and cell lines (FIG. 6). First, 13 introns (internal exons: 220 bp-306 bp) were inserted to a cytoplasmic viral S protein CDS that naturally occurs only as an RNA sequence with no introns. Next, 7 introns (internal exons: 84 bp-127 bp) were added to a synthetic sequence, derived from Discosoma sp red fluorescent gene, using non-endogenous intron location sites (endogenous sites: http://corallimorpharia.reefgenomics.org). Lastly, 9 introns (internal exons: 170 bp-372 bp) were inserted to an endogenous human ACE2 CDS using a selection of its endogenous intron locations (FIG. 6A).

Above three intronized transgenes were first tested against their intronless counterparts in human 293FT cells. Intronized S protein (stained with antibodies), mCherry (direct measurement of fluorescence), and ACE2 (stained with antibodies) showed improvement both in percent of cells expressing the protein as well as in the amount of expression per cell, displayed as fold change difference in population median expression values (FIG. 6B). Expression improvements were also observed in mouse embryonic cell line JM8 (FIG. 6C) and mouse colon adenocarcinoma cell line MC38 lines (FIG. 6D), where none of the intronic or exonic sequences were endogenous.

TABLE 1

| | | | |
|---|---|---|---|
| SEQ | Construct name | 5′ and 3′ elements | plasmid |

SARS-COV-2 S protein cDNA constructs

| | | | |
|---|---|---|---|
| SEQ ID NO: 1 | S_wt_18F_EF1a | CMV promoter + β-globin intron; | pMD2.G |
| (P91) | | β-globin polyA | |
| SEQ ID NO: 2 | S_wt_18F_hybrid_EF1a | CMV promoter + β-globin intron; | pMD2.G |
| (P92) | | β-globin polyA | |
| SEQ ID NO: 3 | S_wt_PRR36 | CMV promoter; β-globin polyA | pMD2.G |
| (P113) | | | |
| SEQ ID NO: 4 | S_wt+ss_PRR36 | CMV promoter; β-globin polyA | pMD2.G |
| (P136) | | | |
| SEQ ID NO: 5 | S_c-o_PRR36 | CMV promoter; β-globin polyA | pMD2.G |
| (P148) | | | |
| SEQ ID NO: 6 | S_wt+ss_18F_TTN13 | EFS promoter; β-globin polyA | pMD2.G |
| (P143) | | | |
| SEQ ID NO: 7 | S_wt+ss_18F_TTN13_GC↑ | EFS promoter; β-globin polyA | pMD2.G |
| (P172) | | | |
| SEQ ID NO: 8 | S_c-o | CMV promoter; β-globin polyA | pMD2.G |
| (P166) | | | |
| SEQ ID NO: 9 | S_c-o_TTN3 | CMV promoter; β-globin polyA | pMD2.G |
| (P205) | | | |
| SEQ ID NO: 10 | S_c-o_TTN7 | CMV promoter; β-globin polyA | pMD2.G |
| (P204) | | | |
| SEQ ID NO: 11 | S_c-o_TTN13 | CMV promoter; β-globin polyA | pMD2.G |
| (P171) | | | |
| SEQ ID NO: 12 | S_c-o_TTN13_MI1 | CMV promoter; β-globin polyA | pMD2.G |
| (P231) | | | |
| SEQ ID NO: 13 | S_c-o_TTN13_MI2 | CMV promoter; β-globin polyA | pMD2.G |
| (P232) | | | |
| SEQ ID NO: 14 | S_c-o_MI13 | CMV promoter; β-globin polyA | pMD2.G |
| (P186) | | | |
| SEQ ID NO: 15 | S_c-o_TTN12_yeast | CMV promoter; β-globin polyA | pMD2.G |
| (P226) | | | |
| SEQ ID NO: 16 | S_c-o_TTN12_worm | CMV promoter; β-globin polyA | pMD2.G |
| (P227) | | | |
| SEQ ID NO: 17 | S_c-o_TTN12_fly | CMV promoter; β-globin polyA | pMD2.G |
| (P228) | | | |
| SEQ ID NO: 18 | S_c-o_TTN12_mouse | CMV promoter; β-globin polyA | pMD2.G |
| (P229) | | | |
| SEQ ID NO: 19 | S_c-o_TTN12_artificial1 | CMV promoter; B-globin polyA | pMD2.G |
| (P230) | | | |
| SEQ ID NO: 20 | S_c-o_TTN12_artificial2 | CMV promoter; β-globin polyA | pMD2.G |
| (P241) | | | |

mCherry constructs

| | | | |
|---|---|---|---|
| SEQ ID NO: 21 | mCherry | PGK promoter; SV40 late polyA | pUC19c |
| (P233) | | | |
| SEQ ID NO: 22 | mCherry_TTN1 | PGK promoter; SV40 late polyA | pUC19c |
| (P234) | | | |
| SEQ ID NO: 23 | mCherry_TTN3 | PGK promoter; SV40 late polyA | pUC19c |
| (P235) | | | |
| SEQ ID NO: 24 | mCherry_TTN4 | PGK promoter; SV40 late polyA | PUC19c |
| (P236) | | | |
| SEQ ID NO: 25 | mCherry_TTN7 | PGK promoter; SV40 late polyA | PUC19c |
| (P237) | | | |
| SEQ ID NO: 26 | mCherry_TTN8 | PGK promoter; SV40 late polyA | PUC19c |
| (P238) | | | |

ACE2 constructs

| | | | |
|---|---|---|---|
| SEQ ID NO: 27 | ACE2 | PGK promoter; bGH polyA | pKLV |
| (SEQ ID NO: 27 | | | (pBluescript) |
| (P95)) | | | |
| SEQ ID NO: 28 | ACE2_TTN3 | PGK promoter; bGH polyA | pKLV |
| (P223) | | | (pBluescript) |
| SEQ ID NO: 29 | ACE2_TTN4_MI2 | PGK promoter; bGH polyA | pKLV |
| (P242) | | | (pBluescript) |
| SEQ ID NO: 30 | ACE2_TTN7_MI2 | PGK promoter; bGH polyA | pKLV |
| (P243) | | | (pBluescript) |

TABLE 2

| | Primer | |
|---|---|---|
| SEQ | name | Sequence (5′ to 3′) |
| SEQ ID NO: 1 (P91), | 5′UTR_ | ATCCACGCTGTTTTGACCT |
| SEQ ID NO: 2 (P92) | β-globin_ | C |
| | intron_F | |
| SEQ ID NO: 1 (P91), | FLAG_R | TCATCGTCATCCTTGTAGT |
| SEQ ID NO: 2 (P92) | | CGA |
| SEQ ID NO: 3 (P113), | 5′UTR_ | GTTTAGTGAACCGTCAGAT |
| SEQ ID NO: 4 (P136), | PRR36_F | CGCCTAGCCCCCTCCCCTC |
| SEQ ID NO: 5 (P1486) | | GCCTCC |
| SEQ ID NO: 3 (P113), | 3′UTR_ | CAATACACTGCACACGGGG |
| SEQ ID NO: 4 (P136), | PRR36_R | |
| SEQ ID NO: 5 (P1486) | | |
| SEQ ID NO: 6 (P143), | 5′UTR_ | GGTACCGCAGAGACAGGAG |
| SEQ ID NO: 7 (P172) | EFS_F | ATCTGCCACCATG |
| SEQ ID NO: 6 (P143), | 3′UTR_ | CAAGGCCCTTCATAATATC |
| SEQ ID NO: 7 (P172) | β-globin | CCC |
| | polyA_R | |

TABLE 3

| | | | Primary | Primary | |
|---|---|---|---|---|---|
| | Staining | Primary | Antibody | antibody | Secondary |
| Target | buffer | Antibody | dilution | incubation | Antibody |
| S | PBS + | Genetex | 1:400 | 30 min, | Goat anti- |
| | 3% FBS | GTX632604 | | 4 degrees | mouse AF488 |
| | | | | | (Invitrogen, |
| | | | | | A11029) |
| ACE2 | PBS + | ACE2-AF594 | 1:40 | 1 h, RT | Goat anti- |
| | 1% BSA | (Bio-Techne, | | | mouse AF594 |
| | | FAB9332T) | | | (Invitrogen |
| | | | | | A11005) |

Sequences

SEQ ID NO: 1 (P91)
GTCAGATCGCCTGGAGACGCCATCCACGCTGTTTTGACCTCCATAGAAGACACCGGGACCGATCCAGCCTCCCCTCGAAGCTTACAT
GTGGTACCGAGCTCGGATCCTGAGAACTTCAGGgtgagtctatgggacccttgatgttttctttccccttcttttctatggttaagt
tcatgtcataggaaggggagaagtaacagggtacacatattgaccaaatcagggtaattttgcatttgtaattttaaaaaatgcttt
cttcttttaatatacttttttgtttatcttatttctaatactttccctaatctctttctttcagggcaataatgatacaatgtatca
tgcctctttgcaccattctaaagaataacagtgataatttctgggttaaggcaatagcaatatttctgcatataaatatttctgcat
ataaattgtaactgatgtaagaggtttcatattgctaatagcagctacaatccagctaccattctgcttttattttatggttgggat
aaggctggattattctgagtccaagctaggcccttttgctaatcatgttcatacctcttatcttcctcccacagCTCCTGGGCAACG
TGCTGGTCTGTGTGCTGGCCCATCACTTTGGCAAAGCACAGGAGATCTGCCACCATGTTTGTTTTTCTTGTTTTATTGCCACTAGTC
TCTAGTCAGTGTGTTAATCTTACAACCAGAACTCAATTACCCCCTGCATACACTAATTCTTTCACACGTGGTGTTTATTACCCTGAC
AAAGTTTTCAGATCCTCAGTTTTACATTCAACTCAGGACTTGTTCTTACCTTTCTTTTCCAATGTTACTTGGTTCCATGCTATACAT
GTCTCTGGGACCAATGGTACTAAGAGGTTTGATAACCCTGTCCTACCATTTAATGATGGTGTTTATTTTGCTTCCACTGAGAAGTCT
AACATAATAAGAGGCTGGATTTTTGGTACTACTTTAGATTCGAAGACCCAGTCCCTACTTATTGTTAATAACGCTACTAATGTTGTT
ATTAAAGTCTGTGAATTTCAATTTTGTAATGATCCATTTTTGGGTGTTTATTACCACAAAAACAACAAAAGTTGGATGGAAAGTGAG
TTCAGAGTTTATTCTAGTGCGAATAATTGCACTTTTGAATATGTCTCTCAGCCTTTTCTTATGGACCTTGAAGGAAAACAGGGTAAT
TTCAAAAATCTTAGGGAATTTGTGTTTAAGAATATTGATGGTTATTTTAAAATATATTCTAAGCACACGCCTATTAATTTAGTGCGT
GATCTCCCTCAGGGTTTTTCGGCTTTAGAACCATTGGTAGATTTGCCAATAGGTATTAACATCACTAGGTTTCAAACTTTACTTGCT
TTACATAGAAGTTATTTGACTCCTGGTGATTCTTCTTCAGGTTGGACAGCTGGTGCTGCAGCTTATTATGTGGGTTATCTTCAACCT
AGGACTTTTCTATTAAAATATAATGAAAATGGAACCATTACAGATGCTGTAGACTGTGCACTTGACCCTCTCTCAGAAACAAAGTGT
ACGTTGAAATCCTTCACTGTAGAAAAAGGAATCTATCAAACTTCTAACTTTAGAGTCCAACCAACAGAATCTATTGTTAGATTTCCT
AATATTACAAACTTGTGCCCTTTTGGTGAAGTTTTTAACGCCACCAGATTTGCATCTGTTTATGCTTGGAACAGGAAGAGAATCAGC
AACTGTGTTGCTGATTATTCTGTCCTATATAATTCCGCATCATTTTCCACTTTTAAGTGTTATGGAGTGTCTCCTACTAAATTAAAT
GATCTCTGCTTTACTAATGTCTATGCAGATTCATTTGTAATTAGAGGTGATGAAGTCAGACAAATCGCTCCAGGGCAAACTGGAAAG
ATTGCTGATTATAATTATAAATTACCAGATGATTTTACAGGCTGCGTTATAGCTTGGAATTCTAACAATCTTGATTCTAAGGTTGGT
GGTAATTATAATTACCTGTATAGATTGTTTAGGAAGTCTAATCTCAAACCTTTTGAGAGAGATATTTCAACTGAAATCTATCAGGCC
GGTAGCACACCTTGTAATGGTGTTGAAGGTTTTAATTGTTACTTTCCTTTACAATCATATGGTTTCCAACCCACTAATGGTGTTGGT
TACCAACCATACAGAGTAGTAGTACTTTCTTTTGAACTTCTACATGCACCAGCAACTGTTTGTGGACCTAAAAAGTCTACTAATTTG
GTTAAAAACAAATGTGTCAATTTCAACTTCAATGGTTTAACAGGCACAGGTGTTCTTACTGAGTCTAACAAAAAGTTTCTGCCTTTC
CAACAATTTGGCAGAGACATTGCTGACACTACTGATGCTGTCCGTGATCCACAGACACTTGAGATTCTTGACATTACACCATGTTCT
TTTGGTGGTGTCAGTGTTATAACACCAGGAACAAATACTTCTAACCAGGTTGCTGTTCTTTATCAGGATGTTAACTGCACAGAAGTC
CCTGTTGCTATTCATGCAGATCAACTTACTCCTACTTGGCGTGTTTATTCTACAGGTTCTAATGTTTTTCAAACACGTGCAGGCTGT
TTAATAGGGGCTGAACATGTCAACAACTCATATGAGTGTGACATACCCATTGGTGCAGGTATATGCGCTAGTTATCAGACTCAGACT
AATTCTCCTCGGCGGGCACGTAGTGTAGCTAGTCAATCCATCATTGCCTACACTATGTCACTTGGTGCAGAAAATTCAGTTGCTTAC
TCTAATAACTCTATTGCCATACCCACAAATTTTACTATTAGTGTTACCACAGAAATTCTACCAGTGTCTATGACCAAGACATCAGTA
GATTGTACAATGTACATTTGTGGTGATTCAACTGAATGCAGCAATCTTTTGTTGCAATATGGCAGTTTTTGTACACAATTAAACCGT
GCTTTAACTGGAATAGCTGTTGAACAAGACAAAAACACCCAAGAAGTTTTTGCACAAGTCAAACAAATTTACAAAACACCACCAATT
AAAGATTTTGGTGGTTTTAATTTTTCACAAATATTACCAGATCCATCAAAACCAAGCAAGAGGTCATTTATTGAAGATCTACTTTTC
AACAAAGTGACACTTGCAGATGCTGGCTTCATCAAACAATATGGTGATTGCCTTGGTGATATTGCTGCTAGAGACCTCATTTGTGCA
CAAAAGTTTAACGGCCTTACTGTTTTGCCACCTTTGCTCACAGATGAAATGATTGCTCAATACACTTCTGCACTGTTAGCGGGTACA
ATCACTTCTGGTTGGACCTTTGGTGCAGGTGCTGCATTACAAATACCATTTGCTATGCAAATGGCTTATAGGTTTAATGGTATTGGA
GTTACACAGAATGTTCTCTATGAGAACCAAAAATTGATTGCCAACCAATTTAATAGTGCTATTGGCAAAATTCAAGACTCACTTTCT
TCCACAGCAAGTGCACTTGGAAAACTTCAAGATGTGGTCAACCAAAATGCACAAGCTTTAAACACGCTTGTTAAACAACTTAGCTCC
AATTTTGGTGCAATTTCAAGTGTTTTAAATGATATCCTTTCACGTCTTGACAAAGTTGAGGCTGAAGTGCAAATTGATAGGTTGATC
ACAGGCAGACTTCAAAGTTTGCAGACATATGTGACTCAACAATTAATTAGAGCTGCAGAAATCAGAGCTTCTGCTAATCTTGCTGCT
ACTAAAATGTCAGAGTGTGTACTTGGACAATCAAAAAGAGTTGATTTTTGTGGAAAGGGCTATCATCTTATGTCCTTCCCTCAGTCA
GCACCTCATGGTGTAGTCTTCTTGCATGTGACTTATGTCCCTGCACAAGAAAAGAACTTCACAACTGCTCCTGCCATTTGTCATGAT
GGAAAAGCACACTTTCCTCGTGAAGGTGTCTTTGTTTCAAATGGCACACACTGGTTTGTAACACAAAGGAATTTTTATGAACCACAA
ATCATTACTACAGACAACACATTTGTGTCTGGTAACTGTGATGTTGTAATAGGAATTGTCAACAACACAGTTTATGATCCTTTGCAA
CCTGAATTAGACTCATTCAAGGAGGAGTTAGATAAATATTTTAAGAATCATACATCACCAGATGTTGATTTAGGTGACATCTCTGGC
ATTAATGCTTCAGTTGTAAACATTCAAAAAGAAATTGACAGgtaagtgccgtgtgtggttcccgcgggcctggcctctttacgggtt
atggcccttgcgtgccttgaattacttccacctggctgcagtacgtgattcttgatcccgagcttcgggttggaagtgggtgggaga
gttcgaggccttgcgcttaaggagccccttcgcctcgtgcttgagttgaggcctggcctgggcgctggggccgccgcgtgcgaatct
ggtggcaccttcgcgcctgtctcgctgctttcgataagtctctagccatttaaaatttttgatgacctgctgcgacgctttttttct
ggcaagatagtcttgtaaatgcgggccaagatctgcacactggtatttcggtttttggggccgcgggcggcgacggggcccgtgcgt
cccagcgcacatgttcggcgaggcggggcctgcgagcgcggccaccgagaatcggacgggggtagtctcaagctggccggcctgctc
tggtgcctggcctcgcgccgccgtgtatcgccccgccctgggcggcaaggctggcccggtcggcaccagttgcgtgagcggaaagat
ggccgcttcccggccctgctgcagggagctcaaaatgaaggacgcggcgctcgggagagcgggcgggtgagtcacccacacaaagga
aaagggcctttccgtcctcagccgtcgcttcatgtgactccacggagtaccgggcgccgtccaggcacctcgattagttctcgagct
tttggagtacgtcgtctttaggttggggggaggggttttatgcgatggagtttccccacactgagtgggtggagactgaagttaggc
cagcttggcacttgatgtaattctccttggaatttgccctttttgagtttggatcttggttcattctcaagcctcagacagtggttc
aaagtttttttcttccatttcagGTTGAATGAGGTTGCCAAGAATTTAAATGAATCTCTCATCGATCTCCAAGAACTTGGAAAGTAT
GAGCAGTATATAAAATGGCCATGGTACATTTGGCTAGGTTTTATAGCTGGCTTGATTGCCATAGTAATGGTGACAATTATGCTTTGC
TGTATGACCAGTTGCTGTAGTTGTCTCAAGGGCTGTTGTTCTTGTGGATCCTGCTGCAAAGGCGGCGGGTCCGGAGGAGACTACAAA
GACCATGACGGTGATTATAAAGATCATGACATCGACTACAAGGATGACGATGACAAGTAG
SEQ ID NO: 2 (P92)
GTCAGATCGCCTGGAGACGCCATCCACGCTGTTTTGACCTCCATAGAAGACACCGGGACCGATCCAGCCTCCCCTCGAAGCTTACAT
GTGGTACCGAGCTCGGATCCTGAGAACTTCAGGgtgagtctatgggacccttgatgttttctttccccttcttttctatggttaagt
tcatgtcataggaaggggagaagtaacagggtacacatattgaccaaatcagggtaattttgcatttgtaattttaaaaaatgcttt
cttcttttaatatacttttttgtttatcttatttctaatactttccctaatctctttctttcagggcaataatgatacaatgtatca
tgcctctttgcaccattctaaagaataacagtgataatttctgggttaaggcaatagcaatatttctgcatataaatatttctgcat
ataaattgtaactgatgtaagaggtttcatattgctaatagcagctacaatccagctaccattctgcttttattttatggttgggat
aaggctggattattctgagtccaagctaggcccttttgctaatcatgttcatacctcttatcttcctcccacagCTCCTGGGCAACG
TGCTGGTCTGTGTGCTGGCCCATCACTTTGGCAAAGCACAGGAGATCTGCCACCATGTTTGTTTTTCTTGTTTTATTGCCACTAGTC
TCTAGTCAGTGTGTTAATCTTACAACCAGAACTCAATTACCCCCTGCATACACTAATTCTTTCACACGTGGTGTTTATTACCCTGAC
AAAGTTTTCAGATCCTCAGTTTTACATTCAACTCAGGACTTGTTCTTACCTTTCTTTTCCAATGTTACTTGGTTCCATGCTATACAT
GTCTCTGGGACCAATGGTACTAAGAGGTTTGATAACCCTGTCCTACCATTTAATGATGGTGTTTATTTTGCTTCCACTGAGAAGTCT
AACATAATAAGAGGCTGGATTTTTGGTACTACTTTAGATTCGAAGACCCAGTCCCTACTTATTGTTAATAACGCTACTAATGTTGTT
ATTAAAGTCTGTGAATTTCAATTTTGTAATGATCCATTTTTGGGTGTTTATTACCACAAAAACAACAAAAGTTGGATGGAAAGTGAG
TTCAGAGTTTATTCTAGTGCGAATAATTGCACTTTTGAATATGTCTCTCAGCCTTTTCTTATGGACCTTGAAGGAAAACAGGGTAAT
TTCAAAAATCTTAGGGAATTTGTGTTTAAGAATATTGATGGTTATTTTAAAATATATTCTAAGCACACGCCTATTAATTTAGTGCGT
GATCTCCCTCAGGGTTTTTCGGCTTTAGAACCATTGGTAGATTTGCCAATAGGTATTAACATCACTAGGTTTCAAACTTTACTTGCT
TTACATAGAAGTTATTTGACTCCTGGTGATTCTTCTTCAGggagtcgctgcgacgctgccttcgccccgtgccccgctccgccgccg
cctcgcgccgcccgccccggctctgactgaccgcgttactcccacaggtgagcgggcgggacggcccttctcctccgggctgtaatt
agctgagcaagaggtaagggtttaagggatggttggttggtggggtattaatgtttaattacctggagcacctgcctgaaatcactt
tttttcagGTTGGACAGCTGGTGCTGCAGCTTATTATGTGGGTTATCTTCAACCTAGGACTTTTCTATTAAAATATAATGAAAATGG
AACCATTACAGATGCTGTAGACTGTGCACTTGACCCTCTCTCAGAAACAAAGTGTACGTTGAAATCCTTCACTGTAGAAAAAGGAAT
CTATCAAACTTCTAACTTTAGAGTCCAACCAACAGAATCTATTGTTAGATTTCCTAATATTACAAACTTGTGCCCTTTTGGTGAAGT
TTTTAACGCCACCAGATTTGCATCTGTTTATGCTTGGAACAGGAAGAGAATCAGCAACTGTGTTGCTGATTATTCTGTCCTATATAA
TTCCGCATCATTTTCCACTTTTAAGTGTTATGGAGTGTCTCCTACTAAATTAAATGATCTCTGCTTTACTAATGTCTATGCAGATTC
ATTTGTAATTAGAGGTGATGAAGTCAGACAAATCGCTCCAGGGCAAACTGGAAAGATTGCTGATTATAATTATAAATTACCAGATGA
TTTTACAGGCTGCGTTATAGCTTGGAATTCTAACAATCTTGATTCTAAGGTTGGTGGTAATTATAATTACCTGTATAGATTGTTTAG
GAAGTCTAATCTCAAACCTTTTGAGAGAGATATTTCAACTGAAATCTATCAGGCCGGTAGCACACCTTGTAATGGTGTTGAAGGTTT
TAATTGTTACTTTCCTTTACAATCATATGGTTTCCAACCCACTAATGGTGTTGGTTACCAACCATACAGAGTAGTAGTACTTTCTTT
TGAACTTCTACATGCACCAGCAACTGTTTGTGGACCTAAAAAGTCTACTAATTTGGTTAAAAACAAATGTGTCAATTTCAACTTCAA
TGGTTTAACAGGCACAGGTGTTCTTACTGAGTCTAACAAAAAGTTTCTGCCTTTCCAACAATTTGGCAGAGACATTGCTGACACTAC
TGATGCTGTCCGTGATCCACAGACACTTGAGATTCTTGACATTACACCATGTTCTTTTGGTGGTGTCAGTGTTATAACACCAGGAAC
AAATACTTCTAACCAGGTTGCTGTTCTTTATCAGGATGTTAACTGCACAGAAGTCCCTGTTGCTATTCATGCAGATCAACTTACTCC
TACTTGGCGTGTTTATTCTACAGGTTCTAATGTTTTTCAAACACGTGCAGGCTGTTTAATAGGGGCTGAACATGTCAACAACTCATA
TGAGTGTGACATACCCATTGGTGCAGGTATATGCGCTAGTTATCAGACTCAGACTAATTCTCCTCGGCGGGCACGTAGTGTAGCTAG
TCAATCCATCATTGCCTACACTATGTCACTTGGTGCAGAAAATTCAGTTGCTTACTCTAATAACTCTATTGCCATACCCACAAATTT
TACTATTAGTGTTACCACAGAAATTCTACCAGTGTCTATGACCAAGACATCAGTAGATTGTACAATGTACATTTGTGGTGATTCAAC
TGAATGCAGCAATCTTTTGTTGCAATATGGCAGTTTTTGTACACAATTAAACCGTGCTTTAACTGGAATAGCTGTTGAACAAGACAA
AAACACCCAAGAAGTTTTTGCACAAGTCAAACAAATTTACAAAACACCACCAATTAAAGATTTTGGTGGTTTTAATTTTTCACAAAT
ATTACCAGATCCATCAAAACCAAGCAAGAGGTCATTTATTGAAGATCTACTTTTCAACAAAGTGACACTTGCAGATGCTGGCTTCAT
CAAACAATATGGTGATTGCCTTGGTGATATTGCTGCTAGAGACCTCATTTGTGCACAAAAGTTTAACGGCCTTACTGTTTTGCCACC
TTTGCTCACAGATGAAATGATTGCTCAATACACTTCTGCACTGTTAGCGGGTACAATCACTTCTGGTTGGACCTTTGGTGCAGGTGC
TGCATTACAAATACCATTTGCTATGCAAATGGCTTATAGGTTTAATGGTATTGGAGTTACACAGAATGTTCTCTATGAGAACCAAAA
ATTGATTGCCAACCAATTTAATAGTGCTATTGGCAAAATTCAAGACTCACTTTCTTCCACAGCAAGTGCACTTGGAAAACTTCAAGA
TGTGGTCAACCAAAATGCACAAGCTTTAAACACGCTTGTTAAACAACTTAGCTCCAATTTTGGTGCAATTTCAAGTGTTTTAAATGA
TATCCTTTCACGTCTTGACAAAGTTGAGGCTGAAGTGCAAATTGATAGGTTGATCACAGGCAGACTTCAAAGTTTGCAGACATATGT
GACTCAACAATTAATTAGAGCTGCAGAAATCAGAGCTTCTGCTAATCTTGCTGCTACTAAAATGTCAGAGTGTGTACTTGGACAATC
AAAAAGAGTTGATTTTTGTGGAAAGGGCTATCATCTTATGTCCTTCCCTCAGTCAGCACCTCATGGTGTAGTCTTCTTGCATGTGAC
TTATGTCCCTGCACAAGAAAAGAACTTCACAACTGCTCCTGCCATTTGTCATGATGGAAAAGCACACTTTCCTCGTGAAGGTGTCTT
TGTTTCAAATGGCACACACTGGTTTGTAACACAAAGGAATTTTTATGAACCACAAATCATTACTACAGACAACACATTTGTGTCTGG
TAACTGTGATGTTGTAATAGGAATTGTCAACAACACAGTTTATGATCCTTTGCAACCTGAATTAGACTCATTCAAGGAGGAGTTAGA
TAAATATTTTAAGAATCATACATCACCAGATGTTGATTTAGGTGACATCTCTGGCATTAATGCTTCAGTTGTAAACATTCAAAAAGA
AATTGACAGgtaagtgccgtgtgtggttcccgcgggcctggcctctttacgggttatggcccttgcgtgccttgaattacttccacg
cccctggctgcagtacgtgattcttgatcccgagcttcgggttggaagtgggtgggagagttcgaggccttgcgcttaaggagcccc
ttcgcctcgtgcttgagttgaggcctggcttgggcgctggggccgccgcgtgcgaatctggtggcaccttcgcgcctgtctcgctgc
tttcgataagtctctagccatttaaaatttttgatgacctgctgcgacgctttttttctggcaagatagtcttgtaaatgcgggcca
agatctgcacactggtatttcggtttttggggccgcgggcggcgacggggcccgtgcgtcccagcgcacatgttcggcgaggcgggg
cctgcgagcgcggccaccgagaatcggacgggggtagtctcaagctggccggcctgctctggtgcctggcctcgcgccgccgtgtat
cgccccgccctgggcggcaaggctggcccggtcggcaccagttgcgtgagcggaaagatggccgcttcccggccctgctgcagggag
ctcaaaatggaggacgcggcgctcgggagagcgggcgggtgagtcacccacacaaaggaaaagggcctttccgtcctcagccgtcgc
ttcatgtgactccacggagtaccgggcgccgtccaggcacctcgattagttctcgagcttttggagtacgtcgtctttaggttgggg
ggaggggttttatgcgatggagtttccccacactgagtgggtggagactgaagttaggccagcttggcacttgatgtaattctcctt
ggaatttgccctttttgagtttggatcttggttcattctcaagcctcagacagtggttcaaagtttttttcttccatttcagGTTGA
ATGAGGTTGCCAAGAATTTAAATGAATCTCTCATCGATCTCCAAGAACTTGGAAAGTATGAGCAGTATATAAAATGGCCATGGTACA
TTTGGCTAGGTTTTATAGCTGGCTTGATTGCCATAGTAATGGTGACAATTATGCTTTGCTGTATGACCAGTTGCTGTAGTTGTCTCA
AGGGCTGTTGTTCTTGTGGATCCTGCTGCAAAGGCGGCGGGTCCGGAGGAGACTACAAAGACCATGACGGTGATTATAAAGATCATG
ACATCGACTACAAGGATGACGATGACAAGTAG
SEQ ID NO: 3 (P113)
GTCAGATCGCCTAGCCCCCTCCCCTCGCCTCCTCGCCGCTGGCGGCCACCGCGTCGCTCCGGCCCGGGCCCCACCCCAGGCGACTCT
GTGAGGAGCGGCCGGAGGCCGGAGGCGGAGgtgagcgcgacgcgagcaggtggagaggctgggcgcgggccaggcccggctggggga
ggggtcgggcccgggacgcggctctttgtctcccggagcccgttcgcgggcagcggggccgctctgcctcccggcaggtgcaggcat
ccctcggggaggccaggggaggccgatgggggctggcggggagacccgggcgtgcgctccgggtctggagggatgcgacatcctgag
cccgtggcagtcccccgctctcgaggctggcggtctgagtccctgaaggggcaaggggcaggggcgtggagatcggtcctgaattgg
agccgaggcgggggaggcggtgggctggggcgggcagggcctcttcgctttagggaaaagcggtggggggtgggacttggggacagc
gaggagcagtggggctggcgagtgggtgtaggtgcgtgggagccgagcggatggaagccgaggccgaggtttgagtgtccatgggtg
gcgatgctgcgaaagggcagtgaggtagcagggtccaggtctctggaggcggcgtagctgtccagaacctgggatgcggaccggttt
gtctcttcagGTGCAAGATGTTTGTTTTTCTTGTTTTATTGCCACTAGTCTCTAGTCAGTGTGTTAATCTTACAACCAGAACTCAAT
TACCCCCTGCATACACTAATTCTTTCACACGTGGTGTTTATTACCCTGACAAAGTTTTCAGATCCTCAGTTTTACATTCAACTCAGG
ACTTGTTCCTGgtaccccagcctccttcctcagctccgcccccatcttccctcccccttccaatacctgtccagtctcacctccact
gccacctctccggggcacctgtgactcggccttctccccgcagCCTTTCTTTTCCAATGTTACTTGGTTCCATGCTATACATGTCTC
AGgtaacctgctccctctcccccagtctcctaagccagggttagcgtcacagagtctggaaccttttattttacacgagttggggcg
cgggagcacttgcaggtcactgggcacaaattgggtgaaagccattattggtcctcagagagggcacatgcccatttcacagatggg
aaaatagagacttgggaagccaaacaaagacctaggcctgagcgtggccccttctgtctccagGCACCAATGGTACTAAGAGGTTTG
ATAACCCTGTCCTACCATTTAATGATGGTGTTTATTTTGCTTCCACTGAGAAGTCTAACATAATAAGAGGCTGGATTTTTGGTACTA
CTTTAGATTCGAAGACCCAGTCCCTACTTATTGTTAATAACGCTACTAATGTTGTTATTAAAGTCTGTGAATTTCAATTTTGTAATG
ATCCATTTTTGGGTGTTTATTACCACAAAAACAACAAAAGTTGGATGGAAAGTGAGTTCAGAGTTTATTCTAGTGCGAATAATTGCA
CTTTTGAATATGTCTCTCAGCCTTTTCTTATGGACCTTGAAGGAAAACAGGGTAATTTCAAAAATCTTAGGGAATTTGTGTTTAAGA
ATATTGATGGTTATTTTAAAATATATTCTAAGCACACGCCTATTAATTTAGTGCGTGATCTCCCTCAGGGTTTTTCGGCTTTAGAAC
CATTGGTAGATTTGCCAATAGGTATTAACATCACTAGGTTTCAAACTTTACTTGCTTTACATAGAAGTTATTTGACTCCTGGTGATT
CTTCTTCAGGTTGGACAGCTGGTGCTGCAGCTTATTATGTGGGTTATCTTCAACCTAGGACTTTTCTATTAAAATATAATGAAAATG
GAACCATTACAGATGCTGTAGACTGTGCACTTGACCCTCTCTCAGAGgtacgtgacctggagaagagtggggttcctgggcagcaag
gggagccgcctcagaggtatcggtgacccttggccttctactttttctccagACAAAGTGTACGTTGAAATCCTTCACTGTAGAAAA
AGGAATCTATCAAACTTCTAACTTTAGAGTCCAACCAACAGAATCTATTGTTAGATTTCCTAATATTACAAACTTGTGCCCTTTTGG
TGAAGTTTTTAACGCCACCAGATTTGCATCTGTTTATGCTTGGAACAGGAAGAGAATCAGCAACTGTGTTGCTGATTATTCTGTCCT
ATATAATTCCGCATCATTTTCCACTTTTAAGTGTTATGGAGTGTCTCCTACTAAATTAAATGATCTCTGCTTTACTAATGTCTATGC
AGATTCATTTGTAATTAGAGGTGATGAAGTCAGACAAATCGCTCCAGGGCAAACTGGAAAGATTGCTGATTATAATTATAAATTACC
AGATGATTTTACAGGCTGCGTTATAGCTTGGAATTCTAACAATCTTGATTCTAAGGTTGGTGGTAATTATAATTACCTGTATAGATT
GTTTAGGAAGTCTAATCTCAAACCTTTTGAGAGAGATATTTCAACTGAAATCTATCAGGCCGGTAGCACACCTTGTAATGGTGTTGA
AGGTTTTAATTGTTACTTTCCTTTACAATCATATGGTTTCCAACCCACTAATGGTGTTGGTTACCAACCATACAGAGTAGTAGTACT
TTCTTTTGAACTTCTACATGCACCAGCAACTGTTTGTGGACCTAAAAAGTCTACTAATTTGGTTAAAAACAAATGTGTCAATTTCAA
CTTCAATGGTTTAACAGGCACAGGTGTTCTTACTGAGTCTAACAAAAAGTTTCTGCCTTTCCAACAATTTGGCAGAGACATTGCTGA
CACTACTGATGCTGTCCGTGATCCACAGACACTTGAGATTCTTGACATTACACCATGTTCTTTTGGTGGTGTCAGTGTTATAACACC
AGGAACAAATACTTCTAACCAGGTTGCTGTTCTTTATCAGGATGTTAACTGCACAGAAGTCCCTGTTGCTATTCATGCAGATCAACT
TACTCCTACTTGGCGTGTTTATTCTACAGGTTCTAATGTTTTTCAAACACGTGCAGGCTGTTTAATAGGGGCTGAACATGTCAACAA
CTCATATGAGTGTGACATACCCATTGGTGCAGGTATATGCGCTAGTTATCAGACTCAGACTAATTCTCCTCGGCGGGCACGTAGTGT
AGCTAGTCAATCCATCATTGCCTACACTATGTCACTTGGTGCAGAAAATTCAGTTGCTTACTCTAATAACTCTATTGCCATACCCAC
AAATTTTACTATTAGTGTTACCACAGAAATTCTACCAGTGTCTATGACCAAGACATCAGTAGATTGTACAATGTACATTTGTGGTGA
TTCAACTGAATGCAGCAATCTTTTGTTGCAATATGGCAGTTTTTGTACACAATTAAACCGTGCTTTAACTGGAATAGCTGTTGAACA
AGACAAAAACACCCAAGAAGTTTTTGCACAAGTCAAACAAATTTACAAAACACCACCAATTAAAGATTTTGGTGGTTTTAATTTTTC
ACAAATATTACCAGATCCATCAAAACCAAGCAAGAGGTCATTTATTGAAGATCTACTTTTCAACAAAGTGACACTTGCAGATGCTGG
CTTCATCAAACAATATGGTGATTGCCTTGGTGATATTGCTGCTAGAGACCTCATTTGTGCACAAAAGTTTAACGGCCTTACTGTTTT
GCCACCTTTGCTCACAGATGAAATGATTGCTCAATACACTTCTGCACTGTTAGCGGGTACAATCACTTCTGGTTGGACCTTTGGTGC
AGgtaaggggcaccccagacccggcctggctgtgggcggggtcggaggcgaggcttcgcagctcaggggcgggacagctgggtccgg
ggcggagcttagacaaggaggcgggaccttgaggcaggggcggggcttatcacccccacggcccacctggcgtctctccccgcagGT
GCTGCATTACAAATACCATTTGCTATGCAAATGGCTTATAGGTTTAATGGTATTGGAGTTACACAGAATGTTCTCTATGAGAACCAA
AAATTGATTGCCAACCAATTTAATAGTGCTATTGGCAAAATTCAAGACTCACTTTCTTCCACAGCAAGTGCACTTGGAAAACTTCAA
GATGTGGTCAACCAAAATGCACAAGCTTTAAACACGCTTGTTAAACAACTTAGCTCCAATTTTGGTGCAATTTCAAGTGTTTTAAAT
GATATCCTTTCACGTCTTGACAAAGTTGAGGCTGAAGTGCAAATTGATAGGTTGATCACAGGCAGACTTCAAAGTTTGCAGACATAT
GTGACTCAACAATTAATTAGAGCTGCAGAAATCAGAGCTTCTGCTAATCTTGCTGCTACTAAAATGTCAGAGTGTGTACTTGGACAA
TCAAAAAGAGTTGATTTTTGTGGAAAGGGCTATCATCTTATGTCCTTCCCTCAGTCAGCACCTCATGGTGTAGTCTTCTTGCATGTG
ACTTATGTCCCTGCACAAGAAAAGAACTTCACAACTGCTCCTGCCATTTGTCATGATGGAAAAGCACACTTTCCTCGTGAAGGTGTC
TTTGTTTCAAATGGCACACACTGGTTTGTAACACAAAGGAATTTTTATGAACCACAAATCATTACTACAGACAACACATTTGTGTCT
GGTAACTGTGATGTTGTAATAGGAATTGTCAACAACACAGTTTATGATCCTTTGCAACCTGAATTAGACTCATTCAAGGAGGAGTTA
GATAAATATTTTAAGAATCATACATCACCAGATGTTGATTTAGGTGACATCTCTGGCATTAATGCTTCAGTTGTAAACATTCAAAAA
GAAATTGACCGCCTCAATGAGGTTGCCAAGAATTTAAATGAATCTCTCATCGATCTCCAAGAACTTGGAAAGTATGAGCAGTATATA
AAATGGCCATGGTACATTTGGCTAGGTTTTATAGCTGGCTTGATTGCCATAGTAATGGTGACAATTATGCTTTGCTGTATGACCAGT
TGCTGTAGTTGTCTCAAGGGCTGTTGTTCTTGTGGATCCTGCTGCAAATTTGATGAAGACGACTCTGAGCCAGTGCTCAAAGGAGTC
AAATTACATTACACATAAGCCAGACCACAGCCCCGCCTGCTACACCCCACCCCTGCCTTAGGATCCGCCCCTCCGGGTACGCCGTTT
GTTTTAGACCCCGCCTCCACTGCCCTGGAGCCCCGCTGGGTGGATTAGTCTTAGCTCCCTAGAGCCTGAGCCTTTGGCCTCGGAGGC
TCGGGACCTACCCACAGCTTTGACCTAGGCCCGCCCCTCGAGCTCCGCCCCTTTGGCCTAGGACACGCCCCGTTTCCCCGAGTCCCG
CCCCGTGTGCAGTGTATTGCCCACCCCGCACAGCCTGAGTTTGCAATAAAACTGGGACACTGGGACTTGCA
SEQ ID NO: 4 (P136)
GTCAGATCGCCTAGCCCCCTCCCCTCGCCTCCTCGCCGCTGGCGGCCACCGCGTCGCTCCGGCCCGGGCCCCACCCCAGGCGACTCT
GTGAGGAGCGGCCGGAGGCCGGAGGCGGAGgtgagcgcgacgcgagcaggtggagaggctgggcgcgggccaggcccggctggggga
ggggtcgggcccgggacgcggctctttgtctcccggagcccgttcgcgggcagcggggccgctctgcctcccggcaggtgcaggcat
ccctcggggaggccaggggaggccgatgggggctggcggggagacccgggcgtgcgctccgggtctggagggatgcgacatcctgag
cccgtggcagtcccccgctctcgaggctggcggtctgagtccctgaaggggcaaggggcaggggcgtggagatcggtcctgaattgg
agccgaggcgggggaggcggtgggctggggcgggcagggcctcttcgctttagggaaaagcggtggggggtgggacttggggacagc
gaggagcagtggggctggcgagtgggtgtaggtgcgtgggagccgagcggatggaagccgaggccgaggtttgagtgtccatgggtg
gcgatgctgcgaaagggcagtgaggtagcagggtccaggtctctggaggcggcgtagctgtccagaacctgggatgcggaccggttt
gtctcttcagGTGCAAGATGTTTGTTTTTCTTGTTTTATTGCCACTAGTCTCTTCCCAATGTGTTAATCTGACAACCAGAACTCAAT
TACCCCCTGCATACACTAATTCTTTCACACGTGGAGTTTATTACCCTGACAAAGTTTTCAGAAGCAGCGTTTTACATTCAACTCAAG
ACTTGTTCCTGgtaccccagcctccttcctcagctccgcccccatcttccctcccccttccaatacctgtccagtctcacctccact
gccacctctccggggcacctgtgactcggccttctccccgcagCCTTTCTTTTCCAATGTTACTTGGTTCCATGCTATACATGTCTC
AGgtaacctgctccctctcccccagtctcctaagccagggttagcgtcacagagtctggaaccttttattttacacgagttggggcg
cgggagcacttgcaggtcactgggcacaaattgggtgaaagccattattggtcctcagagagggcacatgcccatttcacagatggg
aaaatagagacttgggaagccaaacaaagacctaggcctgagcgtggccccttctgtctccagGCACCAATGGGACTAAGAGATTTG
ATAACCCTGTCCTACCATTTAATGATGGGGTTTATTTTGCTTCCACTGAGAAGTCTAACATAATAAGAGGCTGGATTTTTGGGACTA
CTTTGGATTCGAAGACCCAGTCCCTACTTATTGTTAATAACGCTACTAATGTTGTTATCAAAGTTTGCGAATTTCAATTTTGTAATG
ATCCATTTTTGGGTGTTTATTACCACAAAAACAACAAAAGTTGGATGGAATCCGAGTTCAGAGTTTATTCTAGTGCGAATAATTGCA
CTTTTGAATATGTCTCTCAACCTTTTCTTATGGACCTTGAGGGAAAACAGGGGAATTTCAAAAATCTTAGGGAATTTGTGTTTAAGA
ATATTGATGGCTATTTTAAAATATATTCTAAGCACACGCCTATTAATTTAGTCCGTGATCTCCCGCAAGGGTTTTCGGCTCTGGAAC
CATTGGTAGATTTGCCAATCGGGATTAACATCACTAGGTTTCAAACTTTACTTGCTTTACATCGGAGTTATTTGACTCCTGGGGATT
CTTCTTCTGGGTGGACAGCTGGTGCTGCGGCTTATTACGTCGGTTATCTTCAACCTCGGACTTTTCTATTAAAATATAATGAAAATG
GAACCATTACCGATGCTGTAGACTGTGCACTTGACCCTCTCTCAGAGgtacgtgacctggagaagagtggggttcctgggcagcaag
gggagccgcctcagaggtatcggtgacccttggccttctactttttctccagACAAAGTGCACGTTGAAATCCTTCACTGTGGAAAA
AGGAATCTATCAAACTTCTAACTTTCGGGTCCAACCAACAGAATCTATTGTTAGATTTCCTAATATTACAAACTTGTGCCCTTTTGG
GGAAGTTTTTAACGCCACAAGATTTGCATCTGTTTATGCTTGGAATAGGAAGAGAATCAGCAACTGTGTTGCTGATTATTCTGTCCT
ATATAATTCCGCATCATTTTCCACTTTTAAATGTTATGGAGTGTCTCCTACTAAATTAAATGATCTCTGCTTTACTAATGTCTATGC
CGATTCATTTGTAATTCGGGGGGATGAAGTCAGACAAATCGCTCCAGGGCAAACTGGAAAGATTGCTGATTATAATTATAAATTACC
AGATGATTTTACCGGCTGCGTTATCGCTTGGAATTCTAACAATCTTGATAGCAAAGTTGGCGGGAATTATAATTACCTGTATCGGTT
GTTTCGGAAGTCTAATCTCAAACCTTTTGAGAGAGATATTTCAACTGAAATCTATCAGGCCGGGAGCACACCTTGTAATGGGGTTGA
AGGGTTTAATTGTTACTTTCCTTTACAATCATATGGTTTCCAACCCACTAATGGGCTTGGTTACCAACCATATAGAGTAGTAGTACT
TTCTTTTGAACTTCTACATGCACCGGCAACTGTTTGTGGACCTAAAAAGTCTACTAATTTGGTTAAAAACAAATGTGTCAATTTCAA
CTTCAATGGTTTAACGGGCACTGGGGTTCTTACTGAATCTAACAAAAAATTTCTGCCTTTCCAACAATTTGGCCGTGACATTGCTGA
CACTACTGATGCTGTCCGTGATCCACAAACACTTGAGATTCTTGACATTACACCATGTTCTTTTGGGGGGGTCTCCGTTATAACACC
CGGAACAAATACTTCTAACCAAGTTGCTGTGCTGTACCAAGACGTTAACTGCACAGAAGTCCCTGTTGCTATTCATGCCGATCAACT
TACTCCTACTTGGCGTGTTTATTCTACGGGGTCTAATGTTTTTCAAACACGTGCAGGCTGTTTAATCGGGGCTGAACATGTCAACAA
CTCATATGAGTGTGACATACCCATTGGTGCCGGGATATGCGCTAGTTATCAAACTCAGACTAATTCTCCTCGGCGGGCACGTAGTGT
AGCTAGTCAATCCATCATTGCCTACACTATGTCACTTGGTGCCGAAAATTCAGTTGCTTACTCTAATAACTCTATTGCCATACCCAC
AAATTTTACTATTTCTGTTACCACCGAAATTCTACCAGTGTCTATGACCAAGACATCAGTAGATTGTACGATGTATATCTGTGGTGA
TTCAACTGAATGCAGCAATCTTTTGTTGCAATATGGCAGTTTTTGTACACAATTAAACCGTGCTTTAACTGGAATAGCTGTTGAACA
AGACAAAAACACCCAAGAAGTTTTTGCACAAGTCAAACAAATTTACAAAACACCACCAATTAAAGATTTTGGTGGTTTTAATTTTTC
ACAAATATTACCTGATCCATCAAAACCAAGCAAGAGATCATTTATTGAGGATCTACTGTTCAACAAAGTTACACTTGCCGATGCTGG
CTTCATCAAACAATATGGGGATTGCCTTGGTGATATTGCTGCTAGAGACCTCATTTGTGCACAAAAGTTTAACGGCCTTACTGTTTT
GCCACCTTTGCTCACGGATGAAATGATTGCTCAATACACTTCTGCACTGCTGGCGGGGACAATCACTTCTGGTTGGACCTTTGGTGC
AGgtaaggggcaccccagacccggcctggctgtgggcggggtcggaggcgaggcttcgcagctcaggggcgggacagctgggtccgg
ggcggagcttagacaaggaggcgggaccttgaggcaggggcggggcttatcacccccacggcccacctggcgtctctccccgcagGT
GCTGCATTACAAATACCATTTGCTATGCAAATGGCTTATCGATTTAATGGGATTGGAGTTACACAGAATGTTCTCTATGAGAACCAA
AAATTGATTGCCAACCAATTTAATAGTGCTATTGGCAAAATTCAAGACTCACTTTCTTCCACGGCAAGTGCACTTGGAAAACTTCAA
GATGTGGTCAACCAAAATGCACAAGCTTTAAACACGCTTGTTAAACAACTTAGCTCCAATTTTGGTGCAATTTCAAGTGTTTTAAAT
GATATCCTTTCACGTCTTGACAAAGTTGAGGCTGAAGTGCAAATTGATAGGTTGATCACGGGCAGACTTCAAAGTTTGCAAACATAT
GTGACTCAACAATTAATTAGAGCTGCAGAAATCAGAGCTTCTGCTAATCTTGCTGCTACTAAAATGTCCGAATGTGTACTTGGACAA
TCAAAAAGAGTTGATTTTTGTGGAAAGGGCTATCATCTTATGTCCTTCCCTCAATCCGCACCTCATGGAGTAGTCTTCTTGCATGTC
ACTTATGTCCCTGCACAAGAAAAGAACTTCACAACTGCTCCTGCCATTTGTCATGATGGAAAAGCACACTTTCCTCGCGAGGGGGTC
TTTGTTTCAAATGGCACACATTGGTTCGTTACACAACGGAATTTTTATGAACCACAAATCATTACTACGGACAACACATTTGTGTCT
GGGAACTGTGATGTTGTAATAGGAATTGTCAACAACACAGTTTATGATCCTTTGCAACCTGAATTAGACTCATTCAAAGAGGAGTTA
GATAAATATTTTAAAAATCATACATCACCGGATGTTGATTTAGGGGACATCTCTGGCATTAATGCTTCCGTTGTAAACATTCAAAAA
GAAATTGACCGCCTCAATGAGGTTGCCAAGAATTTAAATGAATCTCTCATCGATCTGCAAGAACTTGGAAAATATGAGCAGTATATA
AAGTGGCCTTGGTATATCTGGCTGGGGTTTATCGCTGGCTTGATTGCCATAGTAATGGTCACAATTATGCTTTGCTGTATGACTAGT
TGTTGTAGTTGTTTGAAAGGCTGTTGTTCTTGTGGATCCTGCTGCAAATTTGATGAAGACGACTCTGAGCCAGTGCTCAAAGGAGTC
AAATTACATTACACATAAGCCAGACCACAGCCCCGCCTGCTACACCCCACCCCTGCCTTAGGATCCGCCCCTCCGGGTACGCCGTTT
GTTTTAGACCCCGCCTCCACTGCCCTGGAGCCCCGCTGGGTGGATTAGTCTTAGCTCCCTAGAGCCTGAGCCTTTGGCCTCGGAGGC
TCGGGACCTACCCACAGCTTTGACCTAGGCCCGCCCCTCGAGCTCCGCCCCTTTGGCCTAGGACACGCCCCGTTTCCCCGAGTCCCG
CCCCGTGTGCAGTGTATTGCCCACCCCGCACAGCCTGAGTTTGCAATAAAACTGGGACACTGGGACTTGCA
SEQ ID NO: 5 (P148)
GTCAGATCGCCTAGCCCCCTCCCCTCGCCTCCTCGCCGCTGGCGGCCACCGCGTCGCTCCGGCCCGGGCCCCACCCCAGGCGACTCT
GTGAGGAGCGGCCGGAGGCCGGAGGCGGAGgtgagcgcgacgcgagcaggtggagaggctgggcgcgggccaggcccggctggggga
ggggtcgggcccgggacgcggctctttgtctcccggagcccgttcgcgggcagcggggccgctctgcctcccggcaggtgcaggcat
ccctcggggaggccaggggaggccgatgggggctggcggggagacccgggcgtgcgctccgggtctggagggatgcgacatcctgag
cccgtggcagtcccccgctctcgaggctggcggtctgagtccctgaaggggcaaggggcaggggcgtggagatcggtcctgaattgg
agccgaggcgggggaggcggtgggctggggcgggcagggcctcttcgctttagggaaaagcggtggggggtgggacttggggacagc
gaggagcagtggggctggcgagtgggtgtaggtgcgtgggagccgagcggatggaagccgaggccgaggtttgagtgtccatgggtg
gcgatgctgcgaaagggcagtgaggtagcagggtccaggtctctggaggcggcgtagctgtccagaacctgggatgcggaccggttt
gtctcttcagGTGCAAGATGTTCGTGTTCCTCGTGCTGCTGCCTCTGGTGTCCTCCCAGTGCGTCAATCTGACAACAAGAACACAGC
TGCCCCCCGCCTACACCAATTCCTTCACAAGAGGCGTGTACTACCCCGACAAGGTGTTCAGAAGCTCCGTGCTGCACAGCACCCAAG
ACCTCTTTCTGgtaccccagcctccttcctcagctccgcccccatcttccctcccccttccaatacctgtccagtctcacctccact
gccacctctccggggcacctgtgactcggccttctccccgcagCCTTTTTTCTCCAACGTCACATGGTTCCACGCTATCCACGTGTC
AGgtaacctgctccctctcccccagtctcctaagccagggttagcgtcacagagtctggaaccttttattttacacgagttggggcg
cgggagcacttgcaggtcactgggcacaaattgggtgaaagccattattggtcctcagagagggcacatgcccatttcacagatggg
aaaatagagacttgggaagccaaacaaagacctaggcctgagcgtggccccttctgtctccagGCACCAACGGCACCAAAAGGTTCG
ATAACCCCGTCCTCCCCTTCAACGATGGCGTCTACTTCGCCAGCACCGAGAAGTCCAATATCATCAGAGGCTGGATCTTCGGCACCA
CACTGGATTCCAAGACCCAGTCTCTGCTGATCGTGAATAACGCCACAAACGTGGTCATTAAAGTGTGCGAGTTCCAGTTCTGCAACG
ACCCCTTCCTCGGCGTGTATTACCACAAAAACAACAAGAGCTGGATGGAGTCCGAGTTTAGAGTGTACAGCAGCGCCAACAACTGCA
CATTCGAGTACGTGAGCCAGCCTTTCCTCATGGATCTGGAGGGCAAGCAAGGCAATTTCAAGAATCTGAGAGAGTTTGTCTTCAAGA
ACATCGACGGATACTTCAAGATTTACTCCAAGCACACCCCCATTAACCTCGTCAGAGACCTCCCCCAAGGCTTTTCCGCTCTCGAAC
CTCTGGTGGATCTGCCCATCGGCATCAACATCACAAGATTCCAAACCCTCCTCGCTCTGCATAGAAGCTATCTGACCCCCGGCGATT
CCAGCTCCGGATGGACAGCTGGAGCTGCCGCCTACTATGTGGGATATCTGCAACCTAGAACATTTCTGCTGAAGTACAACGAGAACG
GCACAATCACAGACGCTGTGGATTGTGCTCTGGACCCCCTCTCCGAGgtacgtgacctggagaagagtggggttcctgggcagcaag
gggagccgcctcagaggtatcggtgacccttggccttctactttttctccagACAAAGTGTACCCTCAAGAGCTTTACCGTGGAGAA
GGGAATCTACCAGACCTCCAATTTTAGGGTCCAACCCACCGAGAGCATCGTGAGGTTCCCCAACATCACAAACCTCTGCCCTTTCGG
CGAAGTGTTCAACGCCACAAGGTTTGCTTCCGTGTACGCTTGGAATAGAAAGAGAATCTCCAACTGCGTGGCCGACTATAGCGTGCT
CTATAACAGCGCCTCCTTCAGCACCTTCAAGTGTTACGGCGTGAGCCCCACCAAGCTGAACGATCTGTGTTTCACCAACGTGTACGC
TGACTCCTTCGTCATTAGGGGCGACGAAGTGAGACAAATCGCTCCCGGCCAGACCGGCAAAATCGCTGACTACAACTACAAGCTCCC
CGACGACTTCACCGGCTGTGTGATCGCTTGGAACTCCAACAACCTCGATAGCAAGGTGGGAGGCAACTACAACTATCTGTATAGACT
CTTTAGAAAGTCCAATCTGAAGCCCTTCGAGAGGGACATCAGCACAGAGATCTATCAAGCCGGATCCACACCTTGCAACGGCGTCGA
GGGATTCAACTGCTACTTCCCTCTGCAATCCTACGGCTTCCAGCCCACAAATGGCGTGGGCTACCAGCCTTACAGAGTGGTGGTGCT
GTCCTTTGAACTGCTGCATGCCCCCGCCACAGTGTGCGGACCCAAAAAGAGCACCAACCTCGTGAAGAACAAATGCGTCAATTTCAA
CTTCAATGGACTGACCGGCACCGGCGTGCTCACCGAGTCCAACAAGAAGTTTCTGCCCTTCCAGCAGTTCGGAAGAGACATTGCCGA
TACCACAGACGCCGTGAGGGACCCTCAGACACTGGAGATTCTGGATATCACACCTTGCAGCTTCGGCGGCGTGAGCGTGATCACACC
CGGAACAAACACCAGCAACCAAGTGGCTGTGCTGTACCAAGACGTGAATTGTACAGAGGTACCTGTGGCCATCCATGCCGATCAGCT
GACCCCCACATGGAGGGTCTACAGCACCGGCTCCAATGTCTTTCAGACAAGAGCTGGCTGTCTGATTGGCGCTGAGCACGTGAACAA
CAGCTACGAGTGCGACATCCCTATCGGCGCCGGAATTTGCGCCAGCTACCAAACCCAGACCAATAGCCCTAGGAGGGCCAGATCCGT
CGCCAGCCAGAGCATCATCGCCTATACCATGTCTCTGGGCGCTGAGAACTCCGTGGCCTATAGCAACAACAGCATCGCTATCCCCAC
CAACTTCACAATCTCCGTGACCACCGAGATTCTGCCCGTGAGCATGACCAAGACCAGCGTCGACTGCACCATGTATATCTGCGGCGA
CTCCACAGAGTGCTCCAATCTGCTGCTGCAGTACGGCAGCTTCTGCACCCAACTCAATAGGGCTCTGACCGGAATTGCTGTCGAGCA
AGACAAGAACACCCAAGAGGTGTTTGCCCAAGTGAAACAGATTTACAAGACCCCCCCCATCAAGGACTTCGGAGGCTTCAATTTCTC
CCAAATCCTCCCCGACCCCTCCAAACCCTCCAAGAGGAGCTTTATCGAGGATCTGCTGTTCAACAAGGTGACACTGGCTGATGCCGG
CTTTATCAAGCAGTATGGCGACTGTCTGGGAGACATCGCTGCTAGGGATCTGATCTGTGCCCAGAAGTTTAATGGCCTCACCGTGCT
GCCTCCTCTGCTGACCGACGAGATGATCGCCCAGTATACAAGCGCTCTGCTGGCCGGCACAATTACCAGCGGATGGACATTTGGAGC
AGgtaaggggcaccccagacccggcctggctgtgggcggggtcggaggcgaggcttcgcagctcaggggcgggacagctgggtccgg
ggcggagcttagacaaggaggcgggaccttgaggcaggggcggggcttatcacccccacggcccacctggcgtctctccccgcagGT
GCTGCCCTCCAGATTCCTTTCGCCATGCAGATGGCCTATAGATTCAACGGCATTGGCGTCACACAGAACGTGCTGTACGAGAACCAG
AAGCTGATCGCTAACCAGTTCAACAGCGCCATTGGCAAGATCCAAGATTCCCTCAGCTCCACCGCCAGCGCCCTCGGCAAACTGCAA
GACGTCGTGAATCAGAATGCCCAAGCTCTGAACACACTGGTGAAGCAGCTCAGCAGCAATTTTGGCGCCATCTCCTCCGTGCTCAAT
GATATTCTGTCTAGACTGGACAAGGTGGAGGCCGAAGTCCAGATCGATAGACTGATCACCGGAAGACTGCAGTCCCTCCAGACATAC
GTGACCCAGCAGCTCATTAGAGCTGCCGAGATTAGGGCCTCCGCCAATCTCGCTGCCACAAAAATGAGCGAGTGCGTGCTCGGCCAG
TCCAAAAGAGTGGACTTCTGTGGCAAGGGCTACCATCTGATGTCCTTCCCTCAGAGCGCTCCTCATGGCGTCGTGTTTCTGCATGTG
ACCTACGTGCCCGCCCAAGAGAAGAACTTCACAACAGCCCCCGCTATCTGTCACGACGGAAAGGCCCACTTCCCCAGAGAGGGCGTC
TTTGTGTCCAACGGCACACACTGGTTTGTCACCCAGAGGAACTTCTATGAGCCCCAGATCATCACCACCGACAACACCTTTGTGAGC
GGAAACTGCGATGTGGTCATCGGCATCGTGAATAACACCGTGTACGACCCTCTCCAGCCCGAGCTGGACTCCTTCAAGGAGGAGCTG
GATAAGTACTTTAAGAACCATACAAGCCCCGACGTGGACCTCGGCGACATTAGCGGAATCAACGCCAGCGTCGTGAACATCCAGAAG
GAGATTGATAGACTCAACGAGGTCGCCAAGAATCTGAACGAGTCTCTGATTGATCTGCAAGAGCTGGGCAAGTACGAGCAGTACATC
AAGTGGCCTTGGTACATCTGGCTCGGATTCATTGCCGGACTGATCGCCATCGTCATGGTGACCATCATGCTCTGCTGCATGACAAGC
TGTTGCAGCTGTCTGAAAGGCTGTTGTAGCTGTGGCAGCTGCTGTAAGTTCGATGAGGACGACTCCGAGCCCGTGCTGAAGGGCGTG
AAGCTCCACTACACCTAAGCCAGACCACAGCCCCGCCTGCTACACCCCACCCCTGCCTTAGGATCCGCCCCTCCGGGTACGCCGTTT
GTTTTAGACCCCGCCTCCACTGCCCTGGAGCCCCGCTGGGTGGATTAGTCTTAGCTCCCTAGAGCCTGAGCCTTTGGCCTCGGAGGC
TCGGGACCTACCCACAGCTTTGACCTAGGCCCGCCCCTCGAGCTCCGCCCCTTTGGCCTAGGACACGCCCCGTTTCCCCGAGTCCCG
CCCCGTGTGCAGTGTATTGCCCACCCCGCACAGCCTGAGTTTGCAATAAAACTGGGACACTGGGACTTGCA
SEQ ID NO: 6 (P143)
ATGTTTGTTTTTCTTGTTTTATTGCCACTAGTCTCTTCCCAATGTGTTAATCTGACAACCAGAACTCAATTACCCCCTGCATACACT
AATTCTTTCACACGTGGAGTTTATTACCCTGACAAAGTTTTCAGAAGCAGCGTTTTACATTCAACTCAAGACTTGTTCTTACCTTTC
TTTTCCAATGTTACTTGGTTCCATGCTATACATGTCTCAGgtaagttgatttagaaacacttttcaagcagtcagcccatggttacc
attaagttaaccctatcactgaattgctccaattttcctcttagGTACCAATGGGACTAAGAGATTTGATAACCCTGTCCTACCATT
TAATGATGGGGTTTATTTTGCTTCCACTGAGAAGTCTAACATAATAAGAGGCTGGATTTTTGGGACTACTTTGGATTCGAAGACCCA
GTCCCTACTTATTGTTAATAACGCTACTAATGTTGTTATCAAAGTTTGCGAATTTCAATTTTGTAATGATCCATTTTTGGGTGTTTA
TTACCACAAAAACAACAAAAGTTGGATGGAATCCGAGTTCAGgtaaggaaatttccatgagtttcactcttgaagcattggggttat
ttgtgccagaggctaatgacccatgctggcccttcacttttctagGGTTTATTCTAGTGCGAATAATTGCACTTTTGAATATGTCTC
TCAACCTTTTCTTATGGACCTTGAGGGAAAACAGGGGAATTTCAAAAATCTTAGGGAATTTGTGTTTAAGAATATTGATGGCTATTT
TAAAATATATTCTAAGCACACGCCTATTAATTTAGTCCGTGATCTCCCGCAAGGGTTTTCGGCTCTGGAACCATTGGTAGATTTGCC
AATCGGGATTAACATCACTAGGTTTCAAACTTTACTTGCTTTACATCGGAGTTATTTGACTCCTGGGGATTCTTCTTCAGgtaagta
atttatataccactagagattttttcatcagtttctgttataaaaataattaaaatcaacatatttttctcctttacaacagGTTGG
ACAGCTGGTGCTGCGGCTTATTACGTCGGTTATCTTCAACCTCGGACTTTTCTATTAAAATATAATGAAAATGGAACCATTACAGAT
GCTGTAGACTGTGCACTTGACCCTCTGAGCGAAACAAAGTGCACGTTGAAATCCTTCACTGTGGAAAAAGGAATCTATCAAACTTCT
AACTTTCGGGTCCAACCAACAGAATCTATTGTTAGATTTCCTAATATTACAAACTTGTGCCCTTTTGGGGAAGTTTTTAACGCCACA
AGATTTGCATCTGTTTATGCTTGGAACAGgtaagtagtgctgattatacacaagatattgtctagaacttgatgagactgtggatat
gaatatttcactcttttctcagGAAGAGAATCAGCAACTGTGTTGCTGATTATTCTGTCCTATATAATTCCGCATCATTTTCCACTT
TTAAATGTTATGGAGTGTCTCCTACTAAATTAAATGATCTCTGCTTTACTAATGTCTATGCCGATTCATTTGTAATTCGGGGGGATG
AAGTCAGACAAATCGCTCCAGGGCAAACTGGAAAGATTGCTGATTATAATTATAAATTACCAGATGATTTTACCGGCTGCGTTATCG
CTTGGAATTCTAACAATCTTGATAGCAAAGTTGGCGGGAATTATAATTACCTGTATCGGTTGTTCAGgtaaggaatgttgcactgat
tttcacaggattttcccaagtgatactatcttattacattgatttttggctttgttttgttttcagGAAGTCTAATCTCAAACCTTT
TGAGAGAGATATTTCAACTGAAATCTATCAGGCCGGGAGCACACCTTGTAATGGGGTTGAAGGGTTTAATTGTTACTTTCCTTTACA
ATCATATGGTTTCCAACCCACTAATGGGGTTGGTTACCAACCATATAGAGTAGTAGTACTTTCTTTTGAACTTCTACATGCACCGGC
AACTGTTTGTGGACCTAAAAAGTCTACTAATTTGGTTAAAAACAAATGTGTCAATTTCAACTTCAATGGTTTAACGGGCACAGgtaa
gtgacttgctttcttacatcaaaaaggcatccagtgtctgtttaagaattgccttctcaatattctctgttgattcctttccagGTG
TTCTTACTGAATCTAACAAAAAATTTCTGCCTTTCCAACAATTTGGCCGTGACATTGCTGACACTACTGATGCTGTCCGTGATCCAC
AAACACTTGAGATTCTTGACATTACACCATGTTCTTTTGGGGGGGTCTCCGTTATAACACCCGGAACAAATACTTCTAACCAAGTTG
CTGTGCTGTACCAAGACGTTAACTGCACAGAAGTCCCTGTTGCTATTCATGCCGATCAACTTACTCCTACTTGGCGTGTTTATTCTA
CAGgtaagtaggagaacattttcacatacaaagccatttttactttttttttaaatttcttataatcaatatgatctttttcacagG
TTCTAATGTTTTTCAAACACGTGCAGGCTGTTTAATCGGGGCTGAACATGTCAACAACTCATATGAGTGTGACATACCCATTGGTGC
CGGGATATGCGCTAGTTATCAAACTCAGACTAATTCTCCTCGGCGGGCACGTAGTGTAGCTAGTCAATCCATCATTGCCTACACTAT
GTCACTTGGTGCCGAAAATTCAGTTGCTTACTCTAATAACTCTATTGCCATACCCACAAATTTTACTATTTCTGTTACCACAGgtaa
gttgctttctctgaatacaaaactattgtttgactgtctttaagaatattactttttcatcataacttcttctttgaaaagAAATTC
TACCAGTGTCTATGACCAAGACATCAGTAGATTGTACGATGTATATCTGTGGTGATTCAACTGAATGCAGCAATCTTTTGTTGCAAT
ATGGCAGTTTTTGTACACAATTAAACCGTGCTTTAACTGGAATAGCTGTTGAACAAGACAAAAACACCCAAGAAGTTTTTGCACAAG
TCAAACAAATTTACAAAACACCACCAATTAAAGATTTTGGTGGTTTTAATTTTTCACAAATATTACCTGATCCATCAAAACCAAGCA
AGAGATCATTTATTGAGGATCTACTGTTCAACAAAGTTACACTTGCCGATGCAGgtaagtctatttcaaaaaagaatcatatatatt
ttaaaatagcttatgtattttttacacattcatttcttatttacctactatttatccagGTTTCATCAAACAATATGGGGATTGCCT
TGGTGATATTGCTGCTAGAGACCTCATTTGTGCACAAAAGTTTAACGGCCTTACTGTTTTGCCACCTTTGCTCACGGATGAAATGAT
TGCTCAATACACTTCTGCACTGCTGGCGGGGACAATCACTTCTGGTTGGACCTTTGGTGCTGGGGCTGCATTACAAATACCATTTGC
TATGCAAATGGCTTACAGgtaagcaaatgaaccatcatcccatcattttgagttatatccttcctttgttatatggggcttacactt
atcatttctcctttgctttagGTTTAATGGGATTGGAGTTACACAGAATGTTCTCTATGAGAACCAAAAATTGATTGCCAACCAATT
TAATAGTGCTATTGGCAAAATTCAAGACTCACTTTCTTCCACGGCAAGTGCACTTGGAAAACTTCAAGATGTGGTCAACCAAAATGC
ACAAGCTTTAAACACGCTTGTTAAACAACTTAGCTCCAATTTTGGTGCAATTTCAAGTGTTTTAAATGATATCCTTTCACGTCTTGA
CAAAGTTGAGGCTGAAGTGCAAATTGATAGGTTGATCACAGgtaagtgtcttaaattcagaagacgtaaagcaaaacacggttttga
ggaggcttcttattataaatcttgcattatctacttttttctagGTAGACTTCAAAGTTTGCAAACATATGTGACTCAACAATTAAT
TAGAGCTGCAGAAATCAGAGCTTCTGCTAATCTTGCTGCTACTAAAATGTCCGAATGTGTACTTGGACAATCAAAAAGAGTTGATTT
TTGTGGAAAGGGCTATCATCTTATGTCCTTCCCTCAATCCGCACCTCATGGAGTAGTCTTCTTGCATGTCACTTATGTCCCTGCACA
AGAAAAGAACTTCACAACTGCTCCTGCCATTTGTCATGATGGAAAAGCACACTTTCCCAGgtaagtcattatatgaagaaaaaccca
ggtgcatgttttacatgaagaaaactggtatttgtttgactggttttgcttttatgttttagGGAGGGGGTCTTTGTTTCAAATGGC
ACACATTGGTTCGTTACACAACGGAATTTTTATGAACCACAAATCATTACTACGGACAACACATTTGTGTCTGGGAACTGTGATGTT
GTAATAGGAATTGTCAACAACACAGTTTATGATCCTTTGCAACCTGAATTAGACTCATTCAAAGAGGAGTTAGATAAATATTTTAAA
AATCATACATCACCGGATGTTGATTTAGGGGACATCTCAGgtaagttgtccaacttttcaaagatccaggttttcttttaccataaa
tgtgttattgtctgtactaatctataggatttctctcttttgtagGTATTAATGCTTCCGTTGTAAACATTCAAAAAGAAATTGACC
GCCTCAATGAGGTTGCCAAGAATTTAAATGAATCTCTCATCGATCTGCAAGAACTTGGAAAATATGAGCAGTATATAAAGTGGCCTT
GGTATATCTGGCTGGGGTTTATCGCTGGCTTGATTGCCATAGTAATGGTCACAATTATGCTTTGCTGTATGACTAGTTGTTGTAGTT
GTTTGAAAGGCTGTTGTTCTTGTGGATCCTGCTGCAAAGGCGGCGGGTCCGGAGGAGACTACAAAGACCATGACGGGGATTATAAAG
ATCATGACATCGACTACAAGGATGACGATGACAAGTAG
SEQ ID NO: 7 (P172)
ATGTTTGTTTTTCTTGTTTTATTGCCACTAGTCTCTTCCCAATGTGTTAATCTGACAACCAGAACTCAATTACCCCCTGCATACACT
AATTCTTTCACACGTGGAGTTTATTACCCTGACAAAGTTTTCAGAAGCAGCGTTTTACATTCAACTCAAGACTTGTTCTTACCTTTC
TTTTCCAATGTTACTTGGTTCCATGCTATACATGTCTCAGgtaagttgatttagaaacacttttcaagcagtcagcccatggttacc
attaagttaaccctatcactgaattgctccaattttcctcttagGTACCAACGGGACCAAGCGGTTCGACAACCCCGTCCTCCCCTT
CAACGACGGGGTCTATTTTGCTTCCACTGAGAAGTCTAACATAATAAGAGGCTGGATTTTTGGGACTACTTTGGATTCGAAGACCCA
GTCCCTACTTATTGTTAATAACGCTACTAATGTTGTTATCAAAGTTTGCGAATTTCAATTTTGTAATGATCCATTTTTGGGTGTTTA
TTACCACAAAAACAACAAAAGTTGGATGGAATCCGAGTTCAGgtaaggaaatttccatgagtttcactcttgaagcattggggttat
ttgtgccagaggctaatgacccatgctggcccttcacttttctagGGTTTATTCTAGTGCGAATAATTGCACTTTTGAATATGTCTC
TCAACCTTTTCTTATGGACCTTGAGGGAAAACAGGGGAATTTCAAAAATCTTAGGGAATTTGTGTTTAAGAATATTGATGGCTATTT
TAAAATATATTCTAAGCACACGCCTATTAATTTAGTCCGTGATCTCCCGCAAGGGTTTTCGGCTCTGGAACCATTGGTAGATTTGCC
AATCGGGATTAACATCACTAGGTTTCAAACTTTACTTGCTTTACATCGGAGTTATTTGACTCCTGGGGATTCTTCTTCAGgtaagta
atttatataccactagagattttttcatcagtttctgttataaaaataattaaaatcaacatatttttctcctttacaacagGTTGG
ACAGCTGGTGCTGCGGCTTATTACGTCGGTTATCTTCAACCTCGGACTTTTCTATTAAAATATAATGAAAATGGAACCATTACAGAT
GCTGTAGACTGTGCACTTGACCCTCTGAGCGAAACAAAGTGCACGTTGAAATCCTTCACTGTGGAAAAAGGAATCTATCAAACTTCT
AACTTTCGGGTCCAACCAACAGAATCTATTGTTAGATTTCCTAATATTACAAACTTGTGCCCTTTTGGGGAAGTTTTTAACGCCACA
AGATTTGCATCTGTTTATGCTTGGAACAGgtaagtagtgctgattatacacaagatattgtctagaacttgatgagactgtggatat
gaatatttcactcttttctcagGAAGAGAATCAGCAACTGTGTTGCTGATTATTCTGTCCTATATAATTCCGCATCATTTTCCACTT
TTAAATGTTATGGAGTGTCTCCTACTAAATTAAATGATCTCTGCTTTACTAATGTCTATGCCGATTCATTTGTAATTCGGGGGGATG
AAGTCAGACAAATCGCTCCAGGGCAAACTGGAAAGATTGCTGATTATAATTATAAATTACCAGATGATTTTACCGGCTGCGTTATCG
CTTGGAATTCTAACAATCTTGATAGCAAAGTTGGCGGGAATTATAATTACCTGTATCGGTTGTTCAGgtaaggaatgttgcactgat
tttcacaggattttcccaagtgatactatcttattacattgatttttggctttgttttgttttcagGAAGTCTAATCTCAAACCTTT
TGAGAGAGATATTTCAACTGAAATCTATCAGGCCGGGAGCACACCTTGTAATGGGGTTGAAGGGTTTAATTGTTACTTTCCTTTACA
ATCATATGGTTTCCAACCCACTAATGGGGTTGGTTACCAACCATATAGAGTAGTAGTACTTTCTTTTGAACTTCTACATGCACCGGC
AACTGTTTGTGGACCTAAAAAGTCTACTAATTTGGTTAAAAACAAATGTGTCAATTTCAACTTCAATGGTTTAACGGGCACAGgtaa
gtgacttgctttcttacatcaaaaaggcatccagtgtctgtttaagaattgccttctcaatattctctgttgattcctttccagGTG
TTCTTACTGAATCTAACAAAAAATTTCTGCCTTTCCAACAATTTGGCCGTGACATTGCTGACACTACTGATGCTGTCCGTGATCCAC
AAACACTTGAGATTCTTGACATTACACCATGTTCTTTTGGGGGGGTCTCCGTTATAACACCCGGAACAAATACTTCTAACCAAGTTG
CTGTGCTGTACCAAGACGTTAACTGCACAGAAGTCCCTGTTGCTATTCATGCCGATCAACTTACTCCTACTTGGCGTGTTTATTCTA
CAGgtaagtaggagaacattttcacatacaaagccatttttactttttttttaaatttcttataatcaatatgatctttttcacagG
TTCTAATGTTTTTCAAACACGTGCAGGCTGTTTAATCGGGGCTGAACATGTCAACAACTCATATGAGTGTGACATACCCATTGGTGC
CGGGATATGCGCTAGTTATCAAACTCAGACTAATTCTCCTCGGCGGGCACGTAGTGTAGCTAGTCAATCCATCATTGCCTACACTAT
GTCACTTGGTGCCGAAAATTCAGTTGCTTACTCTAATAACTCTATTGCCATACCCACAAATTTTACTATTTCTGTTACCACAGgtaa
gttgctttctctgaatacaaaactattgtttgactgtctttaagaatattactttttcatcataacttcttctttgaaaagAAATTC
TACCAGTGTCTATGACCAAGACATCAGTAGATTGTACGATGTATATCTGTGGTGATTCAACTGAATGCAGCAATCTTTTGTTGCAAT
ATGGCAGTTTTTGTACACAATTAAACCGTGCTTTAACTGGAATAGCTGTTGAACAAGACAAAAACACCCAAGAAGTTTTTGCACAAG
TCAAACAAATTTACAAAACACCACCAATTAAAGATTTTGGTGGTTTTAATTTTTCACAAATATTACCTGATCCATCAAAACCAAGCA
AGAGATCATTTATTGAGGATCTACTGTTCAACAAAGTTACACTTGCCGATGCAGgtaagtctatttcaaaaaagaatcatatatatt
ttaaaatagcttatgtattttttacacattcatttcttatttacctactatttatccagGTTTCATCAAACAATATGGGGATTGCCT
TGGTGATATTGCTGCTAGAGACCTCATTTGTGCACAAAAGTTTAACGGCCTTACTGTTTTGCCACCTTTGCTCACGGATGAAATGAT
TGCTCAATACACTTCTGCACTGCTGGCGGGGACAATCACTTCTGGTTGGACCTTTGGTGCTGGGGCTGCATTACAAATACCATTTGC
TATGCAAATGGCTTACAGgtaagcaaatgaaccatcatcccatcattttgagttatatccttcctttgttatatggggcttacactt
atcatttctcctttgctttagGTTTAATGGGATTGGAGTTACACAGAATGTTCTCTATGAGAACCAAAAATTGATTGCCAACCAATT
TAATAGTGCTATTGGCAAAATTCAAGACTCACTTTCTTCCACGGCAAGTGCACTTGGAAAACTTCAAGATGTGGTCAACCAAAATGC
ACAAGCTTTAAACACGCTTGTTAAACAACTTAGCTCCAATTTTGGTGCAATTTCAAGTGTTTTAAATGATATCCTTTCACGTCTTGA
CAAAGTTGAGGCTGAAGTGCAAATTGATAGGTTGATCACAGgtaagtgtcttaaattcagaagacgtaaagcaaaacacggttttga
ggaggcttcttattataaatcttgcattatctacttttttctagGTAGACTTCAAAGTTTGCAAACATATGTGACTCAACAATTAAT
TAGAGCTGCAGAAATCAGAGCTTCTGCTAATCTTGCTGCTACTAAAATGTCCGAATGTGTACTTGGACAATCAAAAAGAGTTGATTT
TTGTGGAAAGGGCTATCATCTTATGTCCTTCCCTCAATCCGCACCTCATGGAGTAGTCTTCTTGCATGTCACTTATGTCCCTGCACA
AGAAAAGAACTTCACAACTGCTCCTGCCATTTGTCATGATGGAAAAGCACACTTTCCCAGgtaagtcattatatgaagaaaaaccca
ggtgcatgttttacatgaagaaaactggtatttgtttgactggttttgcttttatgttttagGGAGGGGGTCTTTGTTTCAAATGGC
ACACATTGGTTCGTTACACAACGGAATTTTTATGAACCACAAATCATTACTACGGACAACACATTTGTGTCTGGGAACTGTGATGTT
GTAATAGGAATTGTCAACAACACAGTTTATGATCCTTTGCAACCTGAATTAGACTCATTCAAAGAGGAGTTAGATAAATATTTTAAA
AATCATACATCACCGGATGTTGATTTAGGGGACATCTCAGgtaagttgtccaacttttcaaagatccaggttttcttttaccataaa
tgtgttattgtctgtactaatctataggatttctctcttttgtagGTATTAATGCTTCCGTTGTAAACATTCAAAAAGAAATTGACC
GCCTCAATGAGGTTGCCAAGAATTTAAATGAATCTCTCATCGATCTGCAAGAACTTGGAAAATATGAGCAGTATATAAAGTGGCCTT
GGTATATCTGGCTGGGGTTTATCGCTGGCTTGATTGCCATAGTAATGGTCACAATTATGCTTTGCTGTATGACTAGTTGTTGTAGTT
GTTTGAAAGGCTGTTGTTCTTGTGGATCCTGCTGCAAAGGCGGCGGGTCCGGAGGAGACTACAAAGACCATGACGGGGATTATAAAG
ATCATGACATCGACTACAAGGATGACGATGACAAGTAG
SEQ ID NO: 8 (P166)
ATGTTCGTGTTCCTCGTGCTGCTGCCTCTGGTGTCCTCCCAGTGCGTCAATCTGACAACAAGAACACAGCTGCCCCCCGCCTACACC
AATTCCTTCACAAGAGGCGTGTACTACCCCGACAAGGTGTTCAGAAGCTCCGTGCTGCACAGCACCCAAGACCTCTTTCTGCCCTTT
TTCTCCAACGTCACATGGTTCCACGCTATCCACGTGAGCGGAACCAACGGCACCAAAAGGTTCGATAACCCCGTCCTCCCCTTCAAC
GATGGCGTCTACTTCGCCAGCACCGAGAAGTCCAATATCATCAGAGGCTGGATCTTCGGCACCACACTGGATTCCAAGACCCAGTCT
CTGCTGATCGTGAATAACGCCACAAACGTGGTCATTAAAGTGTGCGAGTTCCAGTTCTGCAACGACCCCTTCCTCGGCGTGTATTAC
CACAAAAACAACAAGAGCTGGATGGAGTCCGAGTTTAGAGTGTACAGCAGCGCCAACAACTGCACATTCGAGTACGTGAGCCAGCCT
TTCCTCATGGATCTGGAGGGCAAGCAAGGCAATTTCAAGAATCTGAGAGAGTTTGTCTTCAAGAACATCGACGGATACTTCAAGATT
TACTCCAAGCACACCCCCATTAACCTCGTCAGAGACCTCCCCCAAGGCTTTTCCGCTCTCGAACCTCTGGTGGATCTGCCCATCGGC
ATCAACATCACAAGATTCCAAACCCTCCTCGCTCTGCATAGAAGCTATCTGACCCCCGGCGATTCCAGCTCCGGATGGACAGCTGGA
GCTGCCGCCTACTATGTGGGATATCTGCAACCTAGAACATTTCTGCTGAAGTACAACGAGAACGGCACAATCACAGACGCTGTGGAT
TGTGCTCTGGACCCCCTCTCCGAGACCAAGTGTACCCTCAAGAGCTTTACCGTGGAGAAGGGAATCTACCAGACCTCCAATTTTAGG
GTCCAACCCACCGAGAGCATCGTGAGGTTCCCCAACATCACAAACCTCTGCCCTTTCGGCGAAGTGTTCAACGCCACAAGGTTTGCT
TCCGTGTACGCTTGGAATAGAAAGAGAATCTCCAACTGCGTGGCCGACTATAGCGTGCTCTATAACAGCGCCTCCTTCAGCACCTTC
AAGTGTTACGGCGTGAGCCCCACCAAGCTGAACGATCTGTGTTTCACCAACGTGTACGCTGACTCCTTCGTCATTAGGGGCGACGAA
GTGAGACAAATCGCTCCCGGCCAGACCGGCAAAATCGCTGACTACAACTACAAGCTCCCCGACGACTTCACCGGCTGTGTGATCGCT
TGGAACTCCAACAACCTCGATAGCAAGGTGGGAGGCAACTACAACTATCTGTATAGACTCTTTAGAAAGTCCAATCTGAAGCCCTTC
GAGAGGGACATCAGCACAGAGATCTATCAAGCCGGATCCACACCTTGCAACGGCGTCGAGGGATTCAACTGCTACTTCCCTCTGCAA
TCCTACGGCTTCCAGCCCACAAATGGCGTGGGCTACCAGCCTTACAGAGTGGTGGTGCTGTCCTTTGAACTGCTGCATGCCCCCGCC
ACAGTGTGCGGACCCAAAAAGAGCACCAACCTCGTGAAGAACAAATGCGTCAATTTCAACTTCAATGGACTGACCGGCACCGGCGTG
CTCACCGAGTCCAACAAGAAGTTTCTGCCCTTCCAGCAGTTCGGAAGAGACATTGCCGATACCACAGACGCCGTGAGGGACCCTCAG
ACACTGGAGATTCTGGATATCACACCTTGCAGCTTCGGCGGCGTGAGCGTGATCACACCCGGAACAAACACCAGCAACCAAGTGGCT
GTGCTGTACCAAGACGTGAATTGTACAGAGGTACCTGTGGCCATCCATGCCGATCAGCTGACCCCCACATGGAGGGTCTACAGCACC
GGCTCCAATGTCTTTCAGACAAGAGCTGGCTGTCTGATTGGCGCTGAGCACGTGAACAACAGCTACGAGTGCGACATCCCTATCGGC
GCCGGAATTTGCGCCAGCTACCAAACCCAGACCAATAGCCCTAGGAGGGCCAGATCCGTCGCCAGCCAGAGCATCATCGCCTATACC
ATGTCTCTGGGCGCTGAGAACTCCGTGGCCTATAGCAACAACAGCATCGCTATCCCCACCAACTTCACAATCTCCGTGACCACCGAG
ATTCTGCCCGTGAGCATGACCAAGACCAGCGTCGACTGCACCATGTATATCTGCGGCGACTCCACAGAGTGCTCCAATCTGCTGCTG
CAGTACGGCAGCTTCTGCACCCAACTCAATAGGGCTCTGACCGGAATTGCTGTCGAGCAAGACAAGAACACCCAAGAGGTGTTTGCC
CAAGTGAAACAGATTTACAAGACCCCCCCCATCAAGGACTTCGGAGGCTTCAATTTCTCCCAAATCCTCCCCGACCCCTCCAAACCC
TCCAAGAGGAGCTTTATCGAGGATCTGCTGTTCAACAAGGTGACACTGGCTGATGCCGGCTTTATCAAGCAGTATGGCGACTGTCTG
GGAGACATCGCTGCTAGGGATCTGATCTGTGCCCAGAAGTTTAATGGCCTCACCGTGCTGCCTCCTCTGCTGACCGACGAGATGATC
GCCCAGTATACAAGCGCTCTGCTGGCCGGCACAATTACCAGCGGATGGACATTTGGAGCCGGCGCTGCCCTCCAGATTCCTTTCGCC
ATGCAGATGGCCTATAGATTCAACGGCATTGGCGTCACACAGAACGTGCTGTACGAGAACCAGAAGCTGATCGCTAACCAGTTCAAC
AGCGCCATTGGCAAGATCCAAGATTCCCTCAGCTCCACCGCCAGCGCCCTCGGCAAACTGCAAGACGTCGTGAATCAGAATGCCCAA
GCTCTGAACACACTGGTGAAGCAGCTCAGCAGCAATTTTGGCGCCATCTCCTCCGTGCTCAATGATATTCTGTCTAGACTGGACAAG
GTGGAGGCCGAAGTCCAGATCGATAGACTGATCACCGGAAGACTGCAGTCCCTCCAGACATACGTGACCCAGCAGCTCATTAGAGCT
GCCGAGATTAGGGCCTCCGCCAATCTCGCTGCCACAAAAATGAGCGAGTGCGTGCTCGGCCAGTCCAAAAGAGTGGACTTCTGTGGC
AAGGGCTACCATCTGATGTCCTTCCCTCAGAGCGCTCCTCATGGCGTCGTGTTTCTGCATGTGACCTACGTGCCCGCCCAAGAGAAG
AACTTCACAACAGCCCCCGCTATCTGTCACGACGGAAAGGCCCACTTCCCCAGAGAGGGCGTCTTTGTGTCCAACGGCACACACTGG
TTTGTCACCCAGAGGAACTTCTATGAGCCCCAGATCATCACCACCGACAACACCTTTGTGAGCGGAAACTGCGATGTGGTCATCGGC
ATCGTGAATAACACCGTGTACGACCCTCTCCAGCCCGAGCTGGACTCCTTCAAGGAGGAGCTGGATAAGTACTTTAAGAACCATACA
AGCCCCGACGTGGACCTCGGCGACATTAGCGGAATCAACGCCAGCGTCGTGAACATCCAGAAGGAGATTGATAGACTCAACGAGGTC
GCCAAGAATCTGAACGAGTCTCTGATTGATCTGCAAGAGCTGGGCAAGTACGAGCAGTACATCAAGTGGCCTTGGTACATCTGGCTC
GGATTCATTGCCGGACTGATCGCCATCGTCATGGTGACCATCATGCTCTGCTGCATGACAAGCTGTTGCAGCTGTCTGAAAGGCTGT
TGTAGCTGTGGCAGCTGCTGTAAGTTCGATGAGGACGACTCCGAGCCCGTGCTGAAGGGCGTGAAGCTCCACTACACCTAA
SEQ ID NO: 9 (P205)
ATGTTCGTGTTCCTCGTGCTGCTGCCTCTGGTGTCCTCCCAGTGCGTCAATCTGACAACAAGAACACAGCTGCCCCCCGCCTACACC
AATTCCTTCACAAGAGGCGTGTACTACCCCGACAAGGTGTTCAGAAGCTCCGTGCTGCACAGCACCCAAGACCTCTTTCTGCCCTTT
TTCTCCAACGTCACATGGTTCCACGCTATCCACGTGAGCGGAACCAACGGCACCAAAAGGTTCGATAACCCCGTCCTCCCCTTCAAC
GATGGCGTCTACTTCGCCAGCACCGAGAAGTCCAATATCATCAGAGGCTGGATCTTCGGCACCACACTGGATTCCAAGACCCAGTCT
CTGCTGATCGTGAATAACGCCACAAACGTGGTCATTAAAGTGTGCGAGTTCCAGTTCTGCAACGACCCCTTCCTCGGCGTGTATTAC
CACAAAAACAACAAGAGCTGGATGGAGTCCGAGTTTAGAGTGTACAGCAGCGCCAACAACTGCACATTCGAGTACGTGAGCCAGCCT
TTCCTCATGGATCTGGAGGGCAAGCAAGGCAATTTCAAGAATCTGAGAGAGTTTGTCTTCAAGAACATCGACGGATACTTCAAGATT
TACTCCAAGCACACCCCCATTAACCTCGTCAGAGACCTCCCCCAAGGCTTTTCCGCTCTCGAACCTCTGGTGGATCTGCCCATCGGC
ATCAACATCACAAGATTCCAAACCCTCCTCGCTCTGCATAGAAGCTATCTGACCCCCGGCGATTCCAGCTCAGgtaagtaatttata
taccactagagattttttcatcagtttctgttataaaaataattaaaatcaacatatttttctcctttacaacagGTTGGACAGCTG
GAGCTGCCGCCTACTATGTGGGATATCTGCAACCTAGAACATTTCTGCTGAAGTACAACGAGAACGGCACAATCACAGACGCTGTGG
ATTGTGCTCTGGACCCCCTCTCCGAGACCAAGTGTACCCTCAAGAGCTTTACCGTGGAGAAGGGAATCTACCAGACCTCCAATTTTA
GGGTCCAACCCACCGAGAGCATCGTGAGGTTCCCCAACATCACAAACCTCTGCCCTTTCGGCGAAGTGTTCAACGCCACAAGGTTTG
CTTCCGTGTACGCTTGGAATAGAAAGAGAATCTCCAACTGCGTGGCCGACTATAGCGTGCTCTATAACAGCGCCTCCTTCAGCACCT
TCAAGTGTTACGGCGTGAGCCCCACCAAGCTGAACGATCTGTGTTTCACCAACGTGTACGCTGACTCCTTCGTCATTAGGGGCGACG
AAGTGAGACAAATCGCTCCCGGCCAGACCGGCAAAATCGCTGACTACAACTACAAGCTCCCCGACGACTTCACCGGCTGTGTGATCG
CTTGGAACTCCAACAACCTCGATAGCAAGGTGGGAGGCAACTACAACTATCTGTATAGACTCTTTAGAAAGTCCAATCTGAAGCCCT
TCGAGAGGGACATCAGCACAGAGATCTATCAAGCCGGATCCACACCTTGCAACGGCGTCGAGGGATTCAACTGCTACTTCCCTCTGC
AATCCTACGGCTTCCAGCCCACAAATGGCGTGGGCTACCAGCCTTACAGAGTGGTGGTGCTGTCCTTTGAACTGCTGCATGCCCCCG
CCACAGTGTGCGGACCCAAAAAGAGCACCAACCTCGTGAAGAACAAATGCGTCAATTTCAACTTCAATGGACTGACCGGCACCGGCG
TGCTCACCGAGTCCAACAAGAAGTTTCTGCCCTTCCAGCAGTTCGGAAGAGACATTGCCGATACCACAGACGCCGTGAGGGACCCTC
AGACACTGGAGATTCTGGATATCACACCTTGCAGCTTCGGCGGCGTGAGCGTGATCACACCCGGAACAAACACCAGCAACCAAGTGG
CTGTGCTGTACCAAGACGTGAATTGTACAGAGGTACCTGTGGCCATCCATGCCGATCAGCTGACCCCCACATGGAGGGTCTACAGCA
CAGgtaagtaggagaacattttcacatacaaagccatttttactttttttttaaatttcttataatcaatatgatctttttcacagG
TTCCAATGTCTTTCAGACAAGAGCTGGCTGTCTGATTGGCGCTGAGCACGTGAACAACAGCTACGAGTGCGACATCCCTATCGGCGC
CGGAATTTGCGCCAGCTACCAAACCCAGACCAATAGCCCTAGGAGGGCCAGATCCGTCGCCAGCCAGAGCATCATCGCCTATACCAT
GTCTCTGGGCGCTGAGAACTCCGTGGCCTATAGCAACAACAGCATCGCTATCCCCACCAACTTCACAATCTCCGTGACCACCGAGAT
TCTGCCCGTGAGCATGACCAAGACCAGCGTCGACTGCACCATGTATATCTGCGGCGACTCCACAGAGTGCTCCAATCTGCTGCTGCA
GTACGGCAGCTTCTGCACCCAACTCAATAGGGCTCTGACCGGAATTGCTGTCGAGCAAGACAAGAACACCCAAGAGGTGTTTGCCCA
AGTGAAACAGATTTACAAGACCCCCCCCATCAAGGACTTCGGAGGCTTCAATTTCTCCCAAATCCTCCCCGACCCCTCCAAACCCTC
CAAGAGGAGCTTTATCGAGGATCTGCTGTTCAACAAGGTGACACTGGCTGATGCCGGCTTTATCAAGCAGTATGGCGACTGTCTGGG
AGACATCGCTGCTAGGGATCTGATCTGTGCCCAGAAGTTTAATGGCCTCACCGTGCTGCCTCCTCTGCTGACCGACGAGATGATCGC
CCAGTATACAAGCGCTCTGCTGGCCGGCACAATTACCAGCGGATGGACATTTGGAGCCGGCGCTGCCCTCCAGATTCCTTTCGCCAT
GCAGATGGCCTATAGATTCAACGGCATTGGCGTCACACAGAACGTGCTGTACGAGAACCAGAAGCTGATCGCTAACCAGTTCAACAG
CGCCATTGGCAAGATCCAAGATTCCCTCAGCTCCACCGCCAGCGCCCTCGGCAAACTGCAAGACGTCGTGAATCAGAATGCCCAAGC
TCTGAACACACTGGTGAAGCAGCTCAGCAGCAATTTTGGCGCCATCTCCTCCGTGCTCAATGATATTCTGTCTAGACTGGACAAGGT
GGAGGCCGAAGTCCAGATCGATAGACTGATCACAGgtaagtgtcttaaattcagaagacgtaaagcaaaacacggttttgaggaggc
ttcttattataaatcttgcattatctacttttttctagGTAGACTGCAGTCCCTCCAGACATACGTGACCCAGCAGCTCATTAGAGC
TGCCGAGATTAGGGCCTCCGCCAATCTCGCTGCCACAAAAATGAGCGAGTGCGTGCTCGGCCAGTCCAAAAGAGTGGACTTCTGTGG
CAAGGGCTACCATCTGATGTCCTTCCCTCAGAGCGCTCCTCATGGCGTCGTGTTTCTGCATGTGACCTACGTGCCCGCCCAAGAGAA
GAACTTCACAACAGCCCCCGCTATCTGTCACGACGGAAAGGCCCACTTCCCCAGAGAGGGCGTCTTTGTGTCCAACGGCACACACTG
GTTTGTCACCCAGAGGAACTTCTATGAGCCCCAGATCATCACCACCGACAACACCTTTGTGAGCGGAAACTGCGATGTGGTCATCGG
CATCGTGAATAACACCGTGTACGACCCTCTCCAGCCCGAGCTGGACTCCTTCAAGGAGGAGCTGGATAAGTACTTTAAGAACCATAC
AAGCCCCGACGTGGACCTCGGCGACATTAGCGGAATCAACGCCAGCGTCGTGAACATCCAGAAGGAGATTGATAGACTCAACGAGGT
CGCCAAGAATCTGAACGAGTCTCTGATTGATCTGCAAGAGCTGGGCAAGTACGAGCAGTACATCAAGTGGCCTTGGTACATCTGGCT
CGGATTCATTGCCGGACTGATCGCCATCGTCATGGTGACCATCATGCTCTGCTGCATGACAAGCTGTTGCAGCTGTCTGAAAGGCTG
TTGTAGCTGTGGCAGCTGCTGTAAGTTCGATGAGGACGACTCCGAGCCCGTGCTGAAGGGCGTGAAGCTCCACTACACCTAA
SEQ ID NO: 10 (P204)
ATGTTCGTGTTCCTCGTGCTGCTGCCTCTGGTGTCCTCCCAGTGCGTCAATCTGACAACAAGAACACAGCTGCCCCCCGCCTACACC
AATTCCTTCACAAGAGGCGTGTACTACCCCGACAAGGTGTTCAGAAGCTCCGTGCTGCACAGCACCCAAGACCTCTTTCTGCCCTTT
TTCTCCAACGTCACATGGTTCCACGCTATCCACGTGTCAGgtaagttgatttagaaacacttttcaagcagtcagcccatggttacc
attaagttaaccctatcactgaattgctccaattttcctcttagGTACCAACGGCACCAAAAGGTTCGATAACCCCGTCCTCCCCTT
CAACGATGGCGTCTACTTCGCCAGCACCGAGAAGTCCAATATCATCAGAGGCTGGATCTTCGGCACCACACTGGATTCCAAGACCCA
GTCTCTGCTGATCGTGAATAACGCCACAAACGTGGTCATTAAAGTGTGCGAGTTCCAGTTCTGCAACGACCCCTTCCTCGGCGTGTA
TTACCACAAAAACAACAAGAGCTGGATGGAGTCCGAGTTTAGAGTGTACAGCAGCGCCAACAACTGCACATTCGAGTACGTGAGCCA
GCCTTTCCTCATGGATCTGGAGGGCAAGCAAGGCAATTTCAAGAATCTGAGAGAGTTTGTCTTCAAGAACATCGACGGATACTTCAA
GATTTACTCCAAGCACACCCCCATTAACCTCGTCAGAGACCTCCCCCAAGGCTTTTCCGCTCTCGAACCTCTGGTGGATCTGCCCAT
CGGCATCAACATCACAAGATTCCAAACCCTCCTCGCTCTGCATAGAAGCTATCTGACCCCCGGCGATTCCAGCTCAGgtaagtaatt
tatataccactagagattttttcatcagtttctgttataaaaataattaaaatcaacatatttttctcctttacaacagGTTGGACA
GCTGGAGCTGCCGCCTACTATGTGGGATATCTGCAACCTAGAACATTTCTGCTGAAGTACAACGAGAACGGCACAATCACAGACGCT
GTGGATTGTGCTCTGGACCCCCTCTCCGAGACCAAGTGTACCCTCAAGAGCTTTACCGTGGAGAAGGGAATCTACCAGACCTCCAAT
TTTAGGGTCCAACCCACCGAGAGCATCGTGAGGTTCCCCAACATCACAAACCTCTGCCCTTTCGGCGAAGTGTTCAACGCCACAAGG
TTTGCTTCCGTGTACGCTTGGAATAGAAAGAGAATCTCCAACTGCGTGGCCGACTATAGCGTGCTCTATAACAGCGCCTCCTTCAGC
ACCTTCAAGTGTTACGGCGTGAGCCCCACCAAGCTGAACGATCTGTGTTTCACCAACGTGTACGCTGACTCCTTCGTCATTAGGGGC
GACGAAGTGAGACAAATCGCTCCCGGCCAGACCGGCAAAATCGCTGACTACAACTACAAGCTCCCCGACGACTTCACCGGCTGTGTG
ATCGCTTGGAACTCCAACAACCTCGATAGCAAGGTGGGAGGCAACTACAACTATCTGTATAGACTCTTCAGgtaaggaatgttgcac
tgattttcacaggattttcccaagtgatactatcttattacattgatttttggctttgttttgttttcagGAAGTCCAATCTGAAGC
CCTTCGAGAGGGACATCAGCACAGAGATCTATCAAGCCGGATCCACACCTTGCAACGGCGTCGAGGGATTCAACTGCTACTTCCCTC
TGCAATCCTACGGCTTCCAGCCCACAAATGGCGTGGGCTACCAGCCTTACAGAGTGGTGGTGCTGTCCTTTGAACTGCTGCATGCCC
CCGCCACAGTGTGCGGACCCAAAAAGAGCACCAACCTCGTGAAGAACAAATGCGTCAATTTCAACTTCAATGGACTGACCGGCACCG
GCGTGCTCACCGAGTCCAACAAGAAGTTTCTGCCCTTCCAGCAGTTCGGAAGAGACATTGCCGATACCACAGACGCCGTGAGGGACC
CTCAGACACTGGAGATTCTGGATATCACACCTTGCAGCTTCGGCGGCGTGAGCGTGATCACACCCGGAACAAACACCAGCAACCAAG
TGGCTGTGCTGTACCAAGACGTGAATTGTACAGAGGTACCTGTGGCCATCCATGCCGATCAGCTGACCCCCACATGGAGGGTCTACA
GCACAGgtaagtaggagaacattttcacatacaaagccatttttactttttttttaaatttcttataatcaatatgatctttttcac
agGTTCCAATGTCTTTCAGACAAGAGCTGGCTGTCTGATTGGCGCTGAGCACGTGAACAACAGCTACGAGTGCGACATCCCTATCGG
CGCCGGAATTTGCGCCAGCTACCAAACCCAGACCAATAGCCCTAGGAGGGCCAGATCCGTCGCCAGCCAGAGCATCATCGCCTATAC
CATGTCTCTGGGCGCTGAGAACTCCGTGGCCTATAGCAACAACAGCATCGCTATCCCCACCAACTTCACAATCTCCGTGACCACCGA
GATTCTGCCCGTGAGCATGACCAAGACCAGCGTCGACTGCACCATGTATATCTGCGGCGACTCCACAGAGTGCTCCAATCTGCTGCT
GCAGTACGGCAGCTTCTGCACCCAACTCAATAGGGCTCTGACCGGAATTGCTGTCGAGCAAGACAAGAACACCCAAGAGGTGTTTGC
CCAAGTGAAACAGATTTACAAGACCCCCCCCATCAAGGACTTCGGAGGCTTCAATTTCTCCCAAATCCTCCCCGACCCCTCCAAACC
CTCCAAGAGGAGCTTTATCGAGGATCTGCTGTTCAACAAGGTGACACTGGCTGATGCAGgtaagtctatttcaaaaaagaatcatat
atattttaaaatagcttatgtattttttacacattcatttcttatttacctactatttatccagGTTTTATCAAGCAGTATGGCGAC
TGTCTGGGAGACATCGCTGCTAGGGATCTGATCTGTGCCCAGAAGTTTAATGGCCTCACCGTGCTGCCTCCTCTGCTGACCGACGAG
ATGATCGCCCAGTATACAAGCGCTCTGCTGGCCGGCACAATTACCAGCGGATGGACATTTGGAGCCGGCGCTGCCCTCCAGATTCCT
TTCGCCATGCAGATGGCCTATAGATTCAACGGCATTGGCGTCACACAGAACGTGCTGTACGAGAACCAGAAGCTGATCGCTAACCAG
TTCAACAGCGCCATTGGCAAGATCCAAGATTCCCTCAGCTCCACCGCCAGCGCCCTCGGCAAACTGCAAGACGTCGTGAATCAGAAT
GCCCAAGCTCTGAACACACTGGTGAAGCAGCTCAGCAGCAATTTTGGCGCCATCTCCTCCGTGCTCAATGATATTCTGTCTAGACTG
GACAAGGTGGAGGCCGAAGTCCAGATCGATAGACTGATCACAGgtaagtgtcttaaattcagaagacgtaaagcaaaacacggtttt
gaggaggcttcttattataaatcttgcattatctacttttttctagGTAGACTGCAGTCCCTCCAGACATACGTGACCCAGCAGCTC
ATTAGAGCTGCCGAGATTAGGGCCTCCGCCAATCTCGCTGCCACAAAAATGAGCGAGTGCGTGCTCGGCCAGTCCAAAAGAGTGGAC
TTCTGTGGCAAGGGCTACCATCTGATGTCCTTCCCTCAGAGCGCTCCTCATGGCGTCGTGTTTCTGCATGTGACCTACGTGCCCGCC
CAAGAGAAGAACTTCACAACAGCCCCCGCTATCTGTCACGACGGAAAGGCCCACTTCCCCAGAGAGGGCGTCTTTGTGTCCAACGGC
ACACACTGGTTTGTCACCCAGAGGAACTTCTATGAGCCCCAGATCATCACCACCGACAACACCTTTGTGAGCGGAAACTGCGATGTG
GTCATCGGCATCGTGAATAACACCGTGTACGACCCTCTCCAGCCCGAGCTGGACTCCTTCAAGGAGGAGCTGGATAAGTACTTTAAG
AACCATACAAGCCCCGACGTGGACCTCGGCGACATTTCAGgtaagttgtccaacttttcaaagatccaggttttcttttaccataaa
tgtgttattgtctgtactaatctataggatttctctcttttgtagGTATCAACGCCAGCGTCGTGAACATCCAGAAGGAGATTGATA
GACTCAACGAGGTCGCCAAGAATCTGAACGAGTCTCTGATTGATCTGCAAGAGCTGGGCAAGTACGAGCAGTACATCAAGTGGCCTT
GGTACATCTGGCTCGGATTCATTGCCGGACTGATCGCCATCGTCATGGTGACCATCATGCTCTGCTGCATGACAAGCTGTTGCAGCT
GTCTGAAAGGCTGTTGTAGCTGTGGCAGCTGCTGTAAGTTCGATGAGGACGACTCCGAGCCCGTGCTGAAGGGCGTGAAGCTCCACT
ACACCTAA
SEQ ID NO: 11 (P171)
ATGTTCGTGTTCCTCGTGCTGCTGCCTCTGGTGTCCTCCCAGTGCGTCAATCTGACAACAAGAACACAGCTGCCCCCCGCCTACACC
AATTCCTTCACAAGAGGCGTGTACTACCCCGACAAGGTGTTCAGAAGCTCCGTGCTGCACAGCACCCAAGACCTCTTTCTGCCCTTT
TTCTCCAACGTCACATGGTTCCACGCTATCCACGTGTCAGgtaagttgatttagaaacacttttcaagcagtcagcccatggttacc
attaagttaaccctatcactgaattgctccaattttcctcttagGTACCAACGGCACCAAAAGGTTCGATAACCCCGTCCTCCCCTT
CAACGATGGCGTCTACTTCGCCAGCACCGAGAAGTCCAATATCATCAGAGGCTGGATCTTCGGCACCACACTGGATTCCAAGACCCA
GTCTCTGCTGATCGTGAATAACGCCACAAACGTGGTCATTAAAGTGTGCGAGTTCCAGTTCTGCAACGACCCCTTCCTCGGCGTGTA
TTACCACAAAAACAACAAGAGCTGGATGGAGTCCGAGTTCAGgtaaggaaatttccatgagtttcactcttgaagcattggggttat
ttgtgccagaggctaatgacccatgctggcccttcacttttctagGGTGTACAGCAGCGCCAACAACTGCACATTCGAGTACGTGAG
CCAGCCTTTCCTCATGGATCTGGAGGGCAAGCAAGGCAATTTCAAGAATCTGAGAGAGTTTGTCTTCAAGAACATCGACGGATACTT
CAAGATTTACTCCAAGCACACCCCCATTAACCTCGTCAGAGACCTCCCCCAAGGCTTTTCCGCTCTCGAACCTCTGGTGGATCTGCC
CATCGGCATCAACATCACAAGATTCCAAACCCTCCTCGCTCTGCATAGAAGCTATCTGACCCCCGGCGATTCCAGCTCAGgtaagta
atttatataccactagagattttttcatcagtttctgttataaaaataattaaaatcaacatatttttctcctttacaacagGTTGG
ACAGCTGGAGCTGCCGCCTACTATGTGGGATATCTGCAACCTAGAACATTTCTGCTGAAGTACAACGAGAACGGCACAATCACAGAC
GCTGTGGATTGTGCTCTGGACCCCCTCTCCGAGACCAAGTGTACCCTCAAGAGCTTTACCGTGGAGAAGGGAATCTACCAGACCTCC
AATTTTAGGGTCCAACCCACCGAGAGCATCGTGAGGTTCCCCAACATCACAAACCTCTGCCCTTTCGGCGAAGTGTTCAACGCCACA
AGGTTTGCTTCCGTGTACGCTTGGAACAGgtaagtagtgctgattatacacaagatattgtctagaacttgatgagactgtggatat
gaatatttcactcttttctcagGAAGAGAATCTCCAACTGCGTGGCCGACTATAGCGTGCTCTATAACAGCGCCTCCTTCAGCACCT
TCAAGTGTTACGGCGTGAGCCCCACCAAGCTGAACGATCTGTGTTTCACCAACGTGTACGCTGACTCCTTCGTCATTAGGGGCGACG
AAGTGAGACAAATCGCTCCCGGCCAGACCGGCAAAATCGCTGACTACAACTACAAGCTCCCCGACGACTTCACCGGCTGTGTGATCG
CTTGGAACTCCAACAACCTCGATAGCAAGGTGGGAGGCAACTACAACTATCTGTATAGACTCTTCAGgtaaggaatgttgcactgat
tttcacaggattttcccaagtgatactatcttattacattgatttttggctttgttttgttttcagGAAGTCCAATCTGAAGCCCTT
CGAGAGGGACATCAGCACAGAGATCTATCAAGCCGGATCCACACCTTGCAACGGCGTCGAGGGATTCAACTGCTACTTCCCTCTGCA
ATCCTACGGCTTCCAGCCCACAAATGGCGTGGGCTACCAGCCTTACAGAGTGGTGGTGCTGTCCTTTGAACTGCTGCATGCCCCCGC
CACAGTGTGCGGACCCAAAAAGAGCACCAACCTCGTGAAGAACAAATGCGTCAATTTCAACTTCAATGGACTGACCGGCACAGgtaa
gtgacttgctttcttacatcaaaaaggcatccagtgtctgtttaagaattgccttctcaatattctctgttgattcctttccagGTG
TGCTCACCGAGTCCAACAAGAAGTTTCTGCCCTTCCAGCAGTTCGGAAGAGACATTGCCGATACCACAGACGCCGTGAGGGACCCTC
AGACACTGGAGATTCTGGATATCACACCTTGCAGCTTCGGCGGCGTGAGCGTGATCACACCCGGAACAAACACCAGCAACCAAGTGG
CTGTGCTGTACCAAGACGTGAATTGTACAGAGGTACCTGTGGCCATCCATGCCGATCAGCTGACCCCCACATGGAGGGTCTACAGCA
CAGgtaagtaggagaacattttcacatacaaagccatttttactttttttttaaatttcttataatcaatatgatctttttcacagG
TTCCAATGTCTTTCAGACAAGAGCTGGCTGTCTGATTGGCGCTGAGCACGTGAACAACAGCTACGAGTGCGACATCCCTATCGGCGC
CGGAATTTGCGCCAGCTACCAAACCCAGACCAATAGCCCTAGGAGGGCCAGATCCGTCGCCAGCCAGAGCATCATCGCCTATACCAT
GTCTCTGGGCGCTGAGAACTCCGTGGCCTATAGCAACAACAGCATCGCTATCCCCACCAACTTCACAATCTCCGTGACCACAGgtaa
gttgctttctctgaatacaaaactattgtttgactgtctttaagaatattactttttcatcataacttcttctttgaaaagAAATTC
TGCCCGTGAGCATGACCAAGACCAGCGTCGACTGCACCATGTATATCTGCGGCGACTCCACAGAGTGCTCCAATCTGCTGCTGCAGT
ACGGCAGCTTCTGCACCCAACTCAATAGGGCTCTGACCGGAATTGCTGTCGAGCAAGACAAGAACACCCAAGAGGTGTTTGCCCAAG
TGAAACAGATTTACAAGACCCCCCCCATCAAGGACTTCGGAGGCTTCAATTTCTCCCAAATCCTCCCCGACCCCTCCAAACCCTCCA
AGAGGAGCTTTATCGAGGATCTGCTGTTCAACAAGGTGACACTGGCTGATGCAGgtaagtctatttcaaaaaagaatcatatatatt
ttaaaatagcttatgtattttttacacattcatttcttatttacctactatttatccagGTTTTATCAAGCAGTATGGCGACTGTCT
GGGAGACATCGCTGCTAGGGATCTGATCTGTGCCCAGAAGTTTAATGGCCTCACCGTGCTGCCTCCTCTGCTGACCGACGAGATGAT
CGCCCAGTATACAAGCGCTCTGCTGGCCGGCACAATTACCAGCGGATGGACATTTGGAGCCGGCGCTGCCCTCCAGATTCCTTTCGC
CATGCAGATGGCCTACAGgtaagcaaatgaaccatcatcccatcattttgagttatatccttcctttgttatatggggcttacactt
atcatttctcctttgctttagGTTCAACGGCATTGGCGTCACACAGAACGTGCTGTACGAGAACCAGAAGCTGATCGCTAACCAGTT
CAACAGCGCCATTGGCAAGATCCAAGATTCCCTCAGCTCCACCGCCAGCGCCCTCGGCAAACTGCAAGACGTCGTGAATCAGAATGC
CCAAGCTCTGAACACACTGGTGAAGCAGCTCAGCAGCAATTTTGGCGCCATCTCCTCCGTGCTCAATGATATTCTGTCTAGACTGGA
CAAGGTGGAGGCCGAAGTCCAGATCGATAGACTGATCACAGgtaagtgtcttaaattcagaagacgtaaagcaaaacacggttttga
ggaggcttcttattataaatcttgcattatctacttttttctagGTAGACTGCAGTCCCTCCAGACATACGTGACCCAGCAGCTCAT
TAGAGCTGCCGAGATTAGGGCCTCCGCCAATCTCGCTGCCACAAAAATGAGCGAGTGCGTGCTCGGCCAGTCCAAAAGAGTGGACTT
CTGTGGCAAGGGCTACCATCTGATGTCCTTCCCTCAGAGCGCTCCTCATGGCGTCGTGTTTCTGCATGTGACCTACGTGCCCGCCCA
AGAGAAGAACTTCACAACAGCCCCCGCTATCTGTCACGACGGAAAGGCCCACTTCCCCAGgtaagtcattatatgaagaaaaaccca
ggtgcatgttttacatgaagaaaactggtatttgtttgactggttttgcttttatgttttagGGAGGGCGTCTTTGTGTCCAACGGC
ACACACTGGTTTGTCACCCAGAGGAACTTCTATGAGCCCCAGATCATCACCACCGACAACACCTTTGTGAGCGGAAACTGCGATGTG
GTCATCGGCATCGTGAATAACACCGTGTACGACCCTCTCCAGCCCGAGCTGGACTCCTTCAAGGAGGAGCTGGATAAGTACTTTAAG
AACCATACAAGCCCCGACGTGGACCTCGGCGACATTTCAGgtaagttgtccaacttttcaaagatccaggttttcttttaccataaa
tgtgttattgtctgtactaatctataggatttctctcttttgtagGTATCAACGCCAGCGTCGTGAACATCCAGAAGGAGATTGATA
GACTCAACGAGGTCGCCAAGAATCTGAACGAGTCTCTGATTGATCTGCAAGAGCTGGGCAAGTACGAGCAGTACATCAAGTGGCCTT
GGTACATCTGGCTCGGATTCATTGCCGGACTGATCGCCATCGTCATGGTGACCATCATGCTCTGCTGCATGACAAGCTGTTGCAGCT
GTCTGAAAGGCTGTTGTAGCTGTGGCAGCTGCTGTAAGTTCGATGAGGACGACTCCGAGCCCGTGCTGAAGGGCGTGAAGCTCCACT
ACACCTAA
SEQ ID NO: 12 (P231)
ATGTTCGTGTTCCTCGTGCTGCTGCCTCTGGTGTCCTCCCAGTGCGTCAATCTGACAACAAGAACACAGCTGCCCCCCGCCTACACC
AATTCCTTCACAAGAGGCGTGTACTACCCCGACAAGGTGTTCAGAAGCTCCGTGCTGCACAGCACCCAAGACCTCTTTCTGCCCTTT
TTCTCCAACGTCACATGGTTCCACGCTATCCACGTGTCAGgtaagttgatttagaaacacttttcaagcagtcagcccatggttacc
attaagttaaccctatcactgaattgctccaattttcctcttagGTACCAACGGCACCAAAAGGTTCGATAACCCCGTCCTCCCCTT
CAACGATGGCGTCTACTTCGCCAGCACCGAGAAGTCCAATATCATCAGAGGCTGGATCTTCGGCACCACACTGGATTCCAAGACCCA
GTCTCTGCTGATCGTGAATAACGCCACAAACGTGGTCATTAAAGTGTGCGAGTTCCAGTTCTGCAACGACCCCTTCCTCGGCGTGTA
TTACCACAAAAACAACAAGAGCTGGATGGAGTCCGAGTTCAGgtaaggaaatttccatgagtttcactcttgaagcattggggttat
ttgtgccagaggctaatgacccatgctggcccttcacttttctagGGTGTACAGCAGCGCCAACAACTGCACATTCGAGTACGTGAG
CCAGCCTTTCCTCATGGATCTGGAGGGCAAGCAAGGCAATTTCAAGAATCTGAGAGAGTTTGTCTTCAAGAACATCGACGGATACTT
CAAGATTTACTCCAAGCACACCCCCATTAACCTCGTCAGAGACCTCCCCCAAGgtaagtgtgatgtggctaataatttatgtgttta
tcaatttgtcgtttatgttaaataaaataatcatatactttttttcagGTTTTTCCGCTCTCGAACCTCTGGTGGATCTGCCCATCG
GCATCAACATCACAAGATTCCAAACCCTCCTCGCTCTGCATAGAAGCTATCTGACCCCCGGCGATTCCAGCTCAGgtaagtaattta
tataccactagagattttttcatcagtttctgttataaaaataattaaaatcaacatatttttctcctttacaacagGTTGGACAGC
TGGAGCTGCCGCCTACTATGTGGGATATCTGCAACCTAGAACATTTCTGCTGAAGTACAACGAGAACGGCACAATCACAGACGCTGT
GGATTGTGCTCTGGACCCCCTCTCCGAGACCAAGTGTACCCTCAAGAGCTTTACCGTGGAGAAGGGAATCTACCAGACCTCCAATTT
TAGGGTCCAACCCACCGAGAGCATCGTGAGGTTCCCCAACATCACAAACCTCTGCCCTTTCGGCGAAGTGTTCAACGCCACAAGGTT
TGCTTCCGTGTACGCTTGGAACAGgtaagtagtgctgattatacacaagatattgtctagaacttgatgagactgtggatatgaata
tttcactcttttctcagGAAGAGAATCTCCAACTGCGTGGCCGACTATAGCGTGCTCTATAACAGCGCCTCCTTCAGCACCTTCAAG
TGTTACGGCGTGAGCCCCACCAAGCTGAACGATCTGTGTTTCACCAACGTGTACGCTGACTCCTTCGTCATTAGGGGCGACGAAGTG
AGACAAATCGCTCCCGGCCAGACCGGCAAAATCGCTGACTACAACTACAAGCTCCCCGACGACTTCACCGGCTGTGTGATCGCTTGG
AACTCCAACAACCTCGATAGCAAGGTGGGAGGCAACTACAACTATCTGTATAGACTCTTCAGgtaaggaatgttgcactgattttca
caggattttcccaagtgatactatcttattacattgatttttggctttgttttgttttcagGAAGTCCAATCTGAAGCCCTTCGAGA
GGGACATCAGCACAGAGATCTATCAAGCCGGATCCACACCTTGCAACGGCGTCGAGGGATTCAACTGCTACTTCCCTCTGCAATCCT
ACGGCTTCCAGCCCACAAATGGCGTGGGCTACCAGCCTTACAGAGTGGTGGTGCTGTCCTTTGAACTGCTGCATGCCCCCGCCACAG
TGTGCGGACCCAAAAAGAGCACCAACCTCGTGAAGAACAAATGCGTCAATTTCAACTTCAATGGACTGACCGGCACAGgtaagtgac
ttgctttcttacatcaaaaaggcatccagtgtctgtttaagaattgccttctcaatattctctgttgattcctttccagGTGTGCTC
ACCGAGTCCAACAAGAAGTTTCTGCCCTTCCAGCAGTTCGGAAGAGACATTGCCGATACCACAGACGCCGTGAGGGACCCTCAGACA
CTGGAGATTCTGGATATCACACCTTGCAGCTTCGGCGGCGTGAGCGTGATCACACCCGGAACAAACACCAGCAACCAAGTGGCTGTG
CTGTACCAAGACGTGAATTGTACAGAGGTACCTGTGGCCATCCATGCCGATCAGCTGACCCCCACATGGAGGGTCTACAGCACAGgt
aagtaggagaacattttcacatacaaagccatttttactttttttttaaatttcttataatcaatatgatctttttcacagGTTCCA
ATGTCTTTCAGACAAGAGCTGGCTGTCTGATTGGCGCTGAGCACGTGAACAACAGCTACGAGTGCGACATCCCTATCGGCGCCGGAA
TTTGCGCCAGCTACCAAACCCAGACCAATAGCCCTAGGAGGGCCAGATCCGTCGCCAGCCAGAGCATCATCGCCTATACCATGTCTC
TGGGCGCTGAGAACTCCGTGGCCTATAGCAACAACAGCATCGCTATCCCCACCAACTTCACAATCTCCGTGACCACAGgtaagttgc
tttctctgaatacaaaactattgtttgactgtctttaagaatattactttttcatcataacttcttctttgaaaagAAATTCTGCCC
GTGAGCATGACCAAGACCAGCGTCGACTGCACCATGTATATCTGCGGCGACTCCACAGAGTGCTCCAATCTGCTGCTGCAGTACGGC
AGCTTCTGCACCCAACTCAATAGGGCTCTGACCGGAATTGCTGTCGAGCAAGACAAGAACACCCAAGAGGTGTTTGCCCAAGTGAAA
CAGATTTACAAGACCCCCCCCATCAAGGACTTCGGAGGCTTCAATTTCTCCCAAATCCTCCCCGACCCCTCCAAACCCTCCAAGAGG
AGCTTTATCGAGGATCTGCTGTTCAACAAGGTGACACTGGCTGATGCAGgtaagtctatttcaaaaaagaatcatatatattttaaa
atagcttatgtattttttacacattcatttcttatttacctactatttatccagGTTTTATCAAGCAGTATGGCGACTGTCTGGGAG
ACATCGCTGCTAGGGATCTGATCTGTGCCCAGAAGTTTAATGGCCTCACCGTGCTGCCTCCTCTGCTGACCGACGAGATGATCGCCC
AGTATACAAGCGCTCTGCTGGCCGGCACAATTACCAGCGGATGGACATTTGGAGCCGGCGCTGCCCTCCAGATTCCTTTCGCCATGC
AGATGGCCTACAGgtaagcaaatgaaccatcatcccatcattttgagttatatccttcctttgttatatggggcttacacttatcat
ttctcctttgctttagGTTCAACGGCATTGGCGTCACACAGAACGTGCTGTACGAGAACCAGAAGCTGATCGCTAACCAGTTCAACA
GCGCCATTGGCAAGATCCAAGATTCCCTCAGCTCCACCGCCAGCGCCCTCGGCAAACTGCAAGACGTCGTGAATCAGAATGCCCAAG
CTCTGAACACACTGGTGAAGCAGCTCAGCAGCAATTTTGGCGCCATCTCCTCCGTGCTCAATGATATTCTGTCTAGACTGGACAAGG
TGGAGGCCGAAGTCCAGATCGATAGACTGATCACAGgtaagtgtcttaaattcagaagacgtaaagcaaaacacggttttgaggagg
cttcttattataaatcttgcattatctacttttttctagGTAGACTGCAGTCCCTCCAGACATACGTGACCCAGCAGCTCATTAGAG
CTGCCGAGATTAGGGCCTCCGCCAATCTCGCTGCCACAAAAATGAGCGAGTGCGTGCTCGGCCAGTCCAAAAGAGTGGACTTCTGTG
GCAAGGGCTACCATCTGATGTCCTTCCCTCAGAGCGCTCCTCATGGCGTCGTGTTTCTGCATGTGACCTACGTGCCCGCCCAAGAGA
AGAACTTCACAACAGCCCCCGCTATCTGTCACGACGGAAAGGCCCACTTCCCCAGgtaagtcattatatgaagaaaaacccaggtgc
atgttttacatgaagaaaactggtatttgtttgactggttttgcttttatgttttagGGAGGGCGTCTTTGTGTCCAACGGCACACA
CTGGTTTGTCACCCAGAGGAACTTCTATGAGCCCCAGATCATCACCACCGACAACACCTTTGTGAGCGGAAACTGCGATGTGGTCAT
CGGCATCGTGAATAACACCGTGTACGACCCTCTCCAGCCCGAGCTGGACTCCTTCAAGGAGGAGCTGGATAAGTACTTTAAGAACCA
TACAAGCCCCGACGTGGACCTCGGCGACATTTCAGgtaagttgtccaacttttcaaagatccaggttttcttttaccataaatgtgt
tattgtctgtactaatctataggatttctctcttttgtagGTATCAACGCCAGCGTCGTGAACATCCAGAAGGAGATTGATAGACTC
AACGAGGTCGCCAAGAATCTGAACGAGTCTCTGATTGATCTGCAAGAGCTGGGCAAGTACGAGCAGTACATCAAGTGGCCTTGGTAC
ATCTGGCTCGGATTCATTGCCGGACTGATCGCCATCGTCATGGTGACCATCATGCTCTGCTGCATGACAAGCTGTTGCAGCTGTCTG
AAAGGCTGTTGTAGCTGTGGCAGCTGCTGTAAGTTCGATGAGGACGACTCCGAGCCCGTGCTGAAGGGCGTGAAGCTCCACTACACC
TAA
SEQ ID NO: 13 (P232)
ATGTTCGTGTTCCTCGTGCTGCTGCCTCTGGTGTCCTCCCAGTGCGTCAATCTGACAACAAGAACACAGCTGCCCCCCGCCTACACC
AATTCCTTCACAAGAGGCGTGTACTACCCCGACAAGGTGTTCAGAAGCTCCGTGCTGCACAGCACCCAAGACCTCTTTCTGCCCTTT
TTCTCCAACGTCACATGGTTCCACGCTATCCACGTGTCAGgtaagttgatttagaaacacttttcaagcagtcagcccatggttacc
attaagttaaccctatcactgaattgctccaattttcctcttagGTACCAACGGCACCAAAAGGTTCGATAACCCCGTCCTCCCCTT
CAACGATGGCGTCTACTTCGCCAGCACCGAGAAGTCCAATATCATCAGAGGCTGGATCTTCGGCACCACACTGGATTCCAAGACCCA
GTCTCTGCTGATCGTGAATAACGCCACAAACGTGGTCATTAAAGTGTGCGAGTTCCAGTTCTGCAACGACCCCTTCCTCGGCGTGTA
TTACCACAAAAACAACAAGAGCTGGATGGAGTCCGAGTTCAGgtaaggaaatttccatgagtttcactcttgaagcattggggttat
ttgtgccagaggctaatgacccatgctggcccttcacttttctagGGTGTACAGCAGCGCCAACAACTGCACATTCGAGTACGTGAG
CCAGCCTTTCCTCATGGATCTGGAGGGCAAGCAAGgtaagttaataccctttttaattaaaatgaattagtatttgccatttacttt
tactatttaagagatgtaaaattgcttttcagGTAATTTCAAGAATCTGAGAGAGTTTGTCTTCAAGAACATCGACGGATACTTCAA
GATTTACTCCAAGCACACCCCCATTAACCTCGTCAGAGACCTCCCCCAAGgtaagtgtgatgtggctaataatttatgtgtttatca
atttgtcgtttatgttaaataaaataatcatatactttttttcagGTTTTTCCGCTCTCGAACCTCTGGTGGATCTGCCCATCGGCA
TCAACATCACCAGgtaagtaataggaagtactgcatttcttcttcaaggacaaaattaatatctagcctaaaaaattaattttcatc
ttttaaatatttcagGTTCCAAACCCTCCTCGCTCTGCATAGAAGCTATCTGACCCCCGGCGATTCCAGCTCAGgtaagtaatttat
ataccactagagattttttcatcagtttctgttataaaaataattaaaatcaacatatttttctcctttacaacagGTTGGACAGCT
GGAGCTGCCGCCTACTATGTGGGATATCTGCAACCTAGAACATTTCTGCTGAAGTACAACGAGAACGGCACAATCACAGACGCTGTG
GATTGTGCTCTGGACCCCCTCTCCGAGACCAAGTGTACCCTCAAGAGCTTTACCGTGGAGAAGGGAATCTACCAGACCTCCAATTTT
AGGGTCCAACCCACCGAGAGCATCGTGAGGTTCCCCAACATCACAAACCTCTGCCCTTTCGGCGAAGTGTTCAACGCCACAAGGTTT
GCTTCCGTGTACGCTTGGAACAGgtaagtagtgctgattatacacaagatattgtctagaacttgatgagactgtggatatgaatat
ttcactcttttctcagGAAGAGAATCTCCAACTGCGTGGCCGACTATAGCGTGCTCTATAACAGCGCCTCCTTCAGCACCTTCAAGT
GTTACGGCGTGAGCCCCACCAAGCTGAACGATCTGTGTTTCACCAACGTGTACGCTGACTCCTTCGTCATTAGGGGCGACGAAGTGA
GACAAATCGCTCCCGGCCAGACCGGCAAAATCGCTGACTACAACTACAAGCTCCCCGACGACTTCACCGGCTGTGTGATCGCTTGGA
ACTCCAACAACCTCGATAGCAAGGTGGGAGGCAACTACAACTATCTGTATAGACTCTTCAGgtaaggaatgttgcactgattttcac
aggattttcccaagtgatactatcttattacattgatttttggctttgttttgttttcagGAAGTCCAATCTGAAGCCCTTCGAGAG
GGACATCAGCACAGAGATCTATCAAGCCGGATCCACACCTTGCAACGGCGTCGAGGGATTCAACTGCTACTTCCCTCTGCAATCCTA
CGGCTTCCAGCCCACAAATGGCGTGGGCTACCAGCCTTACAGAGTGGTGGTGCTGTCCTTTGAACTGCTGCATGCCCCCGCCACAGT
GTGCGGACCCAAAAAGAGCACCAACCTCGTGAAGAACAAATGCGTCAATTTCAACTTCAATGGACTGACCGGCACAGgtaagtgact
tgctttcttacatcaaaaaggcatccagtgtctgtttaagaattgccttctcaatattctctgttgattcctttccagGTGTGCTCA
CCGAGTCCAACAAGAAGTTTCTGCCCTTCCAGCAGTTCGGAAGAGACATTGCCGATACCACAGACGCCGTGAGGGACCCTCAGACAC
TGGAGATTCTGGATATCACACCTTGCAGCTTCGGCGGCGTGAGCGTGATCACACCCGGAACAAACACCAGCAACCAAGTGGCTGTGC
TGTACCAAGACGTGAATTGTACAGAGGTACCTGTGGCCATCCATGCCGATCAGCTGACCCCCACATGGAGGGTCTACAGCACAGgta
agtaggagaacattttcacatacaaagccatttttactttttttttaaatttcttataatcaatatgatctttttcacagGTTCCAA
TGTCTTTCAGACAAGAGCTGGCTGTCTGATTGGCGCTGAGCACGTGAACAACAGCTACGAGTGCGACATCCCTATCGGCGCCGGAAT
TTGCGCCAGCTACCAAACCCAGACCAATAGCCCTAGGAGGGCCAGATCCGTCGCCAGCCAGAGCATCATCGCCTATACCATGTCTCT
GGGCGCTGAGAACTCCGTGGCCTATAGCAACAACAGCATCGCTATCCCCACCAACTTCACAATCTCCGTGACCACAGgtaagttgct
ttctctgaatacaaaactattgtttgactgtctttaagaatattactttttcatcataacttcttctttgaaaagAAATTCTGCCCG
TGAGCATGACCAAGACCAGCGTCGACTGCACCATGTATATCTGCGGCGACTCCACAGAGTGCTCCAATCTGCTGCTGCAGTACGGCA
GCTTCTGCACCCAACTCAATAGGGCTCTGACCGGAATTGCTGTCGAGCAAGACAAGAACACCCAAGAGGTGTTTGCCCAAGTGAAAC
AGATTTACAAGACCCCCCCCATCAAGGACTTCGGAGGCTTCAATTTCTCCCAAATCCTCCCCGACCCCTCCAAACCCTCCAAGAGGA
GCTTTATCGAGGATCTGCTGTTCAACAAGGTGACACTGGCTGATGCAGgtaagtctatttcaaaaaagaatcatatatattttaaaa
tagcttatgtattttttacacattcatttcttatttacctactatttatccagGTTTTATCAAGCAGTATGGCGACTGTCTGGGAGA
CATCGCTGCTAGGGATCTGATCTGTGCCCAGAAGTTTAATGGCCTCACCGTGCTGCCTCCTCTGCTGACCGACGAGATGATCGCCCA
GTATACAAGCGCTCTGCTGGCCGGCACAATTACCAGCGGATGGACATTTGGAGCCGGCGCTGCCCTCCAGATTCCTTTCGCCATGCA
GATGGCCTACAGgtaagcaaatgaaccatcatcccatcattttgagttatatccttcctttgttatatggggcttacacttatcatt
tctcctttgctttagGTTCAACGGCATTGGCGTCACACAGAACGTGCTGTACGAGAACCAGAAGCTGATCGCTAACCAGTTCAACAG
CGCCATTGGCAAGATCCAAGATTCCCTCAGCTCCACCGCCAGCGCCCTCGGCAAACTGCAAGACGTCGTGAATCAGAATGCCCAAGC
TCTGAACACACTGGTGAAGCAGCTCAGCAGCAATTTTGGCGCCATCTCCTCCGTGCTCAATGATATTCTGTCTAGACTGGACAAGGT
GGAGGCCGAAGTCCAGATCGATAGACTGATCACAGgtaagtgtcttaaattcagaagacgtaaagcaaaacacggttttgaggaggc
ttcttattataaatcttgcattatctacttttttctagGTAGACTGCAGTCCCTCCAGACATACGTGACCCAGCAGCTCATTAGAGC
TGCCGAGATTAGGGCCTCCGCCAATCTCGCTGCCACAAAAATGAGCGAGTGCGTGCTCGGCCAGTCCAAAAGAGTGGACTTCTGTGG
CAAGGGCTACCATCTGATGTCCTTCCCTCAGAGCGCTCCTCATGGCGTCGTGTTTCTGCATGTGACCTACGTGCCCGCCCAAGAGAA
GAACTTCACAACAGCCCCCGCTATCTGTCACGACGGAAAGGCCCACTTCCCCAGgtaagtcattatatgaagaaaaacccaggtgca
tgttttacatgaagaaaactggtatttgtttgactggttttgcttttatgttttagGGAGGGCGTCTTTGTGTCCAACGGCACACAC
TGGTTTGTCACCCAGAGGAACTTCTATGAGCCCCAGATCATCACCACCGACAACACCTTTGTGAGCGGAAACTGCGATGTGGTCATC
GGCATCGTGAATAACACCGTGTACGACCCTCTCCAGCCCGAGCTGGACTCCTTCAAGGAGGAGCTGGATAAGTACTTTAAGAACCAT
ACAAGCCCCGACGTGGACCTCGGCGACATTTCAGgtaagttgtccaacttttcaaagatccaggttttcttttaccataaatgtgtt
attgtctgtactaatctataggatttctctcttttgtagGTATCAACGCCAGCGTCGTGAACATCCAGAAGGAGATTGATAGACTCA
ACGAGGTCGCCAAGAATCTGAACGAGTCTCTGATTGATCTGCAAGAGCTGGGCAAGTACGAGCAGTACATCAAGTGGCCTTGGTACA
TCTGGCTCGGATTCATTGCCGGACTGATCGCCATCGTCATGGTGACCATCATGCTCTGCTGCATGACAAGCTGTTGCAGCTGTCTGA
AAGGCTGTTGTAGCTGTGGCAGCTGCTGTAAGTTCGATGAGGACGACTCCGAGCCCGTGCTGAAGGGCGTGAAGCTCCACTACACCT
AA
SEQ ID NO: 14 (P186)
ATGTTCGTGTTCCTCGTGCTGCTGCCTCTGGTGTCCTCCCAGTGCGTCAATCTGACAACAAGAACACAGCTGCCCCCCGCCTACACC
AATTCCTTCACAAGAGGCGTGTACTACCCCGACAAGGTGTTCAGAAGCTCCGTGCTGCACAGCACCCAAGACCTCTTTCTGCCCTTT
TTCTCCAACGTCACATGGTTCCACGCTATCCACGTGTCAGgtaagtatttaaagaagattctatttatactgtatatgtatcattta
tttatttctccaggttcatattgcatgatttttctgttttcagGTACCAACGGCACCAAAAGGTTCGATAACCCCGTCCTCCCCTTC
AACGATGGCGTCTACTTCGCCAGCACCGAGAAGTCCAATATCATCAGAGGCTGGATCTTCGGCACCACACTGGATTCCAAGACCCAG
TCTCTGCTGATCGTGAATAACGCCACAAACGTGGTCATTAAAGTGTGCGAGTTCCAGTTCTGCAACGACCCCTTCCTCGGCGTGTAT
TACCACAAAAACAACAAGAGCTGGATGGAGTCCGAGTTCAGgtaagtacagaagccatcaaacttttatatctgttttattcatttt
caaataattataaaaataatattcttactaatatttatttcagGGTGTACAGCAGCGCCAACAACTGCACATTCGAGTACGTGAGCC
AGCCTTTCCTCATGGATCTGGAGGGCAAGCAAGGCAATTTCAAGAATCTGAGAGAGTTTGTCTTCAAGAACATCGACGGATACTTCA
AGATTTACTCCAAGCACACCCCCATTAACCTCGTCAGAGACCTCCCCCAAGGCTTTTCCGCTCTCGAACCTCTGGTGGATCTGCCCA
TCGGCATCAACATCACAAGATTCCAAACCCTCCTCGCTCTGCATAGAAGCTATCTGACCCCCGGCGATTCCAGCTCAGgtaagtgtg
atgtggctaataatttatgtgtttatcaatttgtcgtttatgttaaataaaataatcatatactttttttcagGTTGGACAGCTGGA
GCTGCCGCCTACTATGTGGGATATCTGCAACCTAGAACATTTCTGCTGAAGTACAACGAGAACGGCACAATCACAGACGCTGTGGAT
TGTGCTCTGGACCCCCTCTCCGAGACCAAGTGTACCCTCAAGAGCTTTACCGTGGAGAAGGGAATCTACCAGACCTCCAATTTTAGG
GTCCAACCCACCGAGAGCATCGTGAGGTTCCCCAACATCACAAACCTCTGCCCTTTCGGCGAAGTGTTCAACGCCACAAGGTTTGCT
TCCGTGTACGCTTGGAACAGgtaagtactttcttaaatcaattctttagagcctttttaatttaaaaaatgtgcatacttcttttaa
aatactatgtatattttcagGAAGAGAATCTCCAACTGCGTGGCCGACTATAGCGTGCTCTATAACAGCGCCTCCTTCAGCACCTTC
AAGTGTTACGGCGTGAGCCCCACCAAGCTGAACGATCTGTGTTTCACCAACGTGTACGCTGACTCCTTCGTCATTAGGGGCGACGAA
GTGAGACAAATCGCTCCCGGCCAGACCGGCAAAATCGCTGACTACAACTACAAGCTCCCCGACGACTTCACCGGCTGTGTGATCGCT
TGGAACTCCAACAACCTCGATAGCAAGGTGGGAGGCAACTACAACTATCTGTATAGACTCTTCAGgtaagtattgatttaaatgtaa
ttacatttccactcatctacttaatttaagaattaggaattcgtatcttctttttgaacctcttaatctctttacagGAAGTCCAAT
CTGAAGCCCTTCGAGAGGGACATCAGCACAGAGATCTATCAAGCCGGATCCACACCTTGCAACGGCGTCGAGGGATTCAACTGCTAC
TTCCCTCTGCAATCCTACGGCTTCCAGCCCACAAATGGCGTGGGCTACCAGCCTTACAGAGTGGTGGTGCTGTCCTTTGAACTGCTG
CATGCCCCCGCCACAGTGTGCGGACCCAAAAAGAGCACCAACCTCGTGAAGAACAAATGCGTCAATTTCAACTTCAATGGACTGACC
GGCACAGgtaagtcgattccttgcttatgtatatatctcacagtttgtattttgaatttttaaaaaatatttttcttttttttcttt
tttcttacagGTGTGCTCACCGAGTCCAACAAGAAGTTTCTGCCCTTCCAGCAGTTCGGAAGAGACATTGCCGATACCACAGACGCC
GTGAGGGACCCTCAGACACTGGAGATTCTGGATATCACACCTTGCAGCTTCGGCGGCGTGAGCGTGATCACACCCGGAACAAACACC
AGCAACCAAGTGGCTGTGCTGTACCAAGACGTGAATTGTACAGAGGTACCTGTGGCCATCCATGCCGATCAGCTGACCCCCACATGG
AGGGTCTACAGCACAGgtaagtagaagcttagattattttataaaactgtatgcacttctttaaaaatacttttactaacataaaat
tgtgattttacagGTTCCAATGTCTTTCAGACAAGAGCTGGCTGTCTGATTGGCGCTGAGCACGTGAACAACAGCTACGAGTGCGAC
ATCCCTATCGGCGCCGGAATTTGCGCCAGCTACCAAACCCAGACCAATAGCCCTAGGAGGGCCAGATCCGTCGCCAGCCAGAGCATC
ATCGCCTATACCATGTCTCTGGGCGCTGAGAACTCCGTGGCCTATAGCAACAACAGCATCGCTATCCCCACCAACTTCACAATCTCC
GTGACCACAGgtaagtgacatgtgtcttaaattaatttattaaaaaacatataaataatttactatatctaaaatctaactgaaatt
cttaacattttctttcagAAATTCTGCCCGTGAGCATGACCAAGACCAGCGTCGACTGCACCATGTATATCTGCGGCGACTCCACAG
AGTGCTCCAATCTGCTGCTGCAGTACGGCAGCTTCTGCACCCAACTCAATAGGGCTCTGACCGGAATTGCTGTCGAGCAAGACAAGA
ACACCCAAGAGGTGTTTGCCCAAGTGAAACAGATTTACAAGACCCCCCCCATCAAGGACTTCGGAGGCTTCAATTTCTCCCAAATCC
TCCCCGACCCCTCCAAACCCTCCAAGAGGAGCTTTATCGAGGATCTGCTGTTCAACAAGGTGACACTGGCTGATGCAGgtaagtagc
ttatttttctttattaaatatttactgagttaatattattcaacttaagtaatgaaaagttttggttcacttacagGTTTTATCAAG
CAGTATGGCGACTGTCTGGGAGACATCGCTGCTAGGGATCTGATCTGTGCCCAGAAGTTTAATGGCCTCACCGTGCTGCCTCCTCTG
CTGACCGACGAGATGATCGCCCAGTATACAAGCGCTCTGCTGGCCGGCACAATTACCAGCGGATGGACATTTGGAGCCGGCGCTGCC
CTCCAGATTCCTTTCGCCATGCAGATGGCCTACAGgtaagttaataccctttttaattaaaatgaattagtatttgccatttacttt
tactatttaagagatgtaaaattgcttttcagGTTCAACGGCATTGGCGTCACACAGAACGTGCTGTACGAGAACCAGAAGCTGATC
GCTAACCAGTTCAACAGCGCCATTGGCAAGATCCAAGATTCCCTCAGCTCCACCGCCAGCGCCCTCGGCAAACTGCAAGACGTCGTG
AATCAGAATGCCCAAGCTCTGAACACACTGGTGAAGCAGCTCAGCAGCAATTTTGGCGCCATCTCCTCCGTGCTCAATGATATTCTG
TCTAGACTGGACAAGGTGGAGGCCGAAGTCCAGATCGATAGACTGATCACAGgtaagtaataggaagtactgcatttcttcttcaag
gacaaaattaatatctagcctaaaaaattaattttcatcttttaaatatttcagGTAGACTGCAGTCCCTCCAGACATACGTGACCC
AGCAGCTCATTAGAGCTGCCGAGATTAGGGCCTCCGCCAATCTCGCTGCCACAAAAATGAGCGAGTGCGTGCTCGGCCAGTCCAAAA
GAGTGGACTTCTGTGGCAAGGGCTACCATCTGATGTCCTTCCCTCAGAGCGCTCCTCATGGCGTCGTGTTTCTGCATGTGACCTACG
TGCCCGCCCAAGAGAAGAACTTCACAACAGCCCCCGCTATCTGTCACGACGGAAAGGCCCACTTCCCCAGgtaagtttaaaaaaaaa
aaaggcatctttttgcaaaggttacaacatgtgtggacttatgtttatgatatttatatttcagGGAGGGCGTCTTTGTGTCCAACG
GCACACACTGGTTTGTCACCCAGAGGAACTTCTATGAGCCCCAGATCATCACCACCGACAACACCTTTGTGAGCGGAAACTGCGATG
TGGTCATCGGCATCGTGAATAACACCGTGTACGACCCTCTCCAGCCCGAGCTGGACTCCTTCAAGGAGGAGCTGGATAAGTACTTTA
AGAACCATACAAGCCCCGACGTGGACCTCGGCGACATTTCAGgtaagttcattcttgaatgatttcaaaacagaagtatttgctttt
ataagaagatcattttacatatattttatcacttacagGTATCAACGCCAGCGTCGTGAACATCCAGAAGGAGATTGATAGACTCAA
CGAGGTCGCCAAGAATCTGAACGAGTCTCTGATTGATCTGCAAGAGCTGGGCAAGTACGAGCAGTACATCAAGTGGCCTTGGTACAT
CTGGCTCGGATTCATTGCCGGACTGATCGCCATCGTCATGGTGACCATCATGCTCTGCTGCATGACAAGCTGTTGCAGCTGTCTGAA
AGGCTGTTGTAGCTGTGGCAGCTGCTGTAAGTTCGATGAGGACGACTCCGAGCCCGTGCTGAAGGGCGTGAAGCTCCACTACACCTA
A
SEQ ID NO: 15 (P226)
ATGTTCGTGTTCCTCGTGCTGCTGCCTCTGGTGTCCTCCCAGTGCGTCAATCTGACAACAAGAACACAGCTGCCCCCCGCCTACACC
AATTCCTTCACAAGAGGCGTGTACTACCCCGACAAGGTGTTCAGAAGCTCCGTGCTGCACAGCACCCAAGACCTCTTTCTGCCCTTT
TTCTCCAACGTCACATGGTTCCACGCTATCCACGTGTCAGgtaagttgatttagaaacacttttcaagcagtcagcccatggttacc
attaagttaaccctatcactgaattgctccaattttcctcttagGTACCAACGGCACCAAAAGGTTCGATAACCCCGTCCTCCCCTT
CAACGATGGCGTCTACTTCGCCAGCACCGAGAAGTCCAATATCATCAGAGGCTGGATCTTCGGCACCACACTGGATTCCAAGACCCA
GTCTCTGCTGATCGTGAATAACGCCACAAACGTGGTCATTAAAGTGTGCGAGTTCCAGTTCTGCAACGACCCCTTCCTCGGCGTGTA
TTACCACAAAAACAACAAGAGCTGGATGGAGTCCGAGTTCAGgtaaggaaatttccatgagtttcactcttgaagcattggggttat
ttgtgccagaggctaatgacccatgctggcccttcacttttctagGGTGTACAGCAGCGCCAACAACTGCACATTCGAGTACGTGAG
CCAGCCTTTCCTCATGGATCTGGAGGGCAAGCAAGGCAATTTCAAGAATCTGAGAGAGTTTGTCTTCAAGAACATCGACGGATACTT
CAAGATTTACTCCAAGCACACCCCCATTAACCTCGTCAGAGACCTCCCCCAAGGCTTTTCCGCTCTCGAACCTCTGGTGGATCTGCC
CATCGGCATCAACATCACAAGATTCCAAACCCTCCTCGCTCTGCATAGAAGCTATCTGACCCCCGGCGATTCCAGCTCAGgtaagta
tccagattttacttcatatatttgcctttttctgtgctccgacttactaacattgtattctccccttcttcattttagGTTGGACAG
CTGGAGCTGCCGCCTACTATGTGGGATATCTGCAACCTAGAACATTTCTGCTGAAGTACAACGAGAACGGCACAATCACAGACGCTG
TGGATTGTGCTCTGGACCCCCTCTCCGAGACCAAGTGTACCCTCAAGAGCTTTACCGTGGAGAAGGGAATCTACCAGACCTCCAATT
TTAGGGTCCAACCCACCGAGAGCATCGTGAGGTTCCCCAACATCACAAACCTCTGCCCTTTCGGCGAAGTGTTCAACGCCACAAGGT
TTGCTTCCGTGTACGCTTGGAACAGgtaagtagtgctgattatacacaagatattgtctagaacttgatgagactgtggatatgaat
atttcactcttttctcagGAAGAGAATCTCCAACTGCGTGGCCGACTATAGCGTGCTCTATAACAGCGCCTCCTTCAGCACCTTCAA
GTGTTACGGCGTGAGCCCCACCAAGCTGAACGATCTGTGTTTCACCAACGTGTACGCTGACTCCTTCGTCATTAGGGGCGACGAAGT
GAGACAAATCGCTCCCGGCCAGACCGGCAAAATCGCTGACTACAACTACAAGCTCCCCGACGACTTCACCGGCTGTGTGATCGCTTG
GAACTCCAACAACCTCGATAGCAAGGTGGGAGGCAACTACAACTATCTGTATAGACTCTTCAGgtaaggaatgttgcactgattttc
acaggattttcccaagtgatactatcttattacattgatttttggctttgttttttttcagGAAGTCCAATCTGAAGCCCTTCGAG
AGGGACATCAGCACAGAGATCTATCAAGCCGGATCCACACCTTGCAACGGCGTCGAGGGATTCAACTGCTACTTCCCTCTGCAATCC
TACGGCTTCCAGCCCACAAATGGCGTGGGCTACCAGCCTTACAGAGTGGTGGTGCTGTCCTTTGAACTGCTGCATGCCCCCGCCACA
GTGTGCGGACCCAAAAAGAGCACCAACCTCGTGAAGAACAAATGCGTCAATTTCAACTTCAATGGACTGACCGGCACAGgtaagtga
cttgctttcttacatcaaaaaggcatccagtgtctgtttaagaattgccttctcaatattctctgttgattcctttccagGTGTGCT
CACCGAGTCCAACAAGAAGTTTCTGCCCTTCCAGCAGTTCGGAAGAGACATTGCCGATACCACAGACGCCGTGAGGGACCCTCAGAC
ACTGGAGATTCTGGATATCACACCTTGCAGCTTCGGCGGCGTGAGCGTGATCACACCCGGAACAAACACCAGCAACCAAGTGGCTGT
GCTGTACCAAGACGTGAATTGTACAGAGGTACCTGTGGCCATCCATGCCGATCAGCTGACCCCCACATGGAGGGTCTACAGCACAGg
taagtaggagaacattttcacatacaaagccatttttactttttttttaaatttcttataatcaatatgatctttttcacagGTTCC
AATGTCTTTCAGACAAGAGCTGGCTGTCTGATTGGCGCTGAGCACGTGAACAACAGCTACGAGTGCGACATCCCTATCGGCGCCGGA
ATTTGCGCCAGCTACCAAACCCAGACCAATAGCCCTAGGAGGGCCAGATCCGTCGCCAGCCAGAGCATCATCGCCTATACCATGTCT
CTGGGCGCTGAGAACTCCGTGGCCTATAGCAACAACAGCATCGCTATCCCCACCAACTTCACAATCTCCGTGACCACAGgtaagttg
ctttctctgaatacaaaactattgtttgactgtctttaagaatattactttttcatcataacttcttctttgaaaagAAATTCTGCC
CGTGAGCATGACCAAGACCAGCGTCGACTGCACCATGTATATCTGCGGCGACTCCACAGAGTGCTCCAATCTGCTGCTGCAGTACGG
CAGCTTCTGCACCCAACTCAATAGGGCTCTGACCGGAATTGCTGTCGAGCAAGACAAGAACACCCAAGAGGTGTTTGCCCAAGTGAA
ACAGATTTACAAGACCCCCCCCATCAAGGACTTCGGAGGCTTCAATTTCTCCCAAATCCTCCCCGACCCCTCCAAACCCTCCAAGAG
GAGCTTTATCGAGGATCTGCTGTTCAACAAGGTGACACTGGCTGATGCAGgtaagtctatttcaaaaaagaatcatatatattttaa
aatagcttatgtattttttacacattcatttcttatttacctactatttatccagGTTTTATCAAGCAGTATGGCGACTGTCTGGGA
GACATCGCTGCTAGGGATCTGATCTGTGCCCAGAAGTTTAATGGCCTCACCGTGCTGCCTCCTCTGCTGACCGACGAGATGATCGCC
CAGTATACAAGCGCTCTGCTGGCCGGCACAATTACCAGCGGATGGACATTTGGAGCCGGCGCTGCCCTCCAGATTCCTTTCGCCATG
CAGATGGCCTACAGgtaagcaaatgaaccatcatcccatcattttgagttatatccttcctttgttatatggggcttacacttatca
tttctcctttgctttagGTTCAACGGCATTGGCGTCACACAGAACGTGCTGTACGAGAACCAGAAGCTGATCGCTAACCAGTTCAAC
AGCGCCATTGGCAAGATCCAAGATTCCCTCAGCTCCACCGCCAGCGCCCTCGGCAAACTGCAAGACGTCGTGAATCAGAATGCCCAA
GCTCTGAACACACTGGTGAAGCAGCTCAGCAGCAATTTTGGCGCCATCTCCTCCGTGCTCAATGATATTCTGTCTAGACTGGACAAG
GTGGAGGCCGAAGTCCAGATCGATAGACTGATCACAGgtaagtgtcttaaattcagaagacgtaaagcaaaacacggttttgaggag
gcttcttattataaatcttgcattatctacttttttctagGTAGACTGCAGTCCCTCCAGACATACGTGACCCAGCAGCTCATTAGA
GCTGCCGAGATTAGGGCCTCCGCCAATCTCGCTGCCACAAAAATGAGCGAGTGCGTGCTCGGCCAGTCCAAAAGAGTGGACTTCTGT
GGCAAGGGCTACCATCTGATGTCCTTCCCTCAGAGCGCTCCTCATGGCGTCGTGTTTCTGCATGTGACCTACGTGCCCGCCCAAGAG
AAGAACTTCACAACAGCCCCCGCTATCTGTCACGACGGAAAGGCCCACTTCCCCAGgtaagtcattatatgaagaaaaacccaggtg
catgttttacatgaagaaaactggtatttgtttgactggttttgcttttatgttttagGGAGGGCGTCTTTGTGTCCAACGGCACAC
ACTGGTTTGTCACCCAGAGGAACTTCTATGAGCCCCAGATCATCACCACCGACAACACCTTTGTGAGCGGAAACTGCGATGTGGTCA
TCGGCATCGTGAATAACACCGTGTACGACCCTCTCCAGCCCGAGCTGGACTCCTTCAAGGAGGAGCTGGATAAGTACTTTAAGAACC
ATACAAGCCCCGACGTGGACCTCGGCGACATTTCAGgtaagttgtccaacttttcaaagatccaggttttcttttaccataaatgtg
ttattgtctgtactaatctataggatttctctcttttgtagGTATCAACGCCAGCGTCGTGAACATCCAGAAGGAGATTGATAGACT
CAACGAGGTCGCCAAGAATCTGAACGAGTCTCTGATTGATCTGCAAGAGCTGGGCAAGTACGAGCAGTACATCAAGTGGCCTTGGTA
CATCTGGCTCGGATTCATTGCCGGACTGATCGCCATCGTCATGGTGACCATCATGCTCTGCTGCATGACAAGCTGTTGCAGCTGTCT
GAAAGGCTGTTGTAGCTGTGGCAGCTGCTGTAAGTTCGATGAGGACGACTCCGAGCCCGTGCTGAAGGGCGTGAAGCTCCACTACAC
CTAA
SEQ ID NO: 16 (P227)
ATGTTCGTGTTCCTCGTGCTGCTGCCTCTGGTGTCCTCCCAGTGCGTCAATCTGACAACAAGAACACAGCTGCCCCCCGCCTACACC
AATTCCTTCACAAGAGGCGTGTACTACCCCGACAAGGTGTTCAGAAGCTCCGTGCTGCACAGCACCCAAGACCTCTTTCTGCCCTTT
TTCTCCAACGTCACATGGTTCCACGCTATCCACGTGTCAGgtaagttgatttagaaacacttttcaagcagtcagcccatggttacc
attaagttaaccctatcactgaattgctccaattttcctcttagGTACCAACGGCACCAAAAGGTTCGATAACCCCGTCCTCCCCTT
CAACGATGGCGTCTACTTCGCCAGCACCGAGAAGTCCAATATCATCAGAGGCTGGATCTTCGGCACCACACTGGATTCCAAGACCCA
GTCTCTGCTGATCGTGAATAACGCCACAAACGTGGTCATTAAAGTGTGCGAGTTCCAGTTCTGCAACGACCCCTTCCTCGGCGTGTA
TTACCACAAAAACAACAAGAGCTGGATGGAGTCCGAGTTCAGgtaaggaaatttccatgagtttcactcttgaagcattggggttat
ttgtgccagaggctaatgacccatgctggcccttcacttttctagGGTGTACAGCAGCGCCAACAACTGCACATTCGAGTACGTGAG
CCAGCCTTTCCTCATGGATCTGGAGGGCAAGCAAGGCAATTTCAAGAATCTGAGAGAGTTTGTCTTCAAGAACATCGACGGATACTT
CAAGATTTACTCCAAGCACACCCCCATTAACCTCGTCAGAGACCTCCCCCAAGGCTTTTCCGCTCTCGAACCTCTGGTGGATCTGCC
CATCGGCATCAACATCACAAGATTCCAAACCCTCCTCGCTCTGCATAGAAGCTATCTGACCCCCGGCGATTCCAGCTCAGgtaagtg
catatgtcaaaaaaagggaatttttgaaaatttaatttaatcataaaaagaaaataaatttcattattttttgcagGTTGGACAGCT
GGAGCTGCCGCCTACTATGTGGGATATCTGCAACCTAGAACATTTCTGCTGAAGTACAACGAGAACGGCACAATCACAGACGCTGTG
GATTGTGCTCTGGACCCCCTCTCCGAGACCAAGTGTACCCTCAAGAGCTTTACCGTGGAGAAGGGAATCTACCAGACCTCCAATTTT
AGGGTCCAACCCACCGAGAGCATCGTGAGGTTCCCCAACATCACAAACCTCTGCCCTTTCGGCGAAGTGTTCAACGCCACAAGGTTT
GCTTCCGTGTACGCTTGGAACAGgtaagtagtgctgattatacacaagatattgtctagaacttgatgagactgtggatatgaatat
ttcactcttttctcagGAAGAGAATCTCCAACTGCGTGGCCGACTATAGCGTGCTCTATAACAGCGCCTCCTTCAGCACCTTCAAGT
GTTACGGCGTGAGCCCCACCAAGCTGAACGATCTGTGTTTCACCAACGTGTACGCTGACTCCTTCGTCATTAGGGGCGACGAAGTGA
GACAAATCGCTCCCGGCCAGACCGGCAAAATCGCTGACTACAACTACAAGCTCCCCGACGACTTCACCGGCTGTGTGATCGCTTGGA
ACTCCAACAACCTCGATAGCAAGGTGGGAGGCAACTACAACTATCTGTATAGACTCTTCAGgtaaggaatgttgcactgattttcac
aggattttcccaagtgatactatcttattacattgatttttggctttgttttgttttcagGAAGTCCAATCTGAAGCCCTTCGAGAG
GGACATCAGCACAGAGATCTATCAAGCCGGATCCACACCTTGCAACGGCGTCGAGGGATTCAACTGCTACTTCCCTCTGCAATCCTA
CGGCTTCCAGCCCACAAATGGCGTGGGCTACCAGCCTTACAGAGTGGTGGTGCTGTCCTTTGAACTGCTGCATGCCCCCGCCACAGT
GTGCGGACCCAAAAAGAGCACCAACCTCGTGAAGAACAAATGCGTCAATTTCAACTTCAATGGACTGACCGGCACAGgtaagtgact
tgctttcttacatcaaaaaggcatccagtgtctgtttaagaattgccttctcaatattctctgttgattcctttccagGTGTGCTCA
CCGAGTCCAACAAGAAGTTTCTGCCCTTCCAGCAGTTCGGAAGAGACATTGCCGATACCACAGACGCCGTGAGGGACCCTCAGACAC
TGGAGATTCTGGATATCACACCTTGCAGCTTCGGCGGCGTGAGCGTGATCACACCCGGAACAAACACCAGCAACCAAGTGGCTGTGC
TGTACCAAGACGTGAATTGTACAGAGGTACCTGTGGCCATCCATGCCGATCAGCTGACCCCCACATGGAGGGTCTACAGCACAGgta
agtaggagaacattttcacatacaaagccatttttactttttttttaaatttcttataatcaatatgatctttttcacagGTTCCAA
TGTCTTTCAGACAAGAGCTGGCTGTCTGATTGGCGCTGAGCACGTGAACAACAGCTACGAGTGCGACATCCCTATCGGCGCCGGAAT
TTGCGCCAGCTACCAAACCCAGACCAATAGCCCTAGGAGGGCCAGATCCGTCGCCAGCCAGAGCATCATCGCCTATACCATGTCTCT
GGGCGCTGAGAACTCCGTGGCCTATAGCAACAACAGCATCGCTATCCCCACCAACTTCACAATCTCCGTGACCACAGgtaagttgct
ttctctgaatacaaaactattgtttgactgtctttaagaatattactttttcatcataacttcttctttgaaaagAAATTCTGCCCG
TGAGCATGACCAAGACCAGCGTCGACTGCACCATGTATATCTGCGGCGACTCCACAGAGTGCTCCAATCTGCTGCTGCAGTACGGCA
GCTTCTGCACCCAACTCAATAGGGCTCTGACCGGAATTGCTGTCGAGCAAGACAAGAACACCCAAGAGGTGTTTGCCCAAGTGAAAC
AGATTTACAAGACCCCCCCCATCAAGGACTTCGGAGGCTTCAATTTCTCCCAAATCCTCCCCGACCCCTCCAAACCCTCCAAGAGGA
GCTTTATCGAGGATCTGCTGTTCAACAAGGTGACACTGGCTGATGCAGgtaagtctatttcaaaaaagaatcatatatattttaaaa
tagcttatgtattttttacacattcatttcttatttacctactatttatccagGTTTTATCAAGCAGTATGGCGACTGTCTGGGAGA
CATCGCTGCTAGGGATCTGATCTGTGCCCAGAAGTTTAATGGCCTCACCGTGCTGCCTCCTCTGCTGACCGACGAGATGATCGCCCA
GTATACAAGCGCTCTGCTGGCCGGCACAATTACCAGCGGATGGACATTTGGAGCCGGCGCTGCCCTCCAGATTCCTTTCGCCATGCA
GATGGCCTACAGgtaagcaaatgaaccatcatcccatcattttgagttatatccttcctttgttatatggggcttacacttatcatt
tctcctttgctttagGTTCAACGGCATTGGCGTCACACAGAACGTGCTGTACGAGAACCAGAAGCTGATCGCTAACCAGTTCAACAG
CGCCATTGGCAAGATCCAAGATTCCCTCAGCTCCACCGCCAGCGCCCTCGGCAAACTGCAAGACGTCGTGAATCAGAATGCCCAAGC
TCTGAACACACTGGTGAAGCAGCTCAGCAGCAATTTTGGCGCCATCTCCTCCGTGCTCAATGATATTCTGTCTAGACTGGACAAGGT
GGAGGCCGAAGTCCAGATCGATAGACTGATCACAGgtaagtgtcttaaattcagaagacgtaaagcaaaacacggttttgaggaggc
ttcttattataaatcttgcattatctacttttttctagGTAGACTGCAGTCCCTCCAGACATACGTGACCCAGCAGCTCATTAGAGC
TGCCGAGATTAGGGCCTCCGCCAATCTCGCTGCCACAAAAATGAGCGAGTGCGTGCTCGGCCAGTCCAAAAGAGTGGACTTCTGTGG
CAAGGGCTACCATCTGATGTCCTTCCCTCAGAGCGCTCCTCATGGCGTCGTGTTTCTGCATGTGACCTACGTGCCCGCCCAAGAGAA
GAACTTCACAACAGCCCCCGCTATCTGTCACGACGGAAAGGCCCACTTCCCCAGgtaagtcattatatgaagaaaaacccaggtgca
tgttttacatgaagaaaactggtatttgtttgactggttttgcttttatgttttagGGAGGGCGTCTTTGTGTCCAACGGCACACAC
TGGTTTGTCACCCAGAGGAACTTCTATGAGCCCCAGATCATCACCACCGACAACACCTTTGTGAGCGGAAACTGCGATGTGGTCATC
GGCATCGTGAATAACACCGTGTACGACCCTCTCCAGCCCGAGCTGGACTCCTTCAAGGAGGAGCTGGATAAGTACTTTAAGAACCAT
ACAAGCCCCGACGTGGACCTCGGCGACATTTCAGgtaagttgtccaacttttcaaagatccaggttttcttttaccataaatgtgtt
attgtctgtactaatctataggatttctctcttttgtagGTATCAACGCCAGCGTCGTGAACATCCAGAAGGAGATTGATAGACTCA
ACGAGGTCGCCAAGAATCTGAACGAGTCTCTGATTGATCTGCAAGAGCTGGGCAAGTACGAGCAGTACATCAAGTGGCCTTGGTACA
TCTGGCTCGGATTCATTGCCGGACTGATCGCCATCGTCATGGTGACCATCATGCTCTGCTGCATGACAAGCTGTTGCAGCTGTCTGA
AAGGCTGTTGTAGCTGTGGCAGCTGCTGTAAGTTCGATGAGGACGACTCCGAGCCCGTGCTGAAGGGCGTGAAGCTCCACTACACCT
AA
ATGTTCGTGTTCCTCGTGCTGCTGCCTCTGGTGTCCTCCCAGTGCGTCAATCTGACAACAAGAACACAGCTGCCCCCCGCCTACACC
AATTCCTTCACAAGAGGCGTGTACTACCCCGACAAGGTGTTCAGAAGCTCCGTGCTGCACAGCACCCAAGACCTCTTTCTGCCCTTT
TTCTCCAACGTCACATGGTTCCACGCTATCCACGTGTCAGgtaagttgatttagaaacacttttcaagcagtcagcccatggttacc
attaagttaaccctatcactgaattgctccaattttcctcttagGTACCAACGGCACCAAAAGGTTCGATAACCCCGTCCTCCCCTT
CAACGATGGCGTCTACTTCGCCAGCACCGAGAAGTCCAATATCATCAGAGGCTGGATCTTCGGCACCACACTGGATTCCAAGACCCA
GTCTCTGCTGATCGTGAATAACGCCACAAACGTGGTCATTAAAGTGTGCGAGTTCCAGTTCTGCAACGACCCCTTCCTCGGCGTGTA
TTACCACAAAAACAACAAGAGCTGGATGGAGTCCGAGTTCAGgtaaggaaatttccatgagtttcactcttgaagcattggggttat
ttgtgccagaggctaatgacccatgctggcccttcacttttctagGGTGTACAGCAGCGCCAACAACTGCACATTCGAGTACGTGAG
CCAGCCTTTCCTCATGGATCTGGAGGGCAAGCAAGGCAATTTCAAGAATCTGAGAGAGTTTGTCTTCAAGAACATCGACGGATACTT
CAAGATTTACTCCAAGCACACCCCCATTAACCTCGTCAGAGACCTCCCCCAAGGCTTTTCCGCTCTCGAACCTCTGGTGGATCTGCC
CATCGGCATCAACATCACAAGATTCCAAACCCTCCTCGCTCTGCATAGAAGCTATCTGACCCCCGGCGATTCCAGCTCAGgtaagta
gtttgggaaaacctttaaatttacgtcaaattttacataagaccgatatgttttaattattaaatattttgcagGTTGGACAGCTGG
AGCTGCCGCCTACTATGTGGGATATCTGCAACCTAGAACATTTCTGCTGAAGTACAACGAGAACGGCACAATCACAGACGCTGTGGA
TTGTGCTCTGGACCCCCTCTCCGAGACCAAGTGTACCCTCAAGAGCTTTACCGTGGAGAAGGGAATCTACCAGACCTCCAATTTTAG
GGTCCAACCCACCGAGAGCATCGTGAGGTTCCCCAACATCACAAACCTCTGCCCTTTCGGCGAAGTGTTCAACGCCACAAGGTTTGC
TTCCGTGTACGCTTGGAACAGgtaagtagtgctgattatacacaagatattgtctagaacttgatgagactgtggatatgaatattt
cactcttttctcagGAAGAGAATCTCCAACTGCGTGGCCGACTATAGCGTGCTCTATAACAGCGCCTCCTTCAGCACCTTCAAGTGT
TACGGCGTGAGCCCCACCAAGCTGAACGATCTGTGTTTCACCAACGTGTACGCTGACTCCTTCGTCATTAGGGGCGACGAAGTGAGA
CAAATCGCTCCCGGCCAGACCGGCAAAATCGCTGACTACAACTACAAGCTCCCCGACGACTTCACCGGCTGTGTGATCGCTTGGAAC
TCCAACAACCTCGATAGCAAGGTGGGAGGCAACTACAACTATCTGTATAGACTCTTCAGgtaaggaatgttgcactgattttcacag
gattttcccaagtgatactatcttattacattgatttttggctttgttttgttttcagGAAGTCCAATCTGAAGCCCTTCGAGAGGG
ACATCAGCACAGAGATCTATCAAGCCGGATCCACACCTTGCAACGGCGTCGAGGGATTCAACTGCTACTTCCCTCTGCAATCCTACG
GCTTCCAGCCCACAAATGGCGTGGGCTACCAGCCTTACAGAGTGGTGGTGCTGTCCTTTGAACTGCTGCATGCCCCCGCCACAGTGT
GCGGACCCAAAAAGAGCACCAACCTCGTGAAGAACAAATGCGTCAATTTCAACTTCAATGGACTGACCGGCACAGgtaagtgacttg
ctttcttacatcaaaaaggcatccagtgtctgtttaagaattgccttctcaatattctctgttgattcctttccagGTGTGCTCACC
GAGTCCAACAAGAAGTTTCTGCCCTTCCAGCAGTTCGGAAGAGACATTGCCGATACCACAGACGCCGTGAGGGACCCTCAGACACTG
GAGATTCTGGATATCACACCTTGCAGCTTCGGCGGCGTGAGCGTGATCACACCCGGAACAAACACCAGCAACCAAGTGGCTGTGCTG
TACCAAGACGTGAATTGTACAGAGGTACCTGTGGCCATCCATGCCGATCAGCTGACCCCCACATGGAGGGTCTACAGCACAGgtaag
taggagaacattttcacatacaaagccatttttactttttttttaaatttcttataatcaatatgatctttttcacagGTTCCAATG
TCTTTCAGACAAGAGCTGGCTGTCTGATTGGCGCTGAGCACGTGAACAACAGCTACGAGTGCGACATCCCTATCGGCGCCGGAATTT
GCGCCAGCTACCAAACCCAGACCAATAGCCCTAGGAGGGCCAGATCCGTCGCCAGCCAGAGCATCATCGCCTATACCATGTCTCTGG
GCGCTGAGAACTCCGTGGCCTATAGCAACAACAGCATCGCTATCCCCACCAACTTCACAATCTCCGTGACCACAGgtaagttgcttt
ctctgaatacaaaactattgtttgactgtctttaagaatattactttttcatcataacttcttctttgaaaagAAATTCTGCCCGTG
AGCATGACCAAGACCAGCGTCGACTGCACCATGTATATCTGCGGCGACTCCACAGAGTGCTCCAATCTGCTGCTGCAGTACGGCAGC
TTCTGCACCCAACTCAATAGGGCTCTGACCGGAATTGCTGTCGAGCAAGACAAGAACACCCAAGAGGTGTTTGCCCAAGTGAAACAG
ATTTACAAGACCCCCCCCATCAAGGACTTCGGAGGCTTCAATTTCTCCCAAATCCTCCCCGACCCCTCCAAACCCTCCAAGAGGAGC
TTTATCGAGGATCTGCTGTTCAACAAGGTGACACTGGCTGATGCAGgtaagtctatttcaaaaaagaatcatatatattttaaaata
gcttatgtattttttacacattcatttcttatttacctactatttatccagGTTTTATCAAGCAGTATGGCGACTGTCTGGGAGACA
TCGCTGCTAGGGATCTGATCTGTGCCCAGAAGTTTAATGGCCTCACCGTGCTGCCTCCTCTGCTGACCGACGAGATGATCGCCCAGT
ATACAAGCGCTCTGCTGGCCGGCACAATTACCAGCGGATGGACATTTGGAGCCGGCGCTGCCCTCCAGATTCCTTTCGCCATGCAGA
TGGCCTACAGgtaagcaaatgaaccatcatcccatcattttgagttatatccttcctttgttatatggggcttacacttatcatttc
tcctttgctttagGTTCAACGGCATTGGCGTCACACAGAACGTGCTGTACGAGAACCAGAAGCTGATCGCTAACCAGTTCAACAGCG
CCATTGGCAAGATCCAAGATTCCCTCAGCTCCACCGCCAGCGCCCTCGGCAAACTGCAAGACGTCGTGAATCAGAATGCCCAAGCTC
TGAACACACTGGTGAAGCAGCTCAGCAGCAATTTTGGCGCCATCTCCTCCGTGCTCAATGATATTCTGTCTAGACTGGACAAGGTGG
AGGCCGAAGTCCAGATCGATAGACTGATCACAGgtaagtgtcttaaattcagaagacgtaaagcaaaacacggttttgaggaggctt
cttattataaatcttgcattatctacttttttctagGTAGACTGCAGTCCCTCCAGACATACGTGACCCAGCAGCTCATTAGAGCTG
CCGAGATTAGGGCCTCCGCCAATCTCGCTGCCACAAAAATGAGCGAGTGCGTGCTCGGCCAGTCCAAAAGAGTGGACTTCTGTGGCA
AGGGCTACCATCTGATGTCCTTCCCTCAGAGCGCTCCTCATGGCGTCGTGTTTCTGCATGTGACCTACGTGCCCGCCCAAGAGAAGA
ACTTCACAACAGCCCCCGCTATCTGTCACGACGGAAAGGCCCACTTCCCCAGgtaagtcattatatgaagaaaaacccaggtgcatg
ttttacatgaagaaaactggtatttgtttgactggttttgcttttatgttttagGGAGGGCGTCTTTGTGTCCAACGGCACACACTG
GTTTGTCACCCAGAGGAACTTCTATGAGCCCCAGATCATCACCACCGACAACACCTTTGTGAGCGGAAACTGCGATGTGGTCATCGG
CATCGTGAATAACACCGTGTACGACCCTCTCCAGCCCGAGCTGGACTCCTTCAAGGAGGAGCTGGATAAGTACTTTAAGAACCATAC
AAGCCCCGACGTGGACCTCGGCGACATTTCAGgtaagttgtccaacttttcaaagatccaggttttcttttaccataaatgtgttat
tgtctgtactaatctataggatttctctcttttgtagGTATCAACGCCAGCGTCGTGAACATCCAGAAGGAGATTGATAGACTCAAC
GAGGTCGCCAAGAATCTGAACGAGTCTCTGATTGATCTGCAAGAGCTGGGCAAGTACGAGCAGTACATCAAGTGGCCTTGGTACATC
TGGCTCGGATTCATTGCCGGACTGATCGCCATCGTCATGGTGACCATCATGCTCTGCTGCATGACAAGCTGTTGCAGCTGTCTGAAA
GGCTGTTGTAGCTGTGGCAGCTGCTGTAAGTTCGATGAGGACGACTCCGAGCCCGTGCTGAAGGGCGTGAAGCTCCACTACACCTAA
SEQ ID NO: 17 (P228)
ATGTTCGTGTTCCTCGTGCTGCTGCCTCTGGTGTCCTCCCAGTGCGTCAATCTGACAACAAGAACACAGCTGCCCCCCGCCTACACC
AATTCCTTCACAAGAGGCGTGTACTACCCCGACAAGGTGTTCAGAAGCTCCGTGCTGCACAGCACCCAAGACCTCTTTCTGCCCTTT
TTCTCCAACGTCACATGGTTCCACGCTATCCACGTGTCAGgtaagttgatttagaaacacttttcaagcagtcagcccatggttacc
attaagttaaccctatcactgaattgctccaattttcctcttagGTACCAACGGCACCAAAAGGTTCGATAACCCCGTCCTCCCCTT
CAACGATGGCGTCTACTTCGCCAGCACCGAGAAGTCCAATATCATCAGAGGCTGGATCTTCGGCACCACACTGGATTCCAAGACCCA
GTCTCTGCTGATCGTGAATAACGCCACAAACGTGGTCATTAAAGTGTGCGAGTTCCAGTTCTGCAACGACCCCTTCCTCGGCGTGTA
TTACCACAAAAACAACAAGAGCTGGATGGAGTCCGAGTTCAGgtaaggaaatttccatgagtttcactcttgaagcattggggttat
ttgtgccagaggctaatgacccatgctggcccttcacttttctagGGTGTACAGCAGCGCCAACAACTGCACATTCGAGTACGTGAG
CCAGCCTTTCCTCATGGATCTGGAGGGCAAGCAAGGCAATTTCAAGAATCTGAGAGAGTTTGTCTTCAAGAACATCGACGGATACTT
CAAGATTTACTCCAAGCACACCCCCATTAACCTCGTCAGAGACCTCCCCCAAGGCTTTTCCGCTCTCGAACCTCTGGTGGATCTGCC
CATCGGCATCAACATCACAAGATTCCAAACCCTCCTCGCTCTGCATAGAAGCTATCTGACCCCCGGCGATTCCAGCTCAGgtaagta
gtttgggaaaacctttaaatttacgtcaaattttacataagaccgatatgttttaattattaaatattttgcagGTTGGACAGCTGG
AGCTGCCGCCTACTATGTGGGATATCTGCAACCTAGAACATTTCTGCTGAAGTACAACGAGAACGGCACAATCACAGACGCTGTGGA
TTGTGCTCTGGACCCCCTCTCCGAGACCAAGTGTACCCTCAAGAGCTTTACCGTGGAGAAGGGAATCTACCAGACCTCCAATTTTAG
GGTCCAACCCACCGAGAGCATCGTGAGGTTCCCCAACATCACAAACCTCTGCCCTTTCGGCGAAGTGTTCAACGCCACAAGGTTTGC
TTCCGTGTACGCTTGGAACAGgtaagtagtgctgattatacacaagatattgtctagaacttgatgagactgtggatatgaatattt
cactcttttctcagGAAGAGAATCTCCAACTGCGTGGCCGACTATAGCGTGCTCTATAACAGCGCCTCCTTCAGCACCTTCAAGTGT
TACGGCGTGAGCCCCACCAAGCTGAACGATCTGTGTTTCACCAACGTGTACGCTGACTCCTTCGTCATTAGGGGCGACGAAGTGAGA
CAAATCGCTCCCGGCCAGACCGGCAAAATCGCTGACTACAACTACAAGCTCCCCGACGACTTCACCGGCTGTGTGATCGCTTGGAAC
TCCAACAACCTCGATAGCAAGGTGGGAGGCAACTACAACTATCTGTATAGACTCTTCAGgtaaggaatgttgcactgattttcacag
gattttcccaagtgatactatcttattacattgatttttggctttgttttgttttcagGAAGTCCAATCTGAAGCCCTTCGAGAGGG
ACATCAGCACAGAGATCTATCAAGCCGGATCCACACCTTGCAACGGCGTCGAGGGATTCAACTGCTACTTCCCTCTGCAATCCTACG
GCTTCCAGCCCACAAATGGCGTGGGCTACCAGCCTTACAGAGTGGTGGTGCTGTCCTTTGAACTGCTGCATGCCCCCGCCACAGTGT
GCGGACCCAAAAAGAGCACCAACCTCGTGAAGAACAAATGCGTCAATTTCAACTTCAATGGACTGACCGGCACAGgtaagtgacttg
ctttcttacatcaaaaaggcatccagtgtctgtttaagaattgccttctcaatattctctgttgattcctttccagGTGTGCTCACC
GAGTCCAACAAGAAGTTTCTGCCCTTCCAGCAGTTCGGAAGAGACATTGCCGATACCACAGACGCCGTGAGGGACCCTCAGACACTG
GAGATTCTGGATATCACACCTTGCAGCTTCGGCGGCGTGAGCGTGATCACACCCGGAACAAACACCAGCAACCAAGTGGCTGTGCTG
TACCAAGACGTGAATTGTACAGAGGTACCTGTGGCCATCCATGCCGATCAGCTGACCCCCACATGGAGGGTCTACAGCACAGgtaag
taggagaacattttcacatacaaagccatttttactttttttttaaatttcttataatcaatatgatctttttcacagGTTCCAATG
TCTTTCAGACAAGAGCTGGCTGTCTGATTGGCGCTGAGCACGTGAACAACAGCTACGAGTGCGACATCCCTATCGGCGCCGGAATTT
GCGCCAGCTACCAAACCCAGACCAATAGCCCTAGGAGGGCCAGATCCGTCGCCAGCCAGAGCATCATCGCCTATACCATGTCTCTGG
GCGCTGAGAACTCCGTGGCCTATAGCAACAACAGCATCGCTATCCCCACCAACTTCACAATCTCCGTGACCACAGgtaagttgcttt
ctctgaatacaaaactattgtttgactgtctttaagaatattactttttcatcataacttcttctttgaaaagAAATTCTGCCCGTG
AGCATGACCAAGACCAGCGTCGACTGCACCATGTATATCTGCGGCGACTCCACAGAGTGCTCCAATCTGCTGCTGCAGTACGGCAGC
TTCTGCACCCAACTCAATAGGGCTCTGACCGGAATTGCTGTCGAGCAAGACAAGAACACCCAAGAGGTGTTTGCCCAAGTGAAACAG
ATTTACAAGACCCCCCCCATCAAGGACTTCGGAGGCTTCAATTTCTCCCAAATCCTCCCCGACCCCTCCAAACCCTCCAAGAGGAGC
TTTATCGAGGATCTGCTGTTCAACAAGGTGACACTGGCTGATGCAGgtaagtctatttcaaaaaagaatcatatatattttaaaata
gcttatgtattttttacacattcatttcttatttacctactatttatccagGTTTTATCAAGCAGTATGGCGACTGTCTGGGAGACA
TCGCTGCTAGGGATCTGATCTGTGCCCAGAAGTTTAATGGCCTCACCGTGCTGCCTCCTCTGCTGACCGACGAGATGATCGCCCAGT
ATACAAGCGCTCTGCTGGCCGGCACAATTACCAGCGGATGGACATTTGGAGCCGGCGCTGCCCTCCAGATTCCTTTCGCCATGCAGA
TGGCCTACAGgtaagcaaatgaaccatcatcccatcattttgagttatatccttcctttgttatatggggcttacacttatcatttc
tcctttgctttagGTTCAACGGCATTGGCGTCACACAGAACGTGCTGTACGAGAACCAGAAGCTGATCGCTAACCAGTTCAACAGCG
CCATTGGCAAGATCCAAGATTCCCTCAGCTCCACCGCCAGCGCCCTCGGCAAACTGCAAGACGTCGTGAATCAGAATGCCCAAGCTC
TGAACACACTGGTGAAGCAGCTCAGCAGCAATTTTGGCGCCATCTCCTCCGTGCTCAATGATATTCTGTCTAGACTGGACAAGGTGG
AGGCCGAAGTCCAGATCGATAGACTGATCACAGgtaagtgtcttaaattcagaagacgtaaagcaaaacacggttttgaggaggctt
cttattataaatcttgcattatctacttttttctagGTAGACTGCAGTCCCTCCAGACATACGTGACCCAGCAGCTCATTAGAGCTG
CCGAGATTAGGGCCTCCGCCAATCTCGCTGCCACAAAAATGAGCGAGTGCGTGCTCGGCCAGTCCAAAAGAGTGGACTTCTGTGGCA
AGGGCTACCATCTGATGTCCTTCCCTCAGAGCGCTCCTCATGGCGTCGTGTTTCTGCATGTGACCTACGTGCCCGCCCAAGAGAAGA
ACTTCACAACAGCCCCCGCTATCTGTCACGACGGAAAGGCCCACTTCCCCAGgtaagtcattatatgaagaaaaacccaggtgcatg
ttttacatgaagaaaactggtatttgtttgactggttttgcttttatgttttagGGAGGGCGTCTTTGTGTCCAACGGCACACACTG
GTTTGTCACCCAGAGGAACTTCTATGAGCCCCAGATCATCACCACCGACAACACCTTTGTGAGCGGAAACTGCGATGTGGTCATCGG
CATCGTGAATAACACCGTGTACGACCCTCTCCAGCCCGAGCTGGACTCCTTCAAGGAGGAGCTGGATAAGTACTTTAAGAACCATAC
AAGCCCCGACGTGGACCTCGGCGACATTTCAGgtaagttgtccaacttttcaaagatccaggttttcttttaccataaatgtgttat
tgtctgtactaatctataggatttctctcttttgtagGTATCAACGCCAGCGTCGTGAACATCCAGAAGGAGATTGATAGACTCAAC
GAGGTCGCCAAGAATCTGAACGAGTCTCTGATTGATCTGCAAGAGCTGGGCAAGTACGAGCAGTACATCAAGTGGCCTTGGTACATC
TGGCTCGGATTCATTGCCGGACTGATCGCCATCGTCATGGTGACCATCATGCTCTGCTGCATGACAAGCTGTTGCAGCTGTCTGAAA
GGCTGTTGTAGCTGTGGCAGCTGCTGTAAGTTCGATGAGGACGACTCCGAGCCCGTGCTGAAGGGCGTGAAGCTCCACTACACCTAA
SEQ ID NO: 18 (P229)
ATGTTCGTGTTCCTCGTGCTGCTGCCTCTGGTGTCCTCCCAGTGCGTCAATCTGACAACAAGAACACAGCTGCCCCCCGCCTACACC
AATTCCTTCACAAGAGGCGTGTACTACCCCGACAAGGTGTTCAGAAGCTCCGTGCTGCACAGCACCCAAGACCTCTTTCTGCCCTTT
TTCTCCAACGTCACATGGTTCCACGCTATCCACGTGTCAGgtaagttgatttagaaacacttttcaagcagtcagcccatggttacc
attaagttaaccctatcactgaattgctccaattttcctcttagGTACCAACGGCACCAAAAGGTTCGATAACCCCGTCCTCCCCTT
CAACGATGGCGTCTACTTCGCCAGCACCGAGAAGTCCAATATCATCAGAGGCTGGATCTTCGGCACCACACTGGATTCCAAGACCCA
GTCTCTGCTGATCGTGAATAACGCCACAAACGTGGTCATTAAAGTGTGCGAGTTCCAGTTCTGCAACGACCCCTTCCTCGGCGTGTA
TTACCACAAAAACAACAAGAGCTGGATGGAGTCCGAGTTCAGgtaaggaaatttccatgagtttcactcttgaagcattggggttat
ttgtgccagaggctaatgacccatgctggcccttcacttttctagGGTGTACAGCAGCGCCAACAACTGCACATTCGAGTACGTGAG
CCAGCCTTTCCTCATGGATCTGGAGGGCAAGCAAGGCAATTTCAAGAATCTGAGAGAGTTTGTCTTCAAGAACATCGACGGATACTT
CAAGATTTACTCCAAGCACACCCCCATTAACCTCGTCAGAGACCTCCCCCAAGGCTTTTCCGCTCTCGAACCTCTGGTGGATCTGCC
CATCGGCATCAACATCACAAGATTCCAAACCCTCCTCGCTCTGCATAGAAGCTATCTGACCCCCGGCGATTCCAGCTCAGgtaagtt
ctatataccaagaaaaatagacccctttgtccttttagacgtagaagtgatgagaacattttatgctattttttatcccttcagGTT
GGACAGCTGGAGCTGCCGCCTACTATGTGGGATATCTGCAACCTAGAACATTTCTGCTGAAGTACAACGAGAACGGCACAATCACAG
ACGCTGTGGATTGTGCTCTGGACCCCCTCTCCGAGACCAAGTGTACCCTCAAGAGCTTTACCGTGGAGAAGGGAATCTACCAGACCT
CCAATTTTAGGGTCCAACCCACCGAGAGCATCGTGAGGTTCCCCAACATCACAAACCTCTGCCCTTTCGGCGAAGTGTTCAACGCCA
CAAGGTTTGCTTCCGTGTACGCTTGGAACAGgtaagtagtgctgattatacacaagatattgtctagaacttgatgagactgtggat
atgaatatttcactcttttctcagGAAGAGAATCTCCAACTGCGTGGCCGACTATAGCGTGCTCTATAACAGCGCCTCCTTCAGCAC
CTTCAAGTGTTACGGCGTGAGCCCCACCAAGCTGAACGATCTGTGTTTCACCAACGTGTACGCTGACTCCTTCGTCATTAGGGGCGA
CGAAGTGAGACAAATCGCTCCCGGCCAGACCGGCAAAATCGCTGACTACAACTACAAGCTCCCCGACGACTTCACCGGCTGTGTGAT
CGCTTGGAACTCCAACAACCTCGATAGCAAGGTGGGAGGCAACTACAACTATCTGTATAGACTCTTCAGgtaaggaatgttgcactg
attttcacaggattttcccaagtgatactatcttattacattgatttttggctttgttttgttttcagGAAGTCCAATCTGAAGCCC
TTCGAGAGGGACATCAGCACAGAGATCTATCAAGCCGGATCCACACCTTGCAACGGCGTCGAGGGATTCAACTGCTACTTCCCTCTG
CAATCCTACGGCTTCCAGCCCACAAATGGCGTGGGCTACCAGCCTTACAGAGTGGTGGTGCTGTCCTTTGAACTGCTGCATGCCCCC
GCCACAGTGTGCGGACCCAAAAAGAGCACCAACCTCGTGAAGAACAAATGCGTCAATTTCAACTTCAATGGACTGACCGGCACAGgt
aagtgacttgctttcttacatcaaaaaggcatccagtgtctgtttaagaattgccttctcaatattctctgttgattcctttccagG
TGTGCTCACCGAGTCCAACAAGAAGTTTCTGCCCTTCCAGCAGTTCGGAAGAGACATTGCCGATACCACAGACGCCGTGAGGGACCC
TCAGACACTGGAGATTCTGGATATCACACCTTGCAGCTTCGGCGGCGTGAGCGTGATCACACCCGGAACAAACACCAGCAACCAAGT
GGCTGTGCTGTACCAAGACGTGAATTGTACAGAGGTACCTGTGGCCATCCATGCCGATCAGCTGACCCCCACATGGAGGGTCTACAG
CACAGgtaagtaggagaacattttcacatacaaagccatttttactttttttttaaatttcttataatcaatatgatctttttcaca
gGTTCCAATGTCTTTCAGACAAGAGCTGGCTGTCTGATTGGCGCTGAGCACGTGAACAACAGCTACGAGTGCGACATCCCTATCGGC
GCCGGAATTTGCGCCAGCTACCAAACCCAGACCAATAGCCCTAGGAGGGCCAGATCCGTCGCCAGCCAGAGCATCATCGCCTATACC
ATGTCTCTGGGCGCTGAGAACTCCGTGGCCTATAGCAACAACAGCATCGCTATCCCCACCAACTTCACAATCTCCGTGACCACAGgt
aagttgctttctctgaatacaaaactattgtttgactgtctttaagaatattactttttcatcataacttcttctttgaaaagAAAT
TCTGCCCGTGAGCATGACCAAGACCAGCGTCGACTGCACCATGTATATCTGCGGCGACTCCACAGAGTGCTCCAATCTGCTGCTGCA
GTACGGCAGCTTCTGCACCCAACTCAATAGGGCTCTGACCGGAATTGCTGTCGAGCAAGACAAGAACACCCAAGAGGTGTTTGCCCA
AGTGAAACAGATTTACAAGACCCCCCCCATCAAGGACTTCGGAGGCTTCAATTTCTCCCAAATCCTCCCCGACCCCTCCAAACCCTC
CAAGAGGAGCTTTATCGAGGATCTGCTGTTCAACAAGGTGACACTGGCTGATGCAGgtaagtctatttcaaaaaagaatcatatata
ttttaaaatagcttatgtattttttacacattcatttcttatttacctactatttatccagGTTTTATCAAGCAGTATGGCGACTGT
CTGGGAGACATCGCTGCTAGGGATCTGATCTGTGCCCAGAAGTTTAATGGCCTCACCGTGCTGCCTCCTCTGCTGACCGACGAGATG
ATCGCCCAGTATACAAGCGCTCTGCTGGCCGGCACAATTACCAGCGGATGGACATTTGGAGCCGGCGCTGCCCTCCAGATTCCTTTC
GCCATGCAGATGGCCTACAGgtaagcaaatgaaccatcatcccatcattttgagttatatccttcctttgttatatggggcttacac
ttatcatttctcctttgctttagGTTCAACGGCATTGGCGTCACACAGAACGTGCTGTACGAGAACCAGAAGCTGATCGCTAACCAG
TTCAACAGCGCCATTGGCAAGATCCAAGATTCCCTCAGCTCCACCGCCAGCGCCCTCGGCAAACTGCAAGACGTCGTGAATCAGAAT
GCCCAAGCTCTGAACACACTGGTGAAGCAGCTCAGCAGCAATTTTGGCGCCATCTCCTCCGTGCTCAATGATATTCTGTCTAGACTG
GACAAGGTGGAGGCCGAAGTCCAGATCGATAGACTGATCACAGgtaagtgtcttaaattcagaagacgtaaagcaaaacacggtttt
gaggaggcttcttattataaatcttgcattatctacttttttctagGTAGACTGCAGTCCCTCCAGACATACGTGACCCAGCAGCTC
ATTAGAGCTGCCGAGATTAGGGCCTCCGCCAATCTCGCTGCCACAAAAATGAGCGAGTGCGTGCTCGGCCAGTCCAAAAGAGTGGAC
TTCTGTGGCAAGGGCTACCATCTGATGTCCTTCCCTCAGAGCGCTCCTCATGGCGTCGTGTTTCTGCATGTGACCTACGTGCCCGCC
CAAGAGAAGAACTTCACAACAGCCCCCGCTATCTGTCACGACGGAAAGGCCCACTTCCCCAGgtaagtcattatatgaagaaaaacc
caggtgcatgttttacatgaagaaaactggtatttgtttgactggttttgcttttatgttttagGGAGGGCGTCTTTGTGTCCAACG
GCACACACTGGTTTGTCACCCAGAGGAACTTCTATGAGCCCCAGATCATCACCACCGACAACACCTTTGTGAGCGGAAACTGCGATG
TGGTCATCGGCATCGTGAATAACACCGTGTACGACCCTCTCCAGCCCGAGCTGGACTCCTTCAAGGAGGAGCTGGATAAGTACTTTA
AGAACCATACAAGCCCCGACGTGGACCTCGGCGACATTTCAGgtaagttgtccaacttttcaaagatccaggttttcttttaccata
aatgtgttattgtctgtactaatctataggatttctctcttttgtagGTATCAACGCCAGCGTCGTGAACATCCAGAAGGAGATTGA
TAGACTCAACGAGGTCGCCAAGAATCTGAACGAGTCTCTGATTGATCTGCAAGAGCTGGGCAAGTACGAGCAGTACATCAAGTGGCC
TTGGTACATCTGGCTCGGATTCATTGCCGGACTGATCGCCATCGTCATGGTGACCATCATGCTCTGCTGCATGACAAGCTGTTGCAG
CTGTCTGAAAGGCTGTTGTAGCTGTGGCAGCTGCTGTAAGTTCGATGAGGACGACTCCGAGCCCGTGCTGAAGGGCGTGAAGCTCCA
CTACACCTAA
SEQ ID NO: 19 (P230)
ATGTTCGTGTTCCTCGTGCTGCTGCCTCTGGTGTCCTCCCAGTGCGTCAATCTGACAACAAGAACACAGCTGCCCCCCGCCTACACC
AATTCCTTCACAAGAGGCGTGTACTACCCCGACAAGGTGTTCAGAAGCTCCGTGCTGCACAGCACCCAAGACCTCTTTCTGCCCTTT
TTCTCCAACGTCACATGGTTCCACGCTATCCACGTGTCAGgtaagttgatttagaaacacttttcaagcagtcagcccatggttacc
attaagttaaccctatcactgaattgctccaattttcctcttagGTACCAACGGCACCAAAAGGTTCGATAACCCCGTCCTCCCCTT
CAACGATGGCGTCTACTTCGCCAGCACCGAGAAGTCCAATATCATCAGAGGCTGGATCTTCGGCACCACACTGGATTCCAAGACCCA
GTCTCTGCTGATCGTGAATAACGCCACAAACGTGGTCATTAAAGTGTGCGAGTTCCAGTTCTGCAACGACCCCTTCCTCGGCGTGTA
TTACCACAAAAACAACAAGAGCTGGATGGAGTCCGAGTTCAGgtaaggaaatttccatgagtttcactcttgaagcattggggttat
ttgtgccagaggctaatgacccatgctggcccttcacttttctagGGTGTACAGCAGCGCCAACAACTGCACATTCGAGTACGTGAG
CCAGCCTTTCCTCATGGATCTGGAGGGCAAGCAAGGCAATTTCAAGAATCTGAGAGAGTTTGTCTTCAAGAACATCGACGGATACTT
CAAGATTTACTCCAAGCACACCCCCATTAACCTCGTCAGAGACCTCCCCCAAGGCTTTTCCGCTCTCGAACCTCTGGTGGATCTGCC
CATCGGCATCAACATCACAAGATTCCAAACCCTCCTCGCTCTGCATAGAAGCTATCTGACCCCCGGCGATTCCAGCTCAGgtaagta
atcgttcataagtaggtaaagctaaagtactaactaatttaaacaacgtaccttttttctttcctttttctcagGTTGGACAGCTGG
AGCTGCCGCCTACTATGTGGGATATCTGCAACCTAGAACATTTCTGCTGAAGTACAACGAGAACGGCACAATCACAGACGCTGTGGA
TTGTGCTCTGGACCCCCTCTCCGAGACCAAGTGTACCCTCAAGAGCTTTACCGTGGAGAAGGGAATCTACCAGACCTCCAATTTTAG
GGTCCAACCCACCGAGAGCATCGTGAGGTTCCCCAACATCACAAACCTCTGCCCTTTCGGCGAAGTGTTCAACGCCACAAGGTTTGC
TTCCGTGTACGCTTGGAACAGgtaagtagtgctgattatacacaagatattgtctagaacttgatgagactgtggatatgaatattt
cactcttttctcagGAAGAGAATCTCCAACTGCGTGGCCGACTATAGCGTGCTCTATAACAGCGCCTCCTTCAGCACCTTCAAGTGT
TACGGCGTGAGCCCCACCAAGCTGAACGATCTGTGTTTCACCAACGTGTACGCTGACTCCTTCGTCATTAGGGGCGACGAAGTGAGA
CAAATCGCTCCCGGCCAGACCGGCAAAATCGCTGACTACAACTACAAGCTCCCCGACGACTTCACCGGCTGTGTGATCGCTTGGAAC
TCCAACAACCTCGATAGCAAGGTGGGAGGCAACTACAACTATCTGTATAGACTCTTCAGgtaaggaatgttgcactgattttcacag
gattttcccaagtgatactatcttattacattgatttttggctttgttttgttttcagGAAGTCCAATCTGAAGCCCTTCGAGAGGG
ACATCAGCACAGAGATCTATCAAGCCGGATCCACACCTTGCAACGGCGTCGAGGGATTCAACTGCTACTTCCCTCTGCAATCCTACG
GCTTCCAGCCCACAAATGGCGTGGGCTACCAGCCTTACAGAGTGGTGGTGCTGTCCTTTGAACTGCTGCATGCCCCCGCCACAGTGT
GCGGACCCAAAAAGAGCACCAACCTCGTGAAGAACAAATGCGTCAATTTCAACTTCAATGGACTGACCGGCACAGgtaagtgacttg
ctttcttacatcaaaaaggcatccagtgtctgtttaagaattgccttctcaatattctctgttgattcctttccagGTGTGCTCACC
GAGTCCAACAAGAAGTTTCTGCCCTTCCAGCAGTTCGGAAGAGACATTGCCGATACCACAGACGCCGTGAGGGACCCTCAGACACTG
GAGATTCTGGATATCACACCTTGCAGCTTCGGCGGCGTGAGCGTGATCACACCCGGAACAAACACCAGCAACCAAGTGGCTGTGCTG
TACCAAGACGTGAATTGTACAGAGGTACCTGTGGCCATCCATGCCGATCAGCTGACCCCCACATGGAGGGTCTACAGCACAGgtaag
taggagaacattttcacatacaaagccatttttactttttttttaaatttcttataatcaatatgatctttttcacagGTTCCAATG
TCTTTCAGACAAGAGCTGGCTGTCTGATTGGCGCTGAGCACGTGAACAACAGCTACGAGTGCGACATCCCTATCGGCGCCGGAATTT
GCGCCAGCTACCAAACCCAGACCAATAGCCCTAGGAGGGCCAGATCCGTCGCCAGCCAGAGCATCATCGCCTATACCATGTCTCTGG
GCGCTGAGAACTCCGTGGCCTATAGCAACAACAGCATCGCTATCCCCACCAACTTCACAATCTCCGTGACCACAGgtaagttgcttt
ctctgaatacaaaactattgtttgactgtctttaagaatattactttttcatcataacttcttctttgaaaagAAATTCTGCCCGTG
AGCATGACCAAGACCAGCGTCGACTGCACCATGTATATCTGCGGCGACTCCACAGAGTGCTCCAATCTGCTGCTGCAGTACGGCAGC
TTCTGCACCCAACTCAATAGGGCTCTGACCGGAATTGCTGTCGAGCAAGACAAGAACACCCAAGAGGTGTTTGCCCAAGTGAAACAG
ATTTACAAGACCCCCCCCATCAAGGACTTCGGAGGCTTCAATTTCTCCCAAATCCTCCCCGACCCCTCCAAACCCTCCAAGAGGAGC
TTTATCGAGGATCTGCTGTTCAACAAGGTGACACTGGCTGATGCAGgtaagtctatttcaaaaaagaatcatatatattttaaaata
gcttatgtattttttacacattcatttcttatttacctactatttatccagGTTTTATCAAGCAGTATGGCGACTGTCTGGGAGACA
TCGCTGCTAGGGATCTGATCTGTGCCCAGAAGTTTAATGGCCTCACCGTGCTGCCTCCTCTGCTGACCGACGAGATGATCGCCCAGT
ATACAAGCGCTCTGCTGGCCGGCACAATTACCAGCGGATGGACATTTGGAGCCGGCGCTGCCCTCCAGATTCCTTTCGCCATGCAGA
TGGCCTACAGgtaagcaaatgaaccatcatcccatcattttgagttatatccttcctttgttatatggggcttacacttatcatttc
tcctttgctttagGTTCAACGGCATTGGCGTCACACAGAACGTGCTGTACGAGAACCAGAAGCTGATCGCTAACCAGTTCAACAGCG
CCATTGGCAAGATCCAAGATTCCCTCAGCTCCACCGCCAGCGCCCTCGGCAAACTGCAAGACGTCGTGAATCAGAATGCCCAAGCTC
TGAACACACTGGTGAAGCAGCTCAGCAGCAATTTTGGCGCCATCTCCTCCGTGCTCAATGATATTCTGTCTAGACTGGACAAGGTGG
AGGCCGAAGTCCAGATCGATAGACTGATCACAGgtaagtgtcttaaattcagaagacgtaaagcaaaacacggttttgaggaggctt
cttattataaatcttgcattatctacttttttctagGTAGACTGCAGTCCCTCCAGACATACGTGACCCAGCAGCTCATTAGAGCTG
CCGAGATTAGGGCCTCCGCCAATCTCGCTGCCACAAAAATGAGCGAGTGCGTGCTCGGCCAGTCCAAAAGAGTGGACTTCTGTGGCA
AGGGCTACCATCTGATGTCCTTCCCTCAGAGCGCTCCTCATGGCGTCGTGTTTCTGCATGTGACCTACGTGCCCGCCCAAGAGAAGA
ACTTCACAACAGCCCCCGCTATCTGTCACGACGGAAAGGCCCACTTCCCCAGgtaagtcattatatgaagaaaaacccaggtgcatg
ttttacatgaagaaaactggtatttgtttgactggttttgcttttatgttttagGGAGGGCGTCTTTGTGTCCAACGGCACACACTG
GTTTGTCACCCAGAGGAACTTCTATGAGCCCCAGATCATCACCACCGACAACACCTTTGTGAGCGGAAACTGCGATGTGGTCATCGG
CATCGTGAATAACACCGTGTACGACCCTCTCCAGCCCGAGCTGGACTCCTTCAAGGAGGAGCTGGATAAGTACTTTAAGAACCATAC
AAGCCCCGACGTGGACCTCGGCGACATTTCAGgtaagttgtccaacttttcaaagatccaggttttcttttaccataaatgtgttat
tgtctgtactaatctataggatttctctcttttgtagGTATCAACGCCAGCGTCGTGAACATCCAGAAGGAGATTGATAGACTCAAC
GAGGTCGCCAAGAATCTGAACGAGTCTCTGATTGATCTGCAAGAGCTGGGCAAGTACGAGCAGTACATCAAGTGGCCTTGGTACATC
TGGCTCGGATTCATTGCCGGACTGATCGCCATCGTCATGGTGACCATCATGCTCTGCTGCATGACAAGCTGTTGCAGCTGTCTGAAA
GGCTGTTGTAGCTGTGGCAGCTGCTGTAAGTTCGATGAGGACGACTCCGAGCCCGTGCTGAAGGGCGTGAAGCTCCACTACACCTAA
SEQ ID NO: 20 (P241)
ATGTTCGTGTTCCTCGTGCTGCTGCCTCTGGTGTCCTCCCAGTGCGTCAATCTGACAACAAGAACACAGCTGCCCCCCGCCTACACC
AATTCCTTCACAAGAGGCGTGTACTACCCCGACAAGGTGTTCAGAAGCTCCGTGCTGCACAGCACCCAAGACCTCTTTCTGCCCTTT
TTCTCCAACGTCACATGGTTCCACGCTATCCACGTGTCAGgtaagttgatttagaaacacttttcaagcagtcagcccatggttacc
attaagttaaccctatcactgaattgctccaattttcctcttagGTACCAACGGCACCAAAAGGTTCGATAACCCCGTCCTCCCCTT
CAACGATGGCGTCTACTTCGCCAGCACCGAGAAGTCCAATATCATCAGAGGCTGGATCTTCGGCACCACACTGGATTCCAAGACCCA
GTCTCTGCTGATCGTGAATAACGCCACAAACGTGGTCATTAAAGTGTGCGAGTTCCAGTTCTGCAACGACCCCTTCCTCGGCGTGTA
TTACCACAAAAACAACAAGAGCTGGATGGAGTCCGAGTTCAGgtaaggaaatttccatgagtttcactcttgaagcattggggttat
ttgtgccagaggctaatgacccatgctggcccttcacttttctagGGTGTACAGCAGCGCCAACAACTGCACATTCGAGTACGTGAG
CCAGCCTTTCCTCATGGATCTGGAGGGCAAGCAAGGCAATTTCAAGAATCTGAGAGAGTTTGTCTTCAAGAACATCGACGGATACTT
CAAGATTTACTCCAAGCACACCCCCATTAACCTCGTCAGAGACCTCCCCCAAGGCTTTTCCGCTCTCGAACCTCTGGTGGATCTGCC
CATCGGCATCAACATCACAAGATTCCAAACCCTCCTCGCTCTGCATAGAAGCTATCTGACCCCCGGCGATTCCAGCTCAGgtaagtt
agaagtattactaatgaagataatatcctgactaatagttaatagataatctttttctttcttttttttcctacagGTTGGACAGCT
GGAGCTGCCGCCTACTATGTGGGATATCTGCAACCTAGAACATTTCTGCTGAAGTACAACGAGAACGGCACAATCACAGACGCTGTG
GATTGTGCTCTGGACCCCCTCTCCGAGACCAAGTGTACCCTCAAGAGCTTTACCGTGGAGAAGGGAATCTACCAGACCTCCAATTTT
AGGGTCCAACCCACCGAGAGCATCGTGAGGTTCCCCAACATCACAAACCTCTGCCCTTTCGGCGAAGTGTTCAACGCCACAAGGTTT
GCTTCCGTGTACGCTTGGAACAGgtaagtagtgctgattatacacaagatattgtctagaacttgatgagactgtggatatgaatat
ttcactcttttctcagGAAGAGAATCTCCAACTGCGTGGCCGACTATAGCGTGCTCTATAACAGCGCCTCCTTCAGCACCTTCAAGT
GTTACGGCGTGAGCCCCACCAAGCTGAACGATCTGTGTTTCACCAACGTGTACGCTGACTCCTTCGTCATTAGGGGCGACGAAGTGA
GACAAATCGCTCCCGGCCAGACCGGCAAAATCGCTGACTACAACTACAAGCTCCCCGACGACTTCACCGGCTGTGTGATCGCTTGGA
ACTCCAACAACCTCGATAGCAAGGTGGGAGGCAACTACAACTATCTGTATAGACTCTTCAGgtaaggaatgttgcactgattttcac
aggattttcccaagtgatactatcttattacattgatttttggctttgttttgttttcagGAAGTCCAATCTGAAGCCCTTCGAGAG
GGACATCAGCACAGAGATCTATCAAGCCGGATCCACACCTTGCAACGGCGTCGAGGGATTCAACTGCTACTTCCCTCTGCAATCCTA
CGGCTTCCAGCCCACAAATGGCGTGGGCTACCAGCCTTACAGAGTGGTGGTGCTGTCCTTTGAACTGCTGCATGCCCCCGCCACAGT
GTGCGGACCCAAAAAGAGCACCAACCTCGTGAAGAACAAATGCGTCAATTTCAACTTCAATGGACTGACCGGCACAGgtaagtgact
tgctttcttacatcaaaaaggcatccagtgtctgtttaagaattgccttctcaatattctctgttgattcctttccagGTGTGCTCA
CCGAGTCCAACAAGAAGTTTCTGCCCTTCCAGCAGTTCGGAAGAGACATTGCCGATACCACAGACGCCGTGAGGGACCCTCAGACAC
TGGAGATTCTGGATATCACACCTTGCAGCTTCGGCGGCGTGAGCGTGATCACACCCGGAACAAACACCAGCAACCAAGTGGCTGTGC
TGTACCAAGACGTGAATTGTACAGAGGTACCTGTGGCCATCCATGCCGATCAGCTGACCCCCACATGGAGGGTCTACAGCACAGgta
agtaggagaacattttcacatacaaagccatttttactttttttttaaatttcttataatcaatatgatctttttcacagGTTCCAA
TGTCTTTCAGACAAGAGCTGGCTGTCTGATTGGCGCTGAGCACGTGAACAACAGCTACGAGTGCGACATCCCTATCGGCGCCGGAAT
TTGCGCCAGCTACCAAACCCAGACCAATAGCCCTAGGAGGGCCAGATCCGTCGCCAGCCAGAGCATCATCGCCTATACCATGTCTCT
GGGCGCTGAGAACTCCGTGGCCTATAGCAACAACAGCATCGCTATCCCCACCAACTTCACAATCTCCGTGACCACAGgtaagttgct
ttctctgaatacaaaactattgtttgactgtctttaagaatattactttttcatcataacttcttctttgaaaagAAATTCTGCCCG
TGAGCATGACCAAGACCAGCGTCGACTGCACCATGTATATCTGCGGCGACTCCACAGAGTGCTCCAATCTGCTGCTGCAGTACGGCA
GCTTCTGCACCCAACTCAATAGGGCTCTGACCGGAATTGCTGTCGAGCAAGACAAGAACACCCAAGAGGTGTTTGCCCAAGTGAAAC
AGATTTACAAGACCCCCCCCATCAAGGACTTCGGAGGCTTCAATTTCTCCCAAATCCTCCCCGACCCCTCCAAACCCTCCAAGAGGA
GCTTTATCGAGGATCTGCTGTTCAACAAGGTGACACTGGCTGATGCAGgtaagtctatttcaaaaaagaatcatatatattttaaaa
tagcttatgtattttttacacattcatttcttatttacctactatttatccagGTTTTATCAAGCAGTATGGCGACTGTCTGGGAGA
CATCGCTGCTAGGGATCTGATCTGTGCCCAGAAGTTTAATGGCCTCACCGTGCTGCCTCCTCTGCTGACCGACGAGATGATCGCCCA
GTATACAAGCGCTCTGCTGGCCGGCACAATTACCAGCGGATGGACATTTGGAGCCGGCGCTGCCCTCCAGATTCCTTTCGCCATGCA
GATGGCCTACAGgtaagcaaatgaaccatcatcccatcattttgagttatatccttcctttgttatatggggcttacacttatcatt
tctcctttgctttagGTTCAACGGCATTGGCGTCACACAGAACGTGCTGTACGAGAACCAGAAGCTGATCGCTAACCAGTTCAACAG
CGCCATTGGCAAGATCCAAGATTCCCTCAGCTCCACCGCCAGCGCCCTCGGCAAACTGCAAGACGTCGTGAATCAGAATGCCCAAGC
TCTGAACACACTGGTGAAGCAGCTCAGCAGCAATTTTGGCGCCATCTCCTCCGTGCTCAATGATATTCTGTCTAGACTGGACAAGGT
GGAGGCCGAAGTCCAGATCGATAGACTGATCACAGgtaagtgtcttaaattcagaagacgtaaagcaaaacacggttttgaggaggc
ttcttattataaatcttgcattatctacttttttctagGTAGACTGCAGTCCCTCCAGACATACGTGACCCAGCAGCTCATTAGAGC
TGCCGAGATTAGGGCCTCCGCCAATCTCGCTGCCACAAAAATGAGCGAGTGCGTGCTCGGCCAGTCCAAAAGAGTGGACTTCTGTGG
CAAGGGCTACCATCTGATGTCCTTCCCTCAGAGCGCTCCTCATGGCGTCGTGTTTCTGCATGTGACCTACGTGCCCGCCCAAGAGAA
GAACTTCACAACAGCCCCCGCTATCTGTCACGACGGAAAGGCCCACTTCCCCAGgtaagtcattatatgaagaaaaacccaggtgca
tgttttacatgaagaaaactggtatttgtttgactggttttgcttttatgttttagGGAGGGCGTCTTTGTGTCCAACGGCACACAC
TGGTTTGTCACCCAGAGGAACTTCTATGAGCCCCAGATCATCACCACCGACAACACCTTTGTGAGCGGAAACTGCGATGTGGTCATC
GGCATCGTGAATAACACCGTGTACGACCCTCTCCAGCCCGAGCTGGACTCCTTCAAGGAGGAGCTGGATAAGTACTTTAAGAACCAT
ACAAGCCCCGACGTGGACCTCGGCGACATTTCAGgtaagttgtccaacttttcaaagatccaggttttcttttaccataaatgtgtt
attgtctgtactaatctataggatttctctcttttgtagGTATCAACGCCAGCGTCGTGAACATCCAGAAGGAGATTGATAGACTCA
ACGAGGTCGCCAAGAATCTGAACGAGTCTCTGATTGATCTGCAAGAGCTGGGCAAGTACGAGCAGTACATCAAGTGGCCTTGGTACA
TCTGGCTCGGATTCATTGCCGGACTGATCGCCATCGTCATGGTGACCATCATGCTCTGCTGCATGACAAGCTGTTGCAGCTGTCTGA
AAGGCTGTTGTAGCTGTGGCAGCTGCTGTAAGTTCGATGAGGACGACTCCGAGCCCGTGCTGAAGGGCGTGAAGCTCCACTACACCT
AA
SEQ ID NO: 21 (P233)
ATGGTGAGCAAGGGCGAGGAGGATAACATGGCCATCATCAAGGAGTTCATGCGCTTCAAGGTGCACATGGAGGGCTCCGTGAACGGC
CACGAGTTCGAGATCGAGGGCGAGGGCGAGGGCCGCCCCTACGAGGGCACCCAGACCGCCAAGCTGAAGGTGACCAAGGGTGGCCCC
CTGCCCTTCGCCTGGGACATCCTGTCCCCTCAGTTCATGTACGGCTCCAAGGCCTACGTGAAGCACCCCGCCGACATCCCCGACTAC
TTGAAGCTGTCCTTCCCCGAGGGCTTCAAGTGGGAGCGCGTGATGAACTTCGAGGACGGCGGCGTGGTGACCGTGACCCAGGACTCC
TCCCTGCAGGACGGCGAGTTCATCTACAAGGTGAAGCTGCGCGGCACCAACTTCCCCTCCGACGGCCCCGTAATGCAGAAGAAGACC
ATGGGCTGGGAGGCCTCCTCCGAGCGGATGTACCCCGAGGACGGCGCCCTGAAGGGCGAGATCAAGCAGAGGCTGAAGCTGAAGGAC
GGCGGCCACTACGACGCTGAGGTCAAGACCACCTACAAGGCCAAGAAGCCCGTGCAGCTGCCCGGCGCCTACAACGTCAACATCAAG
TTGGACATCACCTCCCACAACGAGGACTACACCATCGTGGAACAGTACGAACGCGCCGAGGGCCGCCACTCCACCGGCGGCATGGAC
GAGCTGTACAAGTGA
SEQ ID NO: 22 (P234)
ATGGTGAGCAAGGGCGAGGAGGATAACATGGCCATCATCAAGGAGTTCATGCGCTTCAAGGTGCACATGGAGGGCTCCGTGAACGGC
CACGAGTTCGAGATCGAGGGCGAAGgtaaggaatgttgcactgattttcacaggattttcccaagtgatactatcttattacattga
tttttggctttgttttgttttcagGTGAGGGCCGCCCCTACGAGGGCACCCAGACCGCCAAGCTGAAGGTGACCAAGGGTGGCCCCC
TGCCCTTCGCCTGGGACATCCTGTCCCCTCAGTTCATGTACGGCTCCAAGGCCTACGTGAAGCACCCCGCCGACATCCCCGACTACT
TGAAGCTGTCCTTCCCCGAGGGCTTCAAGTGGGAGCGCGTGATGAACTTCGAGGACGGCGGCGTGGTGACCGTGACCCAGGACTCCT
CCCTGCAGGACGGCGAGTTCATCTACAAGGTGAAGCTGCGCGGCACCAACTTCCCCTCCGACGGCCCCGTAATGCAGAAGAAGACCA
TGGGCTGGGAGGCCTCCTCCGAGCGGATGTACCCCGAGGACGGCGCCCTGAAGGGCGAGATCAAGCAGAGGCTGAAGCTGAAGGACG
GCGGCCACTACGACGCTGAGGTCAAGACCACCTACAAGGCCAAGAAGCCCGTGCAGCTGCCCGGCGCCTACAACGTCAACATCAAGT
TGGACATCACCTCCCACAACGAGGACTACACCATCGTGGAACAGTACGAACGCGCCGAGGGCCGCCACTCCACCGGCGGCATGGACG
AGCTGTACAAGTGA
SEQ ID NO: 23 (P235)
ATGGTGAGCAAGGGCGAGGAGGATAACATGGCCATCATCAAGGAGTTCATGCGCTTCAAGGTGCACATGGAGGGCTCCGTGAACGGC
CACGAGTTCGAGATCGAGGGCGAGGGCGAGGGCCGCCCCTACGAGGGCACCCAGACCGCCAAGCTGAAGgtaagtaggagaacattt
tcacatacaaagccatttttactttttttttaaatttcttataatcaatatgatctttttcacagGTTACCAAGGGTGGCCCCCTGC
CCTTCGCCTGGGACATCCTGTCCCCTCAGTTCATGTACGGCTCCAAGGCCTACGTGAAGCACCCCGCCGACATCCCCGACTACTTGA
AGCTGTCCTTCCCCGAGGGCTTCAAGTGGGAGCGCGTGATGAACTTCGAGGACGGCGGCGTGGTGACCGTGACCCAGGACTCCTCCC
TGCAGGACGGCGAGTTCATCTACAAGgtaagtctatttcaaaaaagaatcatatatattttaaaatagcttatgtattttttacaca
ttcatttcttatttacctactatttatccagGTTAAGCTGCGCGGCACCAACTTCCCCTCCGACGGCCCCGTAATGCAGAAGAAGAC
CATGGGCTGGGAGGCCTCCTCCGAGCGGATGTACCCCGAGGACGGCGCCCTGAAGGGCGAGATCAAGCAGAGGCTGAAGCTGAAGGA
CGGCGGCCACTACGACGCTGAGGTCAAGACCACCTACAAGGCCAAGAAGCCCGTGCAGCTGCCAGgtaagtcattatatgaagaaaa
acccaggtgcatgttttacatgaagaaaactggtatttgtttgactggttttgcttttatgttttagGTGCCTACAACGTCAACATC
AAGTTGGACATCACCTCCCACAACGAGGACTACACCATCGTGGAACAGTACGAACGCGCCGAGGGCCGCCACTCCACCGGCGGCATG
GACGAGCTGTACAAGTGA
SEQ ID NO: 24 (P236)
ATGGTGAGCAAGGGCGAGGAGGATAACATGGCCATCATCAAGGAGTTCATGCGCTTCAAGgtaagtaatttatataccactagagat
tttttcatcagtttctgttataaaaataattaaaatcaacatatttttctcctttacaacagGTTCACATGGAGGGCTCCGTGAACG
GCCACGAGTTCGAGATCGAGGGCGAGGGCGAGGGCCGCCCCTACGAGGGCACCCAGACCGCCAAGCTGAAGGTGACCAAGGGTGGCC
CCCTGCCCTTCGCCTGGGACATCCTGTCCCCTCAGTTCATGTACGGCTCCAAGGCCTACGTGAAGCACCCCGCCGACATCCCCGACT
ACTTGAAGCTGTCCTTCCCCGAAGgtaagttgctttctctgaatacaaaactattgtttgactgtctttaagaatattactttttca
tcataacttcttctttgaaaagGTTTCAAGTGGGAGCGCGTGATGAACTTCGAGGACGGCGGCGTGGTGACCGTGACCCAGGACTCC
TCCCTGCAGGACGGCGAGTTCATCTACAAGGTGAAGCTGCGCGGCACCAACTTCCCCTCCGACGGCCCCGTAATGCAGAAGAAGACC
ATGGGCTGGGAGGCCTCCTCCGAGCGGATGTACCCCGAGGACGGCGCCCTGAAAGgtaagtgtcttaaattcagaagacgtaaagca
aaacacggttttgaggaggcttcttattataaatcttgcattatctacttttttctagGTGAGATCAAGCAGAGGCTGAAGCTGAAG
GACGGCGGCCACTACGACGCTGAGGTCAAGACCACCTACAAGGCCAAGAAGCCCGTGCAGCTGCCCGGCGCCTACAACGTCAACATC
AAGTTGGACATCACCTCCCACAACGAGGACTACACCATCGTGGAACAGTACGAACGCGCCGAAGgtaagttgtccaacttttcaaag
atccaggttttcttttaccataaatgtgttattgtctgtactaatctataggatttctctcttttgtagGTCGCCACTCCACCGGCG
GCATGGACGAGCTGTACAAGTGA
SEQ ID NO: 25 (P237)
ATGGTGAGCAAGGGCGAGGAGGATAACATGGCCATCATCAAGGAGTTCATGCGCTTCAAGgtaagtaatttatataccactagagat
tttttcatcagtttctgttataaaaataattaaaatcaacatatttttctcctttacaacagGTTCACATGGAGGGCTCCGTGAACG
GCCACGAGTTCGAGATCGAGGGCGAGGGCGAGGGCCGCCCCTACGAGGGCACCCAGACCGCCAAGCTGAAGgtaagtaggagaacat
tttcacatacaaagccatttttactttttttttaaatttcttataatcaatatgatctttttcacagGTTACCAAGGGTGGCCCCCT
GCCCTTCGCCTGGGACATCCTGTCCCCTCAGTTCATGTACGGCTCCAAGGCCTACGTGAAGCACCCCGCCGACATCCCCGACTACTT
GAAGCTGTCCTTCCCCGAAGgtaagttgctttctctgaatacaaaactattgtttgactgtctttaagaatattactttttcatcat
aacttcttctttgaaaagGTTTCAAGTGGGAGCGCGTGATGAACTTCGAGGACGGCGGCGTGGTGACCGTGACCCAGGACTCCTCCC
TGCAGGACGGCGAGTTCATCTACAAGgtaagtctatttcaaaaaagaatcatatatattttaaaatagcttatgtattttttacaca
ttcatttcttatttacctactatttatccagGTTAAGCTGCGCGGCACCAACTTCCCCTCCGACGGCCCCGTAATGCAGAAGAAGAC
CATGGGCTGGGAGGCCTCCTCCGAGCGGATGTACCCCGAGGACGGCGCCCTGAAAGgtaagtgtcttaaattcagaagacgtaaagc
aaaacacggttttgaggaggcttcttattataaatcttgcattatctacttttttctagGTGAGATCAAGCAGAGGCTGAAGCTGAA
GGACGGCGGCCACTACGACGCTGAGGTCAAGACCACCTACAAGGCCAAGAAGCCCGTGCAGCTGCCAGgtaagtcattatatgaaga
aaaacccaggtgcatgttttacatgaagaaaactggtatttgtttgactggttttgcttttatgttttagGTGCCTACAACGTCAAC
ATCAAGTTGGACATCACCTCCCACAACGAGGACTACACCATCGTGGAACAGTACGAACGCGCCGAAGgtaagttgtccaacttttca
aagatccaggttttcttttaccataaatgtgttattgtctgtactaatctataggatttctctcttttgtagGTCGCCACTCCACCG
GCGGCATGGACGAGCTGTACAAGTGA
SEQ ID NO: 26 (P238)
ATGGTGAGCAAGGGCGAGGAGGATAACATGGCCATCATCAAGGAGTTCATGCGCTTCAAGgtaagtaatttatataccactagagat
tttttcatcagtttctgttataaaaataattaaaatcaacatatttttctcctttacaacagGTTCACATGGAGGGCTCCGTGAACG
GCCACGAGTTCGAGATCGAGGGCGAAGgtaaggaatgttgcactgattttcacaggattttcccaagtgatactatcttattacatt
gatttttggctttgttttgttttcagGTGAGGGCCGCCCCTACGAGGGCACCCAGACCGCCAAGCTGAAGgtaagtaggagaacatt
ttcacatacaaagccatttttactttttttttaaatttcttataatcaatatgatctttttcacagGTTACCAAGGGTGGCCCCCTG
CCCTTCGCCTGGGACATCCTGTCCCCTCAGTTCATGTACGGCTCCAAGGCCTACGTGAAGCACCCCGCCGACATCCCCGACTACTTG
AAGCTGTCCTTCCCCGAAGgtaagttgctttctctgaatacaaaactattgtttgactgtctttaagaatattactttttcatcata
acttcttctttgaaaagGTTTCAAGTGGGAGCGCGTGATGAACTTCGAGGACGGCGGCGTGGTGACCGTGACCCAGGACTCCTCCCT
GCAGGACGGCGAGTTCATCTACAAGgtaagtctatttcaaaaaagaatcatatatattttaaaatagcttatgtattttttacacat
tcatttcttatttacctactatttatccagGTTAAGCTGCGCGGCACCAACTTCCCCTCCGACGGCCCCGTAATGCAGAAGAAGACC
ATGGGCTGGGAGGCCTCCTCCGAGCGGATGTACCCCGAGGACGGCGCCCTGAAAGgtaagtgtcttaaattcagaagacgtaaagca
aaacacggttttgaggaggcttcttattataaatcttgcattatctacttttttctagGTGAGATCAAGCAGAGGCTGAAGCTGAAG
GACGGCGGCCACTACGACGCTGAGGTCAAGACCACCTACAAGGCCAAGAAGCCCGTGCAGCTGCCAGgtaagtcattatatgaagaa
aaacccaggtgcatgttttacatgaagaaaactggtatttgtttgactggttttgcttttatgttttagGTGCCTACAACGTCAACA
TCAAGTTGGACATCACCTCCCACAACGAGGACTACACCATCGTGGAACAGTACGAACGCGCCGAAGgtaagttgtccaacttttcaa
agatccaggttttcttttaccataaatgtgttattgtctgtactaatctataggatttctctcttttgtagGTCGCCACTCCACCGG
CGGCATGGACGAGCTGTACAAGTGA
SEQ ID NO: 27 (P95)
ATGTCAAGCTCTTCCTGGCTCCTTCTCAGCCTTGTTGCTGTAACTGCTGCTCAGTCCACCATTGAGGAACAGGCCAAGACATTTTTG
GACAAGTTTAACCACGAAGCCGAAGACCTGTTCTATCAAAGTTCACTTGCTTCTTGGAATTATAACACCAATATTACTGAAGAGAAT
GTCCAAAACATGAATAATGCTGGGGACAAATGGTCTGCCTTTTTAAAGGAACAGTCCACACTTGCCCAAATGTATCCACTACAAGAA
ATTCAGAATCTCACAGTCAAGCTTCAGCTGCAGGCTCTTCAGCAAAATGGGTCTTCAGTGCTCTCAGAAGACAAGAGCAAACGGTTG
AACACAATTCTAAATACAATGAGCACCATCTACAGTACTGGAAAAGTTTGTAACCCAGATAATCCACAAGAATGCTTATTACTTGAA
CCAGGTTTGAATGAAATAATGGCAAACAGTTTAGACTACAATGAGAGGCTCTGGGCTTGGGAAAGCTGGAGATCTGAGGTCGGCAAG
CAGCTGAGGCCATTATATGAAGAGTATGTGGTCTTGAAAAATGAGATGGCAAGAGCAAATCATTATGAGGACTATGGGGATTATTGG
AGAGGAGACTATGAAGTAAATGGGGTAGATGGCTATGACTACAGCCGCGGCCAGTTGATTGAAGATGTGGAACATACCTTTGAAGAG
ATTAAACCATTATATGAACATCTTCATGCCTATGTGAGGGCAAAGTTGATGAATGCCTATCCTTCCTATATCAGTCCAATTGGATGC
CTCCCTGCTCATTTGCTTGGTGATATGTGGGGTAGATTTTGGACAAATCTGTACTCTTTGACAGTTCCCTTTGGACAGAAACCAAAC
ATAGATGTTACTGATGCAATGGTGGACCAGGCCTGGGATGCACAGAGAATATTCAAGGAGGCCGAGAAGTTCTTTGTATCTGTTGGT
CTTCCTAATATGACTCAAGGATTCTGGGAAAATTCCATGCTAACGGACCCAGGAAATGTTCAGAAAGCAGTCTGCCATCCCACAGCT
TGGGACCTGGGGAAGGGCGACTTCAGGATCCTTATGTGCACAAAGGTGACAATGGACGACTTCCTGACAGCTCATCATGAGATGGGG
CATATCCAGTATGATATGGCATATGCTGCACAACCTTTTCTGCTAAGAAATGGAGCTAATGAAGGATTCCATGAAGCTGTTGGGGAA
ATCATGTCACTTTCTGCAGCCACACCTAAGCATTTAAAATCCATTGGTCTTCTGTCACCCGATTTTCAAGAAGACAATGAAACAGAA
ATAAACTTCCTGCTCAAACAAGCACTCACGATTGTTGGGACTCTGCCATTTACTTACATGTTAGAGAAGTGGAGGTGGATGGTCTTT
AAAGGGGAAATTCCCAAAGACCAGTGGATGAAAAAGTGGTGGGAGATGAAGCGAGAGATAGTTGGGGTGGTGGAACCTGTGCCCCAT
GATGAAACATACTGTGACCCCGCATCTCTGTTCCATGTTTCTAATGATTACTCATTCATTCGATATTACACAAGGACCCTTTACCAA
TTCCAGTTTCAAGAAGCACTTTGTCAAGCAGCTAAACATGAAGGCCCTCTGCACAAATGTGACATCTCAAACTCTACAGAAGCTGGA
CAGAAACTGTTCAATATGCTGAGGCTTGGAAAATCAGAACCCTGGACCCTAGCATTGGAAAATGTTGTAGGAGCAAAGAACATGAAT
GTAAGGCCACTGCTCAACTACTTTGAGCCCTTATTTACCTGGCTGAAAGACCAGAACAAGAATTCTTTTGTGGGATGGAGTACCGAC
TGGAGTCCATATGCAGACCAAAGCATCAAAGTGAGGATAAGCCTAAAATCAGCTCTTGGAGATAAAGCATATGAATGGAACGACAAT
GAAATGTACCTGTTCCGATCATCTGTTGCATATGCTATGAGGCAGTACTTTTTAAAAGTAAAAAATCAGATGATTCTTTTTGGGGAG
GAGGATGTGCGAGTGGCTAATTTGAAACCAAGAATCTCCTTTAATTTCTTTGTCACTGCACCTAAAAATGTGTCTGATATCATTCCT
AGAACTGAAGTTGAAAAGGCCATCAGGATGTCCCGGAGCCGTATCAATGATGCTTTCCGTCTGAATGACAACAGCCTAGAGTTTCTG
GGGATACAGCCAACACTTGGACCTCCTAACCAGCCCCCTGTTTCCATATGGCTGATTGTTTTTGGAGTTGTGATGGGAGTGATAGTG
GTTGGCATTGTCATCCTGATCTTCACTGGGATCAGAGATCGGAAGAAGAAAAATAAAGCAAGAAGTGGAGAAAATCCTTATGCCTCC
ATCGATATTAGCAAAGGAGAAAATAATCCAGGATTCCAAAACACTGATGATGTTCAGACCTCCTTTTAG
SEQ ID NO: 28 (P223)
ATGTCAAGCTCTTCCTGGCTCCTTCTCAGCCTTGTTGCTGTAACTGCTGCTCAGTCCACCATTGAGGAACAGGCCAAGACATTTTTG
GACAAGTTTAACCACGAAGCCGAAGACCTGTTCTATCAAAGTTCACTTGCTTCTTGGAATTATAACACCAATATTACTGAAGAGAAT
GTCCAAAACATGAATAATGCTGGGGACAAATGGTCTGCCTTTTTAAAGGAACAGTCCACACTTGCCCAAATGTATCCACTACAAGAA
ATTCAGAATCTCACAGTCAAGCTTCAGCTGCAGGCTCTTCAGCAAAATGGGTCTTCAGTGCTCTCAGAAGACAAGAGCAAACGGTTG
AACACAATTCTAAATACAATGAGCACCATCTACAGTACTGGAAAAGTTTGTAACCCAGATAATCCACAAGAATGCTTATTACTTGAA
CCAGgtaagttgtccaacttttcaaagatccaggttttcttttaccataaatgtgttattgtctgtactaatctataggatttctct
cttttgtagGTTTGAATGAAATAATGGCAAACAGTTTAGACTACAATGAGAGGCTCTGGGCTTGGGAAAGCTGGAGATCTGAGGTCG
GCAAGCAGCTGAGGCCATTATATGAAGAGTATGTGGTCTTGAAAAATGAGATGGCAAGAGCAAATCATTATGAGGACTATGGGGATT
ATTGGAGAGGAGACTATGAAGTAAATGGGGTAGATGGCTATGACTACAGCCGCGGCCAGTTGATTGAAGATGTGGAACATACCTTTG
AAGAGATTAAACCATTATATGAACATCTTCATGCCTATGTGAGGGCAAAGTTGATGAATGCCTATCCTTCCTATATCAGTCCAATTG
GATGCCTCCCTGCTCATTTGCTTGGTGATATGTGGGGTAGATTTTGGACAAATCTGTACTCTTTGACAGTTCCCTTTGGACAGAAAC
CAAACATAGATGTTACTGATGCAATGGTGGACCAGGCCTGGGATGCACAGAGAATATTCAAGGAGGCCGAGAAGTTCTTTGTATCTG
TTGGTCTTCCTAATATGACTCAAGGATTCTGGGAAAATTCCATGCTAACGGACCCAGGAAATGTTCAGAAAGCAGTCTGCCATCCCA
CAGCTTGGGACCTGGGGAAGGGCGACTTCAGgtaagttgctttctctgaatacaaaactattgtttgactgtctttaagaatattac
tttttcatcataacttcttctttgaaaagGATCCTTATGTGCACAAAGGTGACAATGGACGACTTCCTGACAGCTCATCATGAGATG
GGGCATATCCAGTATGATATGGCATATGCTGCACAACCTTTTCTGCTAAGAAATGGAGCTAATGAAGGATTCCATGAAGCTGTTGGG
GAAATCATGTCACTTTCTGCAGCCACACCTAAGCATTTAAAATCCATTGGTCTTCTGTCACCCGATTTTCAAGAAGACAATGAAACA
GAAATAAACTTCCTGCTCAAACAAGCACTCACGATTGTTGGGACTCTGCCATTTACTTACATGTTAGAGAAGTGGAGGTGGATGGTC
TTTAAAGGGGAAATTCCCAAAGACCAGTGGATGAAAAAGTGGTGGGAGATGAAGCGAGAGATAGTTGGGGTGGTGGAACCTGTGCCC
CATGATGAAACATACTGTGACCCCGCATCTCTGTTCCATGTTTCTAATGATTACTCATTCATTCGATATTACACAAGGACCCTTTAC
CAATTCCAGTTTCAAGAAGCACTTTGTCAAGCAGCTAAACATGAAGGCCCTCTGCACAAATGTGACATCTCAAACTCTACAGAAGCT
GGACAGAAACTGTTCAATATGCTGAGGCTTGGAAAATCAGAACCCTGGACCCTAGCATTGGAAAATGTTGTAGGAGCAAAGAACATG
AATGTAAGGCCACTGCTCAACTACTTTGAGCCCTTATTTACCTGGCTGAAAGACCAGAACAAGAATTCTTTTGTGGGATGGAGTACC
GACTGGAGTCCATgtaagtctatttcaaaaaagaatcatatatattttaaaatagcttatgtattttttacacattcatttcttatt
tacctactatttatccagATGCAGACCAAAGCATCAAAGTGAGGATAAGCCTAAAATCAGCTCTTGGAGATAAAGCATATGAATGGA
ACGACAATGAAATGTACCTGTTCCGATCATCTGTTGCATATGCTATGAGGCAGTACTTTTTAAAAGTAAAAAATCAGATGATTCTTT
TTGGGGAGGAGGATGTGCGAGTGGCTAATTTGAAACCAAGAATCTCCTTTAATTTCTTTGTCACTGCACCTAAAAATGTGTCTGATA
TCATTCCTAGAACTGAAGTTGAAAAGGCCATCAGGATGTCCCGGAGCCGTATCAATGATGCTTTCCGTCTGAATGACAACAGCCTAG
AGTTTCTGGGGATACAGCCAACACTTGGACCTCCTAACCAGCCCCCTGTTTCCATATGGCTGATTGTTTTTGGAGTTGTGATGGGAG
TGATAGTGGTTGGCATTGTCATCCTGATCTTCACTGGGATCAGAGATCGGAAGAAGAAAAATAAAGCAAGAAGTGGAGAAAATCCTT
ATGCCTCCATCGATATTAGCAAAGGAGAAAATAATCCAGGATTCCAAAACACTGATGATGTTCAGACCTCCTTTTAG
SEQ ID NO: 29 (P242)
ATGTCAAGCTCTTCCTGGCTCCTTCTCAGCCTTGTTGCTGTAACTGCTGCTCAGTCCACCATTGAGGAACAGGCCAAGACATTTTTG
GACAAGTTTAACCACGAAGCCGAAGACCTGTTCTATCAAAGTTCACTTGCTTCTTGGAATTATAACACCAATATTACTGAAGAGAAT
GTCCAAAACATGAATAATGCTGGGGACAAATGGTCTGCCTTTTTAAAGGAACAGTCCACACTTGCCCAAATGTATCCACTACAAGAA
ATTCAGAATCTCACAGTCAAGCTTCAGCTGCAGGCTCTTCAGCAAAATGGGTCTTCAGTGCTCTCAGAAGACAAGAGCAAACGGTTG
AACACAATTCTAAATACAATGAGCACCATCTACAGTACTGGAAAAGTTTGTAACCCAGATAATCCACAAGAATGCTTATTACTTGAA
CCAGgtaagttgtccaacttttcaaagatccaggttttcttttaccataaatgtgttattgtctgtactaatctataggatttctct
cttttgtagGTTTGAATGAAATAATGGCAAACAGTTTAGACTACAATGAGAGGCTCTGGGCTTGGGAAAGCTGGAGATCTGAGGTCG
GCAAGCAGCTGAGGCCATTATATGAAGAGTATGTGGTCTTGAAAAATGAGATGGCAAGAGCAAATCATTATGAGGACTATGGGGATT
ATTGGAGAGGAGACTATGAAGTAAATGGGGTAGATGGCTATGACTACAGCCGCGGCCAGTTGATTGAAGATGTGGAACATACCTTTG
AAGAGgtaagtaggagaacattttcacatacaaagccatttttactttttttttaaatttcttataatcaatatgatctttttcaca
gATTAAACCATTATATGAACATCTTCATGCCTATGTGAGGGCAAAGTTGATGAATGCCTATCCTTCCTATATCAGTCCAATTGGATG
CCTCCCTGCTCATTTGCTTGGTGATATGTGGGGTAGATTTTGGACAAATCTGTACTCTTTGACAGTTCCCTTTGGACAGAAACCAAA
CATAGATGTTACTGATGCAATGGTGGACCAGGCCTGGGATGCACAGAGAATATTCAAGGAGGCCGAGAAGTTCTTTGTATCTGTTGG
TCTTCCTAATATGACTCAAGGATTCTGGGAAAATTCCATGCTAACGGACCCAGGAAATGTTCAGAAAGCAGTCTGCCATCCCACAGC
TTGGGACCTGGGGAAGGGCGACTTCAGgtaagttgctttctctgaatacaaaactattgtttgactgtctttaagaatattactttt
tcatcataacttcttctttgaaaagGATCCTTATGTGCACAAAGGTGACAATGGACGACTTCCTGACAGCTCATCATGAGATGGGGC
ATATCCAGTATGATATGGCATATGCTGCACAACCTTTTCTGCTAAGAAATGGAGCTAATGAAGGATTCCATGAAGCTGTTGGGGAAA
TCATGTCACTTTCTGCAGCCACACCTAAGCATTTAAAATCCATTGGTCTTCTGTCACCCGATTTTCAAGAAGACAATGAAACAGAAA
TAAACTTCCTGCTCAAACAAGCACTCACGATTGTTGGGACTCTGCCATTTACTTACATGTTAGAGAAGTGGAGGTGGATGGTCTTTA
AAGGGGAAATTCCCAAAGACCAGTGGATGAAAAAGTGGTGGGAGATGAAgtaagtacagaagccatcaaacttttatatctgtttta
ttcattttcaaataattataaaaataatattcttactaatatttatttcagGCGAGAGATAGTTGGGGTGGTGGAACCTGTGCCCCA
TGATGAAACATACTGTGACCCCGCATCTCTGTTCCATGTTTCTAATGATTACTCATTCATTCGATATTACACAAGGACCCTTTACCA
ATTCCAGTTTCAAGAAGCACTTTGTCAAGCAGCTAAACATGAAGGCCCTCTGCACAAATGTGACATCTCAAACTCTACAGAAGCTGG
ACAGAAACTGTTCAATATGCTGAGGCTTGGAAAATCAGAACCCTGGACCCTAGCATTGGAAAATGTTGTAGGAGCAAAGAACATGAA
TGTAAGGCCACTGCTCAACTACTTTGAGCCCTTATTTACCTGGCTGAAAGACCAGAACAAGAATTCTTTTGTGGGATGGAGTACCGA
CTGGAGTCCATgtaagtctatttcaaaaaagaatcatatatattttaaaatagcttatgtattttttacacattcatttcttattta
cctactatttatccagATGCAGACCAAAGCATCAAAGTGAGGATAAGCCTAAAATCAGCTCTTGGAGATAAAGCATATGAATGGAAC
GACAATGAAATGTACCTGTTCCGATCATCTGTTGCATATGCTATGAGGCAGTACTTTTTAAAAGTAAAAAATCAGATGATTCTTTTT
GGGGAGGAGGATGTGCGAGTGGCTAATTTGAAACCAAGAATCTCCTTTAATTTCTTTGTCACTGCACCTAAAAATGTGTCTGATATC
ATTCCTAGAACTGAAGTTGAAAAGGCCATCAGgtaagtgtcttaaattcagaagacgtaaagcaaaacacggttttgaggaggcttc
ttattataaatcttgcattatctacttttttctagGATGTCCCGGAGCCGTATCAATGATGCTTTCCGTCTGAATGACAACAGCCTA
GAGTTTCTGGGGATACAGCCAACACTTGGACCTCCTAACCAGCCCCCTGTTTCCATATGGCTGATTGTTTTTGGAGTTGTGATGGGA
GTGATAGTGGTTGGCATTGTCATCCTGATCTTCACTGGGATCAGAGATCGGAAGAAGAAAAATAAAGCAAGAAGTGGAGAAAATCCT
TATGCCTCCATCGATATTAGCAAAGGAGAAAATAATCCAGGATTCCAAAACACTGATGATGTTCAGACCTCCTTTTAG
SEQ ID NO: 30 (P243)
ATGTCAAGCTCTTCCTGGCTCCTTCTCAGCCTTGTTGCTGTAACTGCTGCTCAGTCCACCATTGAGGAACAGGCCAAGACATTTTTG
GACAAGTTTAACCACGAAGCCGAAGACCTGTTCTATCAAAGTTCACTTGCTTCTTGGAATTATAACACCAATATTACTGAAGAGAAT
GTCCAAAACATGgtaagtaatttatataccactagagattttttcatcagtttctgttataaaaataattaaaatcaacatattttt
ctcctttacaacagAATAATGCTGGGGACAAATGGTCTGCCTTTTTAAAGGAACAGTCCACACTTGCCCAAATGTATCCACTACAAG
AAATTCAGAATCTCACAGTCAAGCTTCAGCTGCAGGCTCTTCAGCAAAATGGGTCTTCAGTGCTCTCAGAAGACAAGAGCAAACGGT
TGAACACAATTCTAAATACAATGAGCACCATCTACAGTACTGGAAAAGTTTGTAACCCAGATAATCCACAAGAATGCTTATTACTTG
AACCAGgtaagttgtccaacttttcaaagatccaggttttcttttaccataaatgtgttattgtctgtactaatctataggatttct
ctcttttgtagGTTTGAATGAAATAATGGCAAACAGTTTAGACTACAATGAGAGGCTCTGGGCTTGGGAAAGCTGGAGATCTGAGGT
CGGCAAGCAGCTGAGGCCATTATATGAAGAGTATGTGGTCTTGAAAAATGAGATGGCAAGAGCAAATCATTATGAGGACTATGGGGA
TTATTGGAGAGGAGACTATGAAGTAAATGGGGTAGATGGCTATGACTACAGCCGCGGCCAGTTGATTGAAGATGTGGAACATACCTT
TGAAGAGgtaagtaggagaacattttcacatacaaagccatttttactttttttttaaatttcttataatcaatatgatctttttca
cagATTAAACCATTATATGAACATCTTCATGCCTATGTGAGGGCAAAGTTGATGAATGCCTATCCTTCCTATATCAGTCCAATTGGA
TGCCTCCCTGCTCATTTGCTTGGTGATATGTGGGGTAGATTTTGGACAAATCTGTACTCTTTGACAGTTCCCTTTGGACAGAAACCA
AACATAGATGTTACTGATGCAATGGTGGACCAGgtaaggaatgttgcactgattttcacaggattttcccaagtgatactatcttat
tacattgatttttggctttgttttgttttcagGCCTGGGATGCACAGAGAATATTCAAGGAGGCCGAGAAGTTCTTTGTATCTGTTG
GTCTTCCTAATATGACTCAAGGATTCTGGGAAAATTCCATGCTAACGGACCCAGGAAATGTTCAGAAAGCAGTCTGCCATCCCACAG
CTTGGGACCTGGGGAAGGGCGACTTCAGgtaagttgctttctctgaatacaaaactattgtttgactgtctttaagaatattacttt
ttcatcataacttcttctttgaaaagGATCCTTATGTGCACAAAGGTGACAATGGACGACTTCCTGACAGCTCATCATGAGATGGGG
CATATCCAGTATGATATGGCATATGCTGCACAACCTTTTCTGCTAAGAAATGGAGCTAATGAAGGATTCCATGAAGCTGTTGGGGAA
ATCATGTCACTTTCTGCAGCCACACCTAAGCATTTAAAATCCATTGGTCTTCTGTCACCCGATTTTCAAGAAGACAATGAAACAGAA
ATAAACTTCCTGCTCAAACAAGCACTCACGATTGTTGGGACTCTGCCATTTACTTACATGTTAGAGAAGTGGAGGTGGATGGTCTTT
AAAGGGGAAATTCCCAAAGACCAGTGGATGAAAAAGTGGTGGGAGATGAAgtaagtacagaagccatcaaacttttatatctgtttt
attcattttcaaataattataaaaataatattcttactaatatttatttcagGCGAGAGATAGTTGGGGTGGTGGAACCTGTGCCCC
ATGATGAAACATACTGTGACCCCGCATCTCTGTTCCATGTTTCTAATGATTACTCATTCATTCGATATTACACAAGGACCCTTTACC
AATTCCAGTTTCAAGAAGCACTTTGTCAAGCAGCTAAACATGAAGGCCCTCTGCACAAATGTGACATCTCAAACTCTACAGAAGCTG
GACAGAAACTGTTgtaagtcgattccttgcttatgtatatatctcacagtttgtattttgaatttttaaaaaatatttttctttttt
ttcttttttcttacagCAATATGCTGAGGCTTGGAAAATCAGAACCCTGGACCCTAGCATTGGAAAATGTTGTAGGAGCAAAGAACA
TGAATGTAAGGCCACTGCTCAACTACTTTGAGCCCTTATTTACCTGGCTGAAAGACCAGAACAAGAATTCTTTTGTGGGATGGAGTA
CCGACTGGAGTCCATgtaagtctatttcaaaaaagaatcatatatattttaaaatagcttatgtattttttacacattcatttctta
tttacctactatttatccagATGCAGACCAAAGCATCAAAGTGAGGATAAGCCTAAAATCAGCTCTTGGAGATAAAGCATATGAATG
GAACGACAATGAAATGTACCTGTTCCGATCATCTGTTGCATATGCTATGAGGCAGTACTTTTTAAAAGTAAAAAATCAGATGATTCT
TTTTGGGGAGGAGGATGTGCGAGTGGCTAATTTGAAACCAAGAATCTCCTTTAATTTCTTTGTCACTGCACCTAAAAATGTGTCTGA
TATCATTCCTAGAACTGAAGTTGAAAAGGCCATCAGgtaagtgtcttaaattcagaagacgtaaagcaaaacacggttttgaggagg
cttcttattataaatcttgcattatctacttttttctagGATGTCCCGGAGCCGTATCAATGATGCTTTCCGTCTGAATGACAACAG
CCTAGAGTTTCTGGGGATACAGCCAACACTTGGACCTCCTAACCAGCCCCCTGTTTCCATATGGCTGATTGTTTTTGGAGTTGTGAT
GGGAGTGATAGTGGTTGGCATTGTCATCCTGATCTTCACTGGGATCAGAGATCGGAAGAAGAAAAATAAAGCAAGAAGTGGAGAAAA
TCCTTATGCCTCCATCGATATTAGCAAAGGAGAAAATAATCCAGGATTCCAAAACACTGATGATGTTCAGACCTCCTTTTAG
Lower case: intron
Upper case: exon
Upper case with underline: UTR

### REFERENCES

Amit, M., M. Donyo, D. Hollander, A. Goren, E. Kim, S. Gelfman, G. Lev-Maor, D. Burstein, S. Schwartz, B. Postolsky, T. Pupko and G. Ast (2012). “Differential GC content between exons and introns establishes distinct strategies of splice-site recognition.” Cell Rep 1(5): 543-556.
Bourdon, V., A. Harvey and D. M. Lonsdale (2001). “Introns and their positions affect the translational activity of mRNA in plant cells.” EMBO Rep 2(5): 394-398.
Buchman, A. R. and P. Berg (1988). “Comparison of intron-dependent and intron-independent gene expression.” Mol Cell Biol 8(10): 4395-4405.
Callendret, B., V. Lorin, P. Charneau, P. Marianneau, H. Contamin, J. M. Betton, S. van der Werf and N. Escriou (2007). “Heterologous viral RNA export elements improve expression of severe acute respiratory syndrome (SARS) coronavirus spike protein and protective efficacy of DNA vaccines against SARS.” Virology 363(2): 288-302.
Chalfie, M., Y. Tu, G. Euskirchen, W. W. Ward and D. C. Prasher (1994). “Green fluorescent protein as a marker for gene expression.” Science 263(5148): 802-805.
Chen Ling, Y. C., Liu Xiaolin (2020). “Adenovirus vector vaccine for preventing SARS-COV-2 infection.” CN110974950B
Cockett, M. I., C. R. Bebbington and G. T. Yarranton (1990). “High level expression of tissue inhibitor of metalloproteinases in Chinese hamster ovary cells using glutamine synthetase gene amplification.” Biotechnology (N Y) 8(7): 662-667.
Crane, M. M., B. Sands, C. Battaglia, B. Johnson, S. Yun, M. Kaeberlein, R. Brent and A. Mendenhall (2019). “In vivo measurements reveal a single 5′-intron is sufficient to increase protein expression level in Caenorhabditis elegans.” Sci Rep 9(1): 9192.
Enenkel, B. (2017). “Artificial introns.” U.S. Pat. No. 9,708,636B2
Gray, S. J., S. B. Foti, J. W. Schwartz, L. Bachaboina, B. Taylor-Blake, J. Coleman, M. D. Ehlers, M. J. Zylka, T. J. McCown and R. J. Samulski (2011). “Optimizing promoters for recombinant adeno-associated virus-mediated gene expression in the peripheral and central nervous system using self-complementary vectors.” Hum Gene Ther 22(9): 1143-1153.
Gromak, N. (2012). “Intronic microRNAs: a crossroad in gene regulation.” Biochem Soc Trans 40(4): 759-761.
Grutzner, R., P. Martin, C. Horn, S. Mortensen, E. J. Cram, C. W. T. Lee-Parsons, J. Stuttmann and S. Marillonnet (2021). “High-efficiency genome editing in plants mediated by a Cas9 gene containing multiple introns.” Plant Commun 2(2): 100135.
Gustafsson, C., S. Govindarajan and J. Minshull (2004). “Codon bias and heterologous protein expression.” Trends Biotechnol 22(7): 346-353.
Hamer, D. H. and P. Leder (1979). “Splicing and the formation of stable RNA.” Cell 18(4): 1299-1302.
Haruyama, N., A. Cho and A. B. Kulkarni (2009). “Overview: engineering transgenic constructs and mice.” Curr Protoc Cell Biol Chapter 19: Unit 19 10.
Jin, Y., M. Fei, S. Rosenquist, L. Jin, S. Gohil, C. Sandstrom, H. Olsson, C. Persson, A. S. Hoglund, G. Fransson, Y. Ruan, P. Aman, C. Jansson, C. Liu, R. Andersson and C. Sun (2017). “A Dual-Promoter Gene Orchestrates the Sucrose-Coordinated Synthesis of Starch and Fructan in Barley.” Mol Plant 10(12): 1556-1570.
Kozak, M. (1984). “Compilation and analysis of sequences upstream from the translational start site in eukaryotic mRNAs.” Nucleic Acids Res 12(2): 857-872.
Lacy-Hulbert, A., R. Thomas, X. P. Li, C. E. Lilley, R. S. Coffin and J. Roes (2001). “Interruption of coding sequences by heterologous introns can enhance the functional expression of recombinant genes.” Gene Ther 8(8): 649-653.
Le Hir, H., A. Nott and M. J. Moore (2003). “How introns influence and enhance eukaryotic gene expression.” Trends Biochem Sci 28(4): 215-220.
Lemaire, S., N. Fontrodona, F. Aube, J. B. Claude, H. Polveche, L. Modolo, C. F. Bourgeois, F. Mortreux and D. Auboeuf (2019). “Characterizing the interplay between gene nucleotide composition bias and splicing.” Genome Biol 20(1): 259.
Marillonnet, S., C. Engler, V. Klimyuk and Y. Gleba (2010). “Rna Virus-derived Plant Expression System.” EP2184363
Mordstein, C., R. Savisaar, R. S. Young, J. Bazile, L. Talmane, J. Luft, M. Liss, M. S. Taylor, L. D. Hurst and G. Kudla (2020). “Codon Usage and Splicing Jointly Influence mRNA Localization.” Cell Syst 10(4): 351-362 e358.
Movassat, M., E. Forouzmand, F. Reese and K. J. Hertel (2019). “Exon size and sequence conservation improves identification of splice-altering nucleotides.” RNA 25(12): 1793-1805.
Pettitt, S. J., Q. Liang, X. Y. Rairdan, J. L. Moran, H. M. Prosser, D. R. Beier, K. C. Lloyd, A. Bradley and W. C. Skarnes (2009). “Agouti C57BL/6N embryonic stem cells for mouse genetic resources.” Nat Methods 6(7): 493-495.
Piovesan, A., F. Antonaros, L. Vitale, P. Strippoli, M. C. Pelleri and M. Caracausi (2019). “Human protein-coding genes and gene feature statistics in 2019.” BMC Res Notes 12(1): 315.
Schmidt, A., K. Tief, A. Foletti, A. Hunziker, D. Penna, E. Hummler and F. Beermann (1998). “lacZ transgenic mice to monitor gene expression in embryo and adult.” Brain Res Brain Res Protoc 3(1): 54-60.
Shaul, O. (2017). “How introns enhance gene expression.” Int J Biochem Cell Biol 91(Pt B): 145-155.
Sibley, C. R., L. Blazquez and J. Ule (2016). “Lessons from non-canonical splicing.” Nat Rev Genet 17(7): 407-421.
Urlaub, G. and L. A. Chasin (1980). “Isolation of Chinese hamster cell mutants deficient in dihydrofolate reductase activity.” Proc Natl Acad Sci USA 77(7): 4216-4220.
Virts, E. L. and W. C. Raschke (2001). “The role of intron sequences in high level expression from CD45 cDNA constructs.” J Biol Chem 276(23): 19913-19920.

1. A method of modifying a complementary DNA (cDNA) sequence for expression in a eukaryotic cell comprising;
providing a nucleic acid molecule comprising a cDNA sequence,
wherein the cDNA sequence comprises two or more splicing consensus motifs that divide the cDNA sequence into exon regions of 50 to 1200 nucleotides,
inserting heterologous introns into the splicing consensus motifs of the cDNA sequence,
wherein each heterologous intron comprises a 3′ region having a GC content equal to or lower than the GC content of a 5′ region of the immediately downstream exon region,
thereby producing a nucleic acid molecule comprising a modified cDNA sequence for expression in a eukaryotic cell.

2. A method according to claim 1 wherein the modified cDNA sequence displays increased expression in a eukaryotic cell relative to the non-modified cDNA sequence.

3. A method according to claim 1 or claim 2 wherein the cDNA sequence is 1000 nucleotides or longer.

4. A method according to any one of the preceding claims wherein the cDNA sequence lacks introns.

5. A method according to any one of the preceding claims wherein the method comprises inserting 5 or more heterologous introns into the cDNA sequence.

6. A method according to any one of the preceding claims wherein the 3′ region of each heterologous intron has a GC content that is at least 8% lower than the 5′ region of the immediately downstream exon region

7. A method according to any one of the preceding claims wherein the 3′ region of each heterologous intron has a GC content that is 8% to 46% lower than the 5′ region of the immediately downstream exon region

8. A method according to any one of the preceding claims wherein the 3′ region of the heterologous intron comprises 30 nucleotides or more.

9. A method according to claim 8 wherein the 3′ region of the heterologous intron consists of 30 nucleotides.

10. A method according to any one of the preceding claims wherein 5′ region of the immediately downstream exon region comprises 30 nucleotides or more.

11. A method according to claim 10 wherein 5′ region of the immediately downstream exon region consists of 30 nucleotides.

12. A method according to any one of the preceding claims wherein the two or more splicing consensus motifs divide the cDNA sequence into exon regions of 100 to 150 nucleotides.

13. A method according to any one of the preceding claims wherein the splicing consensus motifs comprise the amino acid sequence (C/A/G)AGG(T/N)(T/N).

14. A method according to claim 13 wherein the splicing consensus motifs comprise the amino acid sequence CAGGTT.

15. A method according to any one of the preceding claims wherein the eukaryotic cell is a higher eukaryotic cell.

16. A method according to any one of the preceding claims wherein the eukaryotic cell is a mammalian cell.

17. A method according to any one of the preceding claims wherein the eukaryotic cell is a CHO cell or HEK cell.

18. A method according to any one of the preceding claims wherein further comprising incorporating the recombinant nucleic acid comprising the modified cDNA sequence into an expression vector.

19. A method according to claim 18 further comprising introducing the expression vector into a eukaryotic cell.

20. A method according to claim 19 further comprising causing or allowing expression from the modified cDNA sequence to produce a gene product.

21. A method according to claim 20 further comprising isolating or purifying the gene product.

22. A recombinant nucleic acid comprising a cDNA sequence for expression in a eukaryotic cell,
wherein the cDNA sequence comprises two or more heterologous introns and three or more exon regions of 50 to 1200 base pairs,
wherein each heterologous intron comprises a 3′ region having a GC content equal or lower than the GC content of a 5′ region of the immediately downstream exon region.

23. A recombinant nucleic acid according to claim 22 produced by a method of any one of claims 1 to 17.

24. An expression vector comprising a recombinant nucleic acid according to claim 22 or 23.

25. A eukaryotic cell comprising a recombinant nucleic acid according to claim 22 or 23 or an expression vector according to claim 24.
